(19) United States
(12) Patent Application Publication
(10) Pub. No.: US 2024/0342126 A1
(43) Pub. Date: Oct. 17, 2024

(54) HALLUCINOGEN-FATTY ACID COMBINATION

(71) Applicant: Mindset Pharma Inc., Toronto (CA)

(72) Inventors: Abdelmalik Slassi, Mississauga (CA); Joseph Araujo, Grimsby (CA); Guy Andrew Higgins, Elora (CA); Joseph Gabriele, Stoney Creek (CA)

(73) Assignee: Mindset Pharma Inc., Toronto, ON (CA)

(21) Appl. No.: 18/563,898

(22) PCT Filed: May 26, 2022
(86) PCT No.: PCT/CA2022/050858

§ 371 Date: Nov. 23, 2023

Related U.S. Application Data
(60) Provisional application No. 63/202,081, filed on May 26, 2021.

(51) Int. Cl. A61K 31/201; A61K 9/00; A61K 31/4045; A61K 31/675; A61K 47/10; A61K 47/26

(52) U.S. Cl. CPC A61K 31/201; A61K 9/0043; A61K 31/4045; A61K 31/675; A61K 47/10; A61K 47/26

(57) ABSTRACT

The present application relates to combination compositions comprising one or more hallucinogens, or a pharmaceutically acceptable salt, prodrug and/or solvate thereof, and one or more fatty acids, or a pharmaceutically acceptable salt, prodrug and/or solvate thereof. The present application also relates to intranasal pharmaceutical compositions comprising one or more hallucinogens, or a pharmaceutically acceptable salt, prodrug and/or solvate thereof, and one or more fatty acids, or a pharmaceutically acceptable salt, prodrug and/or solvate thereof. For example, the one or more hallucinogens is 5-methoxy-N,N-dimethyltryptamine or a pharmaceutically acceptable salt thereof and the one or more fatty acids is linoleic acid.

### RELATED APPLICATIONS

The present application claims the benefit of priority of co-pending U.S. provisional patent application No. 63/202,081 filed on May 26, 2021, the contents of which are incorporated herein by reference in their entirety.

### FIELD

The present application relates to combination treatments comprising hallucinogens. For example, the present application relates to compositions and kits comprising one or more hallucinogens and one or more fatty acids. The present application further relates to the use of these compositions for treatment of, for example, mental illnesses and neurological diseases, in the fields of psychiatry, neurobiology and pharmacotherapy. The present application also relates to intranasal pharmaceutical compositions comprising one or more hallucinogens and one or more fatty acids. For example, the one or more hallucinogens is 5-methoxy-N,N-dimethyltryptamine or a pharmaceutically acceptable salt, prodrug and/or solvate thereof and the one or more fatty acids is linoleic acid.

### BACKGROUND

Mental health disorders, or mental illness, refer to a wide range of disorders that include, but are not limited to, depressive disorders, anxiety and panic disorders, schizophrenia, eating disorders, substance misuse disorders, post-traumatic stress disorder, attention deficit/hyperactivity disorder, addiction disorders, cognitive-aggressive disorders and obsessive-compulsive disorder. The severity of symptoms varies such that some individuals experience debilitating disease that precludes normal social function, while others suffer with intermittent repeated episodes across their lifespan. Although the presentation and diagnostic criteria among mental illness conditions are distinct in part, there are common endophenotypes of note across the diseases, and often comorbidities exist. Specifically, there exist phenotypic endophenotypes associated with alterations in mood, cognition and behavior. Interestingly, many of these endophenotypes extend to neurological conditions as well. For example, attentional deficits are reported in patients with attention deficit disorder, attention deficit hyperactivity disorder, cognitive-aggressive disorders, eating disorders, substance use disorders, schizophrenia, depression, obsessive compulsive disorders, addiction disorders, traumatic brain injury, Fragile X, Alzheimer's disease, mini-strokes, Parkinson's disease and frontotemporal dementia.

Many mental health disorders, as well as neurological disorders, are impacted by alterations, dysfunction, degeneration, and/or damage to the brain's serotonergic system and other neurotransmitter systems (dopamine, glutamate, etc), which may explain, in part, common endophenotypes and comorbidities among neuropsychiatric and neurological diseases. Many therapeutic agents that modulate serotonergic function are commercially available, including serotonin reuptake inhibitors, selective serotonin reuptake inhibitors, antidepressants, monoamine oxidase inhibitors, and, while primarily developed for depressive disorders, many of these therapeutics are used across multiple medical indications including, but not limited to, depression in Alzheimer's disease and other neurodegenerative disease, chronic pain, existential pain, bipolar disorder, obsessive compulsive disorder, anxiety disorders and smoking cessation. However, in many cases, the marketed drugs show limited benefit compared to placebo, can take six weeks to work and for some patients, and are associated with several side effects including trouble sleeping, drowsiness, fatigue, weakness, changes in blood pressure, memory problems, digestive problems, weight gain and sexual problems.

Hallucinogens are a psychoactive agents that often or ordinarily cause hallucinations, perceptual anomalies, and other substantial subjective changes in thought, cognition, emotion and/or consciousness. Hallucinogens are often also referred to as psychedelics, although some consider psychedelics as a class of hallucinogen along with dissociatives, stimulants, enactogens, empathogens, deliriants and other such psychoactive compounds. As used herein, all of these compounds are referred to interchangeably as hallucinogens or psychedelics.

The field of hallucinogen/psychedelic neuroscience has witnessed a recent renaissance following decades of restricted research due to their legal status. Psychedelics are one of the oldest classes of psychopharmacological agents known to humans and cannot be fully understood without reference to various fields of research, including anthropology, ethnopharmacology, psychiatry, psychology, sociology, and others. Psychedelics (serotonergic hallucinogens and classes) are powerful psychoactive substances that alter perception and mood and affect numerous cognitive processes. They are generally considered physiologically safe and do not lead to dependence or addiction. Their origin predates written history, and they were employed by early cultures in many sociocultural and ritual contexts. After the virtually contemporaneous discovery of (5R,8R)-(+)-lysergic acid-N,N-diethylamide (LSD, 4, Scheme 1) and the identification of serotonin in the brain, early research focused intensively on the possibility that LSD and other psychedelics had a serotonergic basis for their action. Today there is a consensus that psychedelics are agonists or partial agonists at brain serotonin 5-hydroxytryptamine 2A (5-HT2A) receptors, with particular importance on those expressed on apical dendrites of neocortical pyramidal cells in layer V, but also may bind with lower affinity to other receptors such as other serotonergic receptors, the sigma-1 receptor and trace amino acid associated receptors. Several useful rodent models have been developed over the years to help unravel the neurochemical correlates of serotonin 5-HT2A receptor activation in the brain, and a variety of imaging techniques have been employed to identify key brain areas that are directly affected by psychedelics.

Psychedelics have both rapid onset and persisting effects long after their acute effects, which includes changes in mood, cognition, brain morphology and brain function. Long lasting effects may result from their unique receptor affinities, which affect neurotransmission via neuromodulatory systems that serve to modulate brain activity, i.e., neuroplasticity, and promote cell survival, are neuroprotective, and modulate brain neuroimmune systems as well as similar systems in the periphery. The mechanisms which lead to these long-term neuromodulatory changes are linked to epigenetic modifications, gene expression changes and modulation of pre—and post-synaptic receptor densities, to name a few. These, previously under-researched, psychedelic drugs may potentially provide the next generation of neurotherapeutics, where treatment resistant psychiatric and neurological diseases, e.g., depression, post-traumatic stress disorder, dementia and addiction, may become treatable with attenuated pharmacological risk profiles.

Although there is a general perception that psychedelic drugs are dangerous, from a physiologic safety standpoint, they are some of the safest known classes of central nervous system (CNS) drugs. They do not cause addiction, and no overdose deaths have occurred after ingestion of typical doses of classical psychedelic agents, such as LSD (4), psilocybin (5), or mescaline (1)(Scheme 1). Preliminary data show that psychedelic administration in humans results in a unique profile of effects and potential adverse reactions that need to be appropriately addressed to maximize safety. The primary safety concerns are largely psychologic, rather than physiologic, in nature. Somatic effects vary but are relatively insignificant, even at doses that elicit powerful psychologic effects. Psilocybin (5), when administered in a controlled setting, has frequently been reported to cause transient, delayed headache, with incidence, duration, and severity increased in a dose-related manner [Johnson et al., Drug Alcohol Depend (2012) 123 (1-3): 132-140]. It has been found that repeated administration of psychedelics leads to a very rapid development of tolerance known as tachyphylaxis, a phenomenon believed to be mediated, in part, by 5-HT2A receptors. In fact, several studies have shown that rapid tolerance to psychedelics correlates with downregulation of 5-HT2A receptors. For example, daily LSD administration selectively decreased 5-HT2 receptor density in the rat brain [Buckholtz et al., Eur. J. Pharmacol. 1990, 109:421-425. 1985; Buckholtz et al., Life Sci. 1985, 42:2439-2445].

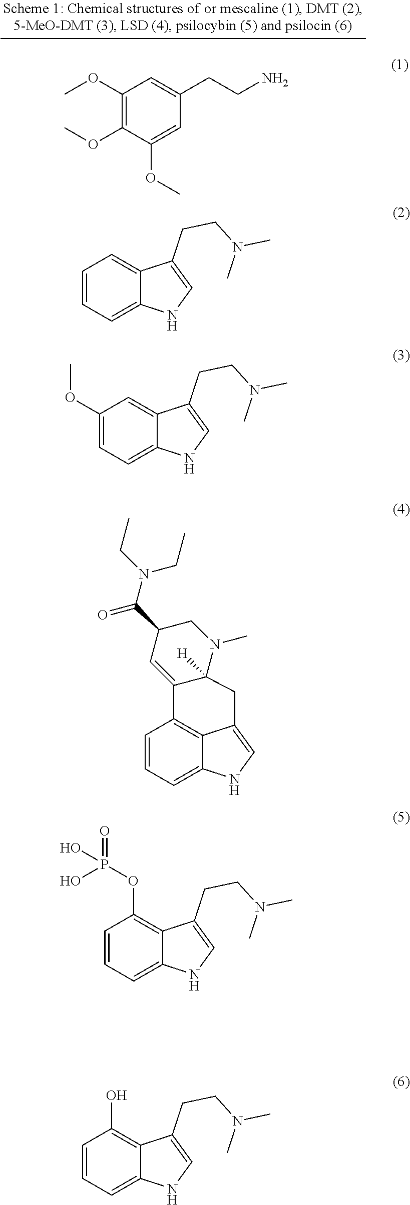

Psilocybin (4-phosphoryloxy-N,N-dimethyltrypatmine (5, Scheme 1) is a tryptamine and is one of the major psychoactive constituents in mushrooms of the psilocybe species. It was first isolated from psilocybe mushrooms by Hofmann in 1957, and later synthesized by him in 1958 [Passie et al. Addict Biol., 2002, 7 (4): 357-364], and was used in psychiatric and psychological research and in psychotherapy during the early to mid-1960s up until its controlled drug scheduling in 1970 in the US, and up until the 1980s in Germany [Passie 2005; Passie et al. Addict Biol., 2002, 7 (4): 357-364]. Research into the effects of psilocybin resumed in the mid-1990s, and it is currently the preferred compound for use in studies of the effects of serotonergic hallucinogens [Carter et al. J. Cogn. Neurosci., 2005 17 (10): 1497-1508; Gouzoulis-Mayfrank et al. Neuropsychopharmacology 1999, 20 (6): 565-581; Hasler et al, Psychopharmacology (Berl) 2004, 172 (2): 145-156], likely because it has a shorter duration of action and suffers from less notoriety than LSD. Like other members of this class, psilocybin induces sometimes profound changes in perception, cognition, satiety and emotion, including emotional lability.

In humans as well as other mammals, psilocybin is transformed into the active metabolite psilocin, or 4-hydroxy-N,N-dimethyltryptamine (6, Scheme 1). It is likely that psilocin partially or wholly produces most of the subjective and physiological effects of psilocybin in humans and non-human animals. Recently, human psilocybin research confirms the 5-HT2A activity of psilocybin and psilocin, and provides some support for indirect effects on dopamine through 5-HT2A activity and possible activity at other serotonin receptors. In fact, the most consistent finding for involvement of other receptors in the actions of psychedelics is the 5-HT1A receptor. That is particularly true for tryptamines and LSD, which generally have significant affinity and functional potency at this receptor. It is known that 5-HT1A receptors are colocalized with 5-HT2A receptors on cortical pyramidal cells [Martin-Ruiz et al. J Neurosci. 2001, 21 (24): 9856-986], where the two receptor types have opposing functional effects [Araneda et al. Neuroscience 1991, 40 (2): 399-412].

Although the exact role of the 5-HT2A receptor, and other 5-HT2 receptor family members, is not well understood with respect to the amygdala, it is evident that the 5-HT2A receptor plays an important role in emotional responses and is an important target to be considered in the actions of 5-HT2A agonist psychedelics. In fact, a majority of known 5-HT2A agonists produce hallucinogenic effects in humans, and rodents generalize from one 5-HT2A agonist to others, as between psilocybin and LSD [Aghajanian et al., Eur J Pharmacol., 1999, 367 (2-3): 197-206; Nichols at al., J Neurochem., 2004, 90 (3): 576-584]. Psilocybin has a stronger affinity for the human 5-HT2A receptor than for the rat receptor and it has a lower Ki for both 5-HT2A and 5-HT2C receptors than LSD. Moreover, results from a series of drug-discrimination studies in rats found that 5-HT2A antagonists, and not 5-HT1A antagonists, prevented rats from recognizing psilocybin [Winter et al., Pharmacol Biochem Behav., 2007, 87 (4): 472-480].

There remains strong research and therapeutic potential for psilocybin as recent studies have shown varying degrees of success in neurotic disorders, alcoholism, depression in terminally ill cancer patients, obsessive compulsive disorder, addiction, anxiety, post-traumatic stress disorder and even cluster headaches. It could also be useful as a psychosis model for the development of new treatments for psychotic disorders. [Dubovyk and Monahan-Vaughn, ACS Chem. Neurosci. (2018), 9 (9): 2241-2251].

Recent and exciting developments in the field have occurred in clinical research, where several double-blind placebo-controlled phase 2 studies of psilocybin-assisted psychotherapy in patients with treatment resistant, major depressive disorder and cancer-related psychosocial distress have demonstrated unprecedented positive relief of anxiety and depression. Two recent small pilot studies of psilocybin assisted psychotherapy also have shown positive benefit in treating both alcohol and nicotine addiction. Recently, blood oxygen level-dependent functional magnetic resonance imaging and magnetoencephalography have been employed for in vivo brain imaging in humans after administration of a psychedelic, and results indicate that intravenously administered psilocybin and LSD produce decreases in oscillatory power in areas of the brain's default mode network [Nichols D E. Pharmacol Rev. (2016) 68 (2): 264-355].

Preliminary studies using positron emission tomography (PET) showed that psilocybin ingestion (15 or 20 mg orally) increased absolute metabolic rate of glucose in frontal, and to a lesser extent, in other cortical regions as well as in striatal and limbic subcortical structures in healthy participants, suggesting that some of the key behavioral effects of psilocybin involve the frontal cortex [Gouzoulis-Mayfrank et al., Neuropsychopharmacology, 1999, 20 (6): 565-581; Vollenweider et al., Brain Res. Bull. 2001, 56 (5): 495-507]. Although 5-HT2A agonism is widely recognized as the primary action of classic psychedelic agents, psilocybin has lesser affinity for a wide range of other pre—and post-synaptic serotonin and dopamine receptors, as well as the serotonin reuptake transporter [Tyls et al., Eur. Neuropsychopharmacol. 2014, 24 (3): 342-356]. Psilocybin activates 5-HT1A receptors, which may contribute to antidepressant/anti-anxiety effects.

Depression and anxiety are two of the most common psychiatric disorders worldwide. Depression is a multifaceted condition characterized by episodes of mood disturbances alongside other symptoms such as anhedonia, psychomotor complaints, feelings of guilt, attentional deficits and suicidal tendencies, all of which can range in severity. According to the World Health Organization, the discovery of mainstream antidepressants has largely revolutionized the management of depression, yet up to 60% of patients remain inadequately treated. This is often due to the drugs' delayed therapeutic effect (generally 6 weeks from treatment onset), side effects leading to non-compliance, or inherent non-responsiveness to them. Similarly, anxiety disorders are a collective of etiologically complex disorders characterized by intense psychosocial distress and other symptoms depending on the subtype. Anxiety associated with life-threatening disease is the only anxiety subtype that has been studied in terms of psychedelic-assisted therapy. This form of anxiety affects up to 40% of individuals diagnosed with life-threatening diseases like cancer. It manifests as apprehension regarding future danger or misfortune accompanied by feelings of dysphoria or somatic symptoms of tension, and often coexists with depression. It is associated with decreased quality of life, reduced treatment adherence, prolonged hospitalization, increased disability, and hopelessness, which overall contribute to decreased survival rates. Pharmacological and psychosocial interventions are commonly used to manage this type of anxiety, but their efficacy is mixed and limited such that they often fail to provide satisfactory emotional relief. Recent interest into the use of psychedelic-assisted therapy may represent a promising alternative for patients with depression and anxiety that are ineffectively managed by conventional methods.

Generally, the psychedelic treatment model consists of administering the orally-active drug to induce a mystical experience lasting 4-9 h depending on the psychedelic [Halberstadt, Behav Brain Res., 2015, 277:99-120; Nichols, Pharmacol Rev., 2016, 68 (2): 264-355]. This enables participants to work through and integrate difficult feelings and situations, leading to enduring anti-depressant and anxiolytic effects. Classical psychedelics like psilocybin and LSD are being studied as potential candidates. In one study with classical psychedelics for the treatment of depression and anxiety associated with life-threatening disease, it was found that, in a supportive setting, psilocybin, and LSD consistently produced significant and sustained anti-depressant and anxiolytic effects.

Further emerging clinical research and evidence suggest psychedelic-assisted therapy also shows potential as an alternative treatment for refractory substance use disorders and mental health conditions, and thus may be an important tool in a crisis where existing approaches have yielded limited success. A recent systematic review of clinical trials published over the last 25 years summarizes some of the anti-depressive, anxiolytic, and anti-addictive effects of classic psychedelics. Among these, are encouraging findings from a meta-analysis of randomized controlled trials of LSD therapy and a recent pilot study of psilocybin-assisted therapy for treating alcohol use disorder [dos Santos et al., Ther Adv Psychopharmacol., 2016, 6 (3): 193-213]. Similarly encouraging, are findings from a recent pilot study of psilocybin-assisted therapy for tobacco use disorder, demonstrating abstinence rates of 80% at six months follow-up and 67% at 12 months follow-up [Johnson et al., J Drug Alcohol Abuse, 2017 43 (1): 55-60; Johnson et al., 2014, Psychopharmacol. 2014, 28 (11): 983-992], such rates are considerably higher than any documented in the tobacco cessation literature. Notably, mystical-type experiences generated from the psilocybin sessions were significantly correlated with positive treatment outcomes. These results coincide with bourgeoning evidence from recent clinical trials lending support to the effectiveness of psilocybin-assisted therapy for treatment-resistant depression and end-of-life anxiety [Carhart-Harris et al. Neuropsychopharmacology, 2017 42 (11): 2105-2113]. Research on the potential benefits of psychedelic-assisted therapy for opioid use disorder (OUD) is beginning to emerge, and accumulating evidence supports a need to advance this line of investigation. Available evidence from earlier randomized clinical trials suggests a promising role for treating OUD: higher rates of abstinence were observed among participants receiving high dose LSD and ketamine-assisted therapies for heroin addiction compared to controls at long-term follow-ups. Recently, a large United States population study among 44,000 individuals found that psychedelic use was associated with 40% reduced risk of opioid abuse and 27% reduced risk of opioid dependence in the following year, as defined by DSM-IV criteria [Pisano et al., J Psychopharmacol., 2017, 31 (5): 606-613]. Similarly, a protective moderating effect of psychedelic use was found on the relationship between prescription opioid use and suicide risk among marginalized women [Argento et al., J. Psychopharmacol., 2018, 32 (12): 1385-1391].

Regular doses of psychedelics also ameliorate sleep disturbances, which are highly prevalent in depressive patients with more than 80% of them having complaints of poor sleep quality. The sleep symptoms are often unresolved by first-line treatment and are associated with a greater risk of relapse and recurrence. Interestingly, sleep problems often appear before other depression symptoms, and subjective sleep quality worsens before the onset of an episode in recurrent depression. Brain areas showing increased functional connectivity with poor sleep scores and higher depressive symptomatology scores included prefrontal and limbic areas, areas involved in the processing of emotions. Sleep disruption in healthy participants has demonstrated that sleep is indeed involved in mood, emotion evaluation processes and brain reactivity to emotional stimuli. An increase in negative mood and a mood-independent mislabeling of neutral stimuli as negative was for example shown by one study while another demonstrated an amplified reactivity in limbic brain regions in response to both negative and positive stimuli. Two other studies assessing electroencephalographic (EEG) brain activity during sleep showed that psychedelics, such as LSD, positively affect sleep patterns. Moreover, it has been shown that partial or a full night of sleep deprivation can alleviate symptoms of depression suggested by resetting circadian rhythms via modification of clock gene expression. It further was suggested that a single dose of a psychedelic causes a reset of the biological clock underlying sleep/wake cycles and thereby enhances cognitive-emotional processes in depressed people but also improving feelings of well-being and enhances mood in healthy individuals [Kuypers, Medical Hypotheses, 2019, 125:21-24].

In a systematic meta-analysis of clinical trials from 1960-2018 researching the therapeutic use of psychedelic treatment in patients with serious or terminal illnesses and related psychiatric illness, it was found that psychedelic therapy (mostly with LSD) may improve cancer-related depression, anxiety, and fear of death. Four randomized controlled clinical trials were published between 2011 and 2016, mostly with psilocybin treatment, that demonstrated psychedelic-assisted treatment can produce rapid, robust, and sustained improvements in cancer-related psychological and existential distress. [Ross, Int. Rev. Psychiatry, 2018, 30 (4): 317-330]. Thus, the use of psychedelics in the fields of oncology and palliative care is intriguing for several reasons. First, many patients facing cancer or other life-threatening illnesses experience significant existential distress related to loss of meaning or purpose in life, which can be associated with hopelessness, demoralization, powerlessness, perceived burdensomeness, and a desire for hastened death. Those features are also often at the core of clinically significant anxiety and depression, and they can substantially diminish quality of life in this patient population. The alleviation of those forms of suffering should be among the central aims of palliative care. Accordingly, several manualized psychotherapies for cancer-related existential distress have been developed in recent years, with an emphasis on dignity and meaning-making. However, there are currently no pharmacologic interventions for existential distress per se, and available pharmacologic treatments for depressive symptoms in patients with cancer have not demonstrated superiority over placebo. There remains a need for additional effective treatments for those conditions [Rosenbaum et al., Curr. Oncol., 2019, 26 (4): 225-226].

Recently, there has been growing interest in a new dosing paradigm for psychedelics such as psilocybin and LSD referred to colloquially as microdosing. Under this paradigm, sub-perceptive doses of the serotonergic hallucinogens, approximately 10% or less of the full dose, are taken on a more consistent basis of once each day, every other day, or every three days, and so on. Not only is this dosing paradigm more consistent with current standards in pharmacological care, but may be particularly beneficial for certain conditions, such as Alzheimer's disease and other neurodegenerative diseases, attention deficit disorder, attention deficit hyperactivity disorder, and for certain patient populations such as elderly, juvenile and patients that are fearful of or opposed to psychedelic assisted therapy. Moreover, this approach may be particularly well suited for managing cognitive deficits and preventing neurodegeneration. For example, subpopulations of low attentive and low motivated rats demonstrate improved performance on a 5-choice serial reaction time and progressive ratio tasks, respectively, following doses of psilocybin below the threshold for eliciting the classical wet dog shake behavioral response associated with hallucinogenic doses (Blumstock et al., WO 2020/157569 A1; Higgins et al., 2021). Similarly, treatment of patients with hallucinogenic doses of 5-HT2A agonists is associated with increased BDNF and activation of the mTOR pathway, which are thought to promote neuroplasticity and are hypothesized to serve as molecular targets for the treatment of dementias and other neurodegenerative disorders (Ly et al. Cell Rep., 2018; 23 (11): 3170-3182). Additionally, several groups have demonstrated that low, non-hallucinogenic and non-psychomimetic, doses of 5-HT2A agonists also show similar neuroprotective and increased neuroplasticity effects (neuroplastogens) and reduced neuroinflammation, which could be beneficial in both neurodegenerative and neurodevelopmental diseases and chronic disorders and may be mediated by other receptors including trace amino acid associated receptors (Manfredi et al., WO 2020/181194, Flanagan et al., Int. Rev. Psychiatry, 2018, 13:1-13; Nichols et al., 2016, Psychedelics as medicines; an emerging new paradigm). This repeated, lower, dose paradigm may extend the utility of these compounds to additional indications and may prove useful for wellness applications.

5-methoxy-N,N-dimethyltryptamine (5-MeO-DMT; 3, Scheme 1) is a tryptamine natural product most commonly identified as the primary psychoactive component of the parotid gland secretions of Incilius alvarius, the Sonoran Desert toad and is present in low concentrations in a variety of plants, shrubs, and seeds [Uthaug, M. V. et al., Psychopharmacology 2019, 236:2653-2666; Weil et al., J. Ethnopharmacol. 1994, 41 (1-2): 1-8]. N,N-dimethyltryptamine (DMT; 2, Scheme 1) is a tryptamine natural product most commonly identified as the primary psychoactive component of various natural plants and vines including Acacia, Desmodium, Mimosa, Virola, Delosperma and Phalaris. Human consumption of these materials for their psychoactive properties has been reported for several 100s of years [Agurell et al., Acta Chem. Scand. 1969, 23 (3): 903-916; Torres et al., Haworth Herbal Press: New York, 2014].

5-MeO-DMT has demonstrated sub-micromolar binding affinity across most serotonin receptor subtypes expressed in the CNS, with about 300-fold selectivity for the human 5-HT1A (3±0.2 nM) versus 5-HT2A (907±170 nM) receptor subtypes [Halberstadt et al., Psychopharmacology, 2012, 221 (4): 709-718]. DMT has greater than 3-fold binding affinity for 5-HT1A (0.075 μM) over 5-HT2A (0.237 μM). Data has suggested that activation of the 5-HT1A receptor may also play a significant role in contributing to the subjective and behavioral effects elicited by psychedelics in a synergistic way with 5-HT2A activation. By contrast to 5-MeO-DMT and DMT, psilocin (the active metabolite of psilocybin) is about 5-fold more selective for human 5-HT2A receptors (107 nM) versus 5-HT1A (567 nM) [Sherwood et al., ACS Omega, 2020, 5 (49): 32067-32075].

It is reported that 5-MeO-DMT consumption leads to a general lack of colorful geometric visual hallucinations typically associated with other psychedelics including DMT. It is also suggested that both 5-MeO-DMT and DMT may be helpful in treating clinical mental health conditions [Barsuglia et al. Front. Psychol. 2018, 9:2459; Davis et al., Am. J. Drug Alcohol Abuse, 2019, 45 (2): 161-169; Malcolm et al., Mental Health Clinician, 2017, 7 (1): 39-45; Uthaug, M. V. et al., Psychopharmacology 2019, 236:2653-2666]. These data suggest that 5-MeO-DMT and DMT produce mystical experiences with intensity comparable or greater than those produced with psilocybin, but with a shorter duration of effect lasting between 10 and 60 min depending on the route of administration.

It has been reported that fatty acids may enhance the activity of certain antidepressant drugs at low non-antidepressant doses [Laino, C. H. et al. European Journal of Pharmacology (2010), 648:117-126; Carlezon, W. A. Jr. et al. Biol. Psychiatry (2005), 57:343-350]. Some reports show that omega-3 fatty acids have antidepressant activity when administered on a chronic basis [Lakhwani, L. et al. Acta Poloniae Pharmaceutica-Drug Research (2007), 64:271-276]. Docosahexanaenoic acid (DHA) has been reported to provide a synergistic effect in enhancing the absorption of carotenoids, such as lutein (US patent application no. US2006/0270739). Moreover, DHA has been implicated in augmented brain and cognitive development and maintenance in aging and neuropsychiatric disorders in both humans and animals [Ciappolino, V. et al., Nutrients (2019), 11:769 doi 10.3990/nu11040769; Lauritzen, L. et al. Nutrients (2016), 8:6 doi 10.3390/nu8010006; Weiser, M. J. et al., Nutrients (2016), 8:99 doi 10.3990/nu8020099].

### SUMMARY

In the present application, the combination of fatty acids and hallucinogens is shown to provide a synergistic effect in animal models for hallucinogenic activity.

According, in some embodiments, the present application includes a pharmaceutical composition comprising one or more hallucinogens, or a pharmaceutically acceptable salt, prodrug and/or solvate thereof, and one or more fatty acids, or a pharmaceutically acceptable salt, prodrug and/or solvate thereof.

The present application also includes a kit comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more hallucinogens, or a pharmaceutically acceptable salt, prodrug and/or solvate thereof, and the one or more fatty acids, or a pharmaceutically acceptable salt, prodrug and/or solvate thereof, are in a single pharmaceutical composition or are in separate pharmaceutical compositions.

In some embodiments, the present application also includes a method of treating or preventing a disease, disorder or condition that is treated by activation of a serotonin receptor comprising administering an effective amount of one or more hallucinogens, or a pharmaceutically acceptable salt, prodrug and/or solvate thereof, and one or more fatty acids, or a pharmaceutically acceptable salt, prodrug and/or solvate thereof, to a subject in need thereof.

In some embodiments, the present application also includes a method of improving the efficacy of one or more hallucinogens, or a pharmaceutically acceptable salt, prodrug and/or solvate thereof, comprising administering an effective amount of the one or hallucinogens, or a pharmaceutically acceptable salt, prodrug and/or solvate thereof, in combination with an effective amount of one or more fatty acids, or a pharmaceutically acceptable salt, prodrug and/or solvate thereof, to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows that linoleic acid (LA) alone produces no significant effect on head twitches up to 10 mg/kg SC.

FIG. 2 shows that the combination of linoleic acid (LA) & compound I-4 or psilocybin produces more head twitches vs. I-4 or psilocybin alone.

FIG. 3 shows increased number of head twitches produced by linoleic acid (LA) in combination with compound 1-4 or psilocybin may be due to extended duration of action.

FIG. 4 shows pretreatment with selective 5-HT2A receptor antagonist M100907 (0.5 mg/kg IP) completely blocked the incidence of head twitches induced by either I-4 (6.2 mg/kg SC) or psilocybin (3 mg/kg SC).

FIG. 5 shows comparison of mean (+SD, n=3) plasma concentration versus time profiles of exemplary 5-MeO-DMT and its metabolite, bufotenine, between Standard and Sponsor formulations following A) intranasal (IN) and B) subcutaneous (SC)) administrations of 5-MeO-DMT to groups of 3 male Sprague-Dawley rats. The Standard formulation included 5-MeO-DMT prepared at the appropriate concentration in 10% dimethyl sulfoxide (DMSO) and 90% (v/v) saline and the sponsor formulation further included 3% linoleic acid.

FIG. 6 show comparison of mean (+SD, n=3) CSF concentration versus time profiles of exemplary 5-MeO-DMT and its metabolite, bufotenine, between in Standard and Sponsor formulations, following A) i.n. and B) s.c. administration of 5-MeO-DMT to groups of 3 male Sprague-Dawley rats.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

The term “composition(s) of the application” or “composition(s) of the present application” and the like as used herein refers to a composition, such a pharmaceutical composition, comprising one or more hallucinogens, or a pharmaceutically acceptable salt, prodrug and/or solvate thereof, and one or more fatty acids, or a pharmaceutically acceptable salt, prodrug and/or solvate thereof.

The term “intranasal composition(s) of the application” or “intranasal composition(s) of the present application” and the like as used herein refers to an intranasal composition, such an intranasal pharmaceutical composition, comprising one or more hallucinogens, or a pharmaceutically acceptable salt, prodrug and/or solvate thereof, and one or more fatty acids, or a pharmaceutically acceptable salt, prodrug and/or solvate thereof.

The term “kit(s) of the application” or “kit(s) of the present application” and the like as used herein refers to a kit, such a pharmaceutical kits, comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more hallucinogens, or a pharmaceutically acceptable salt, prodrug and/or solvate thereof, and the one or more fatty acids, or a pharmaceutically acceptable salt, prodrug and/or solvate thereof, are in a single pharmaceutical composition or are in separate pharmaceutical compositions.

The term “and/or” as used herein means that the listed items are present, or used, individually or in combination. In effect, this term means that “at least one of or “one or more” of the listed items is used or present. The term “and/or” with respect to pharmaceutically acceptable salts and/or solvates thereof means that the compounds of the application exist as individual salts and solvates, as well as a combination of, for example, a salt of a solvate of a compound of the application.

As used herein, the term “one or more” item includes a single item selected from the list as well as mixtures of two or more items selected from the list.

As used in the present application, the singular forms “a”, “an” and “the” include plural references unless the content clearly dictates otherwise. For example, an embodiment including “a fatty acid” should be understood to present certain aspects with one fatty acid, or two or more additional fatty acids.

In embodiments comprising an “additional” or “second” component, such as an additional or second fatty acid, the second component as used herein is chemically different from the other components or first component. A “third” component is different from the other, first and second components and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers and/or steps and also exclude the presence of other unstated features, elements, components, groups, integers and/or steps.

The term “consisting essentially of”, as used herein, is intended to specify the presence of the stated features, elements, components, groups, integers and/or steps as well as those that do not materially affect the basic and novel characteristic(s) of these features, elements, components, groups, integers and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “solvate” as used herein means a compound, or a salt or prodrug of a compound, wherein molecules of a suitable solvent are incorporated in the crystal lattice. A suitable solvent is physiologically tolerable at the dosage administered.

The term a “fatty acid” as used herein refers to a carboxylic acid with a long aliphatic chain, which is either saturated or unsaturated or straight or branched.

The term “hallucinogen” as used herein refers to a compound that is psychoactive and/or that often or ordinarily causes hallucinations, perceptual anomalies, and/or other substantial subjective changes in thought, emotion and/or consciousness. Hallucinogens, as used herein, include compounds that are classified as psychedelics, dissociatives, enactogens, stimulants, empathetics, psychotomimetics and/or deliriants.

The term “psychedelic” as used herein refers to a class of hallucinogenic compounds that exert their primary effects via a serotonin receptor, generally regarded to be the 5-HT2A receptor, but may also exert effects via other serotonin receptors such as 5-HT1A, dopaminergic receptors, N-methyl-D-aspartate receptors, kappa opioid receptors, sigma 1 receptor, or trace amino acid associated receptors, or any combination thereof.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or 6 carbon atoms and includes, for example, any of the hexyl, alkyl, and pentyl alkyl isomers as well as n-, iso-, sec—and tert-butyl, n—and iso-propyl, ethyl and methyl. As another example, “C₄alkyl” refers to n-, iso-, sec—and tert-butyl, n—and isopropyl, ethyl and methyl.

The term “alkenyl” whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains either 6 to 20 carbon atoms.

The term “available”, as in “available hydrogen atoms” or “available atoms” refers to atoms that would be known to a person skilled in the art to be capable of replacement by a substituent.

The term “heterocycloalkyl” as used herein, whether it is used alone or as part of another group, refers to cyclic groups containing at least one non-aromatic ring containing from 3 to 20 atoms in which one or more of the atoms are a heteromoiety selected from O, S, S(O), SO₂ and N and the remaining atoms are C. Heterocycloalkyl groups are either saturated or unsaturated (i.e. contain one or more double bonds). When a heterocycloalkyl group contains the prefix Cₙ₁₋ₙ₂ or “n1 to n2” this prefix indicates the number of carbon atoms in the corresponding carbocyclic group, in which one or more, suitably 1 to 5, of the ring atoms is replaced with a heteromoeity as selected from O, S, S(O), SO₂ and N and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halogen” (or “halo”) whether it is used alone or as part of another group, refers to a halogen atom and includes fluoro, chloro, bromo and iodo.

The term “haloalkyl” as used herein refers to an alkyl group as defined above in which one or more of the available hydrogen atoms have been replaced with a halogen. Thus, for example, “C₁₋₆haloalkyl” (or “C₁-C₆haloalkyl”) refers to a C₁ to C₆ linear or branched alkyl group as defined above with one or more halogen substituents.

As used herein, the term “haloalkenyl” refers to an alkenyl group as defined above in which one or more of the available hydrogen atoms have been replaced with a halogen. Thus, for example, “C₁₋₆haloalkenyl” (or “C₁-C₆haloalkenyl”) refers to a C₁ to C₆ linear or branched alkenyl group as defined above with one or more halogen substituents.

As used herein, the term “haloalkynyl” refers to an alkynyl group as defined above in which one or more of the available hydrogen atoms have been replaced with a halogen. Thus, for example, “C₁₋₆haloalkynyl” (or “C₁-C₆haloalkynyl”) refers to a C₁ to C₆ linear or branched alkynyl group as defined above with one or more halogen substituents.

As used herein, the term “alkoxy” as used herein, alone or in combination, includes an alkyl group connected to an oxygen connecting atom.

The term “substituted” as used herein means, unless otherwise indicated, that the referenced group is substituted with one or more substituents independently selected from halogen, CO₂H, CO₂CH₃, C(O)NH₂, C(O)N(CH₃)₂, C(O)NHCH₃, SO₂CH₃. SOCH₃, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring members selected from O, S, S(O), SO₂, N, NH and NCH₃.

The term “alternate isotope thereof” as used herein refers to an isotope of an element that is other than the isotope that is most abundant in nature.

The term “all available atoms are optionally substituted with alternate isotope” as used herein means that available atoms are optionally substituted with an isotope of that atom of having the same atomic number, but an atomic mass or mass number different from the atomic mass or mass number predominantly found in nature.

The term “compound” refers to the compound and, in certain embodiments, to the extent they are stable, any hydrate and/or solvate thereof. A hydrate is the compound complexed with water and a solvate is the compound complexed with a solvent, which may be an organic solvent or an inorganic solvent. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

The term “pharmaceutically acceptable” means compatible with the treatment of subjects.

The term “pharmaceutically acceptable carrier” means a non-toxic solvent, dispersant, excipient, adjuvant or other material which is mixed with one or more active ingredients in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

The term “subject” as used herein includes all members of the animal kingdom including mammals, and suitably refers to humans. Thus the methods of the present application are applicable to both human therapy and veterinary applications.

The term “treated”, “treating” or “treatment” as used herein and as is well understood in the art, means an approach for obtaining beneficial or desired results, including clinical results.

As used herein, the term “effective amount” or “therapeutically effective amount” means an amount that is effective, at dosages and for periods of time necessary to achieve a desired result. For example, in the context of treating a disease, disorder or condition that is treated by activation of a serotonin receptor, an effective amount is an amount that, for example, increases said activation compared to the activation without administration of the one or more compounds.

“Palliating” a disease, disorder or condition means that the extent and/or undesirable clinical manifestations of a disease, disorder or condition are lessened and/or time course of the progression is slowed or lengthened, as compared to not treating the disorder.

The term “administered” as used herein means administration of a therapeutically effective amount of one or more compositions of the application to a cell, tissue, organ or subject.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The “disease, disorder or condition” as used herein refers to a disease, disorder or condition treated by activation a serotonin receptor, for example 5-HT2A and particularly using a serotonin receptor agonist, such as one or more hallucinogens as herein described.

The term “disease, disorder or condition treated by activation of a serotonin receptor” as used herein means that the disease, disorder or condition to be treated is affected by, modulated by and/or has some biological basis, either direct or indirect, that includes serotonergic activity, in particular increases in serotonergic activity. These diseases respond favorably when serotonergic activity associated with the disease, disorder or condition is agonized by one or more hallucinogens.

The term “activation” as used herein includes agonism, partial agonist and positive allosteric modulation of a serotonin receptor.

The terms “5-HT1A”, “5-HT2A” and “5-HT2C” are used herein mean the 5-HT1A, 5-HT2A and 5-HT2C receptor subtypes of the 5-HT1 and 5-HT2 serotonin receptors, respectively.

The term “therapeutic agent” as used herein refers to any drug or active agent that has a pharmacological effect when administered to a subject.

The term “intranasal composition” as used herein refers to a composition that is delivered to the nasal cavity and/or nasal sinuses.

The term “5-MeO-DMT” as used herein refers to a compound having the chemical name: 5-methoxy-N,N-dimethyltryptamine, and having the chemical structure:

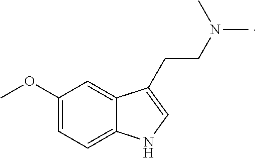

The term “bioavailability” as used herein refers to the rate and extent an active compound reaches the systemic circulation as an intact drug.

The term “Cmax” as used herein refers to the maximum concentration (or peak concentration) of a compound in reference material after a single administration of the compound

The term “Tmax” as used herein refers to the time to reach maximum concentration in reference material following administration of a compound.

The term ““AUC” as used herein, refers to the area under the curve that represents changes in concentration of a compound in reference material over time. The term “increased” or “decreased” as used herein, refers to any detectable increase/decrease in a parameter in the presence of a variable compared to otherwise similar/same conditions except in the absence of the variable “.

### II. Compositions and Kits of the Application

The present application includes a pharmaceutical composition comprising one or more hallucinogens, or a pharmaceutically acceptable salt, prodrug and/or solvate thereof, and one or more fatty acids, or a pharmaceutically acceptable salt, prodrug and/or solvate thereof.

In some embodiments, the one or more hallucinogens, or a pharmaceutically acceptable salt, prodrug and/or solvate thereof, and the one or more fatty acids, or a pharmaceutically acceptable salt, prodrug and/or solvate thereof, are present in an amount effective to treat or prevent a disease, disorder or condition that is treated by activation of a serotonin receptor

The present application also includes a pharmaceutical composition comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more fatty acids are present in amounts that are effective for improving the efficacy of the one or more hallucinogens to treat or prevent a disease, disorder or condition that is treated by activation of a serotonin receptor.

The present application also includes a kit comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more hallucinogens, or a pharmaceutically acceptable salt, prodrug and/or solvate thereof, and the one or more fatty acids, or a pharmaceutically acceptable salt, prodrug and/or solvate thereof, are in a single pharmaceutical composition or are in separate pharmaceutical compositions.

The present application also includes a kit for treating or preventing a disease, disorder or condition that is treated by activation of a serotonin receptor the kit comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more hallucinogens, or a pharmaceutically acceptable salt, prodrug and/or solvate thereof, and the one or more fatty acids, or a pharmaceutically acceptable salt, prodrug and/or solvate thereof, are in a single pharmaceutical composition or are in separate pharmaceutical compositions and the one or more hallucinogens, or a pharmaceutically acceptable salt, prodrug and/or solvate thereof, and one or more fatty acids, or a pharmaceutically acceptable salt, prodrug and/or solvate thereof are present in amounts to treat or prevent the disease, disorder or condition that is treated by activation of a serotonin receptor.

The present application also includes a kit for improving the efficacy of one or more hallucinogens, or a pharmaceutically acceptable salt, prodrug and/or solvate thereof, for treating or preventing a disease, disorder or condition that is treated by activation of a serotonin receptor, the kit comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more hallucinogens, or a pharmaceutically acceptable salt, prodrug and/or solvate thereof, and the one or more fatty acids, or a pharmaceutically acceptable salt, prodrug and/or solvate thereof, are in a single pharmaceutical composition or are in separate pharmaceutical compositions, and the one or more fatty acids are present in amounts that are effective for improving the efficacy of the one or more hallucinogens, or a pharmaceutically acceptable salt, prodrug and/or solvate thereof, to treat or prevent the disease, disorder or condition that is treated by activation of a serotonin receptor.

In some embodiments, the kit further comprises instructions for administration of the one or more hallucinogens, or a pharmaceutically acceptable salt, prodrug and/or solvate thereof, and the one or more fatty acids, or a pharmaceutically acceptable salt, prodrug and/or solvate thereof, to a subject in need thereof.

In some embodiments, the one or more hallucinogens, or a pharmaceutically acceptable salt, prodrug and/or solvate thereof, are selected from any hallucinogens that is known to be used in medical therapy or treatments, for example, for any disease, disorder or condition that is treated by activation of a serotonin receptor. In some embodiments, the one or more hallucinogens are selected from one or more psychedelics. In some embodiments, the one or more hallucinogens are selected from psilocybin, psilocin, dimethyltryptamine (DMT), 5-methoxy-dimethyltryptamine (5-MeO-DMT), mescaline, lysergic acid diethylamide (LSD), 3,4-methylenedioxy methamphetamine (MDMA), ibogaine, ketamine, and salvinorin A, or a pharmaceutically acceptable salt, prodrug and/or solvate thereof. In some embodiments, the composition comprises one psychedelic. In some embodiments, the one or more hallucinogens are phenethylamines, or a pharmaceutically acceptable salt, prodrug and/or solvate thereof. In some embodiments the one or more hallucinogens is psilocybin, or a pharmaceutically acceptable salt, prodrug and/or solvate thereof.

In some embodiments, the one or more hallucinogens, or a pharmaceutically acceptable salt, prodrug and/or solvate thereof, are selected from:
9,10-didehydro-6-allyl-N,N-diethylergoline-8β-carboxamide;
9,10-didehydro-6,N,N-triethylergoline-8β-carboxamide;
N,N-Dibutyltryptamine;
N,N-Diethyltryptamine;
N,N-Diisopropyltryptamine;
5-Methoxy-α-methyltryptamine;
2,α-dimethyltryptamine;
α,N-dimethyltryptamine;
N, N-Dipropyltryptamine;
N-Ethyl-N-Isopropyltryptamine;
α-Ethyltryptamine;
Harmaline (7-Methoxy-1-methyl-β-carboline)(deaminase blocker);
Harmine (7-Methoxy-β-carboline)(deaminase blocker);
4-hydroxy-diethyltryptamine and phosphate ester;
4-hydroxy-diisopropyltryptamine;
4-hydroxy-methyl-tryptamine;
4-hydroxy-tryptamine;
5-hydroxy-tryptamine;
4-hydroxy-dipropyltryptamine;
4-hydroxy-n-methyl-n-ethyl-tryptamine;
4-hydroxy-N-Methyl-N-Isopropyl-tryptamine;
4-hydroxy-N-methyl-N-ethyl-trytamine;
4-hydroxy-N-N-tetramethylene-tryptamine;
d-iso-LSD;
I-LSD;
I-iso-LSD;
N,N-diisopropyl-4,5-methylenedioxy tryptamine;
N,N-diisopropyl-5,6-methylenedioxy tryptamine;
N,N-Diemethyl-4,5-methylenedioxy tryptamine;
N,N-Diemethyl-5,6-methylenedioxy tryptamine;
2-methyl-DMT;
5-MeO-Diethyltryptamine;
5-MeO-Diisopropyltryptamine;
4-MeO-N-isopropyl-N-methyl-tryptamine;
5-MeO-N-isopropyl-N-methyl-tryptamine;
5-MeO-NMT;
5-MeO-2,N,N-trimetyltryptamine;
N-Isopropyl-N-methyl-tryptamine;
Alpha-methyltryptamine;
Alpha-methyl-4-OH-tryptamine;
N-methyl-tryptamine;
5-MeO-α,N-dimethyl-tryptamine;
4-allyloxy-3,5-diemethoxyphenethylamine;
2,5-dimethoxy-4-methylthioamphetamine;
2,5-dimethoxy-4-ethylthioamphetamine;
2,5-dimethoxy-4-(i)-propylthioamphetamine;
2,5-dimethoxy-4-phenylthioamphetamine;
2,5-dimethoxy-4-(n)-propylthioamphetamine;
2,5-dimethoxy-α-ethyl-4-methylphenethylamine;
3,4-diethoxy-5-methoxy-phenethylamine;
4-(n)-butoxy-3,5-dimethoxy-phenethylamine;
2,5-dimethoxy-4,N-dimethylamphetamine4-bromo-2,5-β-trimethoxyphenethylamine;
4-methyl-2,5,β-trimethoxyphenethylamine;
β-methoxy-3,4-methylenedioxyphenethylamine;
3,4,5,β-tetramethoxyphenethylamine;
3,5-dimethoxy-4-bromoamphetamine;
2-bromo-4,5-methylenedioxyamphetamine;
4-bromo-2,5,dimethoxyphenethylamine;
4-benzyloxy-3,5-dimethoxyamphetamine;
2,5-dimethoxy-4-chlorophenethylamine;
2,5-dimethoxy-4-methylphenethylamine;
2,5-dimethoxy-4-ethylphenethylamine;
3,5-dimethoxy-4-ethoxyamphetamine;
2,5-dimethoxy-4-fluorophenethylamine;
2,5-dimethoxy-3,4-dimethylphenethylamine;
2,5-dimethoxy-3,4-(trimethylene)phenethylamine;
2,5-dimethoxy-3,4-(tetramethylene)phenethylamine;
3,6-dimethoxy-4-(2-aminoethyl)benzonorborane;
1,4-dimethoxynapthyl-2-ethylamine;
2,5-dimethoxyphenethylamine;
2,5-dimethoxy-4-iodoophenethylamine;
2,5-dimethoxy-4-nitrophenethylamine;
2,5-dimethoxy-4-(i)-propoxyphenethylamine;
2,5-dimethoxy-4-(n)-propoxyphenethylamine;
4-cyclopropyl-3,5-dimethoxyphenethylamine;
2,5-dimethoxy-4-methylseleneophenethylamine;
2,5-dimethoxy-4-methylthiophenethylamine;
2,5-dimethoxy-4-ethylthiophenethylamine;
2,5-dimethoxy-4-(i)-propylthiophenethylamine;
2,6-dimethoxy-4-(i)-propylthiophenethylamine;
2,5-dimethoxy-4-(n)-propylthiophenethylamine;
2,5-dimethoxy-4-cyclopropylmethylthiothiophenethylamine;
2,5-dimethoxy-4-(t)-butylthiophenethylamine;
2,5-dimethoxy-4-(2-methoxyrthylthio)phenethylamine;
2,5-dimethoxy-4-cyclopropylthiophenethylamine;
2,5-dimethoxy-4-(s)-butylthiophenethylamine;
2,5-dimethoxy-4-(2fluorothio)phenethylamine;
3,5-dimethoxy-4-trideuteromethoxyphenethylamine;
3,4,5-trimethoxy-β,β-dideuterophenethylamine;
3,5-dimethoxy-4-methylphenethylamine;
2,4-dimethoxyamphetamine;
2,5-dimethoxyamphetamine;
3,4-dimethoxyamphetamine;
2,5-dimethoxy-3,4-methylenedioxyamphetamine;
2,5-dimethoxy-4-bromoamphetamine;
2,5-dimethoxy-4-chloroamphetamine;
2,5-dimethoxy-4-(2fluoroethyl)-amphetamine;
2,5-dimethoxy-4-iodoamphetamine;
2,5-dimethoxy-4-methylamphetamine;
2,6-dimethoxy-4-methylamphetamine;
2,5-dimethoxy-4-(n)-propylamphetamine;
3,5-dimethoxy-4-ethoxyphenethylamine;
2,4,5-triethoxyamphetamine;
2,4-diethoxy-5-methoxyamphetamine;
2,5-diethoxy-4-methoxyamphetamine;
4,5-dimethoxy-2-ethoxyamphetamine;
N-hydroxy-N-methyl-3,4-methylenedioxyamphetamine;
2,5-dimethoxy-3,4-(trimethylene) amphetamine;
3,6-dimethoxy-4-(2-aminopropyl)benzonorborane;
2,5-dimethoxy-3,4-dimethylamphetamine;
2,5-dimethoxy-4-ethylthio-N-hydroxyphenethylamine;
2,5-dimethoxy-N-hydroxy-4-(n)-propylthiophenethylamine;
2,5-dimethoxy-4-(s)-butylthio-N-hydroxyphenethylamine;
3,5-dimethoxy-4-(i)-propoxyphenethylamine;
5-ethoxy-2-methoxy-4-methylamphetamine;
2-amino-(3,4-methylenedioxyphenyl) butane;
3-methoxy-4,5-methylenedioxyphenethylamine;
3,4,5-trimethoxyphenethylamine;
3,5-dimethoxy-4-methalloxyphenethylamine;
3,4-methylenedioxyamphetamine;
3,4-methylenedioxy-N-ethylamphetamine;
3,4-methylenedioxy-n-methylamphetamine;
3,4-methylenedioxy-N-hydroxyamphtamine;
3,4-methylenedioxy-5-ethoxyphenethylamine;
2,5-dimethoxy-4-ethoxyapmphetamine;
3-methoxy-4-ethoxyphenethylamine;
2-methylamino-1-(3,4-methylenedioxyphenyl) butane;
3-methoxy-4,5-methylenedioxyamphetamine;
2-methoxy-4,5-methylenedioxyamphetamine;
2-methoxy-3,4-methylenedioxyamphetamine;
4-methoxy-2,3-methylenedioxyamphetamine;
3,5-Dimethoxy-4-(n)-propoxyphenethylamine;
4-ethoxy-5-methoxy-3-methylthiophenethylamine;
3,5-dimethoxy-4-ethylthiophenethylamine;
3,4-dimethoxy-5-methylthiophenethylamine;
3,5-dimethoxy-5-methylthiophenethylamine;
3,4,5-trimethoxyamphetamine;
2,4,5-trimethoxyamphetamine;
2,3,5-trimethoxyamphetamine;
2,3,6-trimethoxyamphetamine;
2,4,6-trimethoxyamphetamine;
4,5-dimethoxy-3-ethylthiophenethylamine;
4-ethyl-2-methoxy-5-methylthioamphetamine;
5-methoxy-4-methyl-2-methylthioamphetamine;
2-methoxy-4-methyl-5-methylthioamphetamine;
2-methoxy-4-methyl-5-methylsulfinylamphetamine; and
3,5-dimethoxy-4-(n)-propylthiophenethylamine,
or a pharmaceutically acceptable salt, prodrug and/or solvate thereof.

In some embodiments, the one or more hallucinogens, or a pharmaceutically acceptable salt, prodrug and/or solvate thereof, are selected from salvinorin A, ibotenic acid, muscimol, dextromethorphan, ketamine, esketamine, phencyclidine, dizocilpine (MK-801), scopolamine, hyoscyamine, aporphine, lysergic acid amide, cathine, cathinone, and voacangine, or a pharmaceutically acceptable salt, prodrug and/or solvate thereof.

In some embodiments, the one or more hallucinogens, or a pharmaceutically acceptable salt, prodrug and/or solvate thereof, are selected from:

| Compound | | |
|---|---|---|
| ID # | IUPAC Name | Chemical Structure |
| 1 | 3-(2-(dimethylamino)ethyl)-1H- indol-4-yl dihydrogen phosphate | 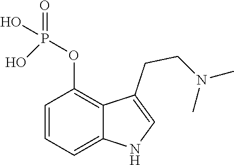 [SMILES: CN(C)CCc1c[nH]c2cccc(OP(=O)(O)O)c12] |
| 2 | 3-(2-aminoethyl)-1H-indol-4-yl dihydrogen phosphate | 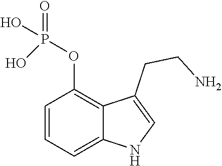 [SMILES: NCCc1c[nH]c2cccc(OP(=O)(O)O)c12] |
| 3 | 3-(2-(diisopropylamino)ethyl)- 1H-indol-4-yl dihydrogen phosphate | 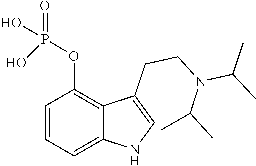 [SMILES: CC(C)N(CCc1c[nH]c2cccc(OP(=O)(O)O)c12)C(C)C] |
| 4 | 3-(2- (isopropyl(methyl)amino)ethyl)- 1H-indol-4-yl dihydrogen phosphate | 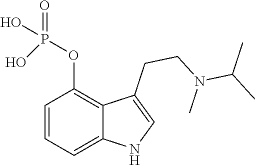 [SMILES: CC(C)N(C)CCc1c[nH]c2cccc(OP(=O)(O)O)c12] |
| 5 | 3-(2-(dimethylamino)ethyl)-1H- indol-4-yl acetate | 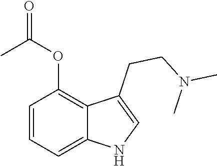 [SMILES: CC(=O)Oc1cccc2[nH]cc(CCN(C)C)c12] |
| 6 | 3-(2-aminoethyl)-1H-indol-4-ol | 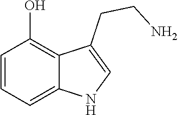 [SMILES: NCCc1c[nH]c2cccc(O)c12] |
| 7 | 3-(2-(dimethylamino)ethyl)-1H- indol-4-ol | 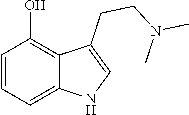 |
| 8 | 3-(2-(diisopropylamino)ethyl)- 1H-indol-4-ol | 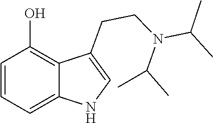 [SMILES: CC(C)N(CCc1c[nH]c2cccc(O)c12)C(C)C] |
| 9 | 3-(2- (isopropyl(methyl)amino)ethyl)- 1H-indol-4-ol | 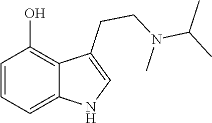 [SMILES: CC(C)N(C)CCc1c[nH]c2cccc(O)c12] |
| 10 | 2-(1H-indol-3-yl)-N,N- dimethylethan-1-amine | 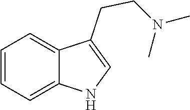 |
| 11 | N,N-diethyl-2-(1H-indol-3- yl)ethan-1-amine | 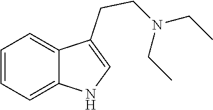 [SMILES: CCN(CC)CCc1c[nH]c2ccccc12] |
| 12 | N-(2-(1H-indol-3-yl)ethyl)-N- butylbutan-1-amine | 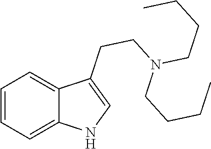 [SMILES: CCCCN(CCCC)CCc1c[nH]c2ccccc12] |
| 13 | N-(2-(1H-indol-3-yl)ethyl)-N- propylpropan-1-amine | 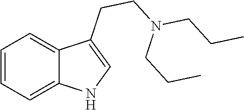 [SMILES: CCCN(CCC)CCc1c[nH]c2ccccc12] |
| 14 | N-(2-(1H-indol-3-yl)ethyl)-N- isopropylpropan-2-amine | 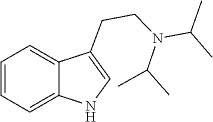 [SMILES: CC(C)N(CCc1c[nH]c2ccccc12)C(C)C] |
| 15 | N-(2-(1H-indol-3-yl)ethyl)-N- ethylpropan-2-amine | 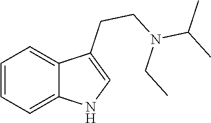 [SMILES: CCN(CCc1c[nH]c2ccccc12)C(C)C] |
| 16 | 2-(5-methoxy-1H-indol-3-yl)- N,N-dimethylethan-1-amine | 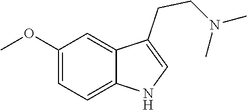 |
| 17 | N-ethyl-N-(2-(5-methoxy-1H- indol-3-yl)ethyl)propan-2-amine | 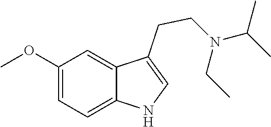 [SMILES: CCN(CCc1c[nH]c2ccc(OC)cc12)C(C)C] |
| 18 | N-isopropyl-N-(2-(5-methoxy- 1H-indol-3-yl)ethyl)propan-2- amine | 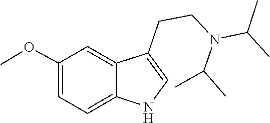 [SMILES: COc1ccc2[nH]cc(CCN(C(C)C)C(C)C)c2c1] |
| 19 | 5-methoxy-3-(2-(pyrrolidin-1- yl)ethyl)-1H-indole | 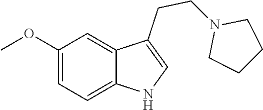 [SMILES: COc1ccc2[nH]cc(CCN3CCCC3)c2c1] |
| 20 | (S)-1-(5-methoxy-1H-indol-3- yl)propan-2-amine | 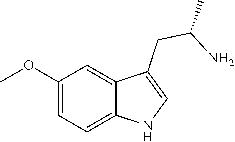 |
| 21 | (S)-1-(3,7,8,9- tetrahydropyrano[3,2-e]indol-1- yl)propan-2-amine | 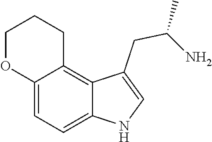 [SMILES: C[C@H](N)Cc1c[nH]c2ccc3c(c12)CCCO3] |
| 22 | (R)-1-((S)-2-aminopropyl)- 3,7,8,9-tetrahydropyrano[3,2- e]indol-8-ol | 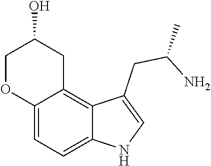 [SMILES: C[C@H](N)Cc1c[nH]c2ccc3c(c12)C[C@@H](O)CO3] |
| 23 | (S)-1-((S)-2-aminopropyl)- 3,7,8,9-tetrahydropyrano[3,2- e]indol-8-ol | 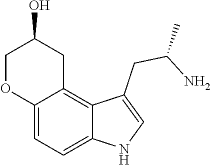 [SMILES: C[C@H](N)Cc1c[nH]c2ccc3c(c12)C[C@H](O)CO3] |
| 24 | 1-(2-(dimethylamino)ethyl)-1H- indazol-6-ol | 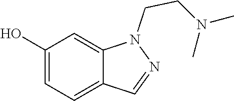 |
| 25 | (S)-1-(2-(dimethylamino)propyl)- 1H-indazol-6-ol | 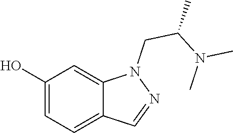 [SMILES: C[C@@H](Cn1ncc2ccc(O)cc21)N(C)C] |
| 26 | (S)-1-(2-aminopropyl)-1H- indazol-6-ol | 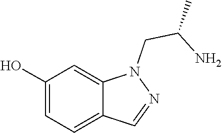 |
| 27 | (S)-1-(2-aminopropyl)-7-fluoro- 1H-indazol-6-ol | 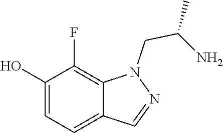 |
| 28 | 1-(2-(dimethylamino)ethyl)-1H- indazol-7-ol | 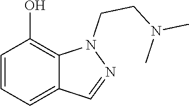 |
| 29 | (S)-1-(6-methoxy-1H-indazol-1- yl)-N,N-dimethylpropan-2-amine | 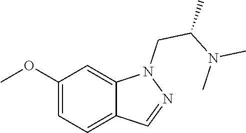 |
| 30 | (S)-1-(6-methoxy-1H-indazol-1- yl)propan-2-amine | 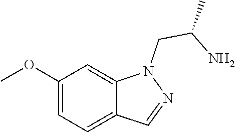 |
| 31 | (R)-6-methoxy-1-((1- methylpyrrolidin-2-yl)methyl)- 1H-indazole | 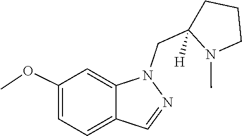 [SMILES: COc1ccc2cnn(C[C@H]3CCCN3C)c2c1] |
| 32 | (S)-6-methoxy-1-((1- methylpyrrolidin-2-yl)methyl)- 1H-indazole | 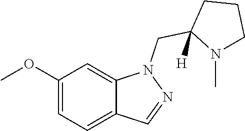 [SMILES: COc1ccc2cnn(C[C@@H]3CCCN3C)c2c1] |
| 33 | (S)-1-(8,9-dihydropyrano[2,3- glindazol-1(7H)-yl)propan-2- amine | 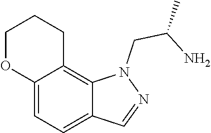 [SMILES: C[C@H](N)Cn1ncc2ccc3c(c21)CCCO3] |
| 34 | (R)-1-((S)-2-aminopropyl)- 1,7,8,9-tetrahydropyrano[2,3- glindazol-8-ol | 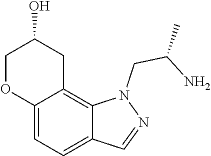 [SMILES: C[C@H](N)Cn1ncc2ccc3c(c21)C[C@@H](O)CO3] |
| 35 | (6aS,9R)-N,N-diethyl-7-methyl- 4,6,6a,7,8,9- hexahydroindolo[4,3- fg]quinoline-9-carboxamide | 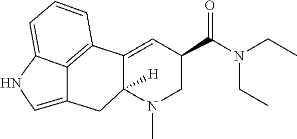 |
| 36 | (6aR,9S)-N,N-diethyl-7-methyl- 4,6,6a,7,8,9- hexahydroindolo[4,3- fg]quinoline-9-carboxamide | 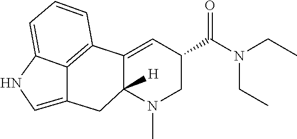 |
| 37 | (6aS,9R)-5-bromo-N,N-diethyl- 7-methyl-4,6,6a,7,8,9- hexahydroindolo[4,3- fg]quinoline-9-carboxamide | 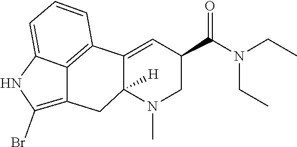 |
| 38 | (6aR,9S)-5-bromo-N,N-diethyl- 7-methyl-4,6,6a,7,8,9- hexahydroindolo[4,3- fg]quinoline-9-carboxamide | 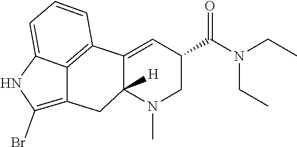 |
| 39 | (6aS,9R)-N,N-diethyl-5-fluoro-7- methyl-4,6,6a,7,8,9- hexahydroindolo[4,3- fg]quinoline-9-carboxamide | 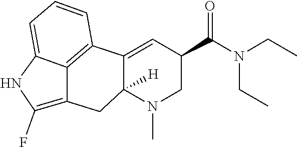 |
| 40 | (6aS,9R)-5-chloro-N,N-diethyl- 7-methyl-4,6,6a,7,8,9- hexahydroindolo[4,3- fg]quinoline-9-carboxamide | 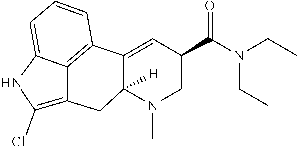 |
| 41 | (6aS,9R)-N-ethyl-7-methyl- 4,6,6a,7,8,9- hexahydroindolo[4,3- fg]quinoline-9-carboxamide | 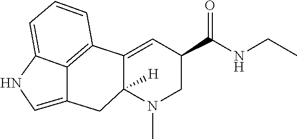 |
| 42 | (6aS,9R)-N-ethyl-4,6,6a,7,8,9- hexahydroindolo[4,3- fg]quinoline-9-carboxamide | 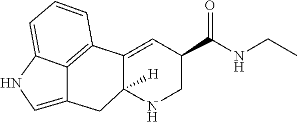 |
| 43 | (6aS,9R)-N-ethyl-N-(2- hydroxyethyl)-7-methyl- 4,6,6a,7,8,9- hexahydroindolo[4,3- fg]quinoline-9-carboxamide | 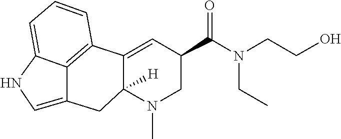 |
| 44 | ((2S,4S)-2,4-dimethylazetidin-1- yl)((6aS,9R)-7-methyl- 4,6,6a,7,8,9- hexahydroindolo[4,3-fg]quinolin- 9-yl)methanone | 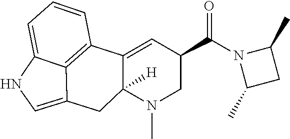 |
| 45 | (6aS,9R)-N,N,7-triethyl- 4,6,6a,7,8,9- hexahydroindolo[4,3- fg]quinoline-9-carboxamide | 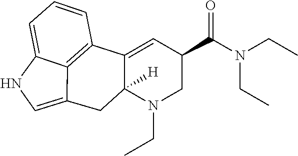 |
| 46 | (6aS,9R)-7-allyl-N,N-diethyl- 4,6,6a,7,8,9- hexahydroindolo[4,3- fg]quinoline-9-carboxamide | 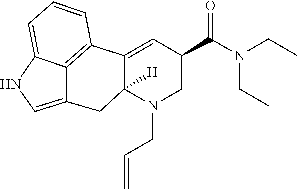 |
| 47 | (6aS,9R)-7-methyl-4,6,6a,7,8,9- hexahydroindolo[4,3- fg]quinoline-9-carboxamide | 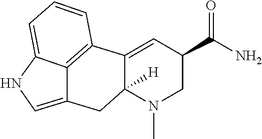 |
| 48 | (R)-1-(4-bromo-2,5- dimethoxyphenyl)propan-2- amine | 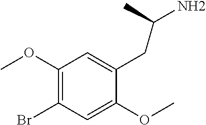 |
| 49 | 2-(3,4,5- trimethoxyphenyl)ethan-1-amine | 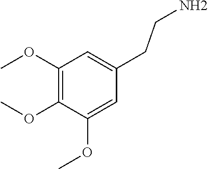 |
| 50 | 2-(3,5-dimethoxy-4- (propylthio)phenyl)ethan-1- amine | 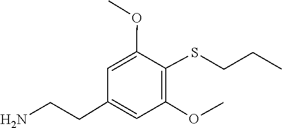 [SMILES: CCCSc1c(OC)cc(CCN)cc1OC] |
| 51 | 2-methoxy-4-methyl-5- methylthioamphetamine | 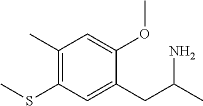 |
| 52 | 1-(2-methoxy-4-methyl-5- (methylsulfinyl)phenyl)propan-2- amine | 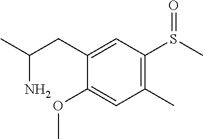 [SMILES: COc1cc(C)c(S(C)=O)cc1CC(C)N] |
| 53 | 4,5-dimethoxy-3- ethylthiophenethylamine | 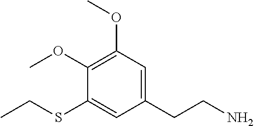 [SMILES: CCSc1cc(CCN)cc(OC)c1OC] |
| 54 | 2-(3,5-dimethoxy-4- propoxyphenyl)ethan-1-amine | 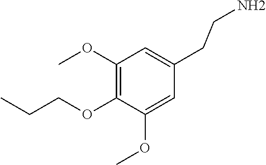 [SMILES: CCCOc1c(OC)cc(CCN)cc1OC] |
| 55 | 2-(6,7-dimethoxy-2,3- dihydrobenzofuran-4-yl)ethan-1- amine | 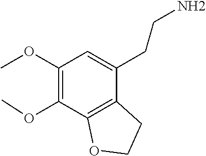 [SMILES: COc1cc(CCN)c2c(c1OC)OCC2] |
| 56 | 2-(8-methoxy-2,3,5,6- tetrahydrobenzo[1,2-b:5,4- b′]difuran-4-yl)ethan-1-amine | 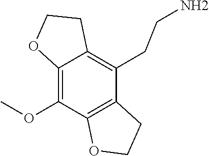 [SMILES: COc1c2c(c(CCN)c3c1OCC3)CCO2] |
| 57 | (R)-(4,5,6-trimethoxy-2,3- dihydro-1H-inden-1- yl)methanamine | 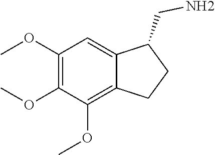 [SMILES: COc1cc2c(c(OC)c1OC)CC[C@H]2CN] |
| 58 | (R)-(3-bromo-2,4- dimethoxybicyclo[4.2.0]octa- 1,3,5-trien-7-yl)methanamine | 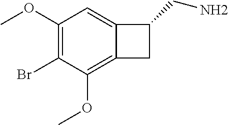 |
| 59 | 2-(4-iodo-3,5- dimethoxyphenyl)ethan-1-amine | 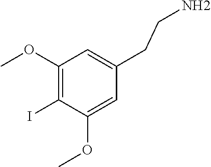 |
| 60 | 2-(4-iodo-3,5-dimethoxyphenyl)- N-(2-methoxybenzyl)ethan-1- amine | 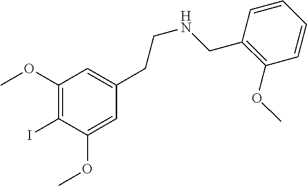 [SMILES: COc1ccccc1CNCCc1cc(OC)c(I)c(OC)c1] |
| 61 | 4-(2-((2- hydroxybenzyl)amino)ethyl)-2,6- dimethoxybenzonitrile | 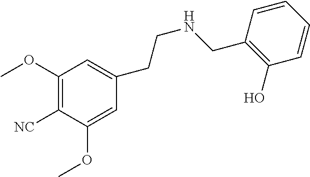 [SMILES: COc1cc(CCNCc2ccccc2O)cc(OC)c1C#N] |
| 62 | 2-(((3,5-dimethoxy-4- (trifluoromethyl)phenethyl) amino)methyl)phenol | 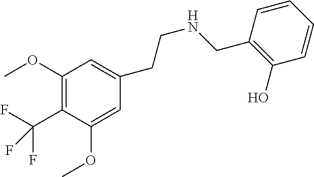 [SMILES: COc1cc(CCNCc2ccccc2O)cc(OC)c1C(F)(F)F] |
| 63 | N-(benzo[d][1,3]dioxol-4- ylmethyl)-2-(3,5-dimethoxy-4- (trifluoromethyl)phenyl)ethan-1- amine | 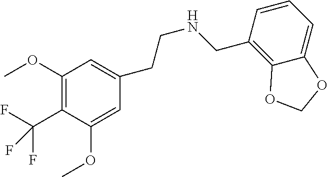 [SMILES: COc1cc(CCNCc2cccc3c2OCO3)cc(OC)c1C(F)(F)F] |
| 64 | (3R,4S, 11bR)-3-ethyl-8- methoxy-1,2,3,3a,5,6,11,11b- octahydro-1,4- methanocyclopenta[2,3]azepino [4,5-b]indole | 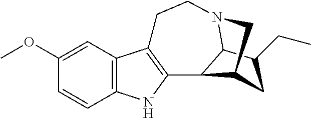 |
| 65 | methyl (3R,4S, 11bS)-3-ethyl-8- methoxy-1,2,3,3a,6,11- hexahydro-1,4- methanocyclopenta[2,3]azepino [4,5-b]indole-11b(5H)- carboxylate | 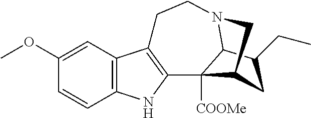 |
| 67 | methyl (3S,4S, 11bS)-8- methoxy-3-(2- (methylamino)ethyl)- 1,2,3,3a,6,11-hexahydro-1,4- methanocyclopenta[2,3]azepino [4,5-b]indole-11b(5H)- carboxylate | 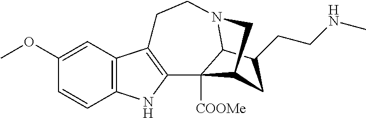 |
| 68 | (3R,4S,11bR)-3-ethyl- 1,2,3,3a,5,6,11,11b-octahydro- 1,4- methanocyclopenta[2,3]azepino [4,5-b]indol-8-ol | 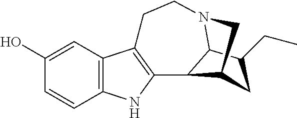 |
| 69 | (4S, 11bR)-8-fluoro- 1,2,3,3a,5,6,11,11b-octahydro- 1,4- methanocyclopenta[2,3]azepino [4,5-b]indole | 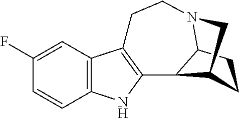 |
| 70 | (4S,11bR)-1,2,3,3a,5,6,11,11b- octahydro-1,4- methanocyclopenta[2,3]azepino [4,5-b]indole | 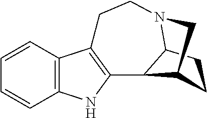 |
| 71 | methyl (3S,4S,11bS)-3-(2- methoxyethyl)-1,2,3,3a,6,11- hexahydro-1,4- methanocyclopenta[2,3]azepino [4,5-b]indole-11b(5H)- carboxylate | 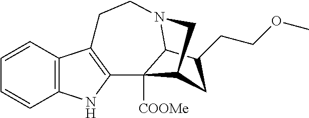 |
| 72 | 2-methoxyethyl (3S,4S,11bS)-3- (2-methoxyethyl)-1,2,3,3a,6,11- hexahydro-1,4- methanocyclopenta[2,3]azepino [4,5-b]indole-11b(5H)- carboxylate | 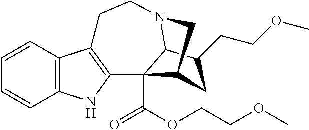 |
| 73 | methyl (3S,4S,11bS)-3-(2- hydroxyethyl)-1,2,3,3a,6,11- hexahydro-1,4- methanocyclopenta[2,3]azepino [4,5-b]indole-11b(5H)- carboxylate | 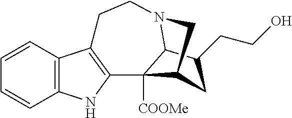 |
| 74 | 9-methyl-3-oxa-9- azatricyclo[3.3.1.02,4]nonan-7- yl 3-hydroxy-2- phenylpropanoate | 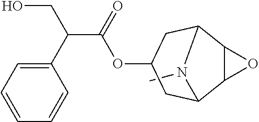 [SMILES: CN1C2CC(OC(=O)C(CO)c3ccccc3)CC1C1OC12] |
| 75 | (1R,3r,5S)-8-methyl-8- azabicyclo[3.2.1]octan-3-yl 3- hydroxy-2-phenylpropanoate | 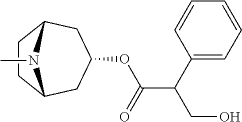 [SMILES: CN1[C@@H]2CC[C@H]1C[C@@H](OC(=O)C(CO)c1ccccc1)C2] |
| 76 | (S)-6-methyl-5,6,6a,7- tetrahydro-4H- dibenzo[de,g]quinoline | 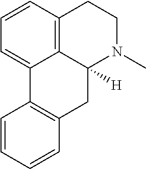 [SMILES: CN1CCc2cccc3c2[C@@H]1Cc1ccccc1-3] |
| 77 | 8-methoxy-3-methyl-1,2,3,4,5,6- hexahydroazepino[4,5-b]indole | 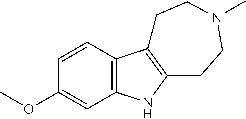 |
| 78 | (R)-2-(2-chlorophenyl)-2- (methylamino)cyclohexan-1-one | 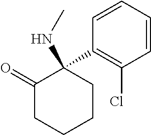 [SMILES: CN[C@@]1(c2ccccc2Cl)CCCCC1=O] |
| 79 | (S)-2-(2-chlorophenyl)-2- (methylamino)cyclohexan-1-one | 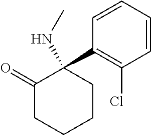 [SMILES: CN[C@]1(c2ccccc2Cl)CCCCC1=O] |
| 80 | (R)-2-(2-methoxyphenyl)-2- (methylamino)cyclohexan-1-one | 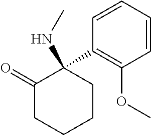 [SMILES: CN[C@@]1(c2ccccc2OC)CCCCC1=O] |
| 81 | (R)-2-(2-methoxyphenyl)-2- (methylamino)cyclohexan-1-one | 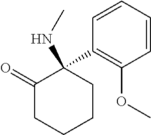 [SMILES: CN[C@@]1(c2ccccc2OC)CCCCC1=O] |
| 82 | (R)-2-amino-2-(2- chlorophenyl)cyclohexan-1-one | 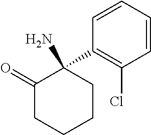 [SMILES: N[C@@]1(c2ccccc2Cl)CCCCC1=O] |
| 83 | (S)-2-amino-2-(2- chlorophenyl)cyclohexan-1-one | 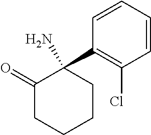 [SMILES: N[C@]1(c2ccccc2Cl)CCCCC1=O] |
| 84 | (2R)-2-amino-2-(2- chlorophenyl)-6- hydroxycyclohexan-1-one | 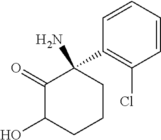 [SMILES: N[C@@]1(c2ccccc2Cl)CCCC(O)C1=O] |
| 85 | (2S)-2-amino-2-(2- chlorophenyl)-6- hydroxycyclohexan-1-one | 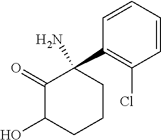 [SMILES: N[C@]1(c2ccccc2Cl)CCCC(O)C1=O] |
| 86 | methyl (R)-5-((1-(2- chlorophenyl)-2- oxocyclohexyl)amino)pentanoate | 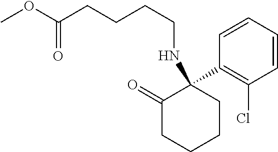 [SMILES: COC(=O)CCCCN[C@@]1(c2ccccc2Cl)CCCCC1=O] |
| 87 | methyl (S)-5-((1-(2- chlorophenyl)-2- oxocyclohexyl)amino)pentanoate | 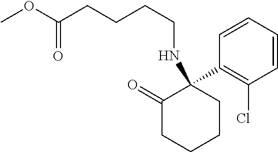 [SMILES: COC(=O)CCCCN[C@@]1(c2ccccc2Cl)CCCCC1=O] |
| 88 | (S)-2-(ethylamino)-2-(3- methoxyphenyl)cyclohexan-1- one | 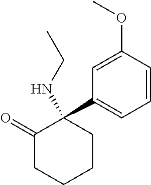 [SMILES: CCN[C@]1(c2cccc(OC)c2)CCCCC1=O] |
| 89 | (R)-2-(ethylamino)-2-(3- methoxyphenyl)cyclohexan-1- one | 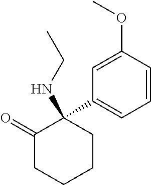 [SMILES: CCN[C@@]1(c2cccc(OC)c2)CCCCC1=O] |
| 90 | 1-(1-phenylcyclohexyl)piperidine | 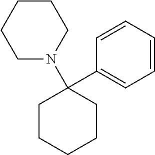 [SMILES: c1ccc(C2(N3CCCCC3)CCCCC2)cc1] |
| 91 | N-ethyl-1-(3- methoxyphenyl)cyclohexan-1- amine | 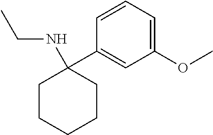 [SMILES: CCNC1(c2cccc(OC)c2)CCCCC1] |
| 92 | (S)-1-(benzo[d][1,3]dioxol-5-yl)- N-methylpropan-2-amine | 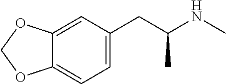 |
| 93 | (R)-1-(benzo[d][1,3]dioxol-5-yl)- N-methylpropan-2-amine | 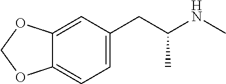 |
| 94 | (S)-1-(benzo[d][1,3]dioxol-5-yl)- 2-(methylamino)propan-1-one | 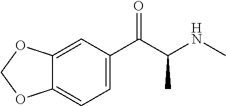 |
| 95 | (R)-1-(benzo[d][1,3]dioxol-5-yl)- 2-(methylamino)propan-1-one | 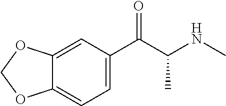 |
| 96 | (S)-2-(methylamino)-1-(p- tolyl)propan-1-one | 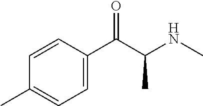 |
| 97 | (R)-2-(methylamino)-1-(p- tolyl)propan-1-one | 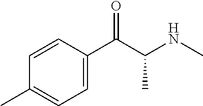 |
| 98 | (1S,2R)-2-amino-1- phenylpropan-1-ol | 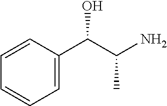 |
| 99 | (S)-1-(benzo[d][1,3]dioxol-5-yl)- N-ethylpropan-2-amine | 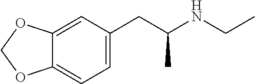 |
| 100 | (R)-1-(benzo[d][1,3]dioxol-5-yl)- N-ethylpropan-2-amine | 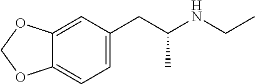 |
| 101 | (S)-1-(benzo[d][1,3]dioxol-5-yl)- 2-(pyrrolidin-1-yl)pentan-1-one | 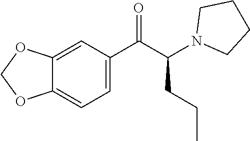 [SMILES: CCC[C@@H](C(=O)c1ccc2c(c1)OCO2)N1CCCC1] |
| 102 | (R)-1-(benzo[d][1,3]dioxol-5-yl)- 2-(pyrrolidin-1-yl)pentan-1-one | 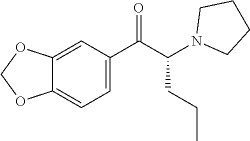 [SMILES: CCC[C@H](C(=O)c1ccc2c(c1)OCO2)N1CCCC1] |
| 103 | (S)-1-(benzo[d][1,3]dioxol-5-yl)- N-methylbutan-2-amine | 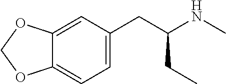 |
| 104 | (R)-1-(benzo[d][1,3]dioxol-5-yl)- N-methylbutan-2-amine | 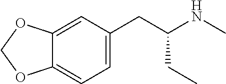 |
| 105 | (R)-1-(benzofuran-6-yl)propan- 2-amine | 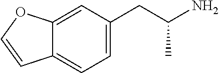 |
| 106 | (S)-1-(benzofuran-6-yl)propan- 2-amine | 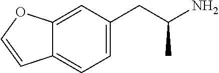 |
| 107 | 5-(aminomethyl)isoxazol-3-ol | 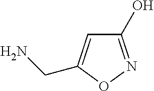 |
| 108 | 5-(aminomethyl)isothiazol-3-ol | 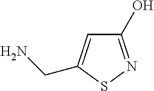 |
| 109 | (S)-2-amino-2-(3- hydroxyisoxazol-5-yl)acetic acid | 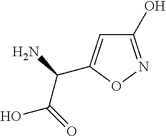 [SMILES: N[C@H](C(=O)O)c1cc(O)no1] |
| 110 | (R)-2-amino-2-(3- hydroxyisoxazol-5-yl)acetic acid | 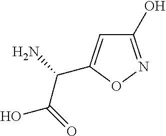 [SMILES: N[C@@H](C(=O)O)c1cc(O)no1] |
| 111 | 4,5,6,7-tetrahydroisoxazolo[5,4- c]pyridin-3(2H)-one | 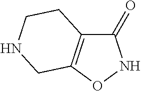 |
| 112 | N-methyl-2-phenyl-N- ((5R,7S,8S)-7-(pyrrolidin-1-yl)-1- oxaspiro[4.5]decan-8- yl)acetamide | 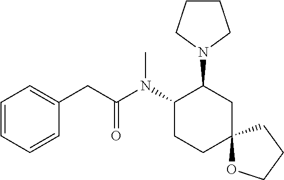 [SMILES: CN(C(=O)Cc1ccccc1)[C@H]1CC[C@@]2(CCCO2)C[C@@H]1N1CCCC1] |
| 113 | 2-(3,4-dichlorophenyl)-N-methyl- N-((1S,2S)-2-(pyrrolidin-1- yl)cyclohexyl)acetamide | 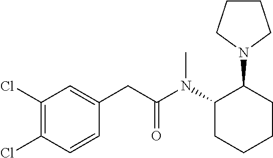 [SMILES: CN(C(=O)Cc1ccc(Cl)c(Cl)c1)[C@H]1CCCC[C@@H]1N1CCCC1] |
| 114 | methyl (2S,4aR,6aR,7R,9S, 10aS, 10bR)-9-acetoxy-2-(furan-3-yl)- 6a,10b-dimethyl-4,10- dioxododecahydro-2H- benzo[f]isochromene-7- carboxylate | 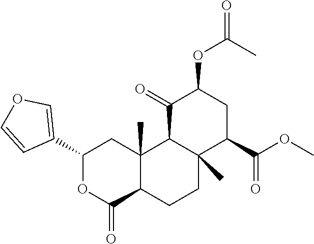 [SMILES: COC(=O)[C@@H]1C[C@H](OC(C)=O)C(=O)[C@H]2[C@@]1(C)CC[C@H]1C(=O)O[C@H](c3ccoc3)C[C@]21C] |
| 115 | methyl (2S,4aR,6aR,7R,9S, 10aS, 10bR)-2-(furan-3-yl)-9-hydroxy- 6a,10b-dimethyl-4,10- dioxododecahydro-2H- benzo[f]isochromene-7- carboxylate | 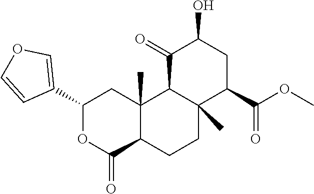 [SMILES: COC(=O)[C@@H]1C[C@H](O)C(=O)[C@H]2[C@@]1(C)CC[C@H]1C(=O)O[C@H](c3ccoc3)C[C@]21C] |
| 116 | 3′,5-diallyl-[1,1′-biphenyl]-2,4′- diol | 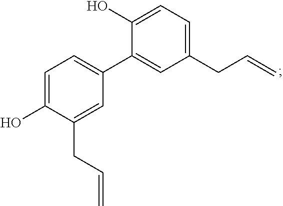 [SMILES: C=CCc1ccc(O)c(-c2ccc(O)c(CC=C)c2)c1] |
| | | and |
| 117 | 3′-allyl-5-propyl-[1,1′-biphenyl]- 2,4′-diol | 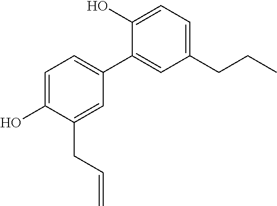 [SMILES: C=CCc1cc(-c2cc(CCC)ccc2O)ccc1O] |

or a pharmaceutically acceptable salt, prodrug and/or solvate thereof.

In some embodiments, the one or more hallucinogens, or a pharmaceutically acceptable salt, prodrug and/or solvate thereof, are selected from the compound disclosed and claimed in Applicant's co-pending PCT patent application serial number PCT/CA2021/050123 filed on Feb. 4, 2021, the contents of which are incorporated by reference in their entirety. Therefore in some embodiments, the one or more hallucinogens are selected from a compound of Formula (I) or a pharmaceutically acceptable salt, prodrug and/or solvate thereof:

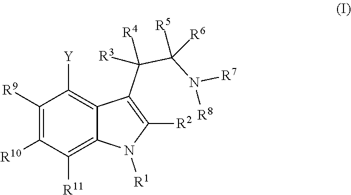

wherein R¹ is selected from hydrogen, C₁-C₃ alkyl, C(O)R¹², CO₂OR¹², C(O)N(R¹²)₂, S(O)R¹² and SO₂R¹²;
R³, R⁴, R⁵ and R⁶ are independently selected from hydrogen and C₁-C₆alkyl;
R⁷ and R⁸ are independently selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl and substituted or unsubstituted heteroaryl, or
R⁷ and R⁸ are taken together with the nitrogen atom therebetween to form a 3- to 7-membered heterocyclic ring optionally including 1 to 2 additional ring heteromoieties selected from O, S, S(O), SO₂, N and NR¹³,
wherein said C₃-C₇cycloalkyl and 3- to 7-membered heterocyclic ring are each further optionally substituted with a substituent selected from, halogen, CO₂R¹³, C(O)N(R¹³)₂, SO₂R¹³, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, N, S(O), SO₂ and NR¹³;
R⁹, R¹⁰, and R¹¹ are independently selected from hydrogen, halogen, CN, OR¹³, N(R¹³)₂, SR¹³, C₁-C₆alkyl, C₁-C₆haloalkyl, C₂-C₆haloalkenyl, CO₂R¹³, C(O)N(R¹³)₂, SOR¹³, SO₂R¹³, C₂-C₆alkenyl, C₂-C₆alkynyl, C₂-C₆haloalkynyl, C₃-C₇cycloalkyl and a 3- to 7-membered heterocyclic ring including 1 to 2 ring heteromoieties selected from O, S, S(O), SO₂, N and NR¹³, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR¹³, N(R¹³)₂ and SR¹³, and wherein said C₃-C₇cycloalkyl and 3- to 7-membered heterocyclic ring are each further optionally substituted with a substituent selected from halogen, CO₂R¹³, C(O)N(R¹³)₂, SO₂R¹³, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, S(O), SO₂, N and NR¹³;
Y is selected from halogen and X-A;
X is selected from O, NR¹³, S, S(O) and SO₂; A is selected from hydrogen, C₁-C₁₀alkyl, C₂-C₁₀alkenyl, C₂-C₁₀alkynyl, C₃-C₇cycloalkyl, C₄-C₆cycloalkenyl, heterocycloalkyl, aryl, heteroaryl and P(O)(OR¹²)₂;
each R¹² is independently selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl;
each R¹³ is independently selected from hydrogen, C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl, and a 3- to 7-membered heterocyclic ring including 1 to 2 ring heteromoieties selected from O, S, S(O), SO₂, N and NR¹⁴, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR¹⁴, N(R¹⁴)₂ and SR¹⁴, and wherein said C₃-C₇cycloalkyl and 3- to 7-membered heterocyclic ring are each further optionally substituted with a substituent selected from halogen, CO₂R¹⁴, C(O)N(R¹⁴)₂, SO₂R¹⁴, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, S(O), SO₂, N and NR¹⁴,
R¹⁴ is selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted heterocycloalkyl, substituted or unsubstituted aryl and substituted or unsubstituted heteroaryl;
wherein at least one of R³, R⁴, R⁵ and Re is deuterium or at least one of R³, R⁴, R⁵ and R⁸ comprises deuterium, and
wherein all available hydrogen atoms are optionally substituted with a halogen atom and/or all available atoms are optionally substituted with an alternate isotope thereof.

In some embodiments, the compound of Formula I is

| Compound | | |
|---|---|---|
| ID # | IUPAC Name | Chemical Structure |
| I-1 | 3-(2-(bis(methyl- d3)amino)ethyl- 1,1,2,2-d4)-1H- indol-4-yl dihydrogen phosphate | 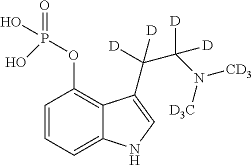 [SMILES: [2H]C([2H])([2H])N(C([2H])([2H])[2H])C([2H])([2H])C([2H])([2H])c1c[nH]c2cccc(OP(=O)(O)O)c12] |
| I-2 | 2-(4-fluoro-1H- indol-3-yl)-N,N- bis(methyl- d3)ethan-1-amine- 1,1,2,2-d4 | 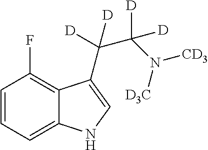 [SMILES: [2H]C([2H])([2H])N(C([2H])([2H])[2H])C([2H])([2H])C([2H])([2H])c1c[nH]c2cccc(F)c12] |
| I-3 | 2-(4-methoxy-1H- indol-3-yl)-N,N- bis(methyl- d3)ethan-1-amine- 1,1,2,2-d4 | 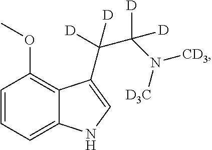 [SMILES: [2H]C([2H])([2H])N(C([2H])([2H])[2H])C([2H])([2H])C([2H])([2H])c1c[nH]c2cccc(OC)c12] |
| | | or |
| I-4 | 3-(2-(bis(methyl- d3)amino)ethyl- 1,1,2,2-d4)-1H- indol-4-ol | 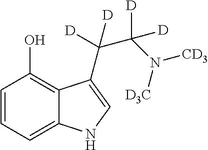 [SMILES: [2H]C([2H])([2H])N(C([2H])([2H])[2H])C([2H])([2H])C([2H])([2H])c1c[nH]c2cccc(O)c12] |

or a pharmaceutically acceptable salt, prodrug and/or solvate thereof.

In some embodiments, the one or more hallucinogens, or a pharmaceutically acceptable salt, prodrug and/or solvate thereof, are selected from the compound disclosed and claimed in Applicant's co-pending PCT patent application serial number PCT/CA2021/050122 filed on Feb. 4, 2021, the contents of which are incorporated by reference in their entirety. Therefore in some embodiments, the one or more hallucinogens are selected from a compound of Formula (II) or a pharmaceutically acceptable salt, prodrug and/or solvate thereof:

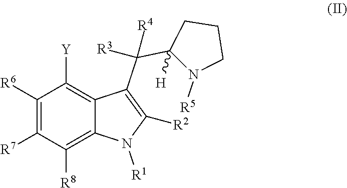

or a pharmaceutically acceptable salt, prodrug and/or solvate thereof, wherein:
R¹ is selected from hydrogen, C₁-C₃alkyl, C₁-C₆alkyleneP(O)(OR⁹)₂, C(O)R⁹, CO₂R⁹, C(O)N(R⁹)₂, S(O)R⁹ and SO₂R⁹;
R², R³ and R⁴ are independently selected from hydrogen and C₁-C₆alkyl;
R⁵ is selected from hydrogen and C₁-C₆alkyl; R⁶, R⁷ and R⁸ are independently selected from hydrogen, halogen, CN, OR⁹, N(R⁹)₂, SR⁹, C₁-C₆alkyl, C₁-C₆haloalkyl, C₂-C₆haloalkenyl, CO₂R⁹, C(O)N(R⁹)₂, S(O)R⁹, SO₂R⁹, C₂-C₆alkenyl, C₂-C₆alkynyl, C₂-C₆haloalkynyl, C₃-C₇cycloalkyl and a 3- to 7-membered heterocyclic ring including 1 to 2 ring heteromoeities selected from O, S, S(O), SO₂, N and NR⁹, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR⁹, N(R⁹)₂ and SR⁹, and wherein said C₃-C₇cycloalkyl and 3- to 7-membered heterocyclic ring are each further optionally substituted with a substituent selected from halogen, CO₂R⁹, C(O)N(R⁹)₂, SO₂R⁹, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoeities selected from O, S, S(O), SO₂, N, and NR⁹,
Y is selected from halogen and Q-A;
Q is selected from O, NR¹⁰, S, S(O) and SO₂;
wherein each R⁹ and R¹⁰ are independently selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl; and
A is selected from hydrogen, C₁-C₆alkyl, C₂-C₆alkenyl, C₂-C₆alkynyl, C₃-C₇cycloalkyl, C₄-C₆cycloalkenyl, heterocycloalkyl, aryl, heteroaryl, P(O)(OR¹¹)₂, C₁-C₆alkyleneP(O)(OR¹¹)₂, C₁-C₆alkyleneC₃-C₇cycloalkyl, C₁-C₆alkyleneC₄-C₆cycloalkenyl, C₁-C₆alkyleneheterocycloalkyl, C₁-C₆alkylenearyl, C₁-C₆alkyleneheteroaryl, C(O) Q′, CO₂Q′, C(O)N(Q′)₂, S(O) Q′, and SO₂Q′,
wherein Q′ is selected from hydrogen, C₁-C₂₀alkyl, C₁-C₂₀haloalkyl, C₂-C₂₀alkenyl, C₂-C₂₀haloalkenyl, C₂-C₂₀alkynyl, C₂-C₂₀haloalkynyl, C₃-C₇cycloalkyl, C₄-C₇cycloalkenyl and a 3- to 7-membered heterocyclic ring including 1 to 2 ring heteromoeities selected from O, S, S(O), SO₂, N and NR¹⁰, wherein said C₁-C₂₀alkyl, C₂-C₂₀haloalkyl, C₂-C₆alkenyl, C₂-C₂₀haloalkenyl, C₂-C₂₀alkynyl, C₂-C₂₀haloalkynyl, C₃-C₇cycloalkyl, C₄-C₇cycloalkenyl and 3- to 7-membered heterocyclic ring groups are optionally substituted by one or more substituents independently selected from CN, OR¹⁰, N(R¹⁰)₂, CO₂R¹⁰ and SR¹⁰, and/or are disubstituted on the same carbon atom with C₁₋₆alkyl, or with C₂-C₆alkylene to form a C₃-C₇cycloalkyl ring, and wherein each of said C₃-C₇cycloalkyl, C₄-C₇cycloalkenyl and 3- to 7-membered heterocyclic ring are further optionally substituted with a substituent selected from C₁-C₃alkyl and C₁-C₆haloalkyl; and
each R¹¹ is independently selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl;
wherein all available hydrogen atoms are optionally substituted with a halogen atom and/or all available atoms are optionally substituted with an alternate isotope thereof.

In some embodiments, the compound of Formula II is:

| Compound | | |
|---|---|---|
| ID # | IUPAC Name | Chemical Structure |
| II-1 | (R)-3-((1- methylpyrrolidin- 2-yl)methyl)- 1H-indol-4-yl dihydrogen phosphate | 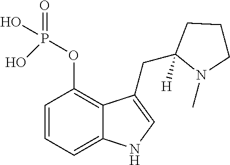 [SMILES: CN1CCC[C@@H]1Cc1c[nH]c2cccc(OP(=O)(O)O)c12] |
| II-2 | (R)-3-((1- (methyl- d3)pyrrolidin-2- yl)methyl)-1H- indol-4-ol | 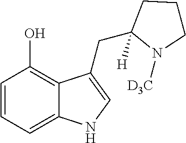 [SMILES: [2H]C([2H])([2H])N1CCC[C@@H]1Cc1c[nH]c2cccc(O)c12] |
| II-3 | ((R)-3- (pyrrolidin-2- ylmethyl)-1H- indol-4-ol | 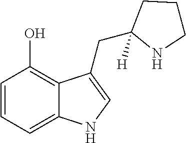 [SMILES: Oc1cccc2[nH]cc(C[C@H]3CCCN3)c12] |
| II-4 | (S)-3-((1- methylpyrrolidin- 2-yl)methyl)- 1H-indol-4-yl dihydrogen phosphate | 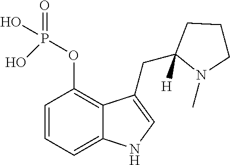 [SMILES: CN1CCC[C@H]1Cc1c[nH]c2cccc(OP(=O)(O)O)c12] |
| II-5 | (R)-3- (pyrrolidin-2- ylmethyl)-1H- indol-4-yl dihydrogen phosphate | 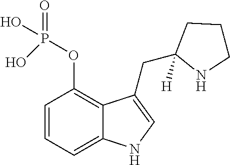 [SMILES: O=P(O)(O)Oc1cccc2[nH]cc(C[C@H]3CCCN3)c12] |
| II-6 | (S)-3- (pyrrolidin-2- ylmethyl)-1H- indol-4-yl dihydrogen phosphate | 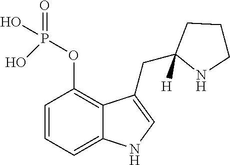 [SMILES: O=P(O)(O)Oc1cccc2[nH]cc(C[C@@H]3CCCN3)c12] |
| II-7 | (R)-3-((1- (methyl- d3)pyrrolidin-2- yl)methyl)-1H- indol-4-yl dihydrogen phosphate | 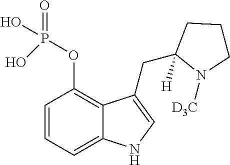 [SMILES: [2H]C([2H])([2H])N1CCC[C@@H]1Cc1c[nH]c2cccc(OP(=O)(O)O)c12] |
| II-8 | (S)-3-((1- (methyl- d3)pyrrolidin-2- yl)methyl)-1H- indol-4-yl dihydrogen phosphate | 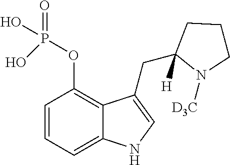 [SMILES: [2H]C([2H])([2H])N1CCC[C@H]1Cc1c[nH]c2cccc(OP(=O)(O)O)c12] |
| II-9 | (S)-3-((1- (methyl- d3)pyrrolidin-2- yl)methyl-d2)- 1H-indol-4-yl dihydrogen phosphate | 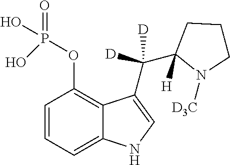 |
| II-10 | (R)-(((3-((1- methylpyrrolidin- 2-yl)methyl)- 1H-indol-4- yl)oxy)methyl) phosphonic acid | 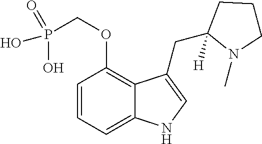 [SMILES: CN1CCC[C@@H]1Cc1c[nH]c2cccc(OCP(=O)(O)O)c12] |
| II-11 | (R)-(((3-((1- (methyl- d3)pyrrolidin-2- yl)methyl)-1H- indol-4- yl)oxy)methyl) phosphonic acid | 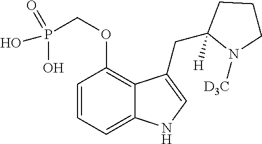 [SMILES: [2H]C([2H])([2H])N1CCC[C@@H]1Cc1c[nH]c2cccc(OCP(=O)(O)O)c12] |
| II-12 | (R)-((4- hydroxy-3-((1- methylpyrrolidin- 2-yl)methyl)- 1H-indol-1- yl)methyl) phosphonic acid | 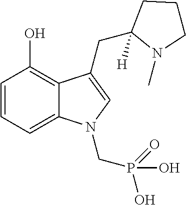 [SMILES: CN1CCC[C@@H]1Cc1cn(CP(=O)(O)O)c2cccc(O)c12] |
| II-13 | (R)-((4- hydroxy-3-((1- (methyl- d3)pyrrolidin-2- yl)methyl)-1H- indol-1- yl)methyl) phosphonic acid | 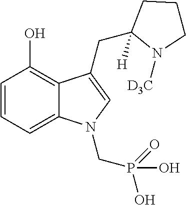 [SMILES: [2H]C([2H])([2H])N1CCC[C@@H]1Cc1cn(CP(=O)(O)O)c2cccc(O)c12] |
| II-14 | (R)-((4- hydroxy-3- (pyrrolidin-2- ylmethyl)-1H- indol-1- yl)methyl)- phosphonic acid | 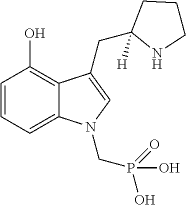 [SMILES: O=P(O)(O)Cn1cc(C[C@H]2CCCN2)c2c(O)cccc21] |
| II-15 | (R)-((3-((1- methylpyrrolidin- 2-yl)methyl)- 4- (phosphonooxy)- 1H-indol-1- yl)methyl) phosphonic acid | 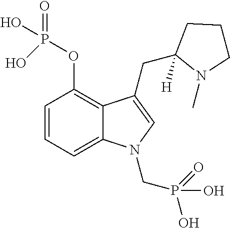 [SMILES: CN1CCC[C@@H]1Cc1cn(CP(=O)(O)O)c2cccc(OP(=O)(O)O)c12] |
| II-16 | (1-((3-(((R)-1- methylpyrrolidin- 2-yl)methyl)- 1H-indol-4- yl)oxy)ethyl) phosphonic acid | 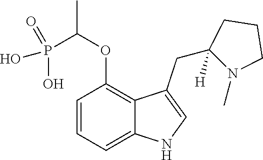 [SMILES: CC(Oc1cccc2[nH]cc(C[C@H]3CCCN3C)c12)P(=O)(O)O] |
| II-17 | (1-((3-(((R)- pyrrolidin-2- yl)methyl)-1H- indol-4- yl)oxy)ethyl) phosphonic acid | 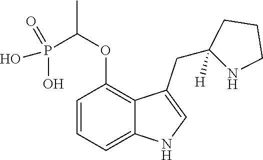 [SMILES: CC(Oc1cccc2[nH]cc(C[C@H]3CCCN3)c12)P(=O)(O)O] |
| II-18 | (R)-3-((1- (methyl- d3)pyrrolidin-2- yl)methyl)-1H- indol-4-yl glycinate | 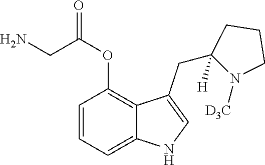 [SMILES: [2H]C([2H])([2H])N1CCC[C@@H]1Cc1c[nH]c2cccc(OC(=O)CN)c12] |
| II-19 | 3-(((R)-1- (methyl- d3)pyrrolidin-2- yl)methyl)-1H- indol-4-yl D- alaninate | 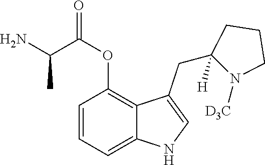 [SMILES: [2H]C([2H])([2H])N1CCC[C@@H]1Cc1c[nH]c2cccc(OC(=O)[C@@H](C)N)c12] |
| II-20 | (R,Z)-4-((3-((1- (methyl- d3)pyrrolidin-2- yl)methyl)-1H- indol-4-yl)oxy)- 4-oxobut-2- enoic acid | 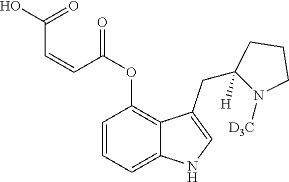 [SMILES: [2H]C([2H])([2H])N1CCC[C@@H]1Cc1c[nH]c2cccc(OC(=O)/C=C\C(=O)O)c12] |
| II-21 | (R,E)-4-((3-((1- (methyl- d3)pyrrolidin-2- yl)methyl)-1H- indol-4-yl)oxy)- 4-oxobut-2- enoic acid | 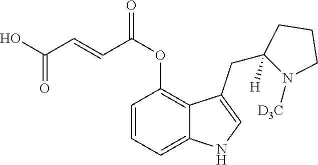 [SMILES: [2H]C([2H])([2H])N1CCC[C@@H]1Cc1c[nH]c2cccc(OC(=O)/C=C/C(=O)O)c12] |
| II-22 | (R)-4-((3-((1- (methyl- d3)pyrrolidin-2- yl)methyl)-1H- indol-4-yl)oxy)- 4-oxobutanoic acid | 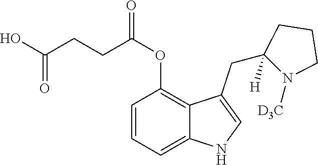 [SMILES: [2H]C([2H])([2H])N1CCC[C@@H]1Cc1c[nH]c2cccc(OC(=O)CCC(=O)O)c12] |
| II-23 | (R)-3-((1- (methyl- d3)pyrrolidin-2- yl)methyl)-1H- indol-4-yl acetate | 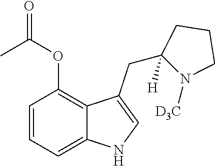 [SMILES: [2H]C([2H])([2H])N1CCC[C@@H]1Cc1c[nH]c2cccc(OC(C)=O)c12] |
| II-24 | (R)-3-((1- (methyl- d3)pyrrolidin-2- yl)methyl-d2)- 1H-indol-4-yl acetate | 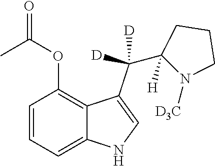 |
| II-25 | (R)-3-((1- methylpyrrolidin- 2-yl)methyl)- 1H-indol-4-yl acetate | 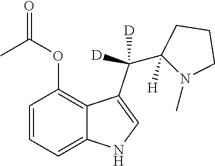 |
| II-26 | (R)-((4- acetoxy-3-((1- methylpyrrolidin- 2-yl)methyl)- 1H-indol-1- yl)methyl) phosphonic acid | 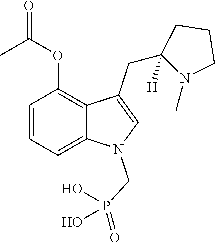 [SMILES: CC(=O)Oc1cccc2c1c(C[C@H]1CCCN1C)cn2CP(=O)(O)O] |
| II-27 | 3-(((R)-1- methylpyrrolidin- 2-yl)methyl)- 1H-indol-4-yl (9Z, 12Z)- octadeca-9,12- dienoate | 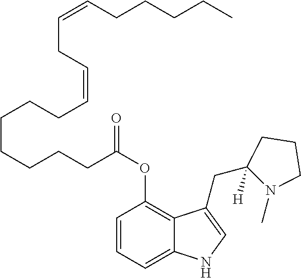 [SMILES: CCCCC/C=C\C/C=C\CCCCCCCC(=O)Oc1cccc2[nH]cc(C[C@H]3CCCN3C)c12] |
| II-28 | 3-(((R)-1- (methyl- d3)pyrrolidin-2- yl)methyl)-1H- indol-4-yl (9Z, 12Z)- octadeca-9,12- dienoate- 11,11-d2 | 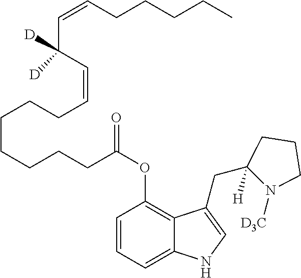 |
| II-29 | 3-(((R)-1- (methyl- d3)pyrrolidin-2- yl)methyl-d2)- 1H-indol-4-yl (9Z, 12Z)- octadeca-9,12- dienoate- 11,11-d2 | 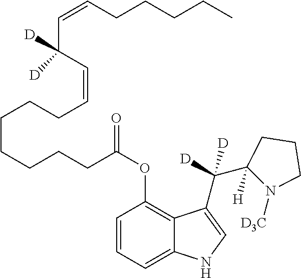 |
| II-30 | 3-(((R)-1- methylpyrrolidin- 2-yl)methyl)- 1H-indol-4-yl (9Z, 12Z)- octadeca-9,12- dienoate- 11,11-d2 | 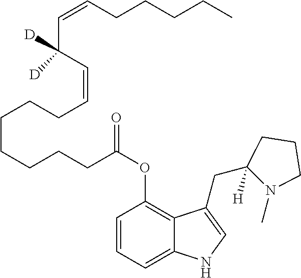 |
| II-31 | 3-(((R)-1- methylpyrrolidin- 2-yl)methyl)- 1H-indol-4-yl (S)-3- (aminomethyl)- 5- methylhexanoate | 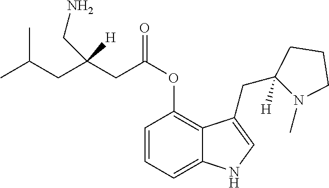 [SMILES: CC(C)C[C@H](CN)CC(=O)Oc1cccc2[nH]cc(C[C@H]3CCCN3C)c12] |
| II-32 | 3-(((R)-1- (methyl- d3)pyrrolidin-2- yl)methyl)-1H- indol-4-yl (S)-3- (aminomethyl)- 5- methylhexanoate | 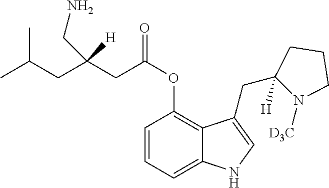 [SMILES: [2H]C([2H])([2H])N1CCC[C@@H]1Cc1c[nH]c2cccc(OC(=O)C[C@@H](CN)CC(C)C)c12] |
| II-33 | (R)-3-((1- methylpyrrolidin- 2-yl)methyl)- 1H-indol-4-yl 2- (1- (aminomethyl)- cyclohexyl) acetate | 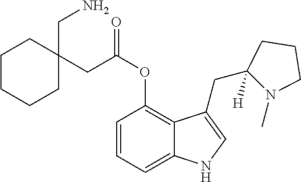 [SMILES: CN1CCC[C@@H]1Cc1c[nH]c2cccc(OC(=O)CC3(CN)CCCCC3)c12] |
| II-34 | R)-3-((1- (methyl- d3)pyrrolidin-2- yl)methyl)-1H- indol-4-yl 2-(1- (aminomethyl) cyclohexyl)- acetate | 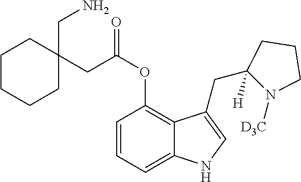 [SMILES: [2H]C([2H])([2H])N1CCC[C@@H]1Cc1c[nH]c2cccc(OC(=O)CC3(CN)CCCCC3)c12] |
| II-35 | (R)-3-((1- methylpyrrolidi n-2-yl)methyl)- 1H-indol-4-yl [1,4′- bipiperidine]-1′- carboxylate | 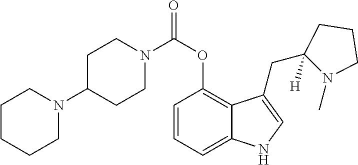 [SMILES: CN1CCC[C@@H]1Cc1c[nH]c2cccc(OC(=O)N3CCC(N4CCCCC4)CC3)c12] |
| II-36 | (R)-3-((1- (methyl- d3)pyrrolidin-2- yl)methyl)-1H- indol-4-yl dimethylcarbamate | 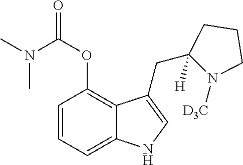 [SMILES: [2H]C([2H])([2H])N1CCC[C@@H]1Cc1c[nH]c2cccc(OC(=O)N(C)C)c12] |
| II-37 | (R)-4-fluoro-3- ((1-(methyl- d3)pyrrolidin- 2-yl)methyl- d2)-1H-indole | 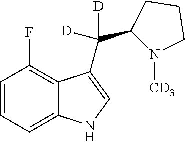 [SMILES: [2H]C([2H])(c1c[nH]c2cccc(F)c12)[C@H]1CCCN1C([2H])([2H])[2H]] |
| II-38 | (R)-4-chloro- 3-((1-(methyl- d₃)pyrrolidin- 2-yl)methyl- d₂)-1H-indole | 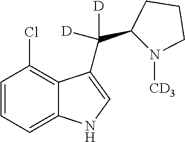 [SMILES: [2H]C([2H])(c1c[nH]c2cccc(Cl)c12)[C@H]1CCCN1C([2H])([2H])[2H]] |
| II-39 | (R)-4- methoxy-3- ((1-(methyl- d₃)pyrrolidin- 2-yl)methyl- d₂)-1H-indole | 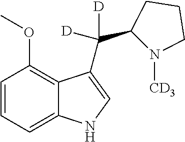 [SMILES: [2H]C([2H])(c1c[nH]c2cccc(OC)c12)[C@H]1CCCN1C([2H])([2H])[2H]] |
| II-40 | (R)-4-fluoro-3- ((1-(methyl- d₃)pyrrolidin-2- yl)methyl)-1H- indole | 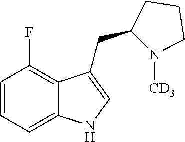 [SMILES: [2H]C([2H])([2H])N1CCC[C@@H]1Cc1c[nH]c2cccc(F)c12] |
| II-41 | (R)-4-fluoro-3- (pyrrolidin-2- ylmethyl-d₂)- 1H-indole | 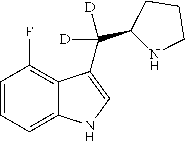 [SMILES: [2H]C([2H])(c1c[nH]c2cccc(F)c12)[C@H]1CCCN1] |
| II-42 | (S)-4-fluoro-3- ((1-(methyl- d₃)pyrrolidin-2- yl)methyl-d₂)- 1H-indole | 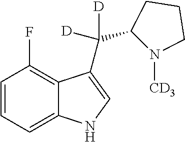 [SMILES: [2H]C([2H])(c1c[nH]c2cccc(F)c12)[C@@H]1CCCN1C([2H])([2H])[2H]] |
| II-43 | (R)-4- (benzyloxy)-3- ((1-(methyl- d₃)pyrrolidin-2- yl)methyl-d₂)- 1H-indole | 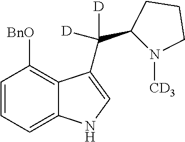 [SMILES: [2H]C([2H])(c1c[nH]c2cccc(OCc3ccccc3)c12)[C@H]1CCCN1C([2H])([2H])[2H]] |
| II-44 | (R)-3-((1- (methyl- d₃)pyrrolidin-2- yl)methyl-d₂)- 1H-indol-4-ol | 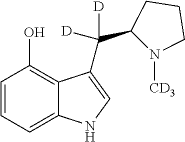 [SMILES: [2H]C([2H])(c1c[nH]c2cccc(O)c12)[C@H]1CCCN1C([2H])([2H])[2H]] |
| II-45 | (R)-4- (benzyloxy)-3- ((1-(methyl- d₃)pyrrolidin-2- yl)methyl)-1H- indole | 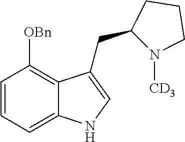 [SMILES: [2H]C([2H])([2H])N1CCC[C@@H]1Cc1c[nH]c2cccc(OCc3ccccc3)c12] |
| II-46 | R)-4- (benzyloxy)-3- ((1- methylpyrrolidin- 2-yl)methyl)- 1H-indole | 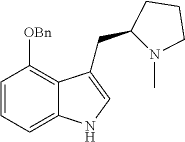 [SMILES: CN1CCC[C@@H]1Cc1c[nH]c2cccc(OCc3ccccc3)c12] |
| II-47 | (R)-3-((1- methylpyrrolidin- 2-yl)methyl)- 1H-indol-4-ol | 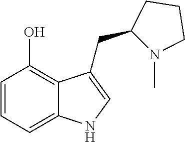 [SMILES: CN1CCC[C@@H]1Cc1c[nH]c2cccc(O)c12] |
| II-48 | 3-(((R)-1- (methyl- d3)pyrrolidin-2- yl)methyl)-1H- indol-4-yl (9Z,12Z)- octadeca-9, 12- dienoate | 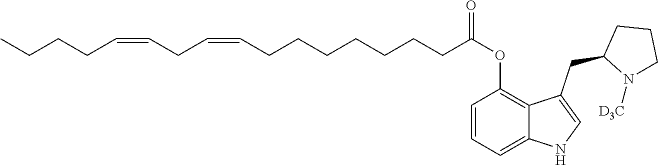 [SMILES: [2H]C([2H])([2H])N1CCC[C@@H]1Cc1c[nH]c2cccc(OC(=O)CCCCCCC/C=C\C/C=C\CCCC)c12] |
| II-49 | 3-(((R)-1- (methyl- d3)pyrrolidin-2- yl)methyl-d2)- 1H-indol-4-yl (9Z, 12Z)- octadeca-9, 12- dienoate | 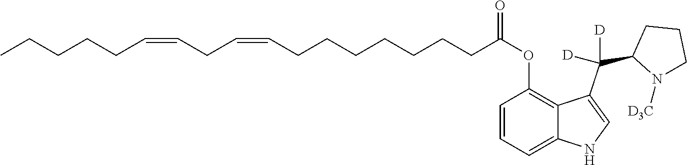 [SMILES: [2H]C([2H])(c1c[nH]c2cccc(OC(=O)CCCCCCC/C=C\C/C=C\CCCCC)c12)[C@H]1CCCN1C([2H])([2H])[2H]] |
| II-50 | (R)-3- (pyrrolidin-2- ylmethyl-d₂)- 1H-indol-4-ol | 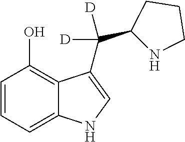 [SMILES: [2H]C([2H])(c1c[nH]c2cccc(O)c12)[C@H]1CCCN1] |
| II-51 | (S)-3- (pyrrolidin-2- ylmethyl-d₂)- 1H-indol-4-ol | 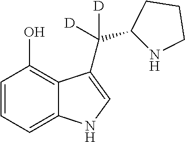 [SMILES: [2H]C([2H])(c1c[nH]c2cccc(O)c12)[C@@H]1CCCN1] |
| II-52 | (S)-4- (benzyloxy)-3- ((1-(methyl- d₃)pyrrolidin-2- yl)methyl)-1H- indole | 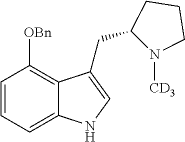 [SMILES: [2H]C([2H])([2H])N1CCC[C@H]1Cc1c[nH]c2cccc(OCc3ccccc3)c12] |
| II-53 | (S)-3-((1- (methyl- d₃)pyrrolidin-2- yl)methyl)-1H- indol-4-ol | 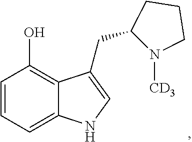 [SMILES: [2H]C([2H])([2H])N1CCC[C@H]1Cc1c[nH]c2cccc(O)c12] |
| | | or |
| II-54 | (R)-3-((1- (methyl- d₃)pyrrolidin-2- yl)methyl-d2)- 1H-indol-4-yl dihydrogen phosphate | 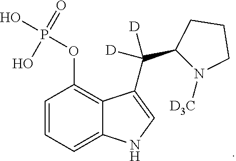 [SMILES: [2H]C([2H])(c1c[nH]c2cccc(OP(=O)(O)O)c12)[C@H]1CCCN1C([2H])([2H])[2H]] |

or a pharmaceutically acceptable salt, prodrug and/or solvate thereof.

In some embodiments, the one or more hallucinogens, or a pharmaceutically acceptable salt, prodrug and/or solvate thereof, are selected from the compound disclosed and claimed in Applicant's co-pending PCT patent application serial number PCT/CA2021/050125 filed on Feb. 4, 2021, the contents of which are incorporated by reference in their entirety. Therefore in some embodiments, the one or more hallucinogens are selected from a compound of Formula (III) or a pharmaceutically acceptable salt, prodrug and/or solvate thereof:

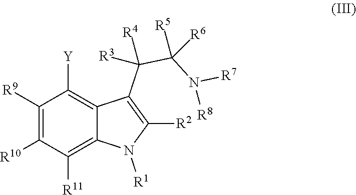

wherein R¹ is selected from hydrogen, C₁-C₃alkyl, C₁-C₆alkyleneP(O)(OR¹²)₂, C(O)R¹², CO₂R¹², C(O)N(R¹²)₂, S(O)R¹² and SO₂R¹²;
R² to R⁶ are independently selected from hydrogen and C₁-C₆alkyl;
R⁷ and R⁸ are independently selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl and substituted or unsubstituted heteroaryl, or
R⁷ and R⁸ are taken together with the nitrogen atom therebetween to form a 3- to 7-membered heterocyclic ring optionally including 1 to 2 additional ring heteromoieties selected from O, S, S(O), SO₂, N and NR¹³,
wherein said C₃-C₇cycloalkyl and 3- to 7-membered heterocyclic ring are each further optionally substituted with a substituent selected from halogen, CO₂R¹³, C(O)N(R¹³)₂, SO₂R¹³, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, N, S(O), SO₂ and NR¹³; R⁹, R¹⁰, and R¹¹ are independently selected from hydrogen, halogen, CN, OR¹³, N(R¹³)₂, SR¹³, C₁-C₆alkyl, C₁-C₆haloalkyl, C₂-C₆haloalkenyl, CO₂R¹³, C(O)N(R¹³)₂, SOR¹³, SO₂R¹³, C₂-C₆alkenyl, C₂-C₆alkynyl, C₂-C₆haloalkynyl, C₃-C₇cycloalkyl and a 3- to 7-membered heterocyclic ring including 1 to 2 ring heteromoieties selected from O, S, S(O), SO₂, N and NR¹³, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR¹³, N(R¹³)₂ and SR¹³, and wherein said C₃-C₇cycloalkyl and 3- to 7-membered heterocyclic ring are each further optionally substituted with a substituent selected from halogen, CO₂R¹³, C(O)N(R¹³)₂, SO₂R¹³, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, S(O), SO₂, N and NR¹³;
Y is selected from halogen and X-A;
X is selected from O, NR¹³, S, S(O) and SO₂;
A is selected from hydrogen, C₁-C₆alkyl, C₂-C₆alkenyl, C₂-C₆alkynyl, C₃-C₇cycloalkyl, C₄-C₆cycloalkenyl, heterocycloalkyl, aryl, heteroaryl, P(O)(OR¹²)₂, C₁-C₆alkyleneP(O)(OR¹²)₂, C₁-C₆alkyleneC₃-C₇cycloalkyl, C₁-C₆alkyleneC₄-C₆cycloalkenyl, C₁-C₆alkyleneheterocycloalkyl, C₁-C₆alkylenearyl, C₁-C₆alkyleneheteroaryl, C(O) Q′, CO₂Q′, C(O)N(Q′)₂, S(O) Q′ and SO₂Q′,
wherein Q′ is selected from C₁-C₂₀alkyl, C₁-C₂₀haloalkyl, C₂-C₂₀alkenyl, C₂-C₂₀haloalkenyl, C₂-C₂₀alkynyl, C₂-C₂₀haloalkynyl, C₃-C₇cycloalkyl, C₄-C₇cycloalkenyl and a 3- to 7-membered heterocyclic ring including 1 to 2 ring heteromoieties selected from O, S, S(O), SO₂, N and NR¹³, wherein said C₁-C₂₀alkyl, C₂-C₂₀haloalkyl, C₂-C₂₀alkenyl, C₂-C₂₀haloalkenyl, C₃-C₇cycloalkyl, C₄-C₇cycloalkenyl and 3- to 7-membered heterocyclic ring groups are optionally substituted by one or more substituents independently selected from CN, OR¹³, N(R¹³)₂, CO₂R¹³, SR¹³, C₃-C₇cycloalkyl, C₄-C₇cycloalkenyl and a 3- to 7-membered heterocyclic ring, and/or are disubstituted on the same carbon atom with C₁₋₆alkyl, or with C₂₋₆alkylene to form a C₃-C₇cycloalkyl ring, and wherein each of said C₃-C₇cycloalkyl, C₄-C₇cycloalkenyl, and 3- to 7-membered heterocyclic ring are each further optionally substituted with a substituent selected from of C₁-C₆alkyl and C₁-C₆haloalkyl;
each R¹² is independently selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl;
each R¹³ is independently selected from hydrogen, C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl, and a 3- to 7-membered heterocyclic ring including 1 to 2 ring heteromoieties selected from O, S, S(O), SO₂, N and NR¹⁴, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR¹⁴, N(R¹⁴)₂ and SR¹⁴, and wherein said C₃-C₇cycloalkyl and 3- to 7-membered heterocyclic ring are each further optionally substituted with a substituent selected from halogen, CO₂R¹⁴, C(O)N(R¹⁴)₂, SO₂R¹⁴, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, S(O), SO₂, N and NR¹⁴
R¹⁴ is selected from hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted heterocycloalkyl, substituted or unsubstituted aryl and substituted or unsubstituted heteroaryl; and
wherein all available hydrogen atoms are optionally substituted with a halogen atom and/or all available atoms are optionally substituted with an alternate isotope thereof,
provided either R¹ is C₁-C₆P(O)(OR¹²)₂ and R², R³, R⁴, R⁵, R⁶, R⁷ R⁸, R⁹, R¹⁰, R¹¹, R¹², R¹³ and R¹⁴, Q′, X, Y and A are as defined above for Formula (I); or
Y is X-A wherein A is selected from C₁-C₆alkyleneP(O)(OR¹²)₂, C₁-C₆alkyleneC₃-C₇cycloalkyl, C₁-C₆alkyleneC₄-C₆cycloalkenyl, C₁-C₆alkyleneheterocycloalkyl, C₁-C₇alkylenearyl, C₁-C₆alkyleneheteroaryl, C(O) Q′, CO₂Q′, C(O)N(Q′)₂, S(O) Q′ and SO₂Q′ and R¹, R², R³, R⁴, R⁵, R⁶, R⁷ R⁸, R⁹, R¹⁰, R¹¹, R¹², R¹³ and R¹⁴, Q′ and X are as defined above for Formula (III).

In some embodiments, the compound of Formula III is:

| Compound | | |
|---|---|---|
| ID # | IUPAC Name | Chemical Structure |
| III-1 | ((3-(2- (dimethylamino)ethyl)- 4-(phosphonooxy)-1H- indol-1- yl)methyl)phosphonic acid | 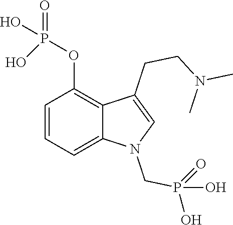 [SMILES: CN(C)CCc1cn(CP(=O)(O)O)c2cccc(OP(=O)(O)O)c12] |
| III-2 | 3-(2- (dimethylamino)ethyl)- 4-hydroxy-1H-indol-1- yl)methyl)phosphonic acid | 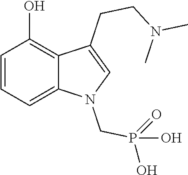 [SMILES: CN(C)CCc1cn(CP(=O)(O)O)c2cccc(O)c12] |
| III-3 | ((3-(2-(bis(methyl- d3)amino)ethyl)-4- (phosphonooxy)-1H- indol-1- yl)methyl)phosphonic acid | 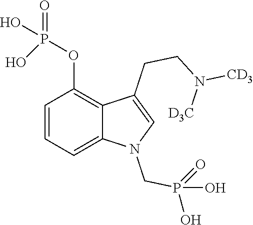 [SMILES: [2H]C([2H])([2H])N(CCc1cn(CP(=O)(O)O)c2cccc(OP(=O)(O)O)c12)C([2H])([2H])[2H]] |
| III-4 | (1-((3-(2- (dimethylamino)ethyl)- 1H-indol-4- yl)oxy)ethyl) phosphonic acid | 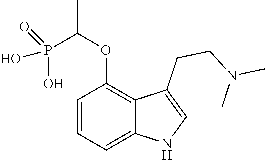 [SMILES: CC(Oc1cccc2[nH]cc(CCN(C)C)c12)P(=O)(O)O] |
| III-5 | (1-((3-(2-(bis(methyl- d3)amino)ethyl)-1H- indol-4- yl)oxy)ethyl) phosphonic acid | 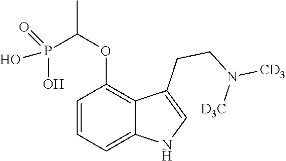 [SMILES: [2H]C([2H])([2H])N(CCc1c[nH]c2cccc(OC(C)P(=O)(O)O)c12)C([2H])([2H])[2H]] |
| III-6 | 3-(2-(bis(methyl- d3)amino)ethyl)-1H- indol-4-yl glycinate | 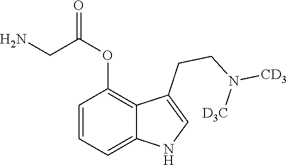 [SMILES: [2H]C([2H])([2H])N(CCc1c[nH]c2cccc(OC(=O)CN)c12)C([2H])([2H])[2H]] |
| III-7 | 3-(2-(bis(methyl- d3)amino)ethyl)-1H- indol-4-yl D-alaninate | 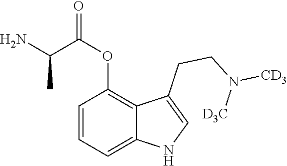 [SMILES: [2H]C([2H])([2H])N(CCc1c[nH]c2cccc(OC(=O)[C@@H](C)N)c12)C([2H])([2H])[2H]] |
| III-8 | (Z)-4-((3-(2- (bis(methyl- d3)amino)ethyl)-1H- indol-4-yl)oxy)-4- oxobut-2-enoic acid | 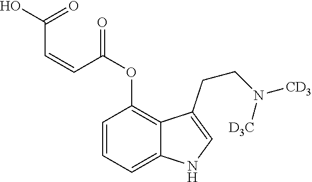 [SMILES: [2H]C([2H])([2H])N(CCc1c[nH]c2cccc(OC(=O)/C=C\C(=O)O)c12)C([2H])([2H])[2H]] |
| III-9 | (E)-4-((3-(2- (bis(methyl- d3)amino)ethyl)-1H- indol-4-yl)oxy)-4- oxobut-2-enoic acid | 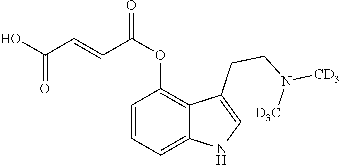 [SMILES: [2H]C([2H])([2H])N(CCc1c[nH]c2cccc(OC(=O)/C=C/C(=O)O)c12)C([2H])([2H])[2H]] |
| III-10 | 4-((3-(2-(bis(methyl- d3)amino)ethyl)-1H- indol-4-yl)oxy)-4- oxobutanoic acid | 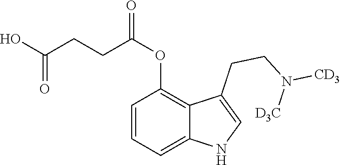 [SMILES: [2H]C([2H])([2H])N(CCc1c[nH]c2cccc(OC(=O)CCC(=O)O)c12)C([2H])([2H])[2H]] |
| III-11 | 3-(2-(bis(methyl- d3)amino)ethyl)-1H- indol-4-yl acetate | 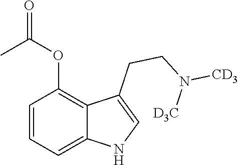 [SMILES: [2H]C([2H])([2H])N(CCc1c[nH]c2cccc(OC(C)=O)c12)C([2H])([2H])[2H]] |
| III-12 | 3-(2-(bis(methyl- d3)amino)ethyl- 1,1,2,2-d4)-1H-indol- 4-yl acetate | 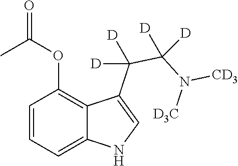 [SMILES: [2H]C([2H])([2H])N(C([2H])([2H])[2H])C([2H])([2H])C([2H])([2H])c1c[nH]c2cccc(OC(C)=O)c12] |
| III-13 | ((4-acetoxy-3-(2- (bis(methyl- d3)amino)ethyl)-1H- indol-1- yl)methyl)phosphonic acid | 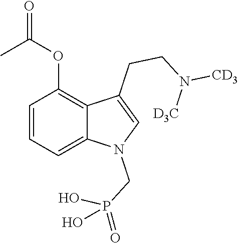 [SMILES: [2H]C([2H])([2H])N(CCc1cn(CP(=O)(O)O)c2cccc(OC(C)=O)c12)C([2H])([2H])[2H]] |
| III-14 | 3-(2- (dimethylamino)ethyl)- 1H-indol-4-yl (9Z,12Z)-octadeca- 9,12-dienoate | 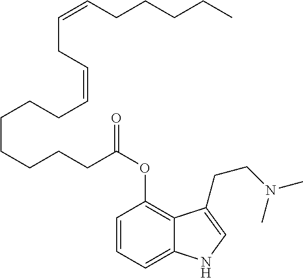 [SMILES: CCCCC/C=C\C/C=C\CCCCCCCC(=O)Oc1cccc2[nH]cc(CCN(C)C)c12] |
| III-15 | 3-(2-(d6- dimethylamino)ethyl)- 1H-indol-4-yl (9Z,12Z)-octadeca- 9,12-dienoate-11,11- d2 | 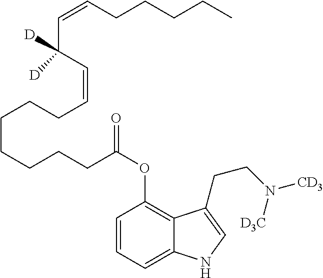 |
| III-16 | 3-(2- (dimethylamino)ethyl)- 1H-indol-4-yl (9Z,12Z)-octadeca- 9,12-dienoate-11,11- d2 | 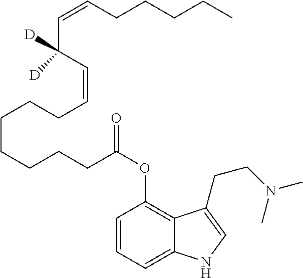 |
| III-17 | 3-(2-(bis(methyl- d3)amino)ethyl)-1H- indol-4-yl (9Z,12Z)- octadeca-9,12- dienoate | 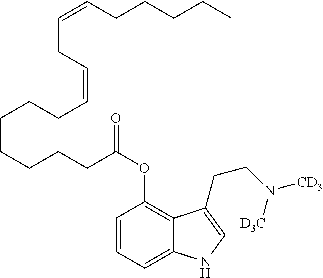 [SMILES: [2H]C([2H])([2H])N(CCc1c[nH]c2cccc(OC(=O)CCCCCCC/C=C\C/C=C\CCCCC)c12)C([2H])([2H])[2H]] |
| III-18 | 3-(2-(bis(methyl- d3)amino)ethyl- 1,1,2,2-d4)-1H-indol- 4-yl (9Z,12Z)- octadeca-9,12- dienoate | 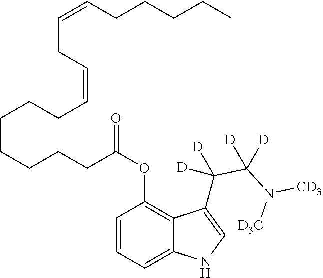 [SMILES: [2H]C([2H])([2H])N(C([2H])([2H])[2H])C([2H])([2H])C([2H])([2H])c1c[nH]c2cccc(OC(=O)CCCCCCC/C=C\C/C=C\CCCCC)c12] |
| III-19 | 3-(2-(bis(methyl- d6)amino)ethyl- 1,1,2,2-d4)-1H-indol- 4-yl (9Z,12Z)- octadeca-9,12- dienoate-11,11-d2 | 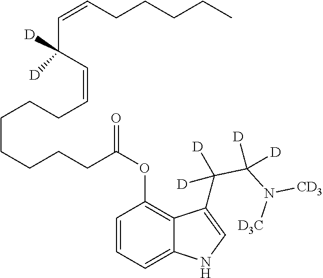 |
| III-20 | 3-(2-(d10- diethylamino)ethyl- d4)-1H-indol-4-yl (9Z,12Z)-octadeca- 9,12-dienoate | 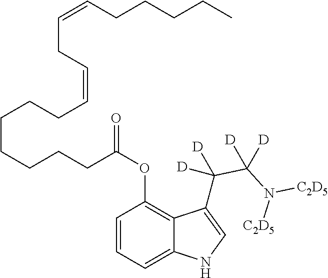 |
| III-21 | 3-(2-(d10- diethylamino)ethyl- d4)-1H-indol-4-yl (9Z,12Z)-octadeca- 9,12-dienoate-11,11- d2 | 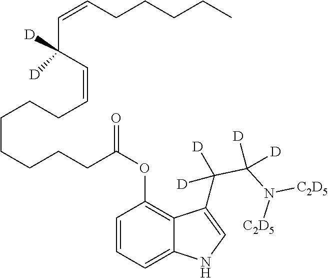 |
| III-22 | 3-(2- (diethylamino)ethyl- d4)-1H-indol-4-yl (9Z,12Z)-octadeca- 9,12-dienoate | 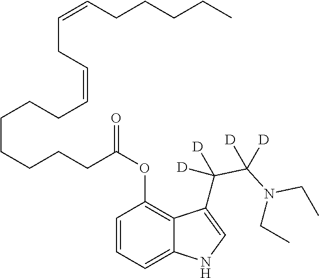 [SMILES: [2H]C([2H])(c1c[nH]c2cccc(OC(=O)CCCCCCC/C=C\C/C=C\CCCCC)c12)C([2H])([2H])N(CC)CC] |
| III-23 | 3-(2- (diethylamino)ethyl- d4)-1H-indol-4-yl (9Z,12Z)-octadeca- 9,12-dienoate-11,11- d2 | 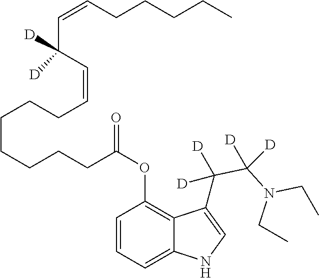 |
| III-24 | 3-(2-(pyrrolidin-1- yl)ethyl-1,1,2,2-d4)- 1H-indol-4-yl (9Z, 12Z)-octadeca- 9, 12-dienoate | 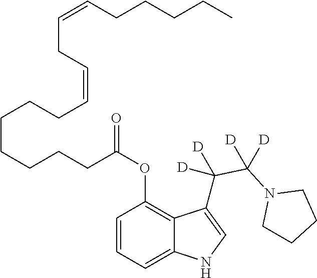 [SMILES: [2H]C([2H])(c1c[nH]c2cccc(OC(=O)CCCCCCC/C=C\C/C=C\CCCCC)c12)C([2H])([2H])N1CCCC1] |
| III-25 | 3-(2-(pyrrolidin-1- yl)ethyl-1,1,2,2-d4)- 1H-indol-4-yl (9Z,12Z)-octadeca- 9,12-dienoate-11,11- d2 | 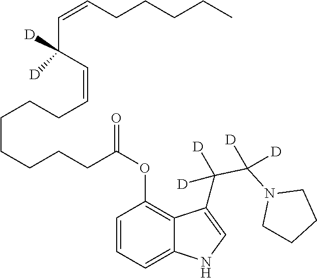 |
| III-26 | 3-(2- (dimethylamino)ethyl)- 1H-indol-4-yl (S)-3- (aminomethyl)-5- methylhexanoate | 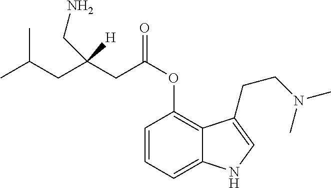 [SMILES: CC(C)C[C@H](CN)CC(=O)Oc1cccc2[nH]cc(CCN(C)C)c12] |
| III-27 | 3-(2-(bis(methyl- d3)amino)ethyl)-1H- indol-4-yl (S)-3- (aminomethyl)-5- methylhexanoate | 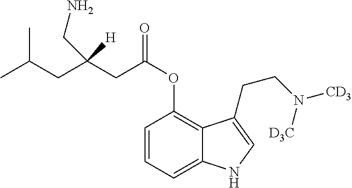 [SMILES: [2H]C([2H])([2H])N(CCc1c[nH]c2cccc(OC(=O)C[C@@H](CN)CC(C)C)c12)C([2H])([2H])[2H]] |
| III-28 | 3-(2-(bis(methyl- d3)amino)ethyl- 1,1,2,2-d4)-1H-indol- 4-yl (S)-3- (aminomethyl)-5- methylhexanoate | 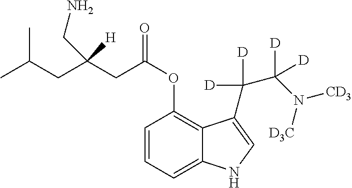 [SMILES: [2H]C([2H])([2H])N(C([2H])([2H])[2H])C([2H])([2H])C([2H])([2H])c1c[nH]c2cccc(OC(=O)C[C@@H](CN)CC(C)C)c12] |
| III-29 | 3-(2- (dimethylamino)ethyl)- 1H-indol-4-yl 2-(1- (aminomethyl) cyclohexyl)acetate | 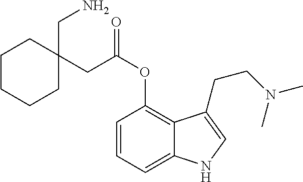 [SMILES: CN(C)CCc1c[nH]c2cccc(OC(=O)CC3(CN)CCCCC3)c12] |
| III-30 | 3-(2-(bis(methyl- d3)amino)ethyl)-1H- indol-4-yl 2-(1- (aminomethyl) cyclohexyl)acetate | 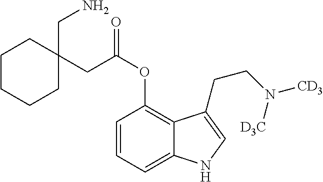 [SMILES: [2H]C([2H])([2H])N(CCc1c[nH]c2cccc(OC(=O)CC3(CN)CCCCC3)c12)C([2H])([2H])[2H]] |
| III-31 | 3-(2-(bis(methyl- d3)amino)ethyl)-1H- indol-4-yl [1,4′- bipiperidine]-1′- carboxylate | 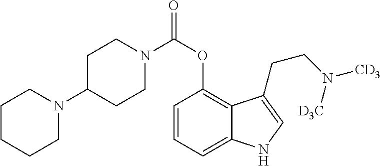 [SMILES: [2H]C([2H])([2H])N(CCc1c[nH]c2cccc(OC(=O)N3CCC(N4CCCCC4)CC3)c12)C([2H])([2H])[2H]] |
| III-32 | 3-(2-(bis(methyl- d3)amino)ethyl)-1H- indol-4-yl dimethylcarbamate | 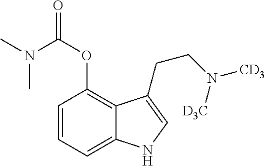 [SMILES: [2H]C([2H])([2H])N(CCc1c[nH]c2cccc(OC(=O)N(C)C)c12)C([2H])([2H])[2H]] |
| III-33 | 2-(4-(benzyloxy)-1H- indol-3-yl)-N,N- bis(methyl-d3)ethan-1- amine-1, 1,2,2-d4 | 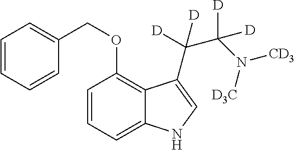 [SMILES: [2H]C([2H])([2H])N(C([2H])([2H])[2H])C([2H])([2H])C([2H])([2H])c1c[nH]c2cccc(OCc3ccccc3)c12] |
| III-34 | 2-(4-(benzyloxy)-1H- indol-3-yl)-N,N- bis(methyl-d3)ethan-1- amine | 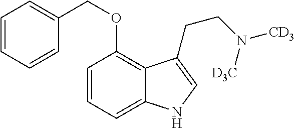 [SMILES: [2H]C([2H])([2H])N(CCc1c[nH]c2cccc(OCc3ccccc3)c12)C([2H])([2H])[2H]] |
| III-35 | dibenzyl (((1- ((bis(benzyloxy) phosphoryl) methyl)-3-(2- (bis(methyl- d3)amino)ethyl)-1H- indol-4- yl)oxy)methyl) phosphonate | 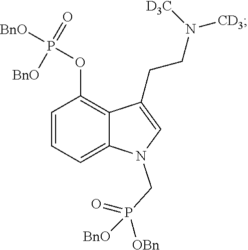 [SMILES: [2H]C([2H])([2H])N(CCc1cn(CP(=O)(OCc2ccccc2)OCc2ccccc2)c2cccc(OP(=O)(OCc3ccccc3)OCc3ccccc3)c12)C([2H])([2H])[2H]] |
| | | or |
| III-36 | 5-((3-(2- (diisopropylamino) ethyl)- 1H-indol-4-yl)oxy)-5- oxopentanoic acid | 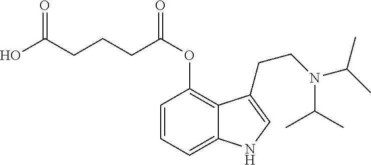 [SMILES: CC(C)N(CCc1c[nH]c2cccc(OC(=O)CCCC(=O)O)c12)C(C)C] |

or a pharmaceutically acceptable salt, prodrug and/or solvate thereof.

In some embodiments, the one or more hallucinogens, or a pharmaceutically acceptable salt, prodrug and/or solvate thereof, are selected from the compound disclosed and claimed in Applicant's co-pending U.S. provisional patent application Ser. No. 63/122,181 filed on Dec. 7, 2021, the contents of which are incorporated by reference in their entirety. Therefore in some embodiments, the one or more hallucinogens are selected from a compound of Formula (IV) or a pharmaceutically acceptable salt, prodrug and/or solvate thereof:

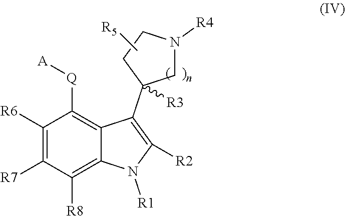

or a pharmaceutically acceptable salt thereof, wherein
R¹ is selected from the group consisting of hydrogen, C₁-C₃ alkyl, —(CH₂) P(O)(OR⁸); CO(R⁹), COO(R⁸), C(O)N(R⁸)₂, SO(R⁸) and SO₂ (R⁸);
R², R³. R⁴ and R⁵ are independently selected from the group consisting of hydrogen, deuterium and lower alkyl;
R⁶, R⁷ and R⁸ are independently selected from the group consisting of hydrogen, halogen, CN, OR⁹, N(R⁹)₂, SR⁹, C₁-C₆ alkyl, C₁-C₆ haloalkyl, C₁-C₆ alkyl substituted by OR⁹, C₁-C₆ alkyl substituted by SR⁹, C₁-C₆ alkyl substituted by N(R⁹)₂, C₂-C₆ haloalkyl, COOR⁹, C(O)N(R⁹)₂, SO₂R⁹, COOR⁹, C(O)N(R⁹)₂, SO₂R⁹, C₁-C₆ alkyl, C₂-C₆, alkenyl, C₂-C₆ haloalkenyl, C₂-C₆ alkynyl, C₂-C₆ haloalkynyl, C₃-C₇ cycloalkyl, and a 3- to 7-membered heterocyclic ring including 1 to 2 ring members selected from the group consisting of O, S, N, and N(R⁹), wherein said C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, and 3- to 7-membered heterocyclic ring groups are optionally substituted by one or more substituents independently selected from the group consisting of CN, OR⁹, N(R⁹)₂, and SR⁹, and wherein said C₃-C₇ cycloalkyl and 3- to 7-membered heterocyclic ring are each further optionally substituted with a member of the group consisting of C₁-C₃ alkyl and C₁-C₃ haloalkyl, halogen, CN, OR⁹, N(R⁹)₂, COOR⁹, C(O)N(R⁹)₂, SR⁹, SO₂R⁹, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, and a 3- to 6-membered heterocyclic ring including 1 to 2 ring members selected from the group consisting of O, S, N, and N(R⁸), wherein said C₁-C₆ alkyl, C₂-C₆ haloalkyl, C₂-C₆ alkenyl, C₂-C₆ haloalkenyl; and
Q is selected from C, O, NR¹⁰, S, SO and SO₂;
wherein R⁹ and R¹⁰ are independently selected from hydrogen, substituted or unsubstituted alkyl, substituted or unsubstituted alkenyl, substituted or unsubstituted alkynyl, substituted or unsubstituted haloalkyl, substituted or unsubstituted cycloalkyl, substituted or unsubstituted heterocycloalkyl, substituted or unsubstituted aryl, and substituted or unsubstituted heteroaryl; and
A is selected from hydrogen, alkyl, alkenyl, alkynyl, cycloalkyl, cycloalkenyl, heterocycloalkyl, heterocycloalkenyl, heterocycloalkynyl, heterocycloalkynyl aryl, heteroaryl, C₀-C₁ P(O)(OR⁹)₂, CO(Q′), COO(Q′), C(O)N(Q′)₂, SO(Q′), SO₂ (Q′), wherein Q′ is selected from hydrogen, C₁-C₂₀ alkyl, C₁-C₂₀ haloalkyl, C₂-C₂₀ alkenyl, C₂-C₂₀ haloalkenyl, C₂-C₂₀ alkynyl, C₂-C₂₀ haloalkynyl, C₃-C₇ cycloalkyl, and a 3- to 7-membered heterocyclic ring including 1 to 2 ring members selected from the group consisting of O, S, N, and N(R¹⁰), wherein said C₁-C₂₀ alkyl, C₂-C₂₀ haloalkyl, C₂-C₆ alkenyl, C₂-C₂₀ haloalkenyl, C₃-C₇ cycloalkyl, and 3- to 7-membered heterocyclic ring groups are optionally substituted by one or more substituents independently selected from the group consisting of CN, OR¹⁰, N(R¹⁰)₂, and SR¹⁰, and wherein said C₃-C₇cycloalkyl and 3- to 7-membered heterocyclic ring are each further optionally substituted with a member of the group consisting of C₁-C₃ alkyl and C₁-C₃ haloalkyl; wherein R⁸ and R⁹ are independently defined as above; and n=1,2.

In some embodiments, the compound of Formula IV is:

| Compound | | |
|---|---|---|
| ID # | Chemical Structure | IUPAC Name |
| IV-1 | 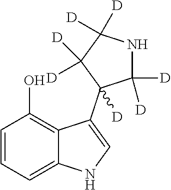 [SMILES: [2H]C1([2H])NC([2H])([2H])C([2H])(c2c[nH]c3cccc(O)c23)C1([2H])[2H]] | 3-(pyrrolidin-3-yl- 2,2,3,4,4,5,5-d7)-1H-indol- 4-ol |
| IV-2 | 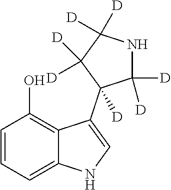 [SMILES: [2H]C1([2H])NC([2H])([2H])[C@@]([2H])(c2c[nH]c3cccc(O)c23)C1([2H])[2H]] | (R)-3-(pyrrolidin-3-yl- 2,2,3,4,4,5,5-d7)-1H-indol- 4-ol |
| IV-3 | 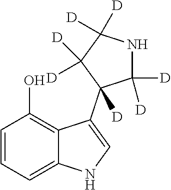 [SMILES: [2H]C1([2H])NC([2H])([2H])[C@]([2H])(c2c[nH]c3cccc(O)c23)C1([2H])[2H]] | (S)-3-(pyrrolidin-3-yl- 2,2,3,4,4,5,5-d7)-1H-indol- 4-ol |
| IV-4 | 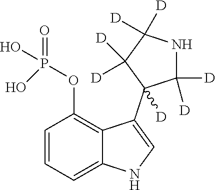 [SMILES: [2H]C1([2H])NC([2H])([2H])C([2H])(c2c[nH]c3cccc(OP(=O)(O)O)c23)C1([2H])[2H]] | 3-(pyrrolidin-3-yl- 2,2,3,4,4,5,5-d7)-1H-indol- 4-yl dihydrogen phosphate |
| IV-5 | 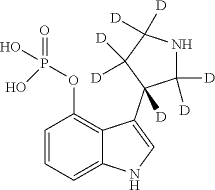 [SMILES: [2H]C1([2H])NC([2H])([2H])[C@]([2H])(c2c[nH]c3cccc(OP(=O)(O)O)c23)C1([2H])[2H]] | (S)-3-(pyrrolidin-3-yl- 2,2,3,4,4,5,5-d7)-1H-indol- 4-yl dihydrogen phosphate |
| IV-6 | 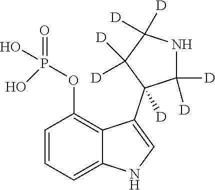 [SMILES: [2H]C1([2H])NC([2H])([2H])[C@@]([2H])(c2c[nH]c3cccc(OP(=O)(O)O)c23)C1([2H])[2H]] | (S)-3-(pyrrolidin-3-yl- 2,2,3,4,4,5,5-d7)-1H-indol- 4-yl dihydrogen phosphate |
| IV-7 | 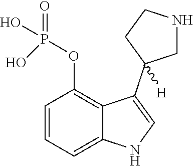 [SMILES: O=P(O)(O)Oc1cccc2[nH]cc(C3CCNC3)c12] | 3-(pyrrolidin-3-yl)-1H- indol-4-yl dihydrogen phosphate |
| IV-8 | 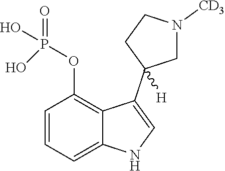 [SMILES: [2H]C([2H])([2H])N1CCC(c2c[nH]c3cccc(OP(=O)(O)O)c23)C1] | 3-(1-(methyl-d3)pyrrolidin- 3-yl)-1H-indol-4-yl dihydrogen phosphate |
| IV-9 | 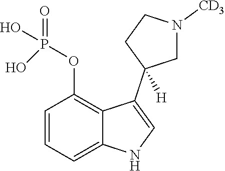 [SMILES: [2H]C([2H])([2H])N1CC[C@H](c2c[nH]c3cccc(OP(=O)(O)O)c23)C1] | (R)-3-(1-(methyl- d3)pyrrolidin-3-yl)-1H- indol-4-yl dihydrogen phosphate |
| IV-10 | 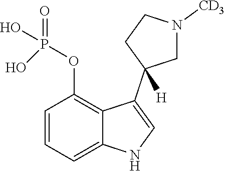 [SMILES: [2H]C([2H])([2H])N1CC[C@@H](c2c[nH]c3cccc(OP(=O)(O)O)c23)C1] | (S)-3-(1-(methyl- d3)pyrrolidin-3-yl)-1H- indol-4-yl dihydrogen phosphate |
| IV-11 | 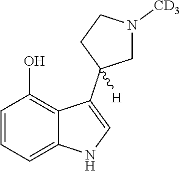 [SMILES: [2H]C([2H])([2H])N1CCC(c2c[nH]c3cccc(O)c23)C1] | 3-(1-(methyl-d3)pyrrolidin- 3-yl)-1H-indol-4-ol |
| IV-12 | 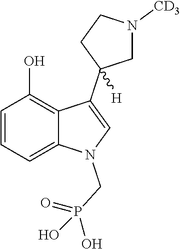 [SMILES: [2H]C([2H])([2H])N1CCC(c2cn(CP(=O)(O)O)c3cccc(O)c23)C1] | ((4-hydroxy-3-(1-methyl- d3)pyrrolidin-3-yl)-1H- indol-1- yl)methyl)phosphonic acid |
| IV-13 | 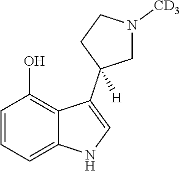 [SMILES: [2H]C([2H])([2H])N1CC[C@H](c2c[nH]c3cccc(O)c23)C1] | (R)-3-(1-(methyl- d3)pyrrolidin-3-yl)-1H- indol-4-ol |
| IV-14 | 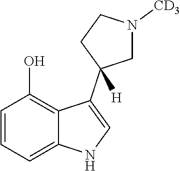 [SMILES: [2H]C([2H])([2H])N1CC[C@@H](c2c[nH]c3cccc(O)c23)C1] | (S)-3-(1-(methyl- d3)pyrrolidin-3-yl)-1H- indol-4-ol |
| IV-15 | 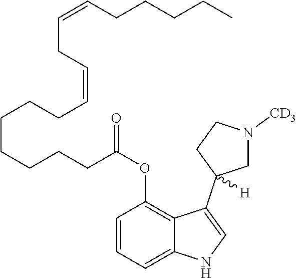 [SMILES: [2H]C([2H])([2H])N1CCC(c2c[nH]c3cccc(OC(=O)CCCCCCC/C=C\C/C=C\CCCCC)c23)C1] | 3-(1-(methyl-d3)pyrrolidin- 3-yl)-1H-indol-4-yl (9Z,12Z)-octadeca-9,12- dienoate |
| IV-16 | 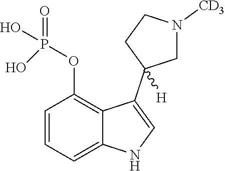 [SMILES: [2H]C([2H])([2H])N1CCC(c2c[nH]c3cccc(OP(=O)(O)O)c23)C1] | 3-(1-(methyl-d3)pyrrolidin- 3-yl)-1H-indol-4-yl dihydrogen phosphate |
| IV-17 | 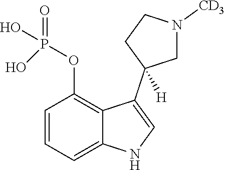 [SMILES: [2H]C([2H])([2H])N1CC[C@H](c2c[nH]c3cccc(OP(=O)(O)O)c23)C1] | (R)-3-(1-(methyl- d3)pyrrolidin-3-yl)-1H- indol-4-yl dihydrogen phosphate |
| IV-18 | 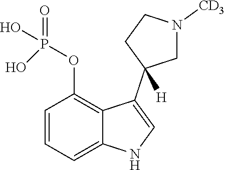 [SMILES: [2H]C([2H])([2H])N1CC[C@@H](c2c[nH]c3cccc(OP(=O)(O)O)c23)C1] | (S)-3-(1-(methyl- d3)pyrrolidin-3-yl)-1H- indol-4-yl dihydrogen phosphate |
| IV-19 | 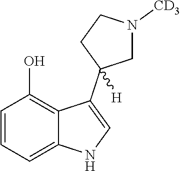 [SMILES: [2H]C([2H])([2H])N1CCC(c2c[nH]c3cccc(O)c23)C1] | 3-(1-(methyl-d3)pyrrolidin- 3-yl)-1H-indol-4-ol |
| IV-20 | 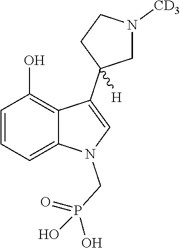 [SMILES: [2H]C([2H])([2H])N1CCC(c2cn(CP(=O)(O)O)c3cccc(O)c23)C1] | ((4-hydroxy-3-(1-(methyl- d3)pyrrolidin-3-yl)-1H- indol-1- yl)methyl)phosphonic acid |
| IV-21 | 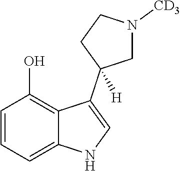 [SMILES: [2H]C([2H])([2H])N1CC[C@H](c2c[nH]c3cccc(O)c23)C1] | (R)-3-(1-(methyl- d3)pyrrolidin-3-yl)-1H- indol-4-ol |
| IV-22 | 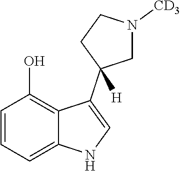 [SMILES: [2H]C([2H])([2H])N1CC[C@@H](c2c[nH]c3cccc(O)c23)C1] | (S)-3-(1-(methyl- d3)pyrrolidin-3-yl)-1H- indol-4-ol |
| IV-23 | 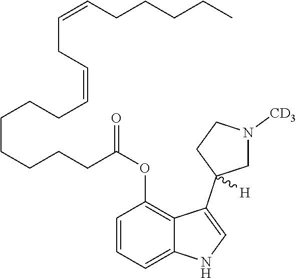 [SMILES: [2H]C([2H])([2H])N1CCC(c2c[nH]c3cccc(OC(=O)CCCCCCC/C=C\C/C=C\CCCCC)c23)C1] | 3-(1-(methyl-d3)pyrrolidin- 3-yl)-1H-indol-4-yl (9Z,12Z)-octadeca-9,12- dienoate |
| IV-24 | 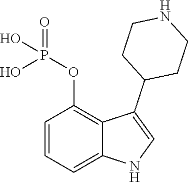 [SMILES: O=P(O)(O)Oc1cccc2[nH]cc(C3CCNCC3)c12] | 3-(piperidin-4-yl)-1H-indol- 4-yl dihydrogen phosphate |
| IV-25 | 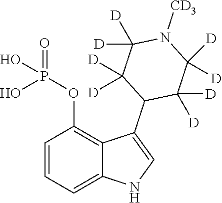 [SMILES: [2H]C([2H])([2H])N1C([2H])([2H])C([2H])([2H])C(c2c[nH]c3cccc(OP(=O)(O)O)c23)C([2H])([2H])C1([2H])[2H]] | 3-(1-(methyl-d3)piperidin- 4-yl-2,2,3,3,5,5,6,6-d8)- 1H-indol-4-yl dihydrogen phosphate |
| IV-26 | 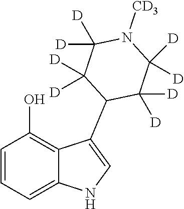 [SMILES: [2H]C([2H])([2H])N1C([2H])([2H])C([2H])([2H])C(c2c[nH]c3cccc(O)c23)C([2H])([2H])C1([2H])[2H]] | 3-(1-(methyl-d3)piperidin- 4-yl-2,2,3,3,5,5,6,6-d8)- 1H-indol-4-ol |
| IV-27 | 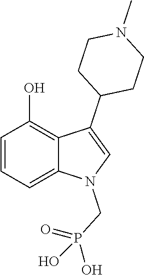 [SMILES: CN1CCC(c2cn(CP(=O)(O)O)c3cccc(O)c23)CC1] | ((4-hydroxy-3-(1- methylpiperidin-4-yl)-1H- indol-1- yl)methyl)phosphonic acid |
| IV-28 | 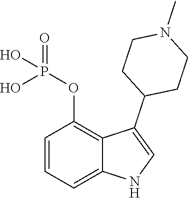 [SMILES: CN1CCC(c2c[nH]c3cccc(OP(=O)(O)O)c23)CC1] | 3-(1-methylpiperidin-4-yl)- 1H-indol-4-yl dihydrogen phosphate |
| IV-29 | 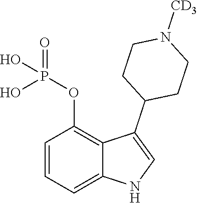 [SMILES: [2H]C([2H])([2H])N1CCC(c2c[nH]c3cccc(OP(=O)(O)O)c23)CC1] | 3-(1-(methyl-d3)piperidin- 4-yl)-1H-indol-4-yl dihydrogen phosphate |
| IV-30 | 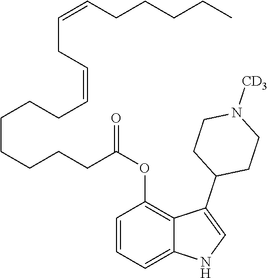 [SMILES: [2H]C([2H])([2H])N1CCC(c2c[nH]c3cccc(OC(=O)CCCCCCC/C=C\C/C=C\CCCCC)c23)CC1] | 3-(1-(methyl-d3)piperidin- 4-yl)-1H-indol-4-yl (9Z,12Z)-octadeca-9,12- dienoate; or |
| IV-31 | 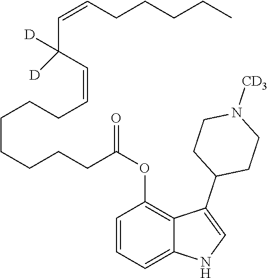 [SMILES: [2H]C([2H])(/C=C\CCCCC)/C=C\CCCCCCCC(=O)Oc1cccc2[nH]cc(C3CCN(C([2H])([2H])[2H])CC3)c12] | 3-(1-(methyl-d3)piperidin- 4-yl)-1H-indol-4-yl (9Z,12Z)-octadeca-9,12- dienoate-11,11-d2 |

or a pharmaceutically acceptable salt, prodrug and/or solvate thereof.

In some embodiments, the one or more hallucinogens, or a pharmaceutically acceptable salt, prodrug and/or solvate thereof, are selected from the compound disclosed and claimed in Applicant's co-pending U.S. provisional patent application Ser. No. 63/155,634 filed on Mar. 2, 2021, the contents of which are incorporated by reference in their entirety. Therefore in some embodiments, the one or more hallucinogens are selected from a compound of Formula (V) or a pharmaceutically acceptable salt, prodrug and/or solvate thereof:

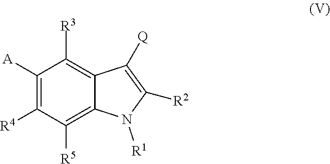

or a pharmaceutically acceptable salt, solvate and/or prodrug thereof, wherein
R¹ is selected from hydrogen, deuterium, C₁-C₆alkyl, CH₂P(O)(OR₆)₂; C(O)R⁶, CO₂R⁶, C(O)N(R)₂, S(O)R⁶ and SO₂R⁶;
Q is independently selected from:

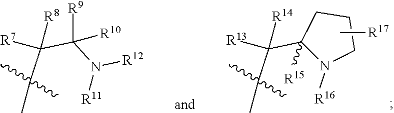

R², R⁷, R⁸, R⁹, R¹⁰, R¹¹, R¹², R¹³, R¹⁴, R¹⁵, R¹⁶ and R¹⁷ are independently selected from hydrogen, deuterium, halogen and C₁-C₆alkyl;
R³, R⁴ and R⁵ are independently selected from hydrogen, deuterium, halogen, CN, OR¹⁸, N(R¹⁸)₂, SR¹⁸, C₁-C₆alkyl, C₁-C₆haloalkyl, C₂-C₆haloalkenyl, CO₂R¹⁸, C(O)N(R¹⁸)₂, S(O)R¹⁸, SO₂R¹⁸, C₂-C₆alkenyl, C₂-C₆alkynyl, C₂-C₆haloalkynyl, C₃-C₇cycloalkyl and a 3- to 7-membered heterocyclic ring comprising 1 to 2 heteromoeities selected from O, S, S(O), SO₂, N and NR¹⁸, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR¹⁸, N(R¹⁸)₂ and SR¹⁸, and wherein said C₃-C₇cycloalkyl and 3- to 7-membered heterocyclic ring are each further optionally substituted with a substituent selected from halogen, CO₂R¹⁸, C(O)N(R¹⁸)₂, SO₂R¹⁸, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, S(O), SO₂, N, and NR¹⁸;
A is selected from selected from hydrogen, deuterium, halogen, OR¹⁹, NR¹⁹, SR¹⁹, S(O)R¹⁹ and S(O₂) R¹⁹;
each R¹⁸ is independently selected from hydrogen, C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl, and a 3- to 7-membered heterocyclic ring including 1 to 2 ring heteromoieties selected from O, S, S(O), SO₂, N and NR²⁰, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and 3- to 7-membered heterocyclic ring groups are optionally substituted by one or more substituents independently selected from CN, OR²⁰, N(R²⁰)₂ and SR²⁰, and wherein said C₃-C₇cycloalkyl and 3- to 7-membered heterocyclic ring are each further optionally substituted with a substituent selected from halogen, CO₂R²⁰, C(O)N(R²⁰)₂, SO₂R²⁰, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and a 3- to 6-membered heterocyclic ring including 1 to 2 ring heteromoieties selected from O, S, S(O), SO₂, N and NR²⁰;
R¹⁹ and R²⁰ are independently selected from hydrogen, deuterium, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, and substituted or unsubstituted heteroaryl; and wherein all available hydrogen atoms are optionally substituted with a halogen atom and/or all available atoms are optionally substituted with an alternate isotope thereof.

In some embodiments, the compound of Formula V is:

| Compound | | |
|---|---|---|
| ID # | IUPAC Name | Chemical Structure |
| V-1 | 2-(1H-indol-3-yl)-N,N-bis(methyl- d3)ethan-1-amine-1,1,2,2-d4 | 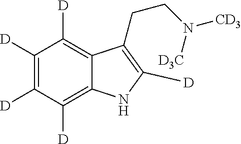 [SMILES: [2H]c1[nH]c2c([2H])c([2H])c([2H])c([2H])c2c1CCN(C([2H])([2H])[2H])C([2H])([2H])[2H]] |
| V-2 | 2-(1H-indol-3-yl-2,4,5,6,7-d5)- N,N-dimethylethan-1-amine | 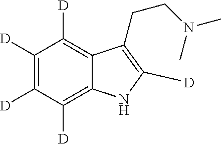 [SMILES: [2H]c1[nH]c2c([2H])c([2H])c([2H])c([2H])c2c1CCN(C)C] |
| V-3 | 22-(1H-indol-3-yl-2,4,5,6,7-d5)- N,N-dimethylethan-1-amine- 1,1,2,2-d4) | 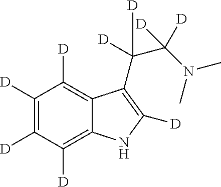 [SMILES: [2H]c1[nH]c2c([2H])c([2H])c([2H])c([2H])c2c1C([2H])([2H])C([2H])([2H])N(C)C] |
| V-4 | 2-(1H-indol-3-yl-2,4,5,6,7-d5)- N,N-dimethylethan-1-amine-2,2- d2 | 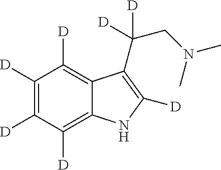 [SMILES: [2H]c1[nH]c2c([2H])c([2H])c([2H])c([2H])c2c1C([2H])([2H])CN(C)C] |
| V-5 | 2-(1H-indol-3-yl-2,4,5,6,7-d5)- N,N-bis(methyl-d3)ethan-1- amine-1,1,2,2-d4 | 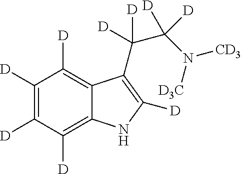 [SMILES: [2H]c1[nH]c2c([2H])c([2H])c([2H])c([2H])c2c1C([2H])([2H])C([2H])([2H])N(C([2H])([2H])[2H])C([2H])([2H])[2H]] |
| V-6 | 2-(5-(methoxy-d3)-1H-indol-3-yl)- N,N-bis(methyl-d3)ethan-1- amine-1,1,2,2-d4 | 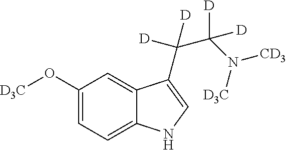 [SMILES: [2H]C([2H])([2H])Oc1ccc2[nH]cc(C([2H])([2H])C([2H])([2H])N(C([2H])([2H])[2H])C([2H])([2H])[2H])c2c1] |
| V-7 | 2-(5-methoxy-1H-indol-3-yl- 2,4,6,7-d4)-N,N-dimethylethan-1- amine | 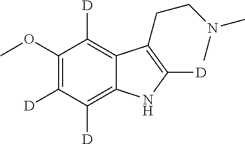 |
| V-8 | 2-(5-(methoxy-d3)-1H-indol-3-yl- 2,4,6,7-d4)-N,N-dimethylethan-1- amine | 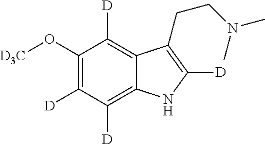 [SMILES: [2H]c1[nH]c2c([2H])c([2H])c(OC([2H])([2H])[2H])c([2H])c2c1CCN(C)C] |
| V-9 | 2-(5-methoxy-1H-indol-3-yl- 2,4,6,7-d4)-N,N-bis(methyl- d3)ethan-1-amine | 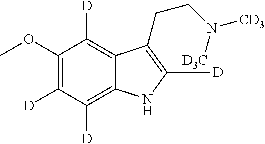 [SMILES: [2H]c1[nH]c2c([2H])c([2H])c(OC)c([2H])c2c1CCN(C([2H])([2H])[2H])C([2H])([2H])[2H]] |
| V-10 | 2-(5-(methoxy-d3)-1H-indol-3-yl- 2,4,6,7-d4)-N,N-bis(methyl- d3)ethan-1-amine-1,1,2,2-d4 | 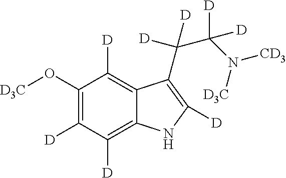 [SMILES: [2H]c1[nH]c2c([2H])c([2H])c(OC([2H])([2H])[2H])c([2H])c2c1C([2H])([2H])C([2H])([2H])N(C([2H])([2H])[2H])C([2H])([2H])[2H]] |
| V-11 | 2-(5-(methoxy-d3)-1H-indol-3-yl- 2,4,6,7-d4)-N,N-dimethylethan-1- amine | 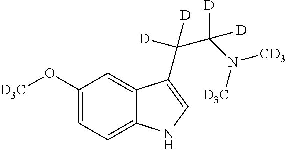 [SMILES: [2H]C([2H])([2H])Oc1ccc2[nH]cc(C([2H])([2H])C([2H])([2H])N(C([2H])([2H])[2H])C([2H])([2H])[2H])c2c1] |
| V-12 | 2-(5-(difluoromethoxy)-1H-indol- 3-yl)-N,N-bis(methyl-d3)ethan-1- amine-1,1,2,2-d4 | 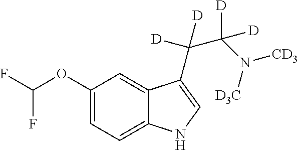 [SMILES: [2H]C([2H])([2H])N(C([2H])([2H])[2H])C([2H])([2H])C([2H])([2H])c1c[nH]c2ccc(OC(F)F)cc12] |
| V-13 | 2-(5-(difluoromethoxy)-1H-indol- 3-yl)-N,N-bis(methyl-d3)ethan-1- amine-1,1,2,2-d4 | 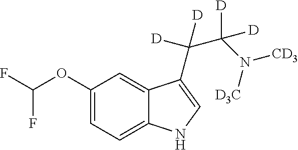 [SMILES: [2H]C([2H])([2H])N(C([2H])([2H])[2H])C([2H])([2H])C([2H])([2H])c1c[nH]c2ccc(OC(F)F)cc12] |
| V-14 | 2-(5-(difluoromethoxy)-1H-indol- 3-yl)-N,N-dimethylethan-1-amine- 1,1,2,2-d4 | 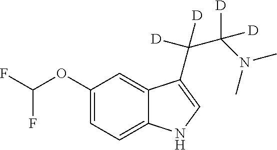 [SMILES: [2H]C([2H])(c1c[nH]c2ccc(OC(F)F)cc12)C([2H])([2H])N(C)C] |
| V-15 | (R)-3-((1-(methyl-d3)pyrrolidin-2- yl)methyl-d2)-1H-indole | 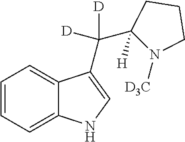 [SMILES: [2H]C([2H])(c1c[nH]c2ccccc12)[C@H]1CCCN1C([2H])([2H])[2H]] |
| V-16 | (R)-3-((1-methylpyrrolidin-2- yl)methyl-d2)-1H-indole | 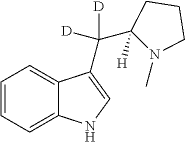 [SMILES: [2H]C([2H])(c1c[nH]c2ccccc12)[C@H]1CCCN1C] |
| V-17 | (S)-3-((1-methylpyrrolidin-2- yl)methyl-d2)-1H-indole | 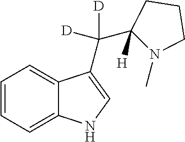 [SMILES: [2H]C([2H])(c1c[nH]c2ccccc12)[C@@H]1CCCN1C] |
| V-18 | (R)-3-((1-(methyl-d3)pyrrolidin-2- yl)methyl)-1H-indole | 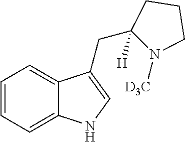 [SMILES: [2H]C([2H])([2H])N1CCC[C@@H]1Cc1c[nH]c2ccccc12] |
| V-19 | (S)-3-((1-(methyl-d3)pyrrolidin-2- yl)methyl)-1H-indole | 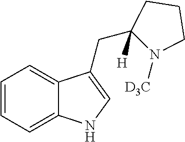 [SMILES: [2H]C([2H])([2H])N1CCC[C@H]1Cc1c[nH]c2ccccc12] |
| V-20 | (R)-3-((1-methylpyrrolidin-2- yl)methyl)-1H-indole-2,4,5,6,7-d5 | 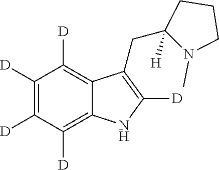 [SMILES: [2H]c1[nH]c2c([2H])c([2H])c([2H])c([2H])c2c1C[C@H]1CCCN1C] |
| V-21 | (R)-3-(pyrrolidin-2-ylmethyl)-1H- indole-2,4,5,6,7-d5 | 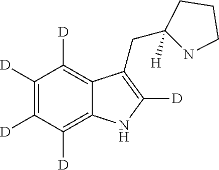 [SMILES: [2H]c1[nH]c2c([2H])c([2H])c([2H])c([2H])c2c1C[C@H]1CCCN1] |
| V-22 | (R)-3-(pyrrolidin-2-ylmethyl-d2)- 1H-indole | 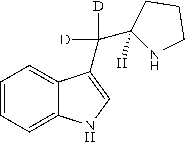 [SMILES: [2H]C([2H])(c1c[nH]c2ccccc12)[C@H]1CCCN1] |
| V-23 | (R)-5-methoxy-3-((1-(methyl- d3)pyrrolidin-2-yl)methyl)-1H- indole | 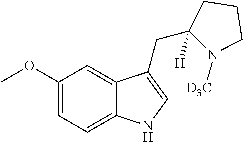 [SMILES: [2H]C([2H])([2H])N1CCC[C@@H]1Cc1c[nH]c2ccc(OC)cc12] |
| V-24 | R)-5-methoxy-3-((1-(methyl- d3)pyrrolidin-2-yl)methyl)-1H- indole | 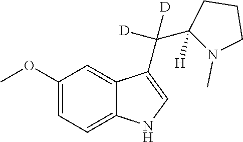 [SMILES: [2H]C([2H])(c1c[nH]c2ccc(OC)cc12)[C@H]1CCCN1C] |
| V-25 | (R)-5-(methoxy-d3)-3-((1-(methyl- d3)pyrrolidin-2-yl)methyl)-1H- indole | 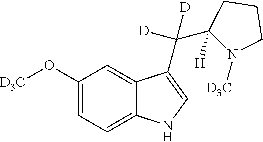 [SMILES: [2H]C([2H])([2H])Oc1ccc2[nH]cc(C([2H])([2H])[C@H]3CCCN3C([2H])([2H])[2H])c2c1] |
| V-26 | (R)-5-(methoxy-d3)-3-((1-(methyl- d3)pyrrolidin-2-yl)methyl-d2)-1H- indole | 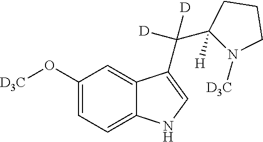 [SMILES: [2H]C([2H])([2H])Oc1ccc2[nH]cc(C([2H])([2H])[C@H]3CCCN3C([2H])([2H])[2H])c2c1] |
| V-27 | (R)-5-methoxy-3-((1-(methyl- d3)pyrrolidin-2-yl)methyl-d2)-1H- indole | 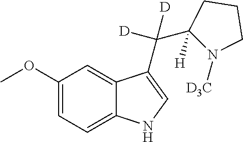 [SMILES: [2H]C([2H])(c1c[nH]c2ccc(OC)cc12)[C@H]1CCCN1C([2H])([2H])[2H]] |
| V-28 | (R)-5-(methoxy-d3)-3-(pyrrolidin- 2-ylmethyl)-1H-indole | 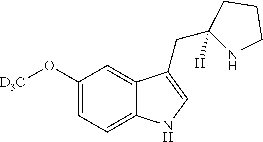 [SMILES: [2H]C([2H])([2H])Oc1ccc2[nH]cc(C[C@H]3CCCN3)c2c1] |
| V-29 | (R)-5-(methoxy-d3)-3-(pyrrolidin- 2-ylmethyl-d2)-1H-indole | 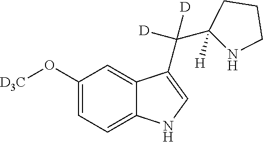 [SMILES: [2H]C([2H])([2H])Oc1ccc2[nH]cc(C([2H])([2H])[C@H]3CCCN3)c2c1] |
| V-30 | (R)-5-(methoxy-d3)-3-(pyrrolidin- 2-ylmethyl-d2)-1H-indole | 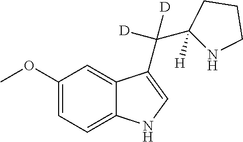 [SMILES: [2H]C([2H])(c1c[nH]c2ccc(OC)cc12)[C@H]1CCCN1] |
| V-31 | (R)-5-(difluoromethoxy)-3-((1- methylpyrrolidin-2-yl)methyl-d2)- 1H-indole | 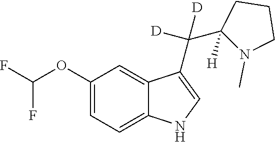 [SMILES: [2H]C([2H])(c1c[nH]c2ccc(OC(F)F)cc12)[C@H]1CCCN1C] |
| V-32 | (S)-5-methoxy-3-((1-(methyl- d3)pyrrolidin-2-yl)methyl-d2)-1H- indole | 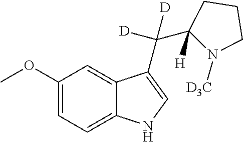 [SMILES: [2H]C([2H])(c1c[nH]c2ccc(OC)cc12)[C@@H]1CCCN1C([2H])([2H])[2H]] |
| V-33 | (S)-5-methoxy-3-((1- methylpyrrolidin-2-yl)methyl-d2)- 1H-indole | 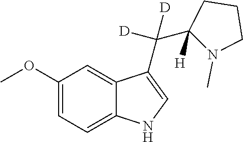 [SMILES: [2H]C([2H])(c1c[nH]c2ccc(OC)cc12)[C@@H]1CCCN1C] |
| V-34 | 2-(1H-indol-3-yl)-N,N- dimethylethan-1-amine-1-d | 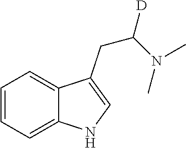 |
| V-35 | 2-(1H-indol-3-yl)-N,N- dimethylethan-1-amine-1,1-d2 | 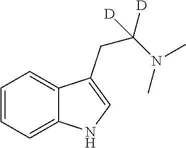 [SMILES: [2H]C([2H])(Cc1c[nH]c2ccccc12)N(C)C] |
| V-36 | 2-(1H-indol-3-yl)-N,N- dimethylethan-1-amine-1,1,2-d3 | 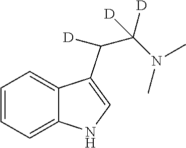 [SMILES: [2H]C(c1c[nH]c2ccccc12)C([2H])([2H])N(C)C] |
| V-37 | 2-(1H-indol-3-yl)-N,N- dimethylethan-1-amine-1,2- d2dimethylethan-1-amine-2,2-d2 | 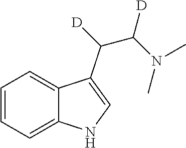 [SMILES: [2H]C(c1c[nH]c2ccccc12)C([2H])N(C)C] |
| V-38 | 2-(1H-indol-3-yl)-N,N- dimethylethan-1-amine-1,2,2- d3d4 | 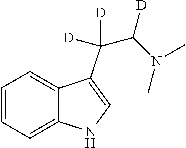 [SMILES: [2H]C(N(C)C)C([2H])([2H])c1c[nH]c2ccccc12] |
| V-39 | 2-(1H-indol-3-yl)-N,N- dimethylethan-1-amine-1,1,2,2- d4 | 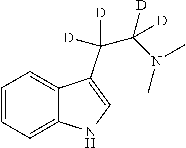 [SMILES: [2H]C([2H])(c1c[nH]c2ccccc12)C([2H])([2H])N(C)C] |
| V-40 | 2-(1H-indol-3-yl)-N,N- dimethylethan-1-amine-2,2-d2 | 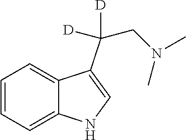 [SMILES: [2H]C([2H])(CN(C)C)c1c[nH]c2ccccc12] |
| V-41 | 2-(5-(methoxy-d3)-1H-indol-3-yl)- N,N-dimethylethan-1-amine- 1,1,2,2-d4 | 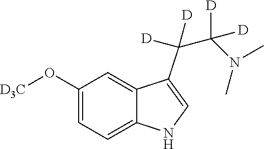 [SMILES: [2H]C([2H])([2H])Oc1ccc2[nH]cc(C([2H])([2H])C([2H])([2H])N(C)C)c2c1] |
| V-42 | 2-(5-(methoxy-d3)-1H-indol-3-yl)- N,N-dimethylethan-1-amine-2,2- d2 | 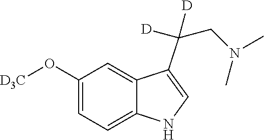 [SMILES: [2H]C([2H])([2H])Oc1ccc2[nH]cc(C([2H])([2H])CN(C)C)c2c1] |
| V-43 | 2-(5-(methoxy-d3)-1H-indol-3-yl)- N,N-dimethylethan-1-amine-1,1- d2 | 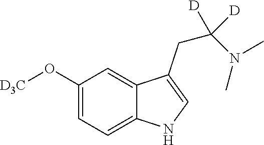 [SMILES: [2H]C([2H])([2H])Oc1ccc2[nH]cc(CC([2H])([2H])N(C)C)c2c1] |
| V-44 | 2-(5-(methoxy-d3)-1H-indol-3-yl)- N,N-dimethylethan-1-amine | 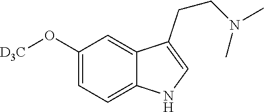 [SMILES: [2H]C([2H])([2H])Oc1ccc2[nH]cc(CCN(C)C)c2c1] |
| V-45 | 2-(5-methoxy-1H-indol-3-yl)-N,N- dimethylethan-1-amine-1,1,2,2- d4 | 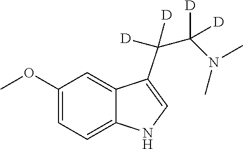 |
| V-46 | 2-(5-methoxy-1H-indol-3-yl)-N,N- dimethylethan-1-amine-2,2-d2 | 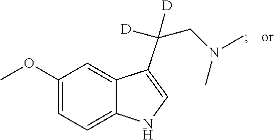 |
| V-47 | 2-(5-methoxy-1H-indol-3-yl)-N,N- dimethylethan-1-amine-1,1-d2 | 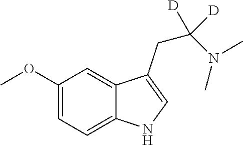 |

or a pharmaceutically acceptable salt, prodrug and/or solvate thereof.

In some embodiments, the one or more hallucinogens are selected from the compounds disclosed and claimed in Applicant's co-pending U.S. provisional patent application Ser. No. 63/260,470 filed on Aug. 20, 2021, the contents of which are incorporated by reference in their entirety. Therefore, in some embodiments, the one or more hallucinogens are selected from a compound of Formula (VI) or a pharmaceutically acceptable salt, prodrug and/or solvate thereof:

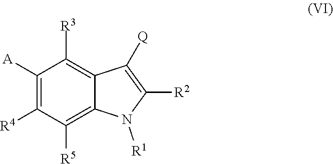

or a pharmaceutically acceptable salt, solvate and/or prodrug thereof, wherein:
R¹ is selected from H, D, C₁₋₆alkyl, C₁₋₆P(O)(OR₆)(OR⁷), C(O)R⁶, CO₂R⁶, C(O)N(R⁶)(R⁷), S(O)R⁶ and SO₂R⁶;
Q is selected from Q1 and Q2:

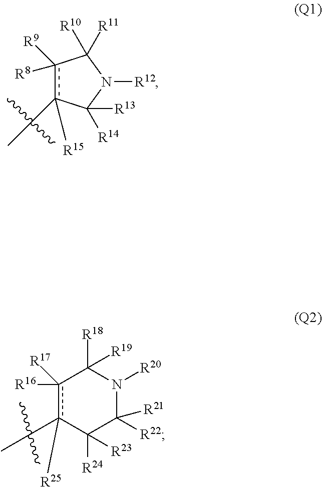

[symbol] is a single bond or a double bond wherein when [symbol] is a double bond in Q1 then R⁹ and R¹⁵ are not present, and when [symbol] is a double bond in Q2 then R¹⁷ and R²⁵ are not present;
R², R³, R⁴, R⁵, R⁸, R⁹, R¹⁰, R¹¹, R¹³, R¹⁴, R¹⁵, R¹⁶, R¹⁷, R¹⁸, R¹⁹, R²¹, R²², R²³, R²⁴ and R²⁵ are independently selected from H, D, halo and C₁₋₆alkyl;
each R⁶ is independently selected from H, D, C₁₋₂₀alkyl, C₂₋₂₀alkenyl, C₂₋₂₀alkynyl, aryl, C₃₋₁₀cycloalkyl, 3- to 10-membered heterocycloalkyl comprising 1 to 4 heteromoeities independently selected from O, S, S(O), SO₂, N and NR²⁶ and 5- to 10-membered heteroaryl comprising 1 to 4 heteromoeities independently selected from O, S, S(O), SO₂, N and NR²⁶, wherein the C₁₋₂₀alkyl, C₂₋₂₀alkenyl, C₂₋₂₀alkynyl, C₃₋₁₀cycloalkyl, aryl, 3- to 10-membered heterocycloalkyl and 5- to 10-membered heteroaryl are optionally substituted with one or more substituents independently selected from halo, CN, OR²⁷, N(R²⁷)(R²⁸) and SR²⁷, and wherein the C₃₋₇cycloalkyl, aryl, 3- to 10-membered heterocycloalkyl and 5- to 10-membered heteroaryl are each further optionally substituted with a substituent selected from CO₂R²⁹, C(O)N(R²⁹)(R³⁰), S(O)R²⁹, SO₂R²⁹, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR³¹ and 5- to 6-membered heteroaryl comprising 1 to 2 ring heteromoieties independently selected from O, S, S(O), SO₂, N, and NR³¹;
each R⁷ is independently selected from H and C₁₋₆alkyl;
R¹² and R²⁰ are independently selected from H, D, C₁₋₆alkyl, C(O) C₁₋₂₀alkyl, C(O) C₂₋₂₀alkenyl and C(O) C₂₋₂₀alkynyl;
A is selected from H, D, halo, C₁₋₆alkyl, C₂₋₆alkenyl, C₂₋₆alkynyl, CN, OR³², N(R³²)(R³³), SR³², S(O)R³², SO₂R³², C(O)R³², CO₂R³², C(O)N(R³²)(R³³), C(NR³⁴) R³², C(NR³⁴) NR³²R³³, C(NR³⁴) OR³², aryl, C₃₋₁₀cycloalkyl, 3- to 10-membered heterocycloalkyl comprising 1 to 4 heteromoeities independently selected from O, S, S(O), SO₂, N and NR³² and 5- to 10-membered heteroaryl comprising 1 to 4 heteromoeities independently selected from O, S, S(O), SO₂, N and NR³², wherein the C₁₋₆alkyl, C₂₋₆alkenyl, C₂₋₆alkynyl, C₃₋₇cycloalkyl, aryl, 3- to 10-membered heterocycloalkyl and 5- to 10-membered heteroaryl are optionally substituted with one or more substituents independently selected from halo, CN, OR³⁵, C(O)₂R³⁵, N(R³⁵)(R³⁶) and SR³⁵, and wherein the C₃₋₁₀cycloalkyl, aryl, 3- to 10-membered heterocycloalkyl and 5- to 10-membered heteroaryl are each further optionally substituted with a substituent selected from CO₂R³⁷, C(O)N(R³⁷)(R³⁸), S(O)R³⁷, SO₂R³⁸, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR³⁹ and 5- to 6-membered heteroaryl comprising 1 to 2 ring heteromoieties independently selected from O, S, S(O), SO₂, N, and NR³⁹;
each R³² is independently selected from H, C₁₋₂₀alkyl, C₂₋₂₀alkenyl, C₂₋₂₀alkynyl, C₃₋₁₀cycloalkyl, aryl, 3- to 10-membered heterocycloalkyl comprising 1 to 4 heteromoeities independently selected from O, S, S(O), SO₂, N and NR⁴⁰ and 5- to 10-membered heteroaryl comprising 1 to 4 heteromoeities independently selected from O, S, S(O), SO₂, N and NR⁴⁰, wherein said C₁₋₂₀alkyl, C₂₋₂₀alkenyl, C₂₋₂₀alkynyl, C₃-C₁₀cycloalkyl, 3- to 10-membered heterocycloalkyl and 5- to 10-membered heteroaryl are optionally substituted by one or more substituents independently selected from CN, OR⁴¹, CO₂R⁴¹ N(R⁴¹)(R⁴²) and SR⁴¹, and wherein the C₃₋₁₀cycloalkyl, aryl, 3- to 10-membered heterocycloalkyl and 5- to 10-membered heteroaryl are each further optionally substituted with a substituent selected from CO₂R⁴³, C(O)N(R⁴³)(R⁴⁴), S(O)R⁴³, SO₂R⁴³, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁴⁵ and 5- to 6-membered heteroaryl comprising 1 to 2 ring heteromoieties independently selected from O, S, S(O), SO₂, N, and NR⁴⁵;
R³⁵, R³⁶, R³⁷, R³⁸, R³⁹, R⁴⁰, R⁴¹, R⁴², R⁴³, R⁴⁴ and R⁴⁵ are independently selected from H and C₁₋₆alkyl; and all available hydrogen atoms are optionally substituted with a halogen atom and/or all available atoms are optionally substituted with an alternate isotope thereof, provided when R¹ is H, then A is not H, OH or OC₁₋₄alkyl.

In some embodiments, the compound of Formula VI is:

| Compound | |
|---|---|
| ID # | Chemical Structure |
| VI-1 | 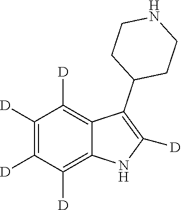 [SMILES: [2H]c1[nH]c2c([2H])c([2H])c([2H])c([2H])c2c1C1CCNCC1] |
| VI-2 | 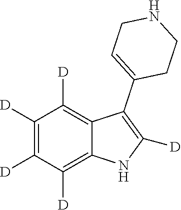 [SMILES: [2H]c1[nH]c2c([2H])c([2H])c([2H])c([2H])c2c1C1=CCNCC1] |
| VI-3 | 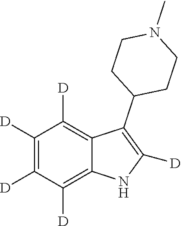 [SMILES: [2H]c1[nH]c2c([2H])c([2H])c([2H])c([2H])c2c1C1CCN(C)CC1] |
| VI-4 | 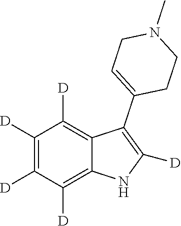 [SMILES: [2H]c1[nH]c2c([2H])c([2H])c([2H])c([2H])c2c1C1=CCN(C)CC1] |
| VI-5 | 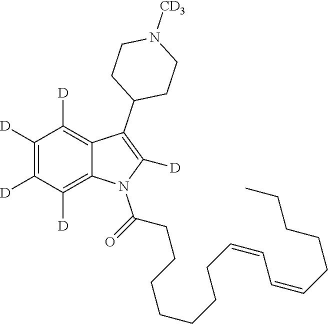 [SMILES: [2H]c1c([2H])c([2H])c2c(c1[2H])c(C1CCN(C([2H])([2H])[2H])CC1)c([2H])n2C(=O)CCCCCCC/C=C\C=C/CCCCC] |
| VI-6 | 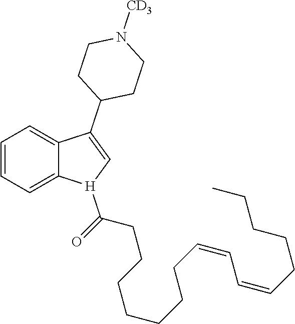 |
| VI-7 | 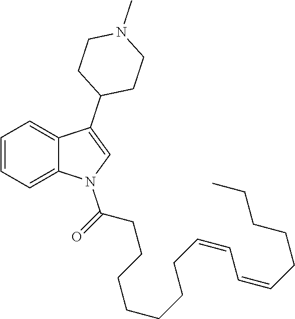 [SMILES: CCCCC/C=C\C=C/CCCCCCCC(=O)n1cc(C2CCN(C)CC2)c2ccccc21] |
| VI-8 | 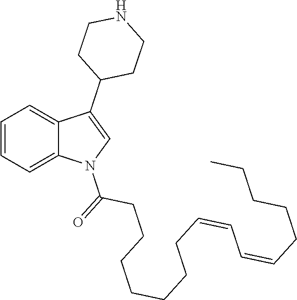 [SMILES: CCCCC/C=C\C=C/CCCCCCCC(=O)n1cc(C2CCNCC2)c2ccccc21] |
| VI-9 | 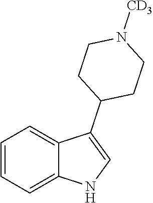 [SMILES: [2H]C([2H])([2H])N1CCC(c2c[nH]c3ccccc23)CC1] |
| VI-10 | 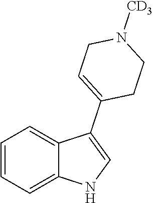 [SMILES: [2H]C([2H])([2H])N1CC=C(c2c[nH]c3ccccc23)CC1] |
| VI-11 | 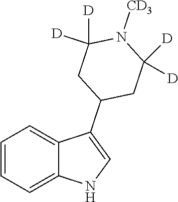 [SMILES: [2H]C([2H])([2H])N1C([2H])([2H])CC(c2c[nH]c3ccccc23)CC1([2H])[2H]] |
| VI-12 | 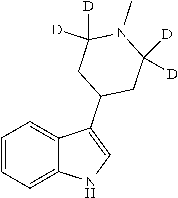 [SMILES: [2H]C1([2H])CC(c2c[nH]c3ccccc23)CC([2H])([2H])N1C] |
| VI-13 | 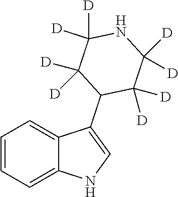 [SMILES: [2H]C1([2H])NC([2H])([2H])C([2H])([2H])C(c2c[nH]c3ccccc23)C1([2H])[2H]] |
| VI-14 | 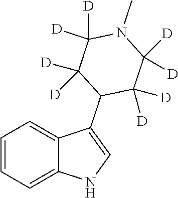 [SMILES: [2H]C1([2H])C(c2c[nH]c3ccccc23)C([2H])([2H])C([2H])([2H])N(C)C1([2H])[2H]] |
| VI-15 | 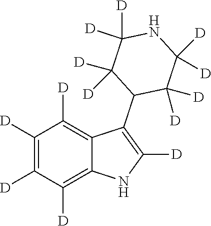 [SMILES: [2H]c1[nH]c2c([2H])c([2H])c([2H])c([2H])c2c1C1C([2H])([2H])C([2H])([2H])NC([2H])([2H])C1([2H])[2H]] |
| VI-16 | 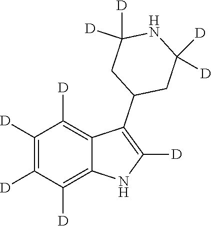 [SMILES: [2H]c1[nH]c2c([2H])c([2H])c([2H])c([2H])c2c1C1CC([2H])([2H])NC([2H])([2H])C1] |
| VI-17 | 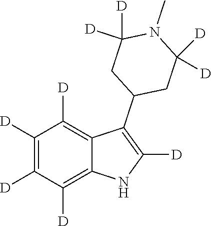 [SMILES: [2H]c1[nH]c2c([2H])c([2H])c([2H])c([2H])c2c1C1CC([2H])([2H])N(C)C([2H])([2H])C1] |
| VI-18 | 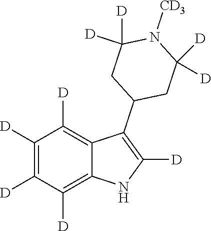 [SMILES: [2H]c1[nH]c2c([2H])c([2H])c([2H])c([2H])c2c1C1CC([2H])([2H])N(C([2H])([2H])[2H])C([2H])([2H])C1] |
| VI-19 | 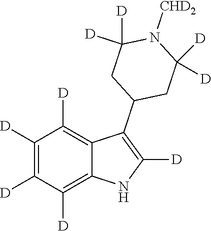 |
| VI-20 | 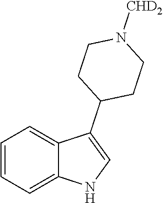 |
| VI-21 | 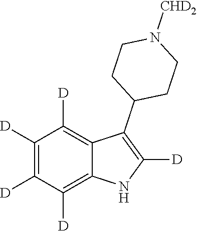 |
| VI-22 | 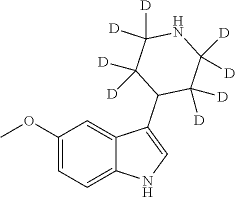 [SMILES: [2H]C1([2H])NC([2H])([2H])C([2H])([2H])C(c2c[nH]c3ccc(OC)cc23)C1([2H])[2H]] |
| VI-23 | 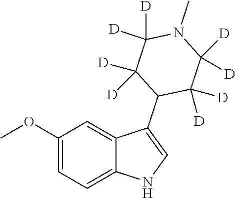 [SMILES: [2H]C1([2H])C(c2c[nH]c3ccc(OC)cc23)C([2H])([2H])C([2H])([2H])N(C)C1([2H])[2H]] |
| VI-24 | 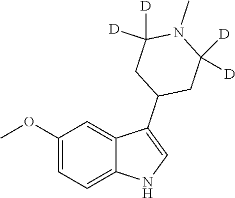 [SMILES: [2H]C1([2H])CC(c2c[nH]c3ccc(OC)cc23)CC([2H])([2H])N1C] |
| VI-25 | 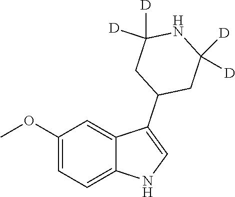 [SMILES: [2H]C1([2H])CC(c2c[nH]c3ccc(OC)cc23)CC([2H])([2H])N1] |
| VI-26 | 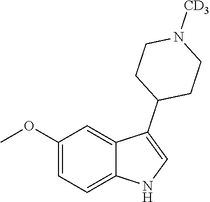 [SMILES: [2H]C([2H])([2H])N1CCC(c2c[nH]c3ccc(OC)cc23)CC1] |
| VI-27 | 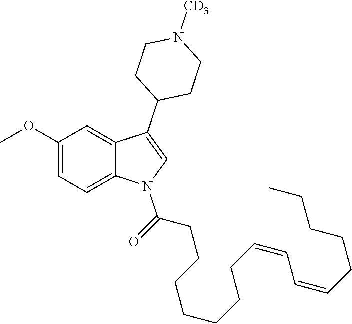 [SMILES: [2H]C([2H])([2H])N1CCC(c2cn(C(=O)CCCCCCC/C=C\C=C/CCCCC)c3ccc(OC)cc23)CC1] |
| VI-28 | 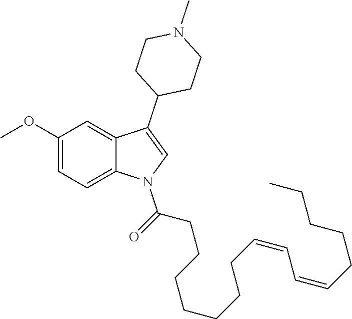 [SMILES: CCCCC/C=C\C=C/CCCCCCCC(=O)n1cc(C2CCN(C)CC2)c2cc(OC)ccc21] |
| VI-29 | 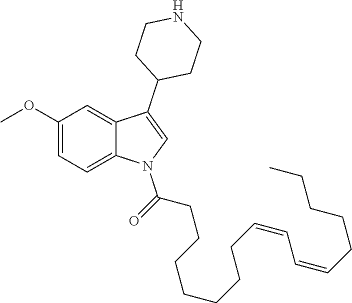 [SMILES: CCCCC/C=C\C=C/CCCCCCCC(=O)n1cc(C2CCNCC2)c2cc(OC)ccc21] |
| VI-30 | 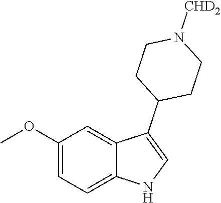 |
| VI-31 | 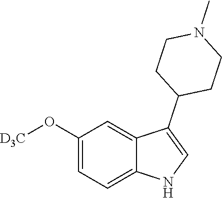 [SMILES: [2H]C([2H])([2H])Oc1ccc2[nH]cc(C3CCN(C)CC3)c2c1] |
| VI-32 | 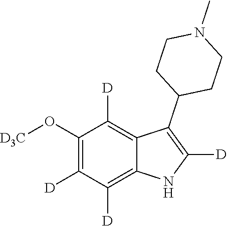 [SMILES: [2H]c1[nH]c2c([2H])c([2H])c(OC([2H])([2H])[2H])c([2H])c2c1C1CCN(C)CC1] |
| VI-33 | 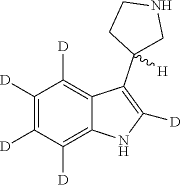 [SMILES: [2H]c1[nH]c2c([2H])c([2H])c([2H])c([2H])c2c1C1CCNC1] |
| VI-34 | 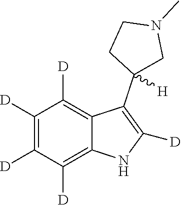 [SMILES: [2H]c1[nH]c2c([2H])c([2H])c([2H])c([2H])c2c1C1CCN(C)C1] |
| VI-35 | 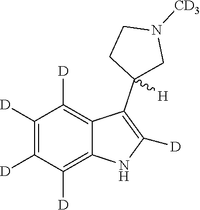 [SMILES: [2H]c1[nH]c2c([2H])c([2H])c([2H])c([2H])c2c1C1CCN(C([2H])([2H])[2H])C1] |
| VI-36 | 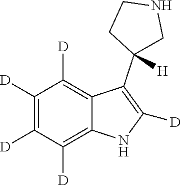 [SMILES: [2H]c1[nH]c2c([2H])c([2H])c([2H])c([2H])c2c1[C@@H]1CCNC1] |
| VI-37 | 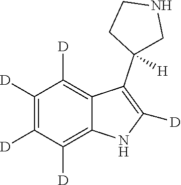 [SMILES: [2H]c1[nH]c2c([2H])c([2H])c([2H])c([2H])c2c1[C@H]1CCNC1] |
| VI-38 | 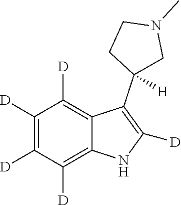 [SMILES: [2H]c1[nH]c2c([2H])c([2H])c([2H])c([2H])c2c1[C@H]1CCN(C)C1] |
| VI-39 | 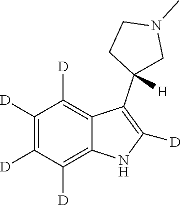 [SMILES: [2H]c1[nH]c2c([2H])c([2H])c([2H])c([2H])c2c1[C@@H]1CCN(C)C1] |
| VI-40 | 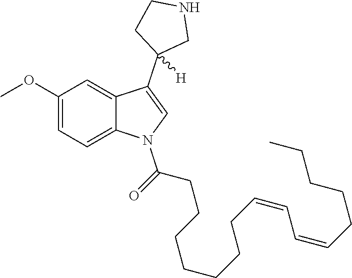 [SMILES: CCCCC/C=C\C=C/CCCCCCCC(=O)n1cc(C2CCNC2)c2cc(OC)ccc21] |
| VI-41 | 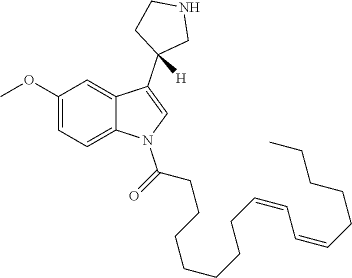 [SMILES: CCCCC/C=C\C=C/CCCCCCCC(=O)n1cc([C@@H]2CCNC2)c2cc(OC)ccc21] |
| VI-42 | 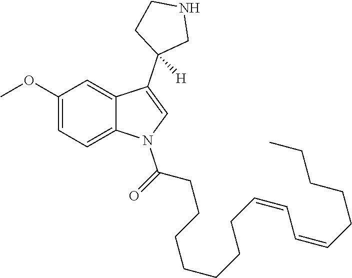 [SMILES: CCCCC/C=C\C=C/CCCCCCCC(=O)n1cc([C@H]2CCNC2)c2cc(OC)ccc21] |
| VI-43 | 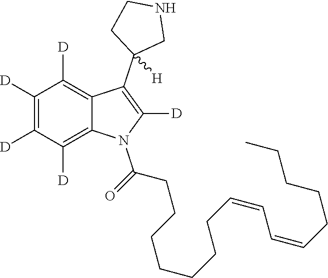 [SMILES: [2H]c1c([2H])c([2H])c2c(c1[2H])c(C1CCNC1)c([2H])n2C(=O)CCCCCCC/C=C\C=C/CCCCC] |
| VI-44 | 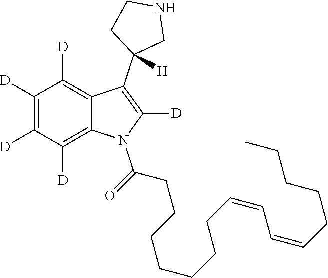 [SMILES: [2H]c1c([2H])c([2H])c2c(c1[2H])c([C@@H]1CCNC1)c([2H])n2C(=O)CCCCCCC/C=C\C=C/CCCCC] |
| VI-45 | 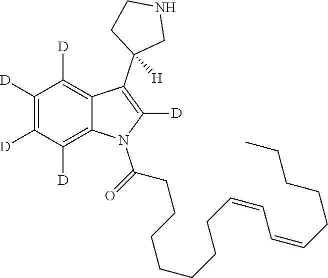 [SMILES: [2H]c1c([2H])c([2H])c2c(c1[2H])c([C@H]1CCNC1)c([2H])n2C(=O)CCCCCCC/C=C\C=C/CCCCC] |
| VI-46 | 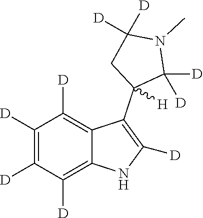 [SMILES: [2H]c1[nH]c2c([2H])c([2H])c([2H])c([2H])c2c1C1CC([2H])([2H])N(C)C1([2H])[2H]] |
| VI-47 | 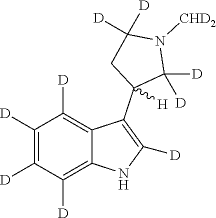 |
| VI-48 | 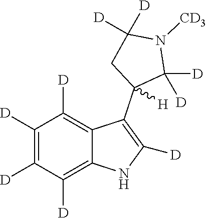 [SMILES: [2H]c1[nH]c2c([2H])c([2H])c([2H])c([2H])c2c1C1CC([2H])([2H])N(C([2H])([2H])[2H])C1([2H])[2H]] |
| VI-49 | 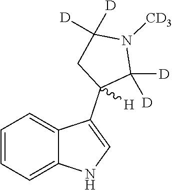 [SMILES: [2H]C([2H])([2H])N1C([2H])([2H])CC(c2c[nH]c3ccccc23)C1([2H])[2H]] |
| VI-50 | 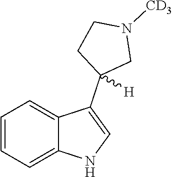 [SMILES: [2H]C([2H])([2H])N1CCC(c2c[nH]c3ccccc23)C1] |
| VI-51 | 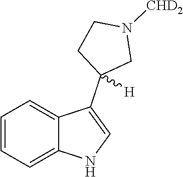 |
| VI-52 | 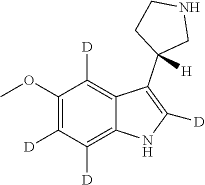 [SMILES: [2H]c1[nH]c2c([2H])c([2H])c(OC)c([2H])c2c1[C@@H]1CCNC1] |
| VI-53 | 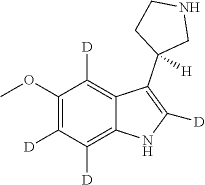 [SMILES: [2H]c1[nH]c2c([2H])c([2H])c(OC)c([2H])c2c1[C@H]1CCNC1] |
| VI-54 | 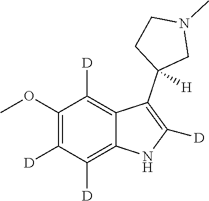 |
| VI-55 | 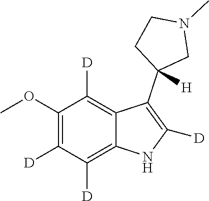 |
| VI-56 | 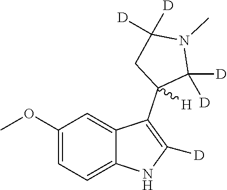 [SMILES: [2H]c1[nH]c2ccc(OC)cc2c1C1CC([2H])([2H])N(C)C1([2H])[2H]] |
| VI-57 | 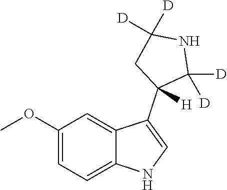 [SMILES: [2H]C1([2H])C[C@@H](c2c[nH]c3ccc(OC)cc23)C([2H])([2H])N1] |
| VI-58 | 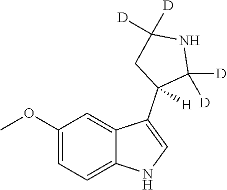 [SMILES: [2H]C1([2H])C[C@H](c2c[nH]c3ccc(OC)cc23)C([2H])([2H])N1] |
| VI-59 | 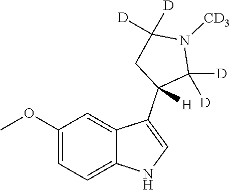 [SMILES: [2H]C([2H])([2H])N1C([2H])([2H])C[C@@H](c2c[nH]c3ccc(OC)cc23)C1([2H])[2H]] |
| VI-60 | 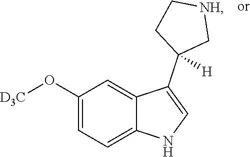 [SMILES: [2H]C([2H])([2H])Oc1ccc2[nH]cc([C@H]3CCNC3)c2c1 ; "or"] |
| VI-61 | 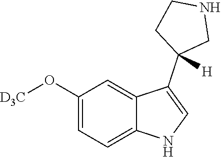 [SMILES: [2H]C([2H])([2H])Oc1ccc2[nH]cc([C@@H]3CCNC3)c2c1] |

or a pharmaceutically acceptable salt, prodrug and/or solvate thereof.

In some embodiments, the one or more hallucinogens are selected from the compound disclosed and claimed in Applicant's co-pending U.S. provisional patent application Ser. No. 63/326,406 filed on Apr. 1, 2022, the contents of which are incorporated by reference in their entirety. Therefore, in some embodiments, the one or more hallucinogens are selected from a compound of Formula (VII) or a pharmaceutically acceptable salt, prodrug and/or solvate thereof:

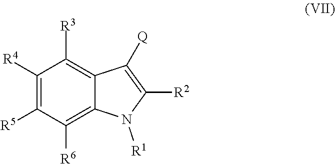

or a pharmaceutically acceptable salt and/or solvate thereof, wherein:
R¹ is selected from C(O)R⁷, CO₂R⁷ and C(O)N(R⁷)(R⁷); S(O)R⁷ and SO₂R⁷; Q is selected from Q1, Q2, Q3, Q4 and Q5:

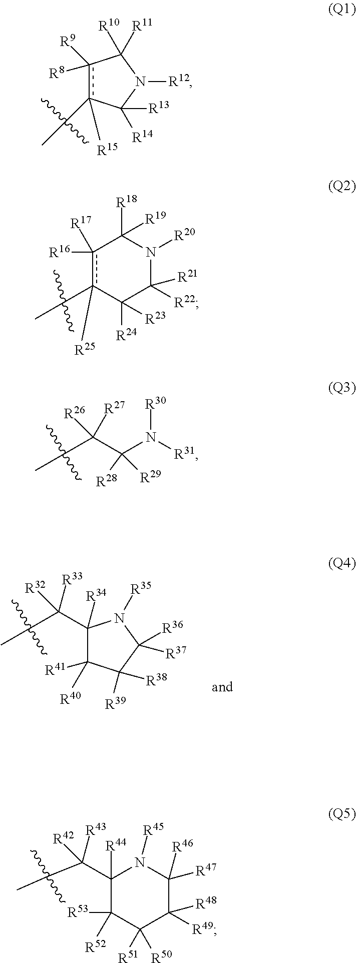

[symbol] is a single bond or a double bond provided that when [symbol] in Q1 is a double bond then R⁹ and R¹⁵ are not present, and when [symbol] in Q2 is a double bond then R¹⁷ and R²⁵ are not present;
R², R⁵ and R⁶ are independently selected from H, halo, CN, C₁₋₆alkyl, C₂₋₆alkenyl, C₂-C₆alkynyl and C₁₋₆alkoxy;
one of R³ and R⁴ is selected from H, halo, C₁₋₆alkyl, C₂₋₆alkenyl, C₂₋₆alkynyl and C₁-C₆alkoxy and the other of R³ and R⁴ is selected from A, H, halo, C₁₋₆alkyl, C₂₋₆alkenyl, C₂₋₆alkynyl and C₁₋₆alkoxy;
A is selected from OR⁵⁴, OP(O)(OR⁵⁴)(OR⁵⁵), N(R⁵⁴)(R⁵⁵), SR⁵⁴, S(O)R⁵⁴, SO₂R⁵⁴, C(O)R⁵⁴, CO₂R⁵⁴, C(O)N(R⁵⁴)(R⁵⁵), C(NR⁵⁶) R⁵⁴, C(NR⁵⁶) NR⁵⁴R⁵⁵, C(NR⁵⁶) OR⁵⁴, aryl, C₃₋₁₀cycloalkyl, 3- to 10-membered heterocycloalkyl comprising 1 to 4 heteromoeities independently selected from O, S, S(O), SO₂, N and NR⁵⁴ and 5- to 10-membered heteroaryl comprising 1 to 4 heteromoeities independently selected from O, S, S(O), SO₂, N and NR⁵⁴, wherein the C₁₋₆alkyl, C₂₋₆alkenyl, C₂₋₆alkynyl, C₃₋₇cycloalkyl, aryl, 3- to 10-membered heterocycloalkyl and 5- to 10-membered heteroaryl are optionally substituted with one or more substituents independently selected from halo, CN, OR⁵⁷, CO₂R⁵⁷, N(R⁵⁷)(R⁵⁸) and SR⁵⁷, and wherein the C₃₋₁₀cycloalkyl, aryl, 3- to 10-membered heterocycloalkyl and 5- to 10-membered heteroaryl are each further optionally substituted with a substituent selected from CO₂R⁵⁹, C(O)N(R⁵⁹)(R⁶⁰), S(O)R⁵⁹, SO₂R⁵⁹, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR₆₀ and 5- to 6-membered heteroaryl comprising 1 to 2 ring heteromoieties independently selected from O, S, S(O), SO₂, N, and NR₆₀;
R⁷ is selected from C₇₋₃₀alkyl, C₇₋₃₀alkenyl and C₇₋₃₀alkynyl, wherein the C₇₋₃₀alkyl, C₇₋₃₀alkenyl and C₇₋₃₀alkynyl, are optionally substituted with one or more substituents independently selected from halo, OR⁶¹, N(R⁶¹)(R⁶²) and SR⁶¹ and/or are optionally interrupted by one to six heteromoieties independently selected from O, C(O), CO₂ and NR⁶³;
R⁷ is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹ R⁵² and R⁵³ are independently selected from H, halo and C₁₋₆alkyl;
R⁵⁴ is selected from H, C₁₋₆alkyl, C₂₋₆alkenyl, C₂₋₆alkynyl, C₃₋₁₀cycloalkyl, aryl, 3- to 10-membered heterocycloalkyl comprising 1 to 4 heteromoeities independently selected from O, S, S(O), SO₂, N and NR⁶⁴ and 5- to 10-membered heteroaryl comprising 1 to 4 heteromoeities independently selected from O, S, S(O), SO₂, N and NR⁶⁴, wherein said C₁₋₆alkyl, C₂₋₆alkenyl, C₂₋₆alkynyl, C₃₋₁₀cycloalkyl, 3- to 10-membered heterocycloalkyl and 5- to 10-membered heteroaryl are optionally substituted by one or more substituents independently selected from CN, OR⁶⁵, CO₂R⁶⁵ N(R⁶⁵)(R⁶⁶) and SR⁶⁵, and wherein the C₃₋₁₀cycloalkyl, aryl, 3- to 10-membered heterocycloalkyl and 5- to 10-membered heteroaryl are each further optionally substituted with a substituent selected from CO₂R⁶⁷, C(O)N(R⁶⁷)(R⁶⁸), S(O)R⁶⁷, SO₂R⁶⁷, C₁₋₆alkyl, C₂₋₆alkenyl, C₂-C₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁹ and 5- to 6-membered heteroaryl comprising 1 to 2 ring heteromoieties independently selected from O, S, S(O), SO₂, N, and NR⁶⁹;
R¹², R²⁰, R³⁵ and R⁴⁵ are independently selected from H, C₁₋₆alkyl and C(O) C₁₋₆alkyl; R³⁰ and R³¹ are independently selected from H, C₁₋₆alkyl and C(O) C₁₋₆alkyl, or
R³⁰ and R³¹, together with the N atom to which they are bound, form a 3- to 8-membered heterocyclic ring which optionally comprises one or two additional heteromoieties independently selected from O, S, S(O), SO₂, N, and NR⁷⁰;
R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H and C₁₋₆alkyl; and
wherein all available hydrogen atoms are optionally and independently substituted with a fluorine atom or chlorine atom and all available atoms are optionally substituted with alternate isotope thereof.

In some embodiments, the compound of Formula VII is:

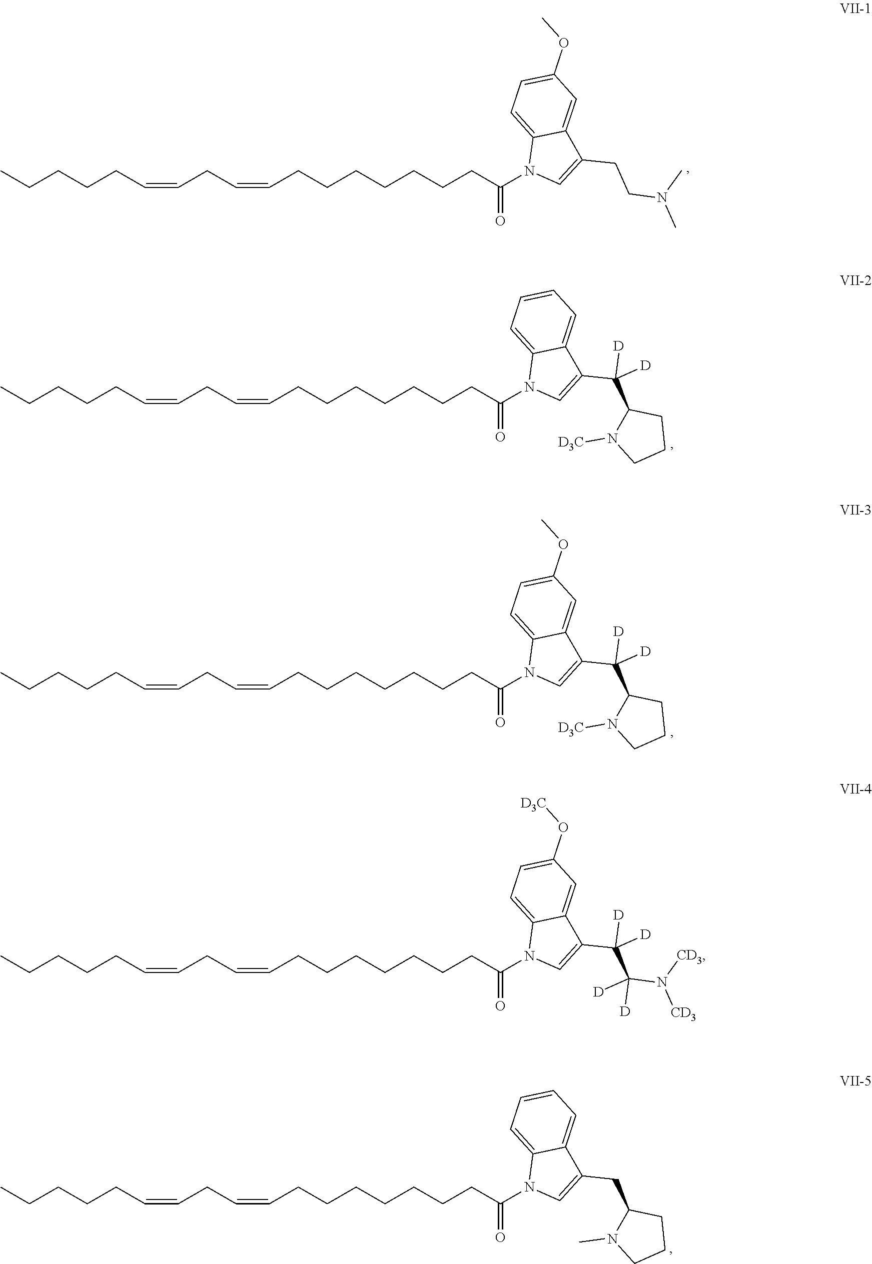

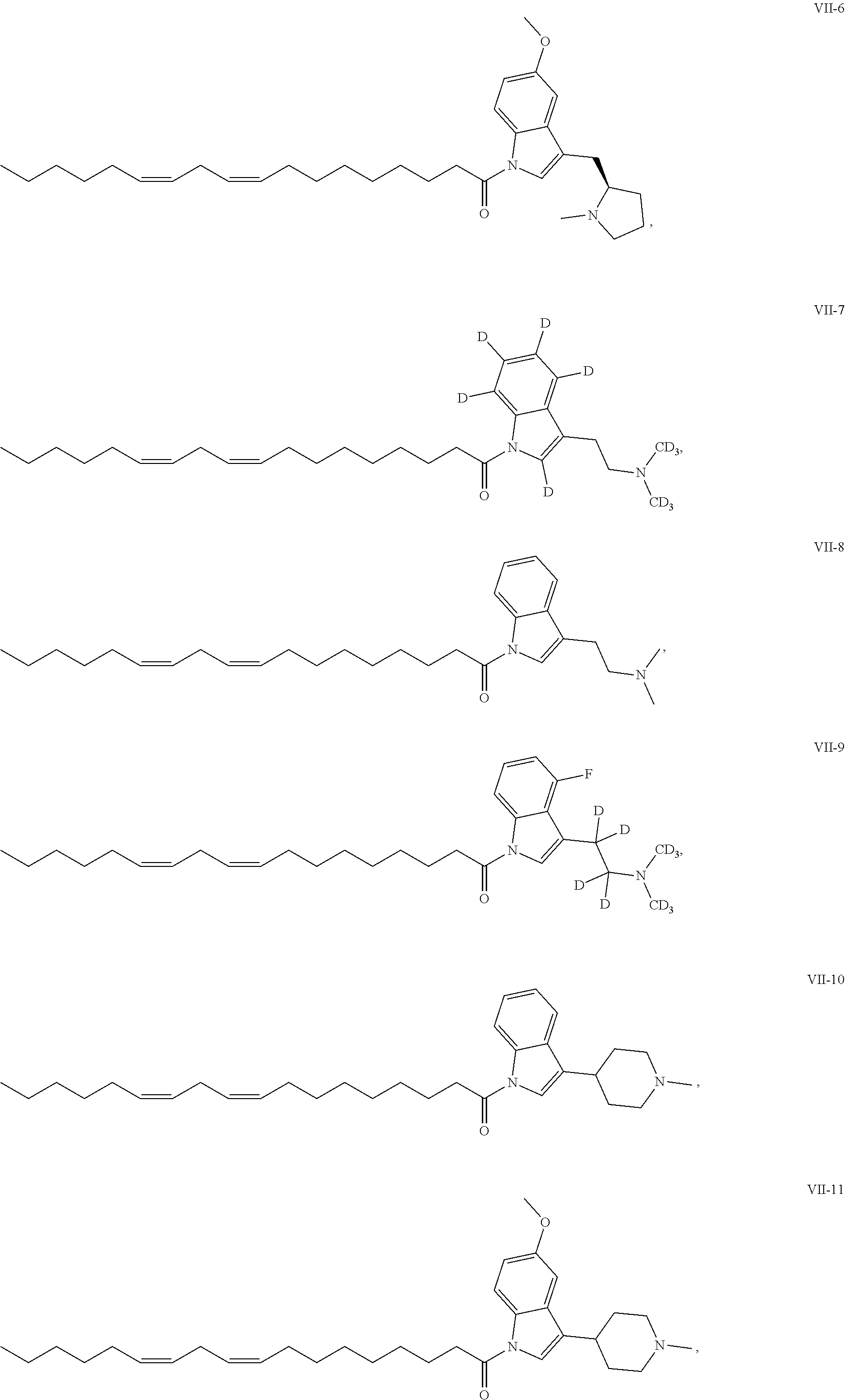

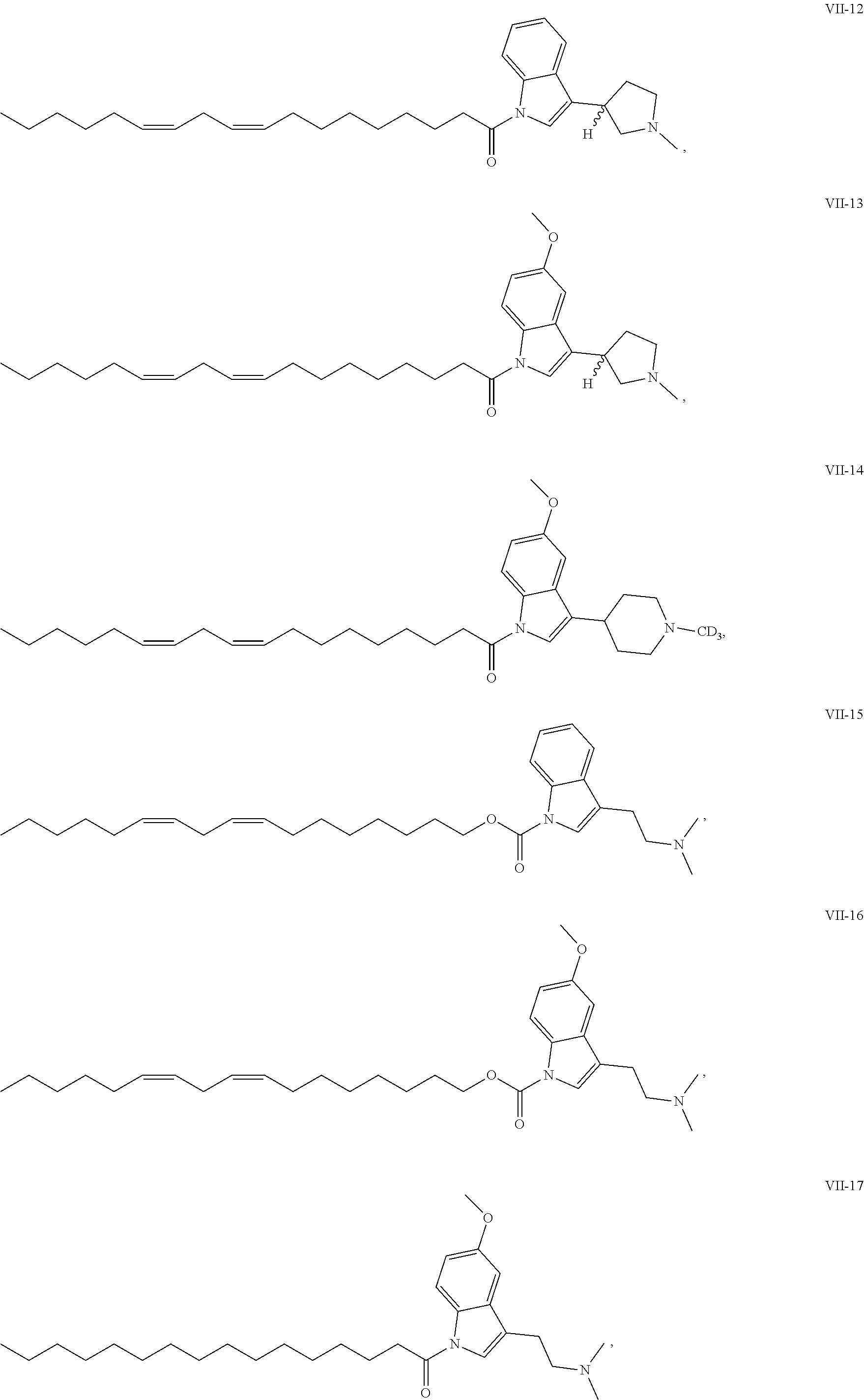

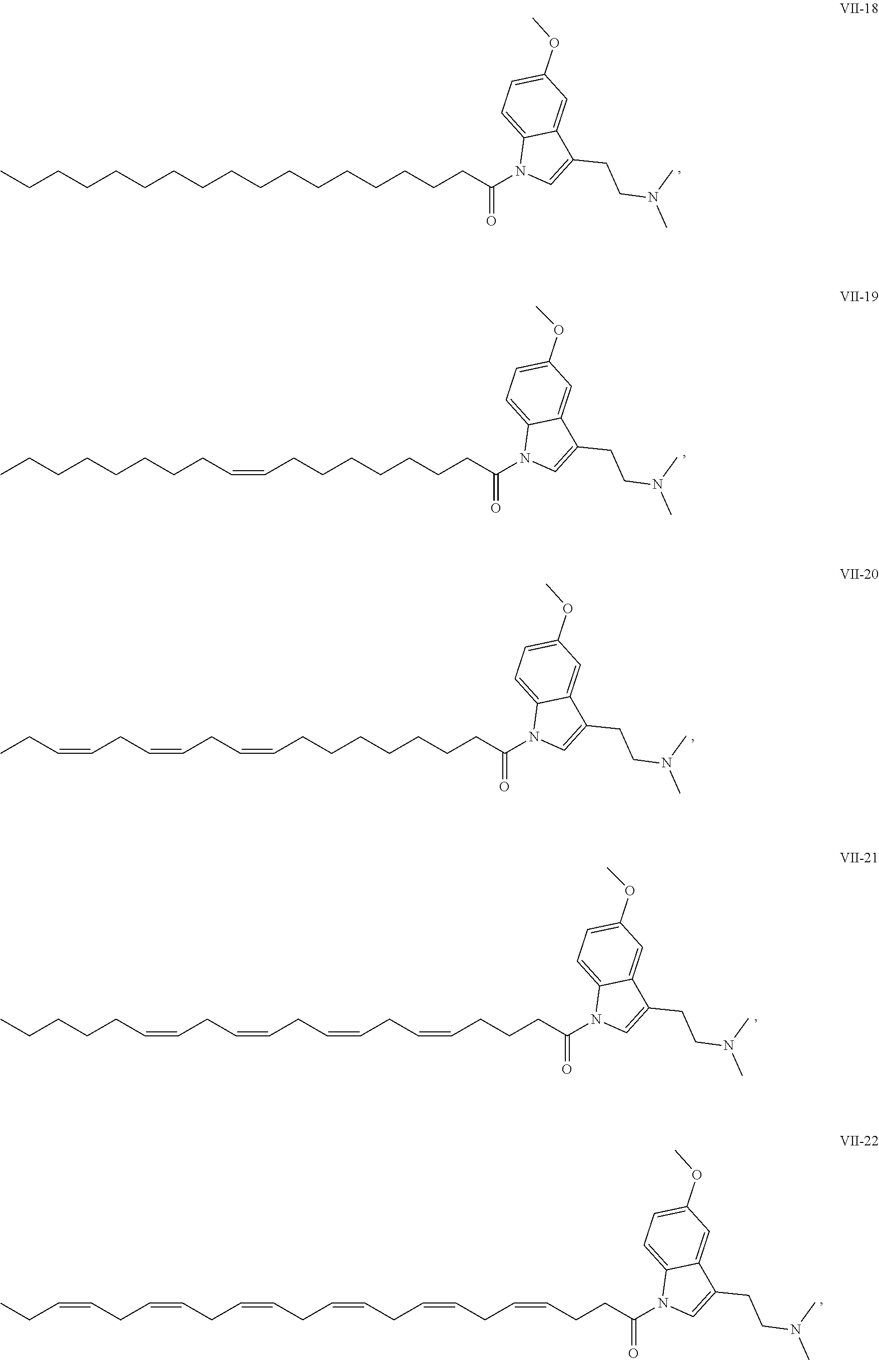

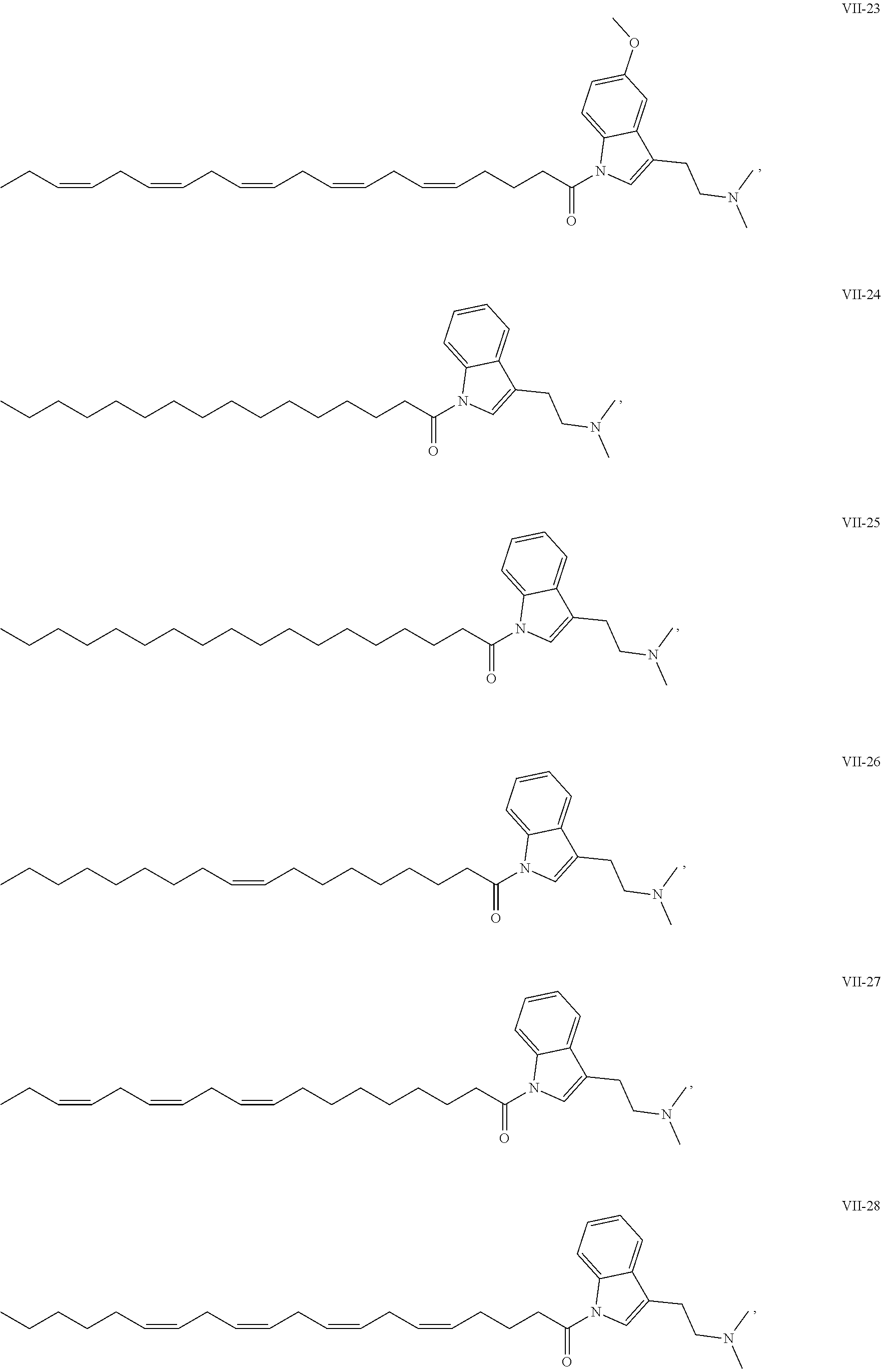

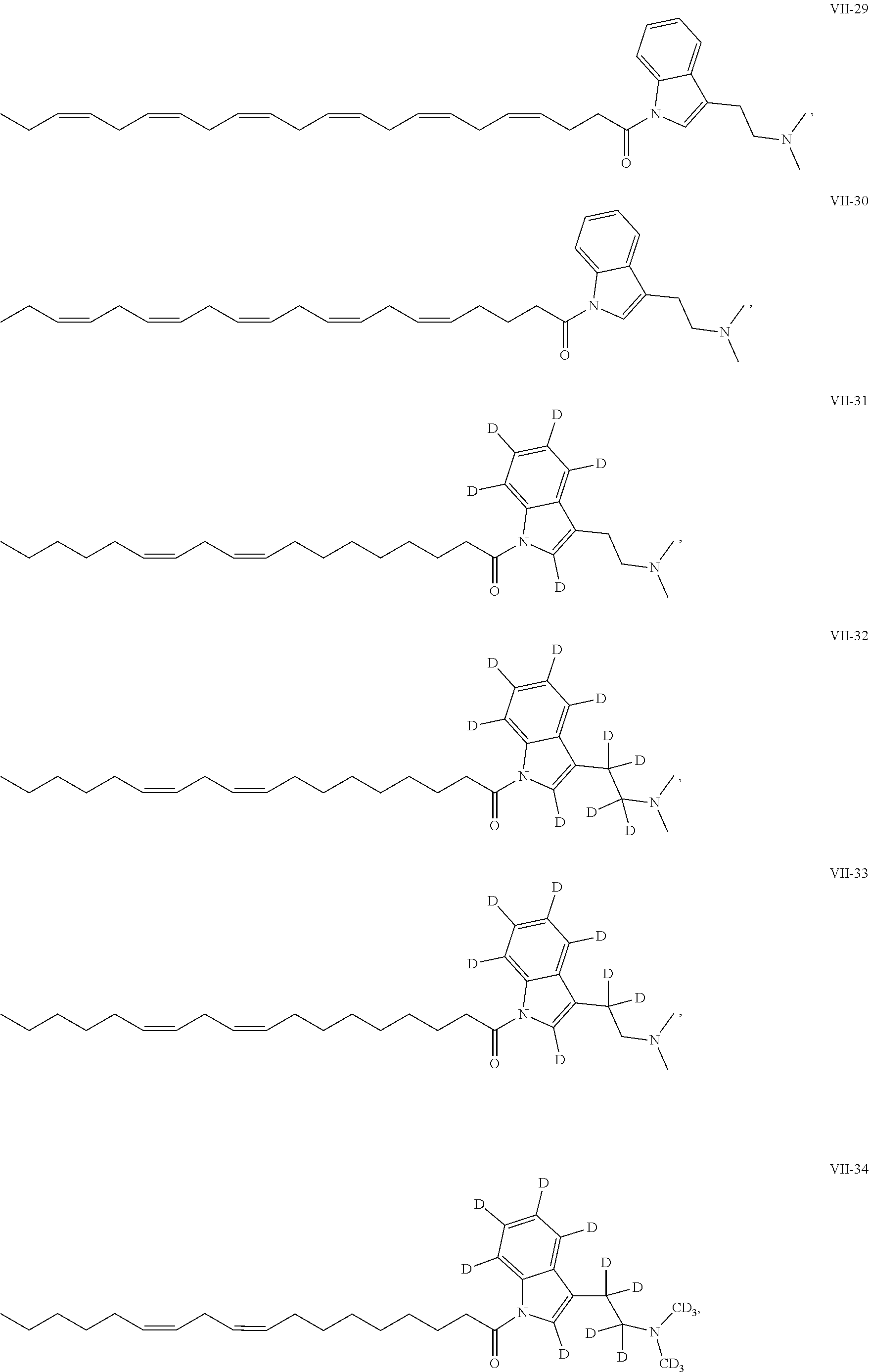

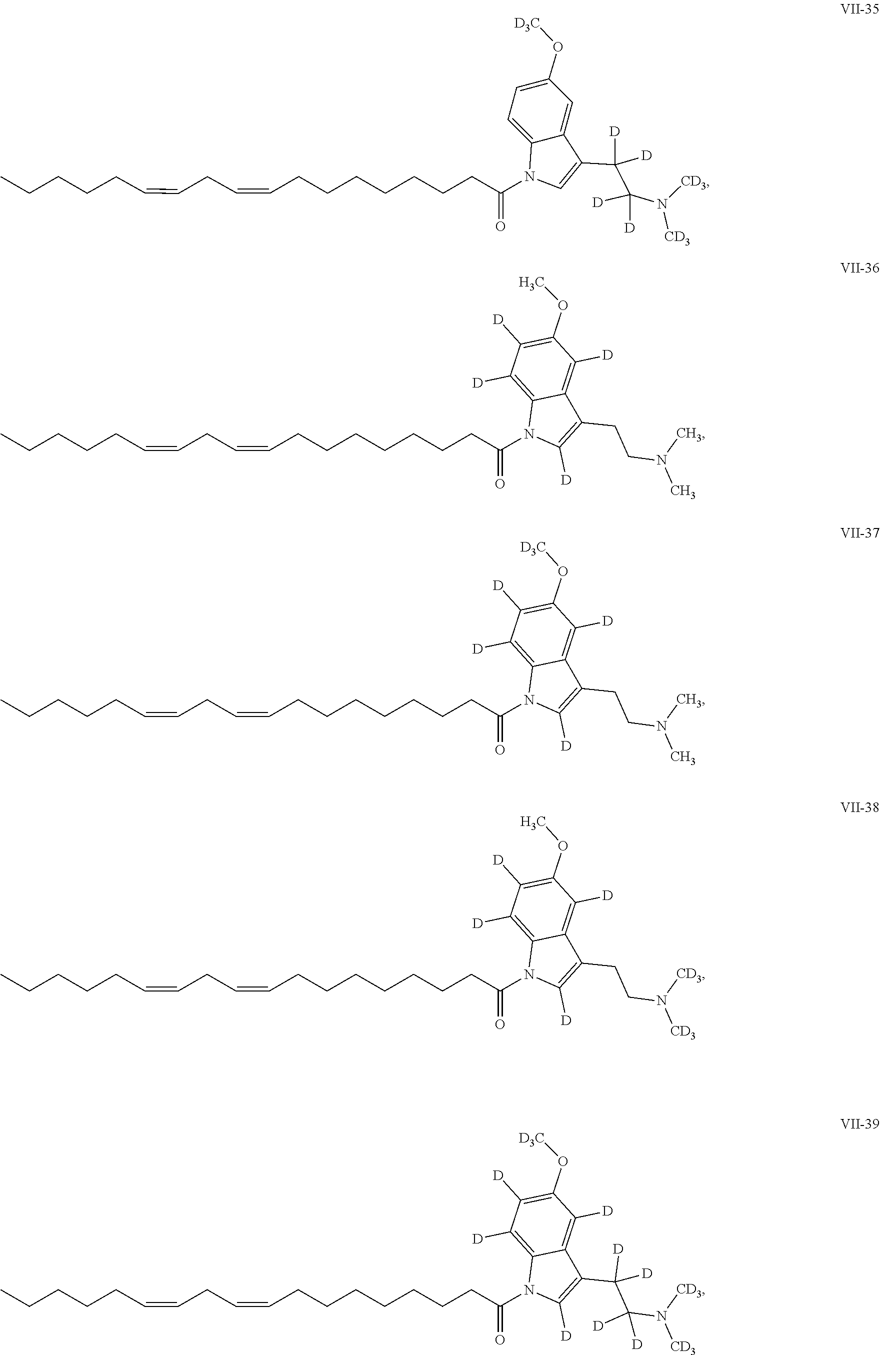

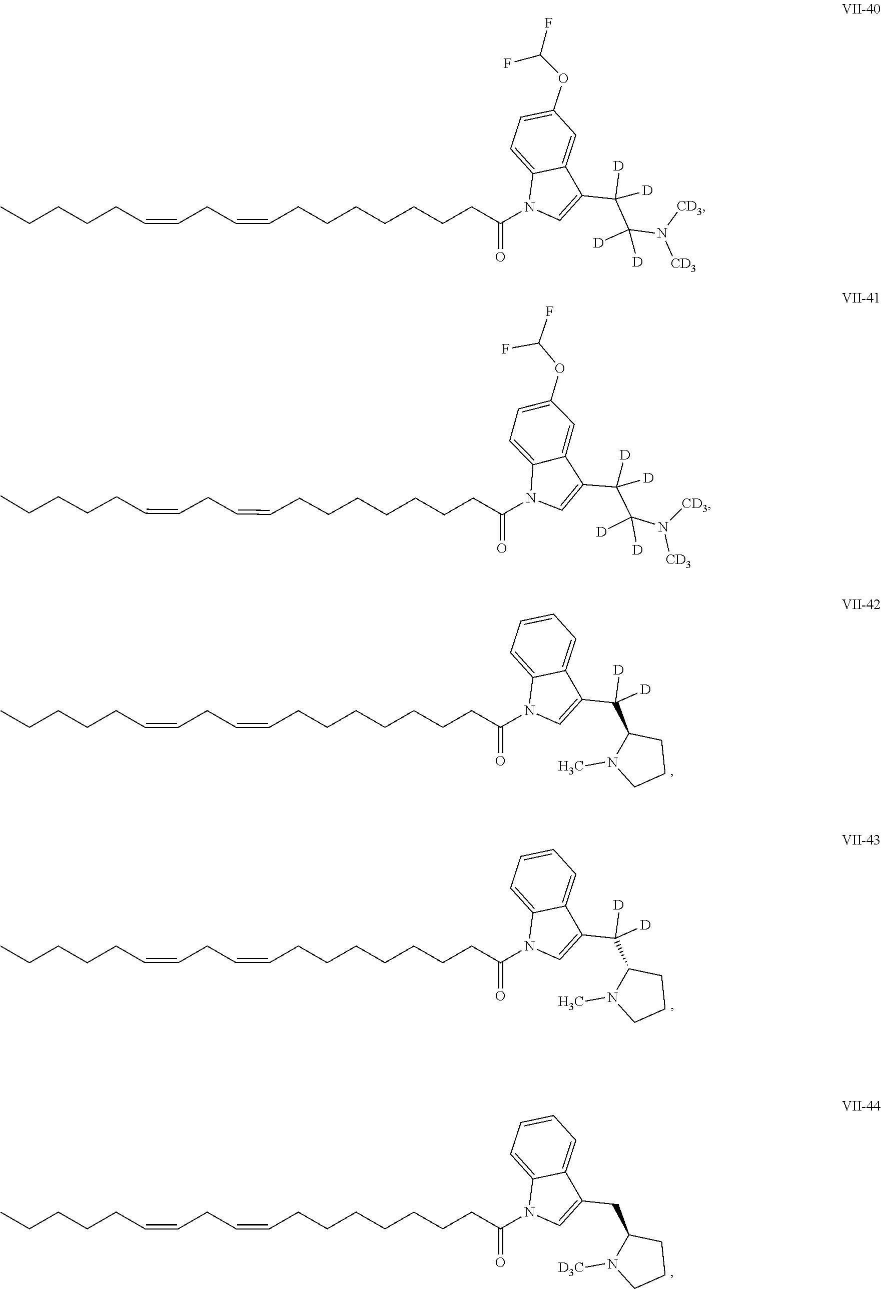 [SMILES: [2H]C([2H])([2H])N(C([2H])([2H])[2H])C([2H])([2H])C([2H])([2H])c1cn(C(=O)CCCCCCC/C=C\C/C=C\CCCCC)c2ccc(OC(F)F)cc12 ; [2H]C([2H])([2H])N(C([2H])([2H])[2H])C([2H])([2H])C([2H])([2H])c1cn(C(=O)CCCCCCC/C=C\C/C=C\CCCCC)c2ccc(OC(F)F)cc12 ; [2H]C([2H])(c1cn(C(=O)CCCCCCC/C=C\C/C=C\CCCCC)c2ccccc12)[C@H]1CCCN1C ; [2H]C([2H])(c1cn(C(=O)CCCCCCC/C=C\C/C=C\CCCCC)c2ccccc12)[C@@H]1CCCN1C ; [2H]C([2H])([2H])N1CCC[C@@H]1Cc1cn(C(=O)CCCCCCC/C=C\C/C=C\CCCCC)c2ccccc12]

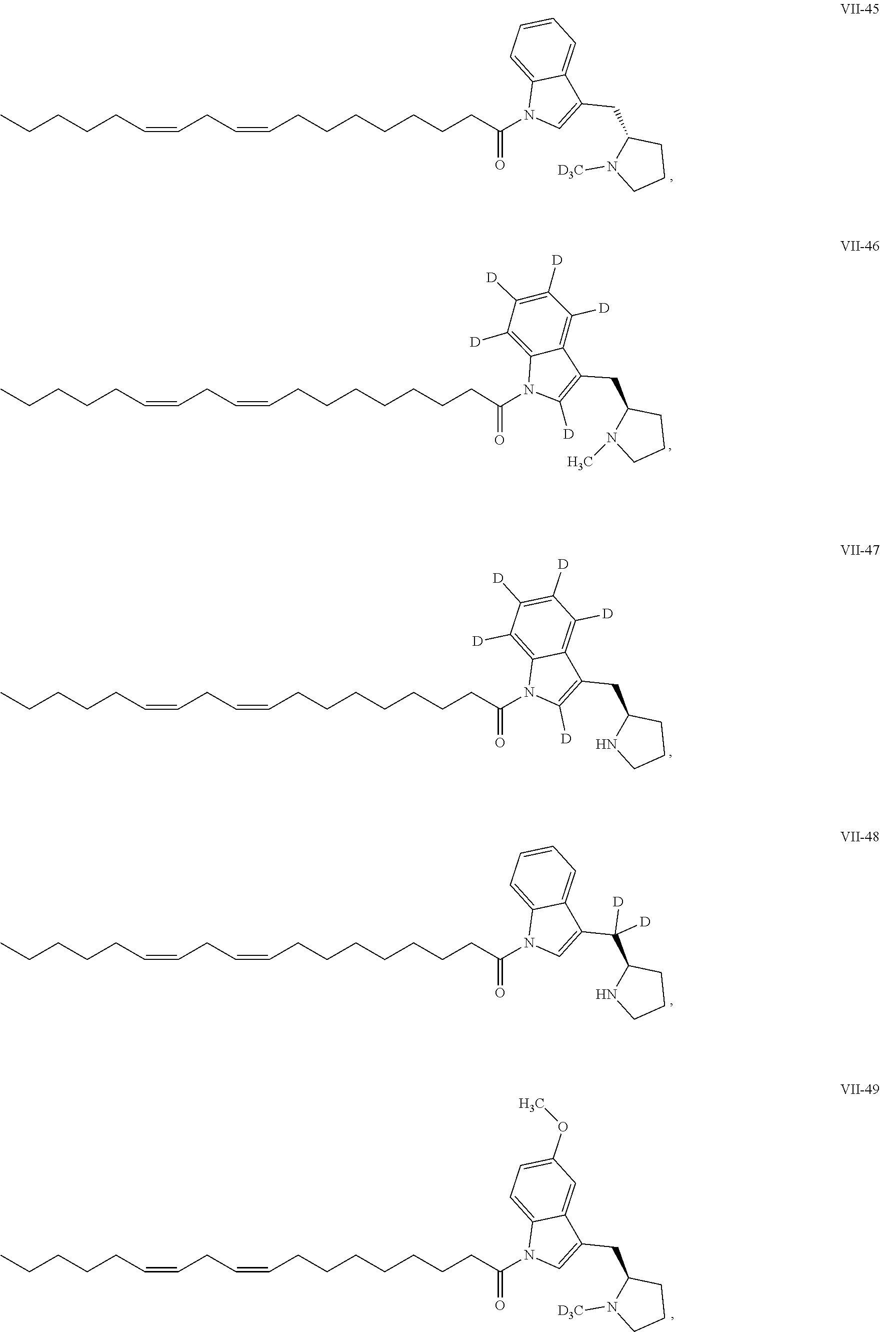 [SMILES: [2H]C([2H])([2H])N1CCC[C@H]1Cc1cn(C(=O)CCCCCCC/C=C\C/C=C\CCCCC)c2ccccc12 ; [2H]c1c([2H])c([2H])c2c(c1[2H])c(C[C@H]1CCCN1C)c([2H])n2C(=O)CCCCCCC/C=C\C/C=C\CCCCC ; [2H]c1c([2H])c([2H])c2c(c1[2H])c(C[C@H]1CCCN1)c([2H])n2C(=O)CCCCCCC/C=C\C/C=C\CCCCC ; [2H]C([2H])(c1cn(C(=O)CCCCCCC/C=C\C/C=C\CCCCC)c2ccccc12)[C@H]1CCCN1 ; [2H]C([2H])([2H])N1CCC[C@@H]1Cc1cn(C(=O)CCCCCCC/C=C\C/C=C\CCCCC)c2ccc(OC)cc12]

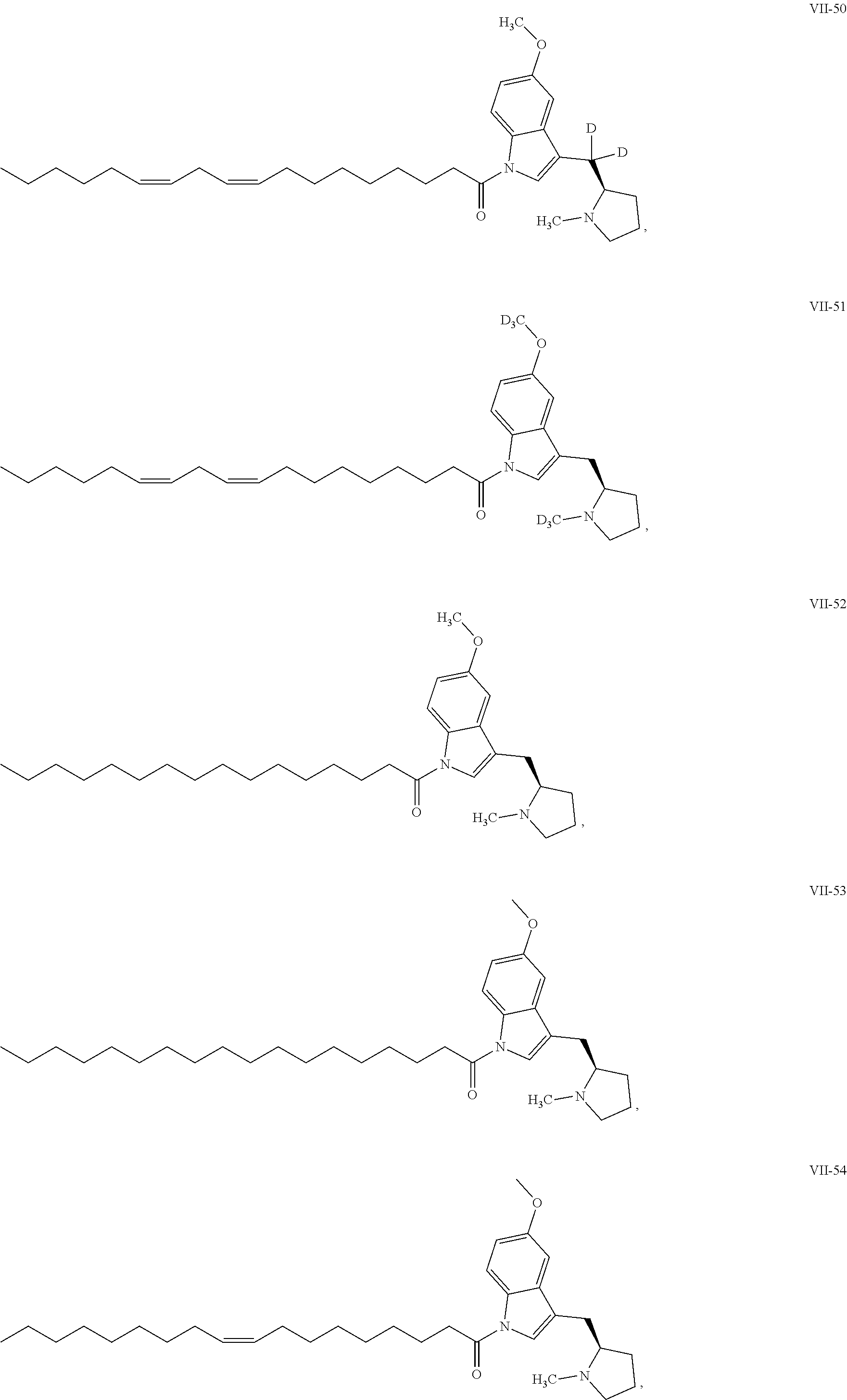 [SMILES: [2H]C([2H])(c1cn(C(=O)CCCCCCC/C=C\C/C=C\CCCCC)c2ccc(OC)cc12)[C@H]1CCCN1C ; [2H]C([2H])([2H])Oc1ccc2c(c1)c(C[C@H]1CCCN1C([2H])([2H])[2H])cn2C(=O)CCCCCCC/C=C\C/C=C\CCCCC ; CCCCCCCCCCCCCCCC(=O)n1cc(C[C@H]2CCCN2C)c2cc(OC)ccc21 ; CCCCCCCCCCCCCCCCCC(=O)n1cc(C[C@H]2CCCN2C)c2cc(OC)ccc21 ; CCCCCCCC/C=C\CCCCCCCC(=O)n1cc(C[C@H]2CCCN2C)c2cc(OC)ccc21]

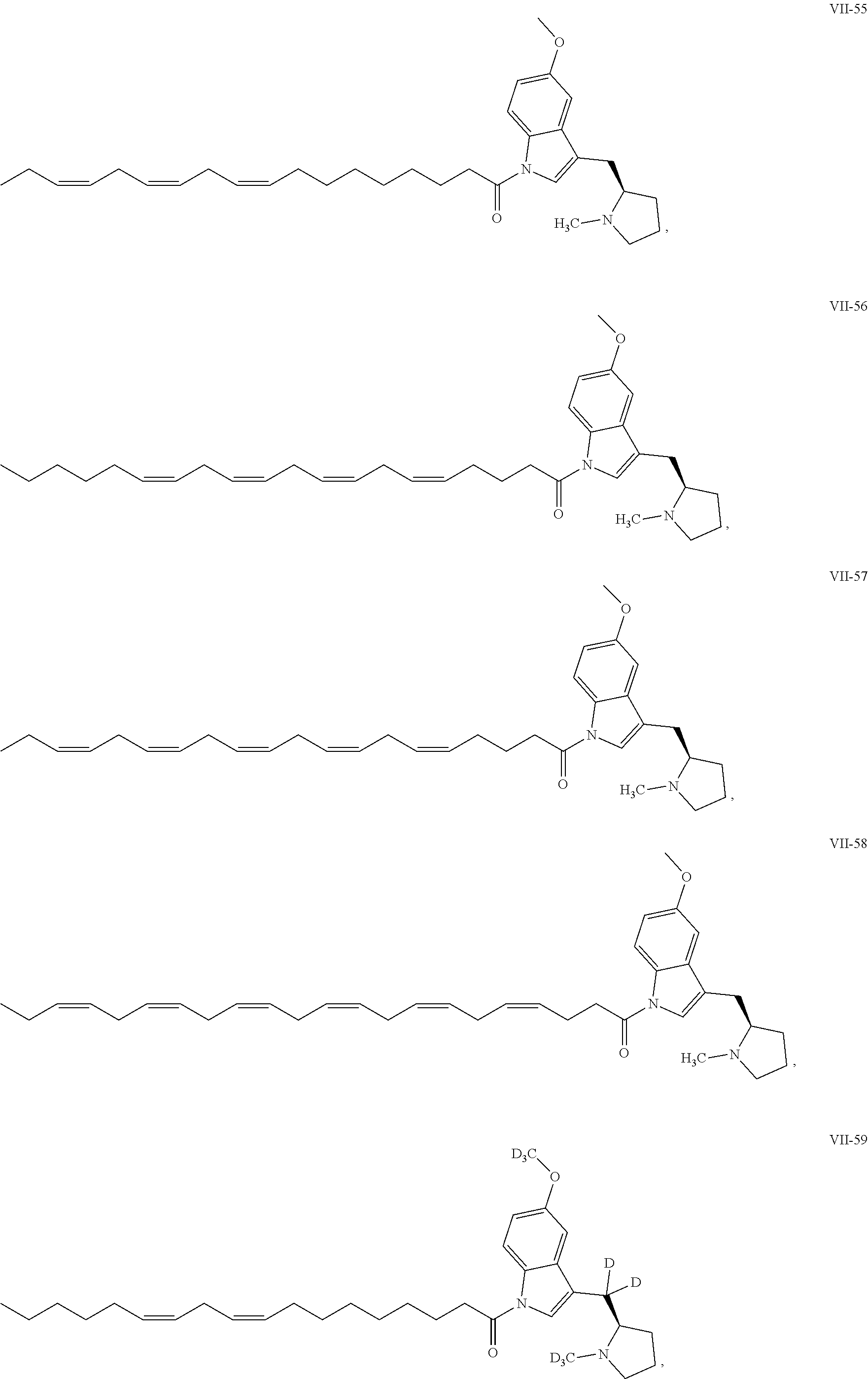 [SMILES: CC/C=C\C/C=C\C/C=C\CCCCCCCC(=O)n1cc(C[C@H]2CCCN2C)c2cc(OC)ccc21 ; CCCCC/C=C\C/C=C\C/C=C\C/C=C\CCCC(=O)n1cc(C[C@H]2CCCN2C)c2cc(OC)ccc21 ; CC/C=C\C/C=C\C/C=C\C/C=C\C/C=C\CCCC(=O)n1cc(C[C@H]2CCCN2C)c2cc(OC)ccc21 ; CC/C=C\C/C=C\C/C=C\C/C=C\C/C=C\C/C=C\CCC(=O)n1cc(C[C@H]2CCCN2C)c2cc(OC)ccc21 ; [2H]C([2H])([2H])Oc1ccc2c(c1)c(C([2H])([2H])[C@H]1CCCN1C([2H])([2H])[2H])cn2C(=O)CCCCCCC/C=C\C/C=C\CCCCC]

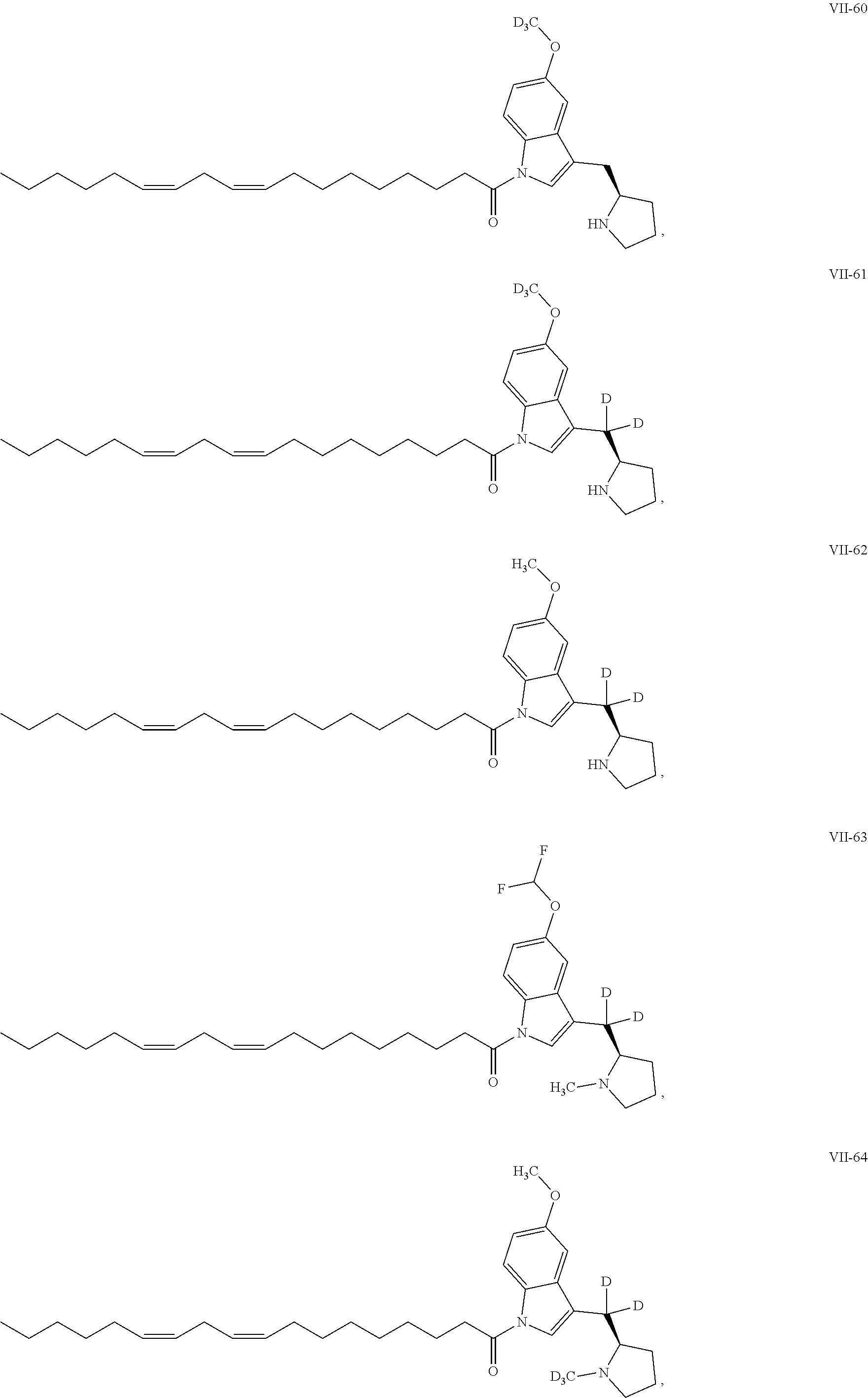 [SMILES: [2H]C([2H])([2H])Oc1ccc2c(c1)c(C[C@H]1CCCN1)cn2C(=O)CCCCCCC/C=C\C/C=C\CCCCC ; [2H]C([2H])([2H])Oc1ccc2c(c1)c(C([2H])([2H])[C@H]1CCCN1)cn2C(=O)CCCCCCC/C=C\C/C=C\CCCCC ; [2H]C([2H])(c1cn(C(=O)CCCCCCC/C=C\C/C=C\CCCCC)c2ccc(OC)cc12)[C@H]1CCCN1 ; [2H]C([2H])(c1cn(C(=O)CCCCCCC/C=C\C/C=C\CCCCC)c2ccc(OC(F)F)cc12)[C@H]1CCCN1C ; [2H]C([2H])(c1cn(C(=O)CCCCCCC/C=C\C/C=C\CCCCC)c2ccc(OC)cc12)[C@H]1CCCN1C([2H])([2H])[2H]]

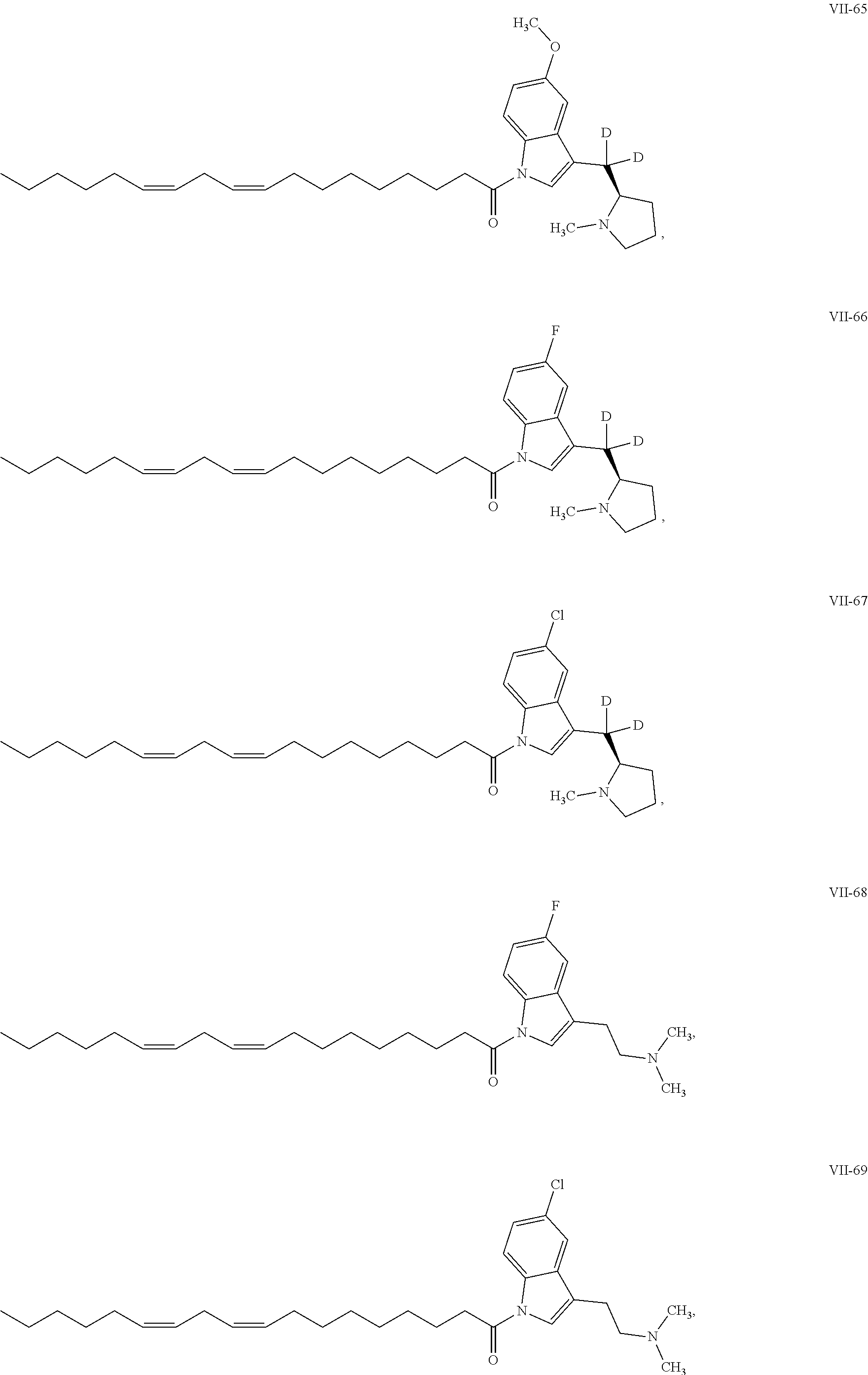 [SMILES: [2H]C([2H])(c1cn(C(=O)CCCCCCC/C=C\C/C=C\CCCCC)c2ccc(OC)cc12)[C@H]1CCCN1C ; [2H]C([2H])(c1cn(C(=O)CCCCCCC/C=C\C/C=C\CCCCC)c2ccc(F)cc12)[C@H]1CCCN1C ; [2H]C([2H])(c1cn(C(=O)CCCCCCC/C=C\C/C=C\CCCCC)c2ccc(Cl)cc12)[C@H]1CCCN1C ; CCCCC/C=C\C/C=C\CCCCCCCC(=O)n1cc(CCN(C)C)c2cc(F)ccc21 ; CCCCC/C=C\C/C=C\CCCCCCCC(=O)n1cc(CCN(C)C)c2cc(Cl)ccc21]

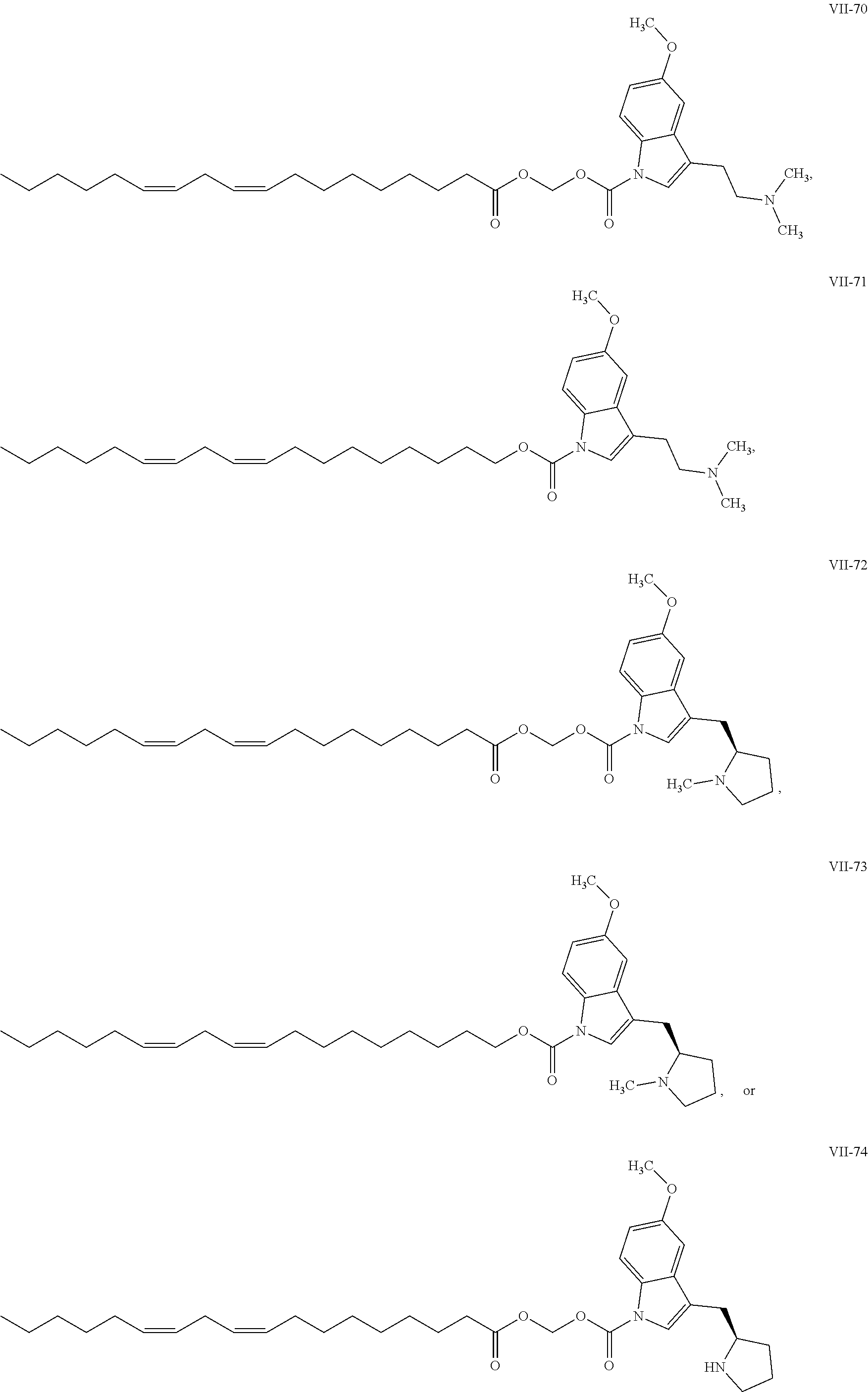

or a pharmaceutically acceptable salt and/or solvate thereof.

In some embodiments, the one or more hallucinogens are selected from the compound disclosed and claimed in Applicant's co-pending U.S. provisional patent application Ser. No. 63/332,450 filed on Apr. 19, 2022, the contents of which are incorporated by reference in their entirety. Therefore, in some embodiments, the one or more hallucinogens are selected from a compound of Formula (VIII) or a pharmaceutically acceptable salt, prodrug and/or solvate thereof:

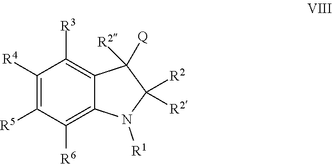

or a pharmaceutically acceptable salt, solvate and/or prodrug thereof, wherein:
R¹ is selected from H, C(O)R⁷, CO₂R⁷, C(O)N(R⁷)(R⁷), S(O)R⁷ and SO₂R⁷;
Q is selected from Q1, Q2, Q3, Q4 and Q5:

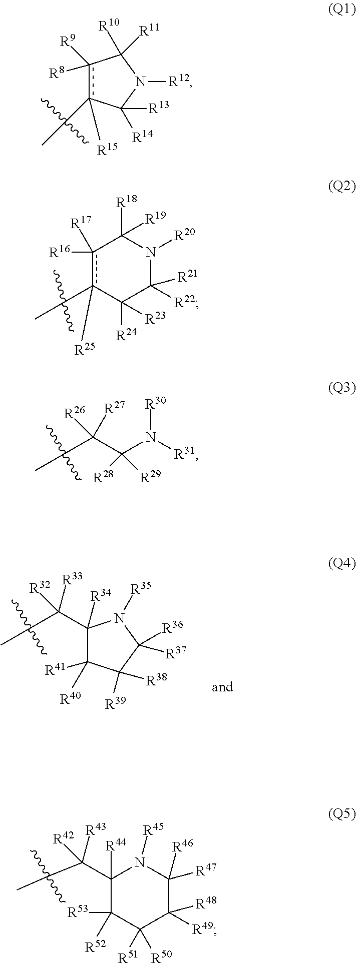

[symbol] is a single bond or a double bond provided that when [symbol] in Q1 is a double bond then R⁹ and R¹⁵ are not present, and when [symbol] in Q2 is a double bond then R¹⁷ and R²⁵ are not present;
R², R², R²″, R³ and R⁶ are independently selected from H, halo, C₁₋₆alkyl and C₁-C₆alkoxy;
one or both of R⁴ and R⁵ is independently selected from H, halo, C₁₋₆alkyl and C₁-C₆alkoxy, or
R⁴ and R⁵ are linked together to form O—(CH₂) 1-20, or one of R⁴ and R⁵ is selected from A, O-A and C₁₋₄alkylene A and the other of R⁴ and R⁵ is H;
A is selected from phenyl, C₃₋₆cycloalkyl, 3- to 6-membered heterocycloalkyl comprising 1 to 4 heteromoeities independently selected from O, S, S(O), SO₂, N and NR⁵⁴ and 5- to 6-membered heteroaryl comprising 1 to 4 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or more substituents independently selected from halo, C₁-4alkyl and OC₁₋₄alkyl;
R⁷ is selected from H and C₁₋₆alkyl, wherein the C₁₋₆alkyl is optionally substituted with one or more substituents independently selected from halo, OR⁵⁵, N(R⁵⁵)(R⁵⁶) and SR⁵⁵ and/or are optionally interrupted by one to three heteromoieties independently selected from O, C(O), CO₂ and NR⁵⁷;
R⁷ is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸ R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹ R⁵² and R⁵³ are independently selected from H, halo and C₁₋₆alkyl;
R¹², R²⁰, R³⁵ and R⁴⁵ are independently selected from H, C₁₋₆alkyl and C(O) C₁₋₆alkyl; R³⁰ and R³¹ are independently selected from H, C₁₋₆alkyl and C(O) C₁₋₆alkyl, or R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁶, R⁵⁷ and R⁵⁸ are independently selected from H and C₁₋₆alkyl; and all available hydrogen atoms are optionally and independently substituted with a fluorine atom or chlorine atom and all available atoms are optionally substituted with alternate isotope thereof, provided that when Q is Q3, R²⁶, R²⁷, R²⁸ and R²⁹ are all H and R³⁰ and R³¹ are H or CH₃, and:
R¹, R², R², R²″, R³, R⁴, R⁵ and R⁶ are all H,
R¹, R², R², R²″, R³, R⁵ and R⁶ are all H and R⁴ is OCH₃, or
R¹, R², R², R²″, R³, R⁴, R⁵ and R⁶ are all H and R⁵ is OCH₃,
then the compound of Formula I is an (R)-or(S)-enantiomer of the carbon to which Q is attached.

In some embodiments, the compound of Formula VIII is:

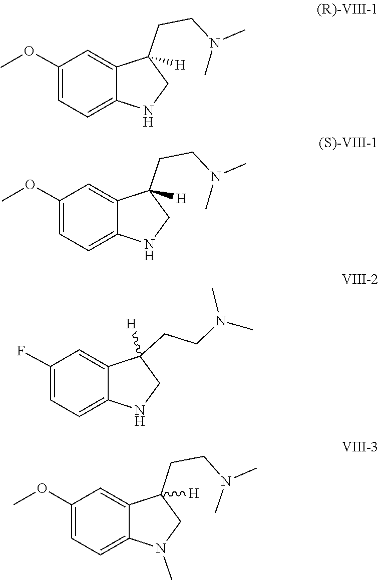

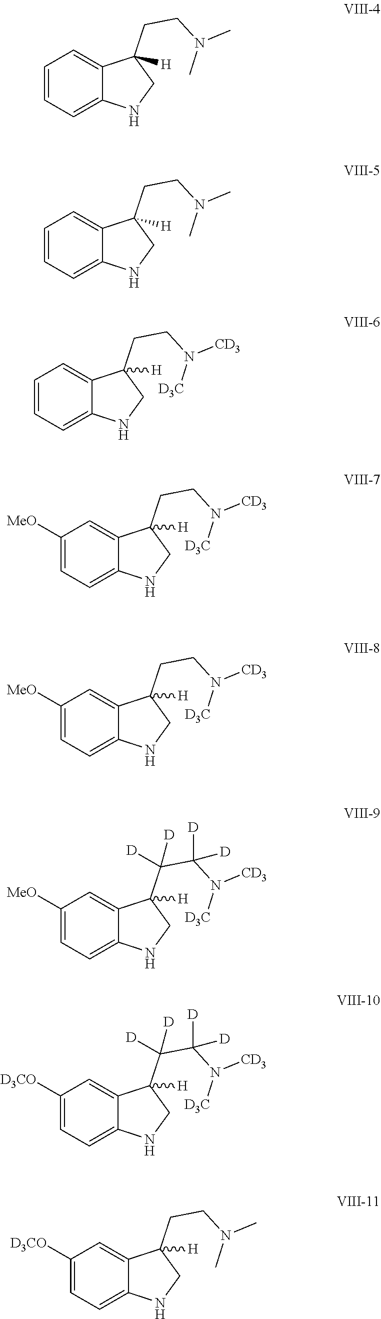

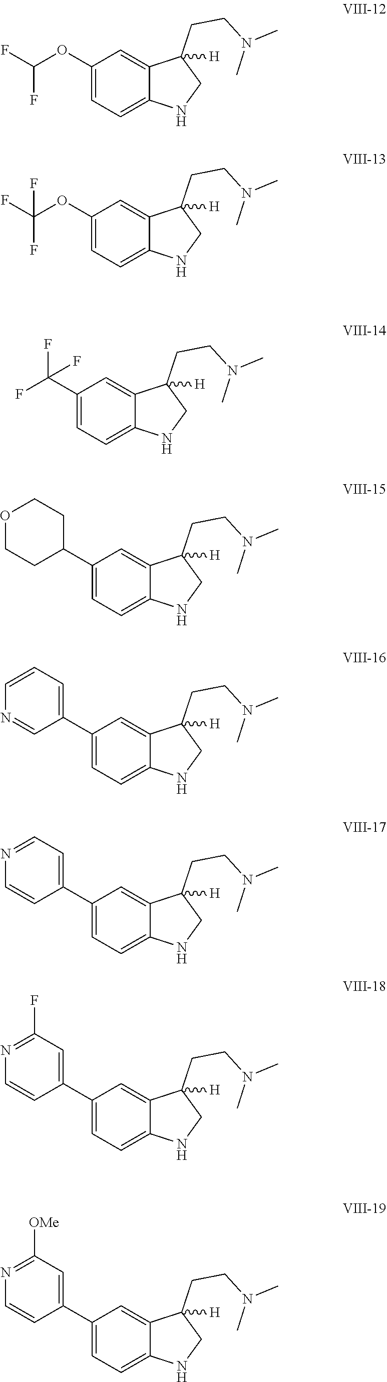

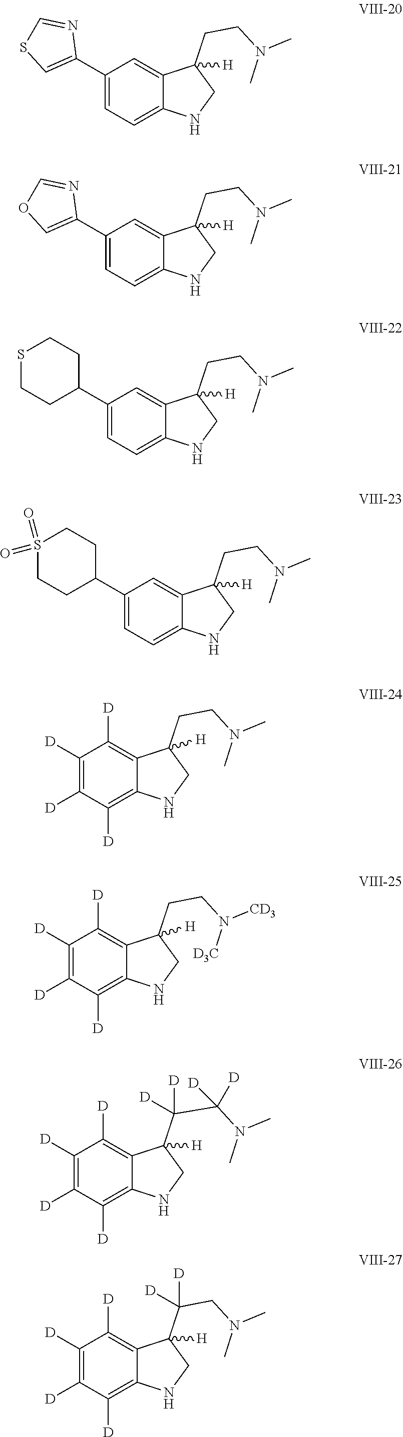

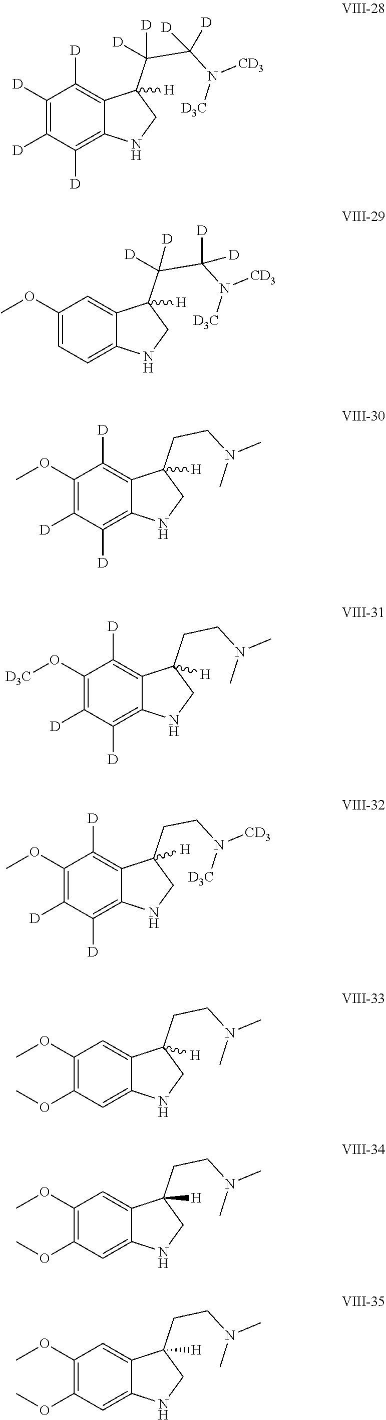

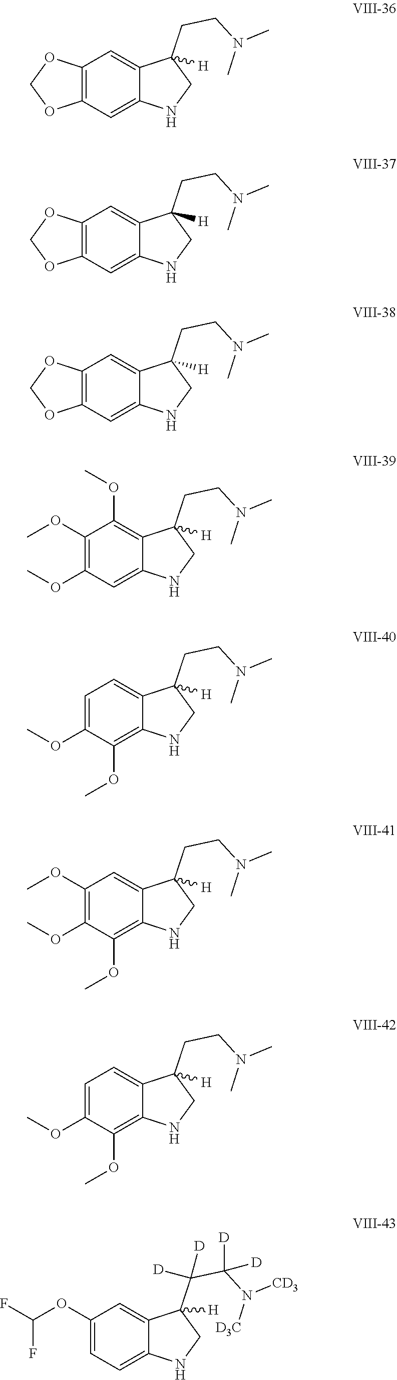

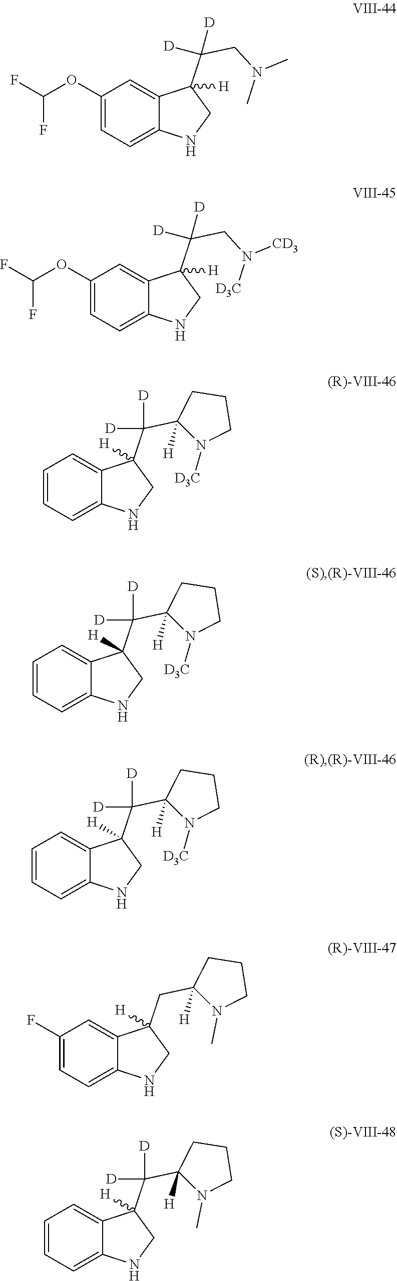

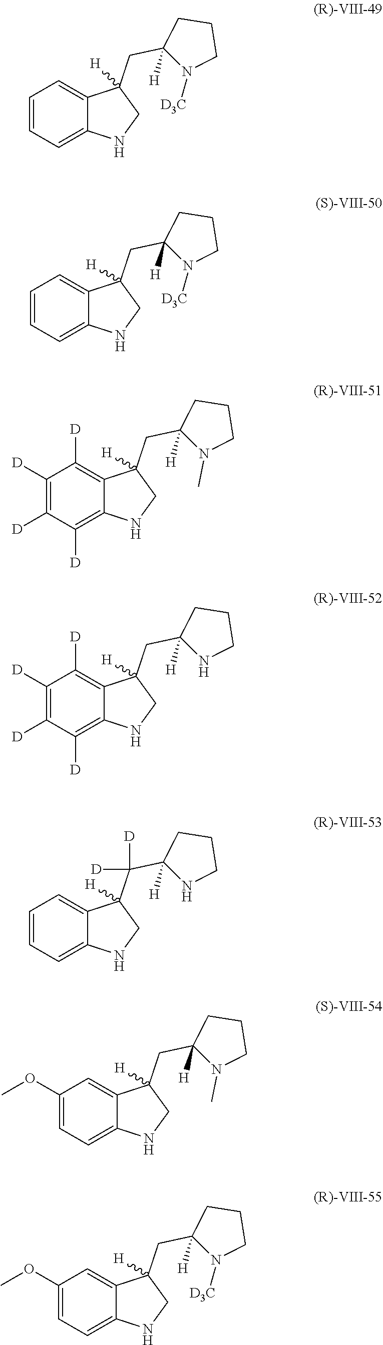

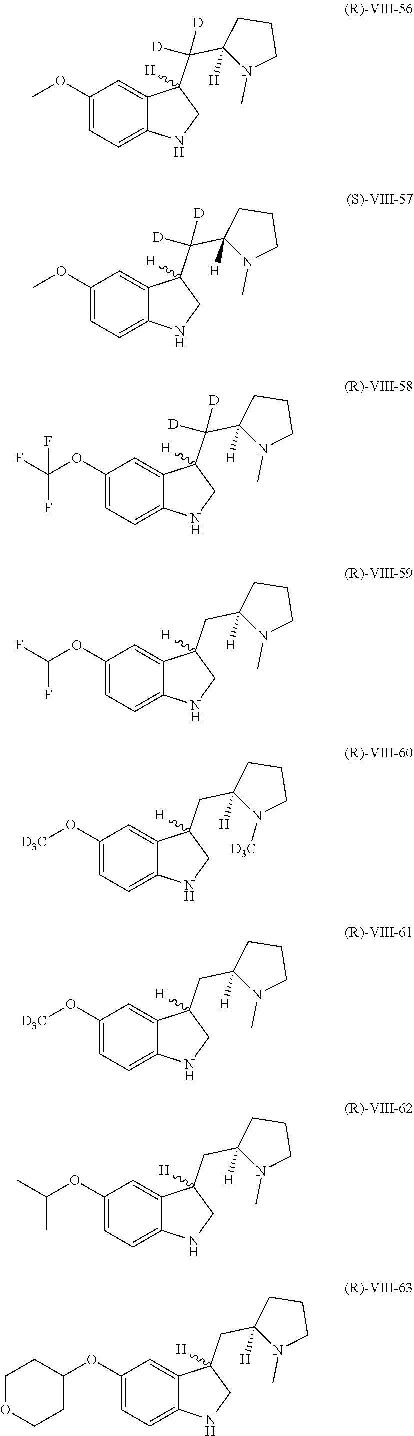

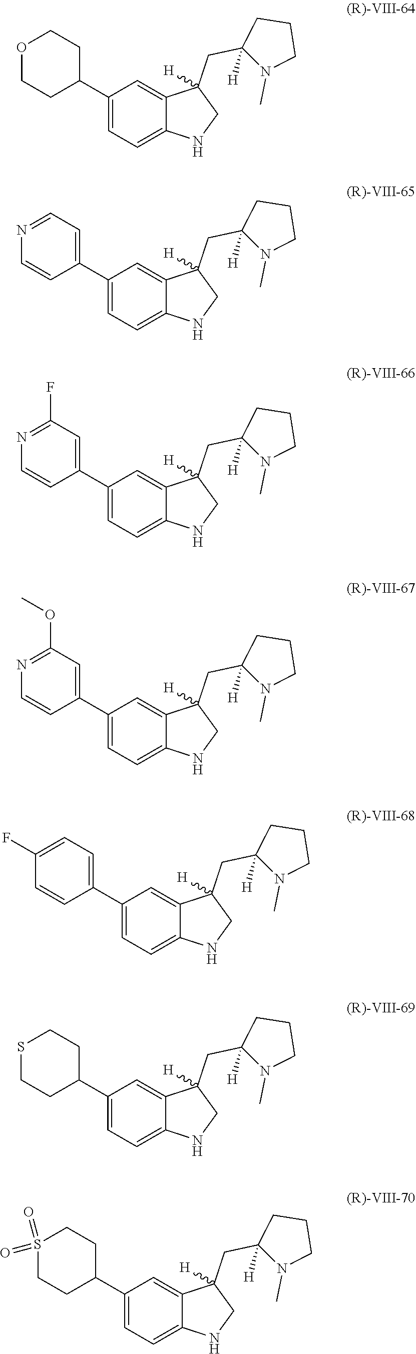

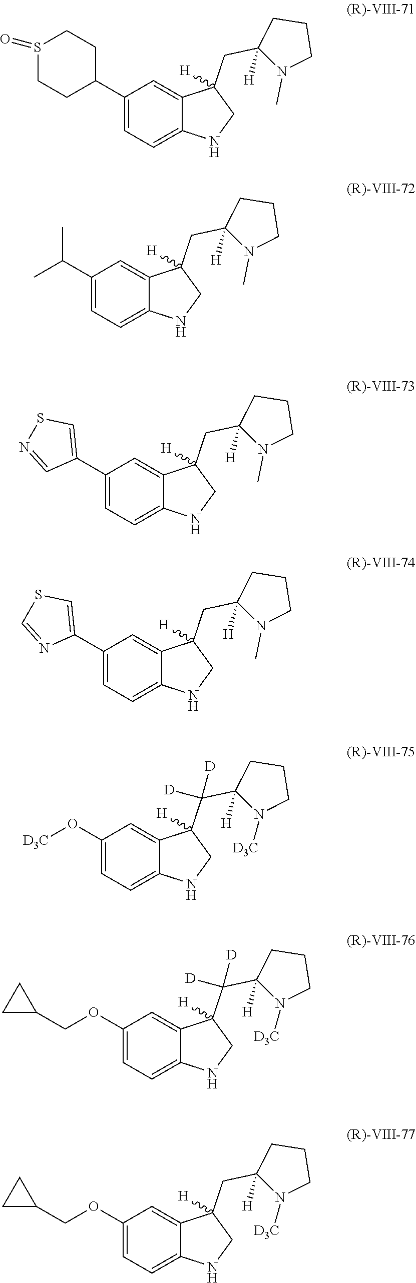

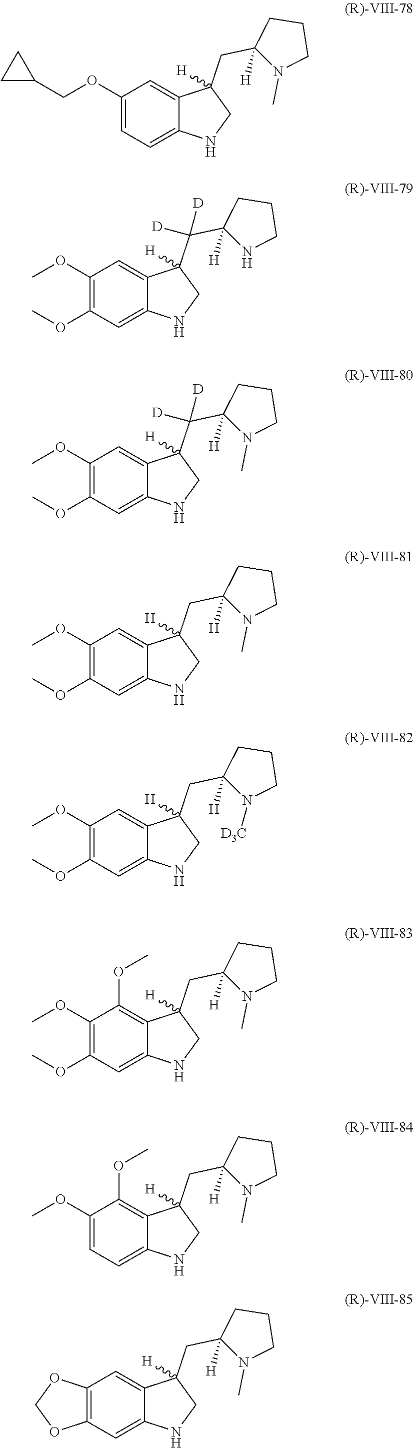

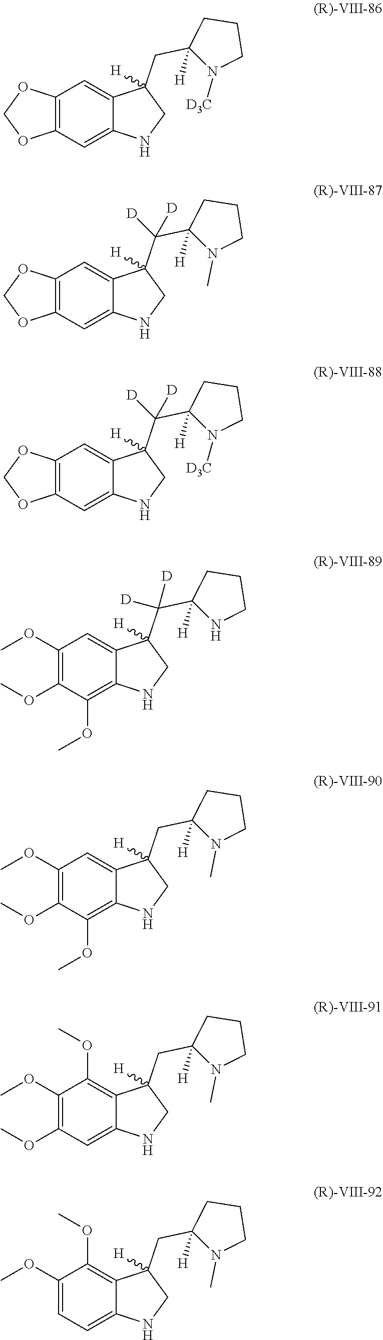

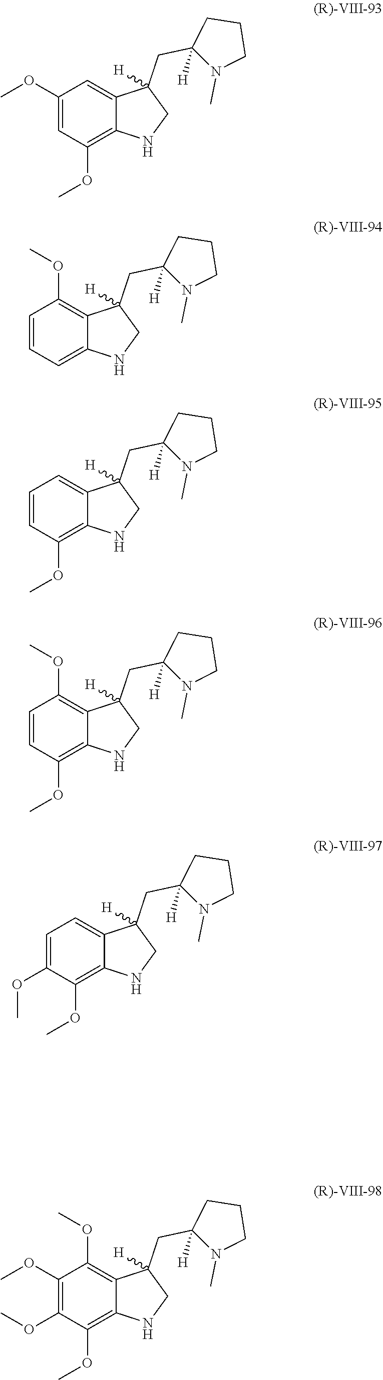 [SMILES: COc1cc(OC)c2c(c1)C(C[C@H]1CCCN1C)CN2 ; COc1cccc2c1C(C[C@H]1CCCN1C)CN2 ; COc1cccc2c1NCC2C[C@H]1CCCN1C ; COc1ccc(OC)c2c1NCC2C[C@H]1CCCN1C ; COc1ccc2c(c1OC)NCC2C[C@H]1CCCN1C ; COc1c2c(c(OC)c(OC)c1OC)C(C[C@H]1CCCN1C)CN2]

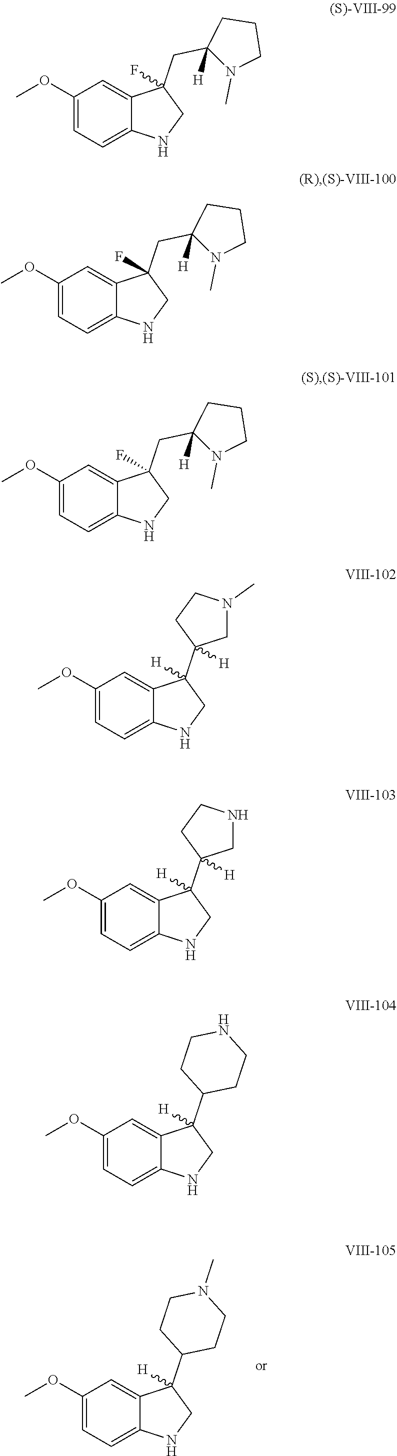

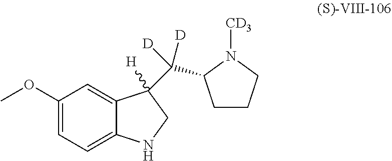 [SMILES: [2H]C([2H])(C1CNc2ccc(OC)cc21)[C@H]1CCCN1C([2H])([2H])[2H]]

or a pharmaceutically acceptable salt, solvate and/or prodrug thereof.

In some embodiments, the one or more fatty acids are selected from any such acid derived from fats by hydrolysis and having from 4 to 30 carbon atoms, 6 to 28 carbon atoms or 6 to 24 carbon atoms. In some embodiments, the fatty acids are selected from myristic acid, caproic acid, caprylic acid, capric acid, lauric acid, palmitic acid, stearic acid, arachidic acid, behenic acid, lignoceric acid, palmitolic acid, oleic acid, linoleic acid, linolenic acid, arachidonic acid, eicosapentaenoic acid, docosahexaenoic acid and combinations thereof.

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66 (1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di—and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono-or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts.

In some embodiments, in the case of an acid (—COOH) or alcohol group being present, pharmaceutically acceptable esters can be employed to modify the solubility or hydrolysis characteristics of a compound. In some embodiments, the esters are alkyl esters. In some embodiments, the alkyl esters are selected from isopropyl esters, methyl esters, ethyl esters, propyl esters and mixtures thereof.

Formation of a pharmaceutically acceptable salt may be achieved using standard techniques. For example, a neutral compound is treated with an acid or base in a suitable solvent and the formed salt is isolated by filtration, extraction or any other suitable method.

Examples of suitable solvate solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

In some embodiments, the one or more hallucinogens, or a pharmaceutically acceptable salt, prodrug and/or solvate thereof, and the one or more fatty acids, or a pharmaceutically acceptable salt, prodrug and/or solvate thereof, in the compositions and kits of the present application are formulated as separate pharmaceutical compositions, for separate administration to, or use in, subjects. In this embodiment, the separate pharmaceutical compositions are formulated independently of each other and in accordance with the desired mode of administration, which may be the same or different, for each of the one or more hallucinogens, or a pharmaceutically acceptable salt, prodrug and/or solvate thereof, and the one or more fatty acids, or a pharmaceutically acceptable salt, prodrug and/or solvate thereof.

In some embodiments, one or more hallucinogens, or a pharmaceutically acceptable salt, prodrug and/or solvate thereof, and the one or more fatty acids, or a pharmaceutically acceptable salt, prodrug and/or solvate thereof, in the compositions and kits of the present application are formulated in a single pharmaceutical composition, for administration to, or use in, subjects.

The one or more hallucinogens, or a salt, prodrug and/or solvate thereof, and the one or more fatty acids, or a salt, prodrug and/or solvate thereof, are administered to a subject or are used in a variety of forms depending on the selected route of administration, as will be understood by those skilled in the art. For example, the one or more hallucinogens, or a salt, prodrug and/or solvate thereof, and/or the one or more fatty acids, or a salt, prodrug and/or solvate thereof, are administered by oral, inhalation, parenteral, buccal, sublingual, insufflation, epidurally, nasal, rectal, vaginal, patch, pump, minipump, topical or transdermal administration and the pharmaceutical compositions formulated accordingly. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, the one or more hallucinogens, or a salt, prodrug and/or solvate thereof, and/or the one or more fatty acids, or a salt, prodrug and/or solvate thereof, are orally administered, for example, with an inert diluent or with an assimilable edible carrier, or are enclosed in hard or soft shell gelatin capsules, or are compressed into tablets, or are incorporated directly with the food of the diet. In some embodiments, the one or more hallucinogens, or a salt, prodrug and/or solvate thereof, and/or the one or more fatty acids, or a salt, prodrug and/or solvate thereof, are incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, com starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., talc or silica); disintegrants (e.g., potato starch or sodium starch glycolate); or wetting agents (e.g., sodium lauryl sulphate), or solvents (e.g., water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. For oral administration in a capsule form, useful carriers, solvents or diluents include, but are not limited to, lactose, ethanol and dried com starch.

In some embodiments, liquid preparations for oral administration take the form of, for example, solutions, syrups or suspensions, or they are suitably presented as a dry product for constitution with water or other suitable vehicle before use. If desired, certain sweetening and/or flavoring and/or coloring agents are added. Such liquid preparations for oral administration are prepared by conventional means with pharmaceutically acceptable additives such as suspending agents (e.g., sorbitol syrup, or methyl cellulose); emulsifying agents (e.g., lecithin or acacia); non-aqueous vehicles; and preservatives (e.g., methyl or propyl p-hydroxybenzoates or sorbic acid). Useful diluents include lactose and high molecular weight polyethylene glycols.

It is also possible to freeze-dry the one or more hallucinogens, or a salt, prodrug and/or solvate thereof, and/or the one or more fatty acids, or a salt, prodrug and/or solvate thereof, in the same or separate compositions, and use the lyophilizates obtained, for example, for the preparation of products for injection.

In some embodiments, the one or more hallucinogens, or a salt, prodrug and/or solvate thereof, and/or the one or more fatty acids, or a salt, prodrug and/or solvate thereof, area administered or used parenterally. For example, solutions are prepared in water suitably mixed with a surfactant such as hydroxypropylcellulose. In some embodiments, dispersions are prepared in glycerol, liquid polyethylene glycols, DMSO and mixtures thereof with or without alcohol. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, the one or more hallucinogens, or a salt, prodrug and/or solvate thereof, and/or the one or more fatty acids, or a salt, prodrug and/or solvate thereof, are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions and contain formulating agents such as suspending, stabilizing and/or dispersing agents. In all cases, the form must be sterile and must be fluid to the extent that easy syringability exists. Alternatively, the one or more hallucinogens, or a salt, prodrug and/or solvate thereof, and/or the one or more fatty acids, or a salt, prodrug and/or solvate thereof, are suitably in a sterile powder form for reconstitution with a suitable vehicle, e.g., sterile pyrogen-free water, before use.

In some embodiments, compositions for nasal administration are conveniently formulated as aerosols, sprays, drops, gels and powders. For intranasal administration or administration by inhalation, the compositions of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of the one or more hallucinogens, or a salt, prodrug and/or solvate thereof, and/or the one or more fatty acids, or a salt, prodrug and/or solvate thereof, and a suitable powder base such as lactose or starch. The aerosol dosage forms can also take the form of a pump-atomizer.

Compositions suitable for buccal or sublingual administration include tablets, lozenges and pastilles, wherein a compound of the application is formulated with a carrier such as sugar, acacia, tragacanth, or gelatin and glycerine. Compositions for rectal administration are conveniently in the form of suppositories containing a conventional suppository base.

Suppository forms are useful for vaginal, urethral and rectal administrations. Such suppositories will generally be constructed of a mixture of substances that is solid at room temperature but melts at body temperature. The substances commonly used to create such vehicles include but are not limited to glycerinated gelatin, mixtures of polyethylene glycols of various molecular weights and fatty acid esters of polyethylene glycol. See, for example: Remington's Pharmaceutical Sciences, 16th Ed., Mack Publishing, Easton, P A, 1980, pp. 1530-1533 for further discussion of suppository dosage forms.

The one or more hallucinogens, or a salt, prodrug and/or solvate thereof, and/or the one or more fatty acids, or a salt, prodrug and/or solvate thereof, will generally be administered in the form of a pharmaceutical composition in which the one or more hallucinogens, or a pharmaceutically acceptable salt, prodrug and/or solvate thereof, and/or the one or more fatty acids, or a pharmaceutically acceptable salt, prodrug and/or solvate thereof, are in association with a pharmaceutically acceptable carrier. Depending on the mode of administration, the pharmaceutical composition will comprise from about 0.05 wt % to about 99 wt % or about 0.10 wt % to about 70 wt %, of the one or more hallucinogens, or a pharmaceutically acceptable salt, prodrug and/or solvate thereof, and the one or more fatty acids, or a pharmaceutically acceptable salt, prodrug and/or solvate thereof, and from about 1 wt % to about 99.95 wt % or about 30 wt % to about 99.90 wt % of a pharmaceutically acceptable carrier, all percentages by weight being based on the total composition.

In some embodiments, the one or more hallucinogens, or a salt, prodrug and/or solvate thereof, and/or the one or more fatty acids, or a salt, prodrug and/or solvate thereof, are present in the compositions in an effective amount, for example an effective amount to treat or prevent a disease, disorder or condition that is treated by activation of a serotonin receptor. In some embodiments the effective amounts are determined as described in the Methods and Uses section below.

In some embodiments, the one or more hallucinogens, or a salt, prodrug and/or solvate thereof, and/or the one or more fatty acids, or a salt, prodrug and/or solvate thereof, are used or administered in a composition comprising an additional therapeutic agent. Therefore the present application also includes a pharmaceutical composition comprising the one or more hallucinogens, or a salt, prodrug and/or solvate thereof, and/or the one or more fatty acids, or a salt, prodrug and/or solvate thereof, thereof and an additional therapeutic agent, and optionally one or more pharmaceutically acceptable excipients. In some embodiments, the additional therapeutic agent is another known agent useful for treatment of a disease, disorder or condition by activation of a serotonin receptor. In some embodiments, the additional therapeutic agent is a psychoactive drug.

### III. Methods and Uses of the Application

The present application includes a method of treating or preventing a disease, disorder or condition that is treated by activation of a serotonin receptor comprising administering an effective amount of one or more hallucinogens, or a pharmaceutically acceptable salt, prodrug and/or solvate thereof, and one or more fatty acids, or a pharmaceutically acceptable salt, prodrug and/or solvate thereof, to a subject in need thereof.

Also included in the present application is a use of one or more hallucinogens, or a pharmaceutically acceptable salt, prodrug and/or solvate thereof, and one or more fatty acids, or a pharmaceutically acceptable salt, prodrug and/or solvate thereof, to treat or prevent a disease, disorder or condition that is treated by activation of a serotonin receptor as well as a use of one or more hallucinogens, or a pharmaceutically acceptable salt, prodrug and/or solvate thereof, and one or more fatty acids, or a pharmaceutically acceptable salt, prodrug and/or solvate thereof, in the preparation of a medicament to treat or prevent a disease, disorder or condition that is treated by activation of a serotonin receptor. Also included in the present application is one or more hallucinogens, or a pharmaceutically acceptable salt, prodrug and/or solvate thereof, and one or more fatty acids, or a pharmaceutically acceptable salt, prodrug and/or solvate thereof, for use to treat or prevent a disease, disorder or condition that is treated by activation of a serotonin receptor.

In some embodiments, the present application also includes a method of improving the efficacy of one or more hallucinogens, or a pharmaceutically acceptable salt, prodrug and/or solvate thereof, comprising administering an effective amount of the one or hallucinogens, or a pharmaceutically acceptable salt, prodrug and/or solvate thereof, in combination with an effective amount of one or more fatty acids, or a pharmaceutically acceptable salt, prodrug and/or solvate thereof, to a subject in need thereof.

Also included in the present application is a use of one or more fatty acids, or a pharmaceutically acceptable salt, prodrug and/or solvate thereof, to improve the efficacy of one or more hallucinogens, or a pharmaceutically acceptable salt, prodrug and/or solvate thereof, as well as a use of one or more fatty acids, or a pharmaceutically acceptable salt, prodrug and/or solvate thereof, in the preparation of a medicament to improve the efficacy of one or more hallucinogens, or a pharmaceutically acceptable salt, prodrug and/or solvate thereof. Also included in the present application is one or more fatty acids, or a pharmaceutically acceptable salt, prodrug and/or solvate thereof, for use to improve the efficacy of one or more hallucinogens, or a pharmaceutically acceptable salt, prodrug and/or solvate thereof.

In some embodiments, the efficacy of the one or more hallucinogens, or a pharmaceutically acceptable salt, prodrug and/or solvate thereof, is improved in the treatment or prevention of a disease, disorder or condition that is treated by activation of a serotonin receptor.

In some embodiments, the serotonin receptor that is activated is 5-HT2A. In some embodiments, the disease, disorder or condition that is treated by activation of a serotonin receptor is a mental illness.

In some embodiments, the mental illness is anxiety disorders such as generalized anxiety disorder, panic disorder, social anxiety disorder or specific phobias; depression such as, hopelessness, loss of pleasure, fatigue or suicidal thoughts; mood disorders, such as depression, bipolar disorder, cancer-related depression, anxiety or cyclothymic disorder; psychotic disorders, such as hallucinations, delusions, schizophrenia; impulse control or addiction disorders, such as pyromania (starting fires), kleptomania (stealing) or compulsive gambling; alcohol addiction; drug addiction, such as opioid addiction; personality disorders, such as antisocial personality disorder, obsessive-compulsive personality disorder or paranoid personality disorder; obsessive-compulsive disorders (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” or depersonalization disorder; factitious disorders; sexual or gender disorders, such as sexual dysfunction, gender identity disorder or the paraphilia's; somatic symptom disorders, formerly known as a psychosomatic disorder or somatoform disorder; and combinations thereof.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches or migraine; obesity or eating disorders; epilepsies or seizure disorders; neuronal cell death; excitotoxic cell death; or a combination thereof.

In some embodiments, the mental illness is hallucinations or delusions or a combination thereof.

In some embodiments, the hallucinations are visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations or chronoceptive hallucinations, or a combination thereof.

In some embodiments, the disease, disorder or condition that is treated by activation of a serotonin receptor is psychosis or psychotic symptoms.

In some embodiments, administering to said subject in need thereof a therapeutically effective amount of the compositions of the application does not result in a worsening of psychosis or psychotic symptoms such as, but not limited to, hallucinations and/or delusions. In some embodiments, administering to said subject in need thereof a therapeutically effective amount of the compositions of the application results in an improvement of psychosis or psychotic symptoms such as, but not limited to, hallucinations and/or delusions. In some embodiments, administering to said subject in need thereof a therapeutically effective amount of the compounds of the application results in an improvement of psychosis or psychotic symptoms.

In some embodiments, the disease, disorder or condition that is treated by activation of a serotonin receptor is a CNS disease, disorder or condition and/or neurological disease, disorder or condition. In some embodiments the CNS disease, disorder or condition and/or neurological disease, disorder or condition is selected from Alzheimer's disease, presenile dementia, senile dementia, vascular dementia, Lewy body dementia, cognitive impairment, Parkinson's disease, Parkinsonian related disorders (such as Parkinson dementia, corticobasal degeneration, or supranuclear palsy), epilepsy, CNS trauma, CNS infections, CNS inflammation, stroke, multiple sclerosis, Huntington's disease, mitochondrial disorders, Fragile X syndrome, Angelman syndrome, hereditary ataxias, neuro-otological movement disorders, eye movement disorders, neurodegenerative diseases of the retina, amyotrophic lateral sclerosis, tardive dyskinesias, hyperkinetic disorders, attention deficit hyperactivity disorder, attention deficit disorders, restless leg syndrome, Tourette's syndrome, schizophrenia, autism spectrum disorders, tuberous sclerosis, Rett syndrome, cerebral palsy, disorders of the reward system including eating disorders such as anorexia nervosa (“AN”) or bulimia nervosa (“BN”), binge eating disorder (“BED”), trichotillomania, dermotillomania, nail biting; migraine, fibromyalgia, or peripheral neuropathy of any etiology, or combinations thereof.

In some embodiments, the subject is a mammal. In another embodiment, the subject is human. In some embodiments, the subject is a non-human animal. In some embodiments, the subject is canine. In some embodiments, the subject is feline. Accordingly, the compositions, kits, methods and uses of the present application are directed to both human and veterinary diseases, disorders and conditions.

In some embodiments, the compositions of the application are useful for treating behavioral problems in subjects that are felines or canines.

Therefore, in some embodiments, the disease, disorder or condition that is treated by activation of a serotonin receptor is behavioral problems in subjects that are felines or canines.

In some embodiments, the behavioral problems are selected from, but are not limited to, anxiety, fear, stress, sleep disturbances, cognitive dysfunction, aggression, excessive noise making, scratching, biting and a combination thereof.

In some embodiments, the non-human subject is canine. In some embodiments, the non-human subject is feline.

In the context of treating a disease, disorder or condition that is treated by activation of a serotonin receptor, an effective amount of the one or more hallucinogens, or a salt, prodrug and/or solvate thereof, and/or the one or more fatty acids, or a salt, prodrug and/or solvate thereof, is an amount that, for example, treats the disease, disorder or condition compared to the disease, disorder or conditions without administration of the one or more hallucinogens, or a salt, prodrug and/or solvate thereof, and the one or more fatty acids, or a salt, prodrug and/or solvate thereof. Further, in the context of improving the efficacy of one or more hallucinogens, or a salt, prodrug and/or solvate thereof, for the treatment of a disease, disorder or condition that is treated by activation of a serotonin receptor an effective amount the one or more fatty acids, or a salt, prodrug and/or solvate thereof, is, for example, an amount that, improves the efficacy of the one or more hallucinogens, or a salt, prodrug and/or solvate thereof compared to without administration of the one or more fatty acids, or a salt, prodrug and/or solvate thereof.

Effective amounts may vary according to factors such as the disease state, age, sex and/or weight of the subject. The amount of a given compound or composition that will correspond to such an amount will vary depending upon various factors, such as the given compound or composition, the pharmaceutical formulation, the route of administration, the type of condition, disease or disorder, the identity of the subject being treated, and the like, but can nevertheless be routinely determined by one skilled in the art.

The term “treated”, “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early depression can be treated to prevent progression, or alternatively a subject in remission can be treated with a composition of the application to prevent recurrence.

Treatment methods comprise administering to a subject the one or more hallucinogens, or a salt, prodrug and/or solvate thereof, and the one or more fatty acids, or a salt, prodrug and/or solvate thereof, and optionally consists of a single administration, or alternatively comprises a series of administrations. The length of the treatment period depends on a variety of factors, such as the severity of the disease, disorder or condition, the age of the subject, the dosage of the one or more hallucinogens, or a salt, prodrug and/or solvate thereof, and/or the one or more fatty acids, or a salt, prodrug and/or solvate thereof, the activity of one or more hallucinogens, or a salt, prodrug and/or solvate thereof, and/or the one or more fatty acids, or a salt, prodrug and/or solvate thereof, or a combination thereof.

In some embodiments the one or more hallucinogens, or a salt, prodrug and/or solvate thereof, is administered or used according to treatment protocol that is known for the one or more hallucinogens in the treatment of the disease, disorder or condition that is treated by activation of a serotonin receptor.

In some embodiments, the dosage of the one or more fatty acids, or a salt, prodrug and/or solvate thereof, and/or the one or more hallucinogens, or a salt, prodrug and/or solvate thereof, varies depending on many factors such as the pharmacodynamic properties thereof, the mode of administration, the age, health and weight of the subject, the nature and extent of the symptoms, the frequency of the treatment and the type of concurrent treatment, if any, and the clearance rate in the subject to be treated. One of skill in the art can determine the appropriate dosage based on the above factors.

In some embodiments, the dosage the one or more hallucinogens, or a salt, prodrug and/or solvate thereof, is equal to or less than the dosage of such agents when used alone or without the one or more fatty acids, or a salt, prodrug and/or solvate thereof. Such dosages are known to or readily determined by those skilled in the art.

In some embodiments, the one or more fatty acids, or a salt, prodrug and/or solvate thereof, and/or the one or more hallucinogens, or a salt, prodrug and/or solvate thereof, are administered or used one, two, three or four times a year. In some embodiments, the one or more fatty acids, or a salt, prodrug and/or solvate thereof, and/or the one or more hallucinogens, or a salt, prodrug and/or solvate thereof, are administered or used at least once a week. In some embodiments, the one or more fatty acids, or a salt, prodrug and/or solvate thereof, and/or the one or more hallucinogens, or a salt, prodrug and/or solvate thereof, are administered or used from about one time per two weeks, three weeks or one month. In some embodiments, the compounds are administered about one time per week to about once daily. In another embodiment, the one or more fatty acids, or a salt, prodrug and/or solvate thereof, and/or the one or more hallucinogens, or a salt, prodrug and/or solvate thereof, are administered or used 1, 2, 3, 4, 5 or 6 times daily. The length of the treatment period depends on a variety of factors, such as the severity of the disease, disorder or condition, the age of the subject, the concentration and/or the activity of the one or more fatty acids, or a salt, prodrug and/or solvate thereof, and/or the one or more hallucinogens, or a salt, prodrug and/or solvate thereof, or a combination thereof. It will also be appreciated that the effective dosage of the one or more fatty acids, or a salt, prodrug and/or solvate thereof, and/or the one or more hallucinogens, or a salt, prodrug and/or solvate thereof, used for the treatment or prevention may increase or decrease over the course of a particular treatment regime. Changes in dosage may result and become apparent by standard diagnostic assays known in the art. In some instances, chronic administration or use is required. For example, the one or more fatty acids, or a salt, prodrug and/or solvate thereof, and/or the one or more hallucinogens, or a salt, prodrug and/or solvate thereof, are administered to the subject or used in an amount and for duration sufficient to treat the subject.

In some embodiments, the one or more hallucinogens, or a salt, prodrug and/or solvate thereof, are administered at doses that are hallucinogenic or psychotomimetic and taken in conjunction with psychotherapy or therapy. In some embodiments such psychotherapy or therapy occurs once, twice, three, or four times a year. In some embodiments, the one or more hallucinogens, or a salt, prodrug and/or solvate thereof, and the one or more fatty acids, or a salt, prodrug and/or solvate thereof, are administered to the subject once daily, once every two days, once every 3 days, once a week, once every two weeks, once a month, once every two months, or once every three months and the one or more hallucinogens, or a salt, prodrug and/or solvate thereof, are administered or used at doses that are not hallucinogenic or psychotomimetic.

The dosage of the one or more fatty acids, or a salt, prodrug and/or solvate thereof, and/or the one or more hallucinogens, or a salt, prodrug and/or solvate thereof, varies depending on many factors such as the pharmacodynamic properties of the compound, the mode of administration, the age, health and weight of the recipient, the nature and extent of the symptoms, the frequency of the treatment and the type of concurrent treatment, if any and the clearance rate of the compound in the subject to be treated. Appropriate dosage can be readily determined based on the above factors. In some embodiments, the one or more fatty acids, or a salt, prodrug and/or solvate thereof, and/or the one or more hallucinogens, or a salt, prodrug and/or solvate thereof, are administered initially in a suitable dosage that is adjusted as required, depending on the clinical response. Dosages will generally be selected to achieve or maintain a serum level of the one or more fatty acids, or a salt, prodrug and/or solvate thereof, and/or the one or more hallucinogens, or a salt, prodrug and/or solvate thereof, from about 0.01 μg/cc to about 1000 μg/cc, or about 0.1 μg/cc to about 100 μg/cc. As a representative example, oral dosages of the one or more hallucinogens, or a salt, prodrug and/or solvate thereof, or the one or more fatty acids, or a salt, prodrug and/or solvate thereof, will range between about 10 μg per day to about 1000 mg per day for an adult, suitably about 10 μg per day to about 500 mg per day, more suitably about 10 μg per day to about 200 mg per day. For parenteral administration, a representative dosage of the one or more hallucinogens, or a salt, prodrug and/or solvate thereof, or the one or more fatty acids, or a salt, prodrug and/or solvate thereof, is from about 0.0001 mg/kg to about 10 mg/kg, about 0.0001 mg/kg to about 1 mg/kg, about 0.01 mg/kg to about 0.1 mg/kg or about 0.0001 mg/kg to about 0.01 mg/kg will be administered. For oral administration, a representative dosage of the one or more hallucinogens, or a salt, prodrug and/or solvate thereof, or the one or more fatty acids, or a salt, prodrug and/or solvate thereof, is from about 0.001 μg/kg to about 10 mg/kg, about 0.1 μg/kg to about 10 mg/kg, about 0.01 μg/kg to about 1 mg/kg or about 0.1 μg/kg to about 1 mg/kg. For administration in suppository form, a representative dosage of the one or more hallucinogens, or a salt, prodrug and/or solvate thereof, or the one or more fatty acids, or a salt, prodrug and/or solvate thereof, is from about 0.1 mg/kg to about 10 mg/kg or about 0.1 mg/kg to about 1 mg/kg.

In some embodiments, the weight ratio of the amount or dosage of the one or more fatty acids, or a salt, prodrug and/or solvate thereof, to the one or more hallucinogens, or a salt, prodrug and/or solvate thereof, that is in a single composition or that are administered in separate compositions, is about 0.1:1 to about 5:1.

In some embodiments of the application, compositions are formulated for oral administration and the one or more hallucinogens, or a salt, prodrug and/or solvate thereof, and/or the one or more fatty acids, or a salt, prodrug and/or solvate thereof, are suitably in the form of tablets containing 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredients per tablet. In some embodiments of the application the one or more hallucinogens, or a salt, prodrug and/or solvate thereof, and/or the one or more fatty acids, or a salt, prodrug and/or solvate thereof, are administered or used in a single daily, weekly or monthly dose or the total daily dose is divided into two, three or four daily doses.

In some embodiments, the one or more hallucinogens, or a salt, prodrug and/or solvate thereof, and/or the one or more fatty acids, or a salt, prodrug and/or solvate thereof, are used or administered in an effective amount which comprises administration of doses or dosage regimens that are devoid of clinically meaningful hallucinogenic/psychotomimetic actions. In some embodiments, the one or more hallucinogens, or a salt, prodrug and/or solvate thereof, and/or the one or more fatty acids, or a salt, prodrug and/or solvate thereof, are used or administered in an effective amount which comprises administration of doses or dosage regimens that provide clinical effects similar to those exhibited by a human plasma psilocin Cmax of about 1 ng/ml to about 5 ng/mL or less and/or human 5-HT2A human CNS receptor occupancy of 40% or less or those exhibited by a human plasma psilocin Cmax of 1 ng/ml or less and/or human 5-HT2A human CNS receptor occupancy of 30% or less. In some embodiments, the one or more hallucinogens, or a salt, prodrug and/or solvate thereof, and/or the one or more fatty acids, or a salt, prodrug and/or solvate thereof, are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

In some embodiments the one or more hallucinogens, or a salt, prodrug and/or solvate thereof, and/or the one or more fatty acids, or a salt, prodrug and/or solvate thereof, are used or administered in an effective amount which comprises administration of doses or dosage regimens that provide clinical effects similar to those exhibited by a human plasma psilocin Cmax of 1 ng/ml or more and/or human 5-HT2A human CNS receptor occupancy of 40% or more or those exhibited by a human plasma psilocin Cmax of about 1 ng/ml to about 50 ng/mL, or about 20 ng/ml to about 50 ng/ml, or about 40 ng/ml to about 50 ng/ml.

### IV. Intranasal Compositions of the Application and Methods and Uses Thereof

### Intranasal Compositions of the Application

The present application also includes an intranasal pharmaceutical composition comprising one or more hallucinogens, or a pharmaceutically acceptable salt, prodrug and/or solvate thereof, and one or more fatty acids, or a pharmaceutically acceptable salt, prodrug and/or solvate thereof.

In some embodiments, the one or more hallucinogens, or a pharmaceutically acceptable salt, prodrug and/or solvate thereof, and the one or more fatty acids, or a pharmaceutically acceptable salt, prodrug and/or solvate thereof, are present in an amount effective to treat or prevent a disease, disorder or condition that is treated by activation of a serotonin receptor.

The present application also includes an intranasal pharmaceutical composition comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more fatty acids are present in amounts that are effective for improving the efficacy of the one or more hallucinogens to treat or prevent a disease, disorder or condition that is treated by activation of a serotonin receptor.

The present application also includes a kit comprising one or more intranasal pharmaceutical compositions comprising one or more hallucinogens, or a pharmaceutically acceptable salt, prodrug and/or solvate thereof, and one or more fatty acids, or a pharmaceutically acceptable salt, prodrug and/or solvate thereof, and instructions for administration of the one or more one or more intranasal pharmaceutical compositions, to a subject in need thereof.

The present application also includes a kit for treating or preventing a disease, disorder or condition that is treated by activation of a serotonin receptor the kit comprising one or more intranasal pharmaceutical compositions comprising one or more hallucinogens, or a pharmaceutically acceptable salt, prodrug and/or solvate thereof, and one or more fatty acids, or a pharmaceutically acceptable salt, prodrug and/or solvate thereof, and instructions for administration of the one or more one or more intranasal pharmaceutical compositions, to a subject in need thereof, wherein the one or more hallucinogens, or a pharmaceutically acceptable salt, prodrug and/or solvate thereof, and one or more fatty acids, or a pharmaceutically acceptable salt, prodrug and/or solvate thereof are present in amounts to treat or prevent the disease, disorder or condition that is treated by activation of a serotonin receptor.

The present application also includes a kit for improving the efficacy of one or more hallucinogens, or a pharmaceutically acceptable salt, prodrug and/or solvate thereof, for treating or preventing a disease, disorder or condition that is treated by activation of a serotonin receptor, the kit comprising one or more intranasal pharmaceutical compositions comprising one or more hallucinogens, or a pharmaceutically acceptable salt, prodrug and/or solvate thereof, and one or more fatty acids, or a pharmaceutically acceptable salt, prodrug and/or solvate thereof, and instructions for administration of the one or more one or more intranasal pharmaceutical compositions, to a subject in need thereof, wherein the one or more fatty acids are present in amounts that are effective for improving the efficacy of the one or more hallucinogens, or a pharmaceutically acceptable salt, prodrug and/or solvate thereof, to treat or prevent the disease, disorder or condition that is treated by activation of a serotonin receptor.

In some embodiments, the one or more hallucinogens, or a pharmaceutically acceptable salt, prodrug and/or solvate thereof, are selected from any hallucinogens that is known to be used in medical therapy or treatments, for example, for any disease, disorder or condition that is treated by activation of a serotonin receptor. In some embodiments, the one or more hallucinogens are selected from one or more hallucinogens as described in “Compositions and Kits of the application” above.

In some embodiments, the one or more hallucinogens is in the form of a free base or a pharmaceutically acceptable salt.

In some embodiments, the one or more hallucinogens is 5-methoxy-N,N-dimethyltryptamine (5-MeO-DMT) or a pharmaceutically acceptable salt, prodrug and/or solvate thereof. In some embodiments, the one or more hallucinogens is methoxy-N,N-dimethyltryptamine (5-MeO-DMT) or a pharmaceutically acceptable salt thereof. In some embodiments, the one or more hallucinogens is methoxy-N,N-dimethyltryptamine (5-MeO-DMT)(free base).

In some embodiments, the one or more fatty acids are selected from any such acid derived from fats by hydrolysis and having from 4 to 30 carbon atoms, 6 to 28 carbon atoms or 6 to 24 carbon atoms. In some embodiments, the fatty acids are selected from myristic acid, caproic acid, caprylic acid, capric acid, lauric acid, palmitic acid, stearic acid, arachidic acid, behenic acid, lignoceric acid, palmitolic acid, oleic acid, linoleic acid, linolenic acid, arachidonic acid, eicosapentaenoic acid, docosahexaenoic acid and combinations thereof.

In some embodiments, the one or more fatty acids or a pharmaceutically acceptable salt, prodrug and/or solvate thereof is linoleic acid or a pharmaceutically acceptable salt, prodrug and/or solvate thereof. In some embodiments, the one or more fatty acids or a pharmaceutically acceptable salt, prodrug and/or solvate thereof is linoleic acid (free acid). A person skilled in the art would appreciate that the one or more fatty acids will be in the composition in an acid form or as a salt of the acid, depending on the pH of the composition.

Therefore, in some embodiments, the one or more hallucinogens is methoxy-N,N-dimethyltryptamine (5-MeO-DMT) or a pharmaceutically acceptable salt thereof and the one or more fatty acids or a pharmaceutically acceptable salt, prodrug and/or solvate thereof is linoleic acid.

Accordingly, the present application also includes an intranasal pharmaceutical composition comprising a hallucinogen, or a pharmaceutically acceptable salt thereof, and a fatty acid. The present application also includes an intranasal pharmaceutical composition comprising 5-methoxy-N,N-dimethyltryptamine (5-MeO-DMT) or a pharmaceutically acceptable salt thereof, and a fatty acid. The present application also includes an intranasal pharmaceutical composition comprising 5-methoxy-N,N-dimethyltryptamine (5-MeO-DMT) or a pharmaceutically acceptable salt thereof, and linoleic acid.

In some embodiments, compositions for nasal administration are conveniently formulated as aerosols, sprays, drops, gels and powders. For intranasal administration or administration by inhalation, the compositions of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of the one or more hallucinogens, or a salt, prodrug and/or solvate thereof, and/or the one or more fatty acids, or a salt, prodrug and/or solvate thereof, and a suitable powder base such as lactose or starch. The aerosol dosage forms can also take the form of a pump-atomizer.

The aerosol dosage forms can also take the form of a pump-atomizer.

Therefore, in some embodiments, the intranasal pharmaceutical composition is formulated as a solution, aerosol, spray, drop, gel or a powder.

In some embodiments, the powder is a free-flowing powder or inhalable powder.

In some embodiments, the inhalable powder is formulated for administration via a medicament dispenser selected from a reservoir dry powder inhaler, a unit-dose dry powder inhaler, a pre-metered multi-dose dry powder inhaler, a nasal inhaler or a pressurized metered dose inhaler.

In some embodiments, the intranasal pharmaceutical composition is delivered in the form of a solution, dry powder formulation or suspension from a pump spray container that is squeezed or pumped by the patient or as an aerosol spray presentation from a pressurized container or a nebulizer.

In some embodiments, intranasal pharmaceutical composition is formulated as an aerosol for use with a pump-atomizer.

In some embodiments, the intranasal pharmaceutical composition is a powder. In some embodiments, the intranasal pharmaceutical composition is a dry powder. In some embodiment, the dry powder is formulated to be reconstituted with a suitable vehicle before use or administration. In some embodiments the suitable vehicle is sterile pyrogen-free water.

In some embodiments, the powder is formulated for use or administration with an inhaler or insufflator. Accordingly, in some embodiments, the dry powder is formulated for use or administration as a capsule and cartridge for use with an inhaler or insufflator.

In some embodiments, the powder comprises a weight ratio of the amount or dosage of the one or more fatty acids, or a salt, prodrug and/or solvate thereof, to the one or more hallucinogens, or a salt, prodrug and/or solvate thereof, of about 0.1:1 to about 5:1, about 0.5:1 to about 1:1.5, about 0.75:1.25 to about 1:1.25 or about 1:1.2. In some embodiments, the dry powder comprises a weight ratio of the amount or dosage of the one or more fatty acids, or a salt, prodrug and/or solvate thereof, to the one or more hallucinogens, or a salt, prodrug and/or solvate thereof of about, about 0.5:1 to about 1:1.5, about 0.75:1.25 to about 1:1.25 or about 1:1.2.

In some embodiments, the dry powder further comprises a suitable powder base. In some embodiment, the suitable powder based comprise lactose or starch.

In some embodiments, the intranasal pharmaceutical composition further comprises water. Therefore, in some embodiments, the intranasal pharmaceutical composition further comprises water and is an aqueous intranasal pharmaceutical composition.

In some embodiments, the intranasal pharmaceutical composition is a solution, suspension or emulsion. In some embodiments, the intranasal pharmaceutical composition is a solution.

In some embodiments, the aqueous intranasal pharmaceutical composition is formulated for administration into nose in the form of drops. In some embodiments, the aqueous intranasal pharmaceutical composition is formulated for administration as a nasal spray. In some embodiments, the nasal spray is delivered in the form of a solution or suspension from a pump spray container that is squeezed or pumped by the patient or as an aerosol spray presentation from a pressurized container or a nebulizer. In some embodiments, the aqueous intranasal pharmaceutical composition is formulated as an aerosol for use with a pump-atomizer.

In some embodiments, the water is present in an amount of about about 50% to about 75%, about 50% to about 70%, about 50% to about 65%, about 33% to about 75%, about 55% to about 70% or about 55% to about 65% by weight of the composition. In some embodiments, the water is present in an amount of about 50%, about 60%, about 65% or about 70% by weight of the composition. In some embodiments, the water is present in an amount of about 55% to about 65% by weight of the composition. In some embodiments, the water is about 60% by weight of the composition.

In some embodiments, the one or more hallucinogens is 5-methoxy-N,N-dimethyltryptamine (5-MeO-DMT) or a pharmaceutically acceptable salt, prodrug and/or solvate thereof and the 5-MeO-DMT or a pharmaceutically acceptable salt, prodrug and/or solvate thereof, is present in an amount of about 1% to about 10%, about 1% to about 9%, about 1% to about 8, about 1% to about 7%, about 1% to about 6%, about 1% to about 5%, about 1% to about 4%, about 1% to about 3%, about 2% to about 8%, about 2% to about 7%, about 2% to about 6%, about 2% to about 5%, about 2% to about 4%, about 2% to about 3% or about 3% to about 4% by weight of the composition. In some embodiments, the 5-MeO-DMT or a pharmaceutically acceptable salt, prodrug and/or solvate thereof, is present in an amount of about 1%, about 1.5%, about 2%, about 2.5%, about 3%, about 3.5%, about 4%, about 4.5%, about 5%, about 5.5%, or about 6% by weight of the composition. In some embodiments, the 5-MeO-DMT or a pharmaceutically acceptable salt, prodrug and/or solvate thereof, is present in an amount of about 3% to about 4% by weight of the composition. In some embodiments, the 5-MeO-DMT or a pharmaceutically acceptable salt, prodrug and/or solvate thereof is about 3.5% by weight of the composition.

In some embodiments, the pharmaceutically acceptable salts of 5-MeO-DMT are selected from acetates, ascorbates, benzoates, benzenesulfonates, bisulfates, borates, butyrates, citrates, camphorates, camphorsulfonates, fumarates, hydrochlorides, hydrobromides, hydroiodides, lactates, lysates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and tartrates.

In some embodiments, the pharmaceutically acceptable salts of 5-MeO-DMT are selected from hydrochlorides, sulfates, fumarates, succinates, maleates, lysates, oxalates, benzoates, tartrates, mesylates, and acetates. In some embodiments, the pharmaceutically acceptable salt of 5-MeO-DMT is selected from a benzoate and a succinate. In some embodiments, the pharmaceutically acceptable salt of 5-MeO-DMT is a succinate.

In some embodiments, the one or more fatty acids or a pharmaceutically acceptable salt, prodrug and/or solvate thereof is linoleic acid or a pharmaceutically acceptable salt, prodrug and/or solvate thereof and the linoleic acid or a pharmaceutically acceptable salt, prodrug and/or solvate thereof is present in an amount of about 1% to about 10%, about 1% to about 9%, about 1% to about 8, about 1% to about 7%, about 1% to about 6%, about 1% to about 5%, about 1% to about 4%, about 1% to about 3%, about 2% to about 8%, about 2% to about 7%, about 2% to about 6%, about 3% to about 6%, about 3% to about 5%, about 2% to about 5% or about 2% to about 4%, by weight of the composition. In some embodiments, the linoleic acid or a pharmaceutically acceptable salt, prodrug and/or solvate thereof is present in an amount of about 1%, about 1.5%, about 2%, about 2.5%, about 3%, about 3.5%, about 4%, about 4.5%, about 5%, about 5.5%, or about 6% by weight of the composition. In some embodiments, the linoleic acid or a pharmaceutically acceptable salt, prodrug and/or solvate thereof is present in an amount of about 2% to about 4% by weight of the composition. In some embodiments, the linoleic acid is about 3% by weight of the composition.

In some embodiments, the aqueous intranasal pharmaceutical composition comprises a weight ratio of the amount or dosage of the one or more fatty acids, or a salt, prodrug and/or solvate thereof, to the one or more hallucinogens, or a salt, prodrug and/or solvate thereof of about 0.1:1 to about 5:1, about 0.5:1 to about 1:1.5, about 0.75:1.25 to about 1:1.25 or about 1:1.2. In some embodiments, the aqueous intranasal composition formulation comprises a weight ratio of the amount or dosage of the one or more fatty acids, or a salt, prodrug and/or solvate thereof, to the one or more hallucinogens, or a salt, prodrug and/or solvate thereof of about 0.5:1 to about 1:1.5, about 0.75:1.25 to about 1:1.25 or about 1:1.2.

In some embodiments, the aqueous intranasal pharmaceutical has a pH of about 4 to about 8, about 4 to about 7, about 4.5 to about 6, or about 4.5 to about 5.5. In some embodiments, the aqueous intranasal pharmaceutical has a pH of about 4, about 4.5, about 5, about 5.5 or about 6. In some embodiments, the aqueous intranasal pharmaceutical has a pH of about 4.5 to about 5.5 or of about 5.

In some embodiments, the intranasal pharmaceutical composition is stable. In some embodiments, the physical characteristics and pH of the intranasal pharmaceutical composition remains substantially unchanged after 90 days at about 45° C. In some embodiments, appearance (i.e. liquid), colour and/or pH of the intranasal pharmaceutical composition of the remains substantially unchanged after 90 days at about 45° C.

In some embodiments, the intranasal pharmaceutical composition further comprises one or more excipients. In some embodiments, the one or more excipients are selected from pH adjusters, buffering agents, surfactants, humectants, co-solvents, emulsifiers, preservatives, gelling agents, tonicity agent, antioxidants, stabilizing agents and sweetening agents. In some embodiments, the one or more excipients are selected from buffering agents, surfactants, co-solvents, humectants and sweetening agents.

In some embodiments, the intranasal pharmaceutical composition further comprises about 10% to about 35%, or about 10% to about 30%, or about 10% to about 25%, or about 15% to about 25% of the one or more surfactants by weight of the composition. In some embodiments, the intranasal pharmaceutical composition further comprises about 15% to about 25% of one or more surfactants by weight of the composition. In some embodiments, the intranasal pharmaceutical composition further comprises about 10%, about 15%, about 20%, about 25%, or about 30% of the one or more surfactants by weight of the composition. In some embodiments, the intranasal pharmaceutical composition further comprises about 20%, about 25%, or about 30% of the one or more surfactants by weight of the composition.

In some embodiments, the one or more surfactants are one or more non-ionic surfactants. In some embodiments, the one or more non-ionic surfactants are selected from but not limited to, tyloxapol, polyoxyethylene-sorbitan-fatty acid esters (polysorbates), products of hydrogenated vegetable oils, polyethoxylated castor oils, polyethoxylated hydrogenated castor oil, polyoxyethylene castor oil derivatives and poloxamers and mixtures thereof.

In some embodiments, the polyoxyethylene sorbitan fatty esters are selected from but not limited to, polyethylene sorbitan monooleate (Polysorbate 80), polyoxyethylene (20) sorbitan monolaurate (polysorbate 20), polyoxyethylene (20) sorbitan tristearate (polysorbate 65), polyoxyethylene (20) sorbitan monooleate, polyoxyethylene (20) sorbitan monopalmitate, and polyoxyethylene (20) sorbitan monostearate and mixtures thereof. In some embodiments, the polyoxyethylene sorbitan fatty ester is polyoxyethylene (20) sorbitan monolaurate (polysorbate 20, Tween® 20). Therefore, in some embodiments, the surfactant is polyoxyethylene (20) sorbitan monolaurate (polysorbate 20, Tween® 20) and mixtures thereof.

In some embodiments, the intranasal pharmaceutical composition further comprises about 1% to about 10%, about 2% to about 8%, about 2% to about 7%, about 3% to about 7%, about 3% to about 6%, or about 4% to about 6% of the one or more co-solvents by weight of the composition. In some embodiments, the intranasal pharmaceutical composition further comprises about 4% to about 6% of the one or more co-solvents by weight of the composition. In some embodiments, the intranasal pharmaceutical composition further comprises about 1%, about 2%, about 3%, about 4%, about 5%, about 6%, about 7%, about 8%, about 9% or about 10% of the one or more co-solvents by weight of the composition. In some embodiments, the intranasal pharmaceutical composition further comprises about 5% of one or more co-solvents by weight of the composition.

In some embodiments, the one or more co-solvents are selected from hydroxylated solvents such as alcohols including isopropyl alcohol; glycols such as propylene glycol, polyethylene glycol, polypropylene glycol, glycol ether, and glycerol; polyoxyethylene alcohols; medium chain glycerides and diethylene glycol monoethyl ether (2-(2-ethoxyethoxy) ethanol) and mixtures thereof. In some embodiments, the co-solvent is diethylene glycol monoethyl ether (2-(2-ethoxyethoxy) ethanol, Transcutanol®)

In some embodiments, the intranasal pharmaceutical composition further comprises about 0.1% to about 5%, about 0.5% to about 5%, about 0.5% to about 4%, about 0.5% to about 3%, about 1% to about 3% or about 2% to about 3% of one or more buffering agents by weight of the composition. In some embodiments, the intranasal pharmaceutical composition further comprises about 1% to about 3% of the one or more buffering agents by weight of the composition. In some embodiments, the intranasal pharmaceutical composition further comprises about 0.1%, about 0.5%, about 1%, about 1.5%, about 2%, about 2.5%, about 3%, about 3.5%, about 4%, about 4.5% or about 5% of the one or more buffering agents by weight of the composition. In some embodiments, the intranasal pharmaceutical composition further comprises about 3% of the one or more buffering agents by weight of the composition.

In some embodiments, the one or more buffering agents is selected from sodium phosphate, sodium citrate and citric acid and mixtures thereof. In some embodiments, the one or more buffering agents are selected from sodium citrate and citric acid and mixtures thereof.

In some embodiments, the intranasal pharmaceutical composition further comprises about 1% to about 10%, about 2% to about 8%, about 2% to about 7%, about 3% to about 7%, about 3% to about 6%, or about 4% to about 6% of one or more humectants by weight of the composition. In some embodiments, the intranasal pharmaceutical composition further comprises about 4% to about 6% of the one or more humectants by weight of the composition. In some embodiments, the intranasal pharmaceutical composition further comprises about 1%, about 2%, about 3%, about 4%, about 5%, about 6%, about 7%, about 8%, about 9% or about 10% of the one or more humectants by weight of the composition. In some embodiments, the intranasal pharmaceutical composition further comprises about 5% of the one or more humectants by weight of the composition

In some embodiments, one or more humectants are selected from glycerin, sorbitol, mannitol and xylitol and mixtures thereof. In some embodiments, one or more humectants is xylitol.

In some embodiments, the intranasal pharmaceutical composition further comprises the one or more preservatives. In some embodiments, the one or more preservatives are selected from but are not limited to, phenylethyl alcohol, benzalkonium chloride, benzoic acid, benzoates such as sodium benzoate, and phenylethyl alcohol. In some embodiments, the intranasal pharmaceutical composition is preservative free.

n some embodiments, the intranasal pharmaceutical composition further comprises the one or more sweetening agents. In some embodiments, the one or more sweetening agents are selected from sugar alcohols including glycerol, sorbitol, xylitol, mannitol, galactitol, maltitol, hydrogenated isomaltulose (isomalt), lactitol, erythritol, glucitol, ribitol; sugar sweeteners including saccharides, such as mono-saccharides, di-saccharides and poly-saccharides such as sucrose, dextrose, maltose, dextrin, maltodextrin, xylose, ribose, glucose including liquid glucose, mannose, galactose, fructose (levulose), lactose, invert sugar, fructo oligo saccharide syrups, trehalose, tagatose, fucose, gulose, raffinose, ribulose, rufinose, stachyose, xylulose, adonose, amylase, arabinose, deoxyribose, corn syrup solids, such as high fructose corn syrup, or a combination thereof; artificial sweeteners such as soluble saccharin salts, i.e., sodium or calcium saccharin salts, the potassium salt of 3,4-dihydro-6-methyl-1,2,3-oxathiazine-4-one-2,2-dioxide (Acesulfame-K), the free acid form of saccharin, L-aspartic acid derived sweeteners, such as L-aspartyl-L-phenylalanine methyl ester (Aspartame), L-alphaaspartyl-N-(2,2,4,4-tetramethyl-3-thietanyl)-D-alaninamide hydrate (Alitame), N-[N-(3,3-dimethylbutyl)-L-aspartyl]-L-phenylalanine 1-methyl ester (Neotame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanineor a combination thereof; sucralose; maltol; stevia; ammonium glycyrrhizate glycerin (MagnaSweet®); or a combination thereof. In some embodiments, the one or more sweetening agents are selected from sugar alcohols, stevia and ammonium glycyrrhizate glycerin.

Therefore, in some embodiments, the present application includes an intranasal pharmaceutical composition comprising about 3% to about 6% of 5-methoxy-N,N-dimethyltryptamine (5-MeO-DMT) or a pharmaceutically acceptable salt, prodrug and/or solvate thereof, and 2% to about 4% linoleic acid, or a pharmaceutically acceptable salt, prodrug and/or solvate thereof and about 55% to about 65% water by weight of the composition.

In some embodiments, the intranasal pharmaceutical composition further optionally comprises one or more excipients selected from buffering agents, surfactants, co-solvents and humectants. In some embodiments, the intranasal pharmaceutical composition further optionally comprises about 15% to about 25% of the one or more surfactants by weight of the composition. In some embodiments, the intranasal pharmaceutical composition further optionally comprises about 4% to about 6% of the one or more co-solvents by weight of the composition. In some embodiments, the intranasal pharmaceutical composition further optionally comprises about 1% to about 3% of the one or more buffering agents by weight of the composition. In some embodiments, the intranasal pharmaceutical composition further optionally comprises about 4% to about 6% of the one or more humectants by weight of the composition.

In some embodiments, the intranasal pharmaceutical composition further comprises about 15% to about 25% of the one or more surfactants by weight of the composition. In some embodiments, the intranasal pharmaceutical composition further comprises about 15% to about 25% of the one or more surfactants and about 4% to about 6% of the one or more co-solvents by weight of the composition. In some embodiments, the intranasal pharmaceutical composition further comprises about 15% to about 25% of the one or more surfactants, about 4% to about 6% of the one or more co-solvents and about 1% to about 3% of the one or more buffering agents by weight of the composition. In some embodiments, the intranasal pharmaceutical composition further comprises about 15% to about 25% of the one or more surfactants, about 4% to about 6% of the one or more co-solvents, about 1% to about 3% of the one or more buffering agents and about 4% to about 6% of the one or more humectants by weight of the composition.

Therefore, in some embodiments, the present application includes an intranasal pharmaceutical composition comprising about 3% to about 6% of 5-methoxy-N,N-dimethyltryptamine (5-MeO-DMT) or a pharmaceutically acceptable salt, thereof, and 2% to about 4% linoleic acid, about 55% to about 65% water, about 15% to about 25% of the one or more surfactants, about 4% to about 6% of one or more co-solvents, about 1% to about 3% of the one or more buffering agents by weight of the composition and about 4% to about 6% of the one or more humectants by weight of the composition.

In an exemplary embodiment, the surfactant is polyoxyethylene (20) sorbitan monolaurate (polysorbate 20), the co-solvent is diethylene glycol monoethyl ether (2-(2-ethoxyethoxy) ethanol), the one or more buffering agents are selected from sodium citrate and citric acid and/or the one or more humectants is xylitol. In some embodiments, intranasal pharmaceutical composition further comprises one or more sweetening agents selected from sugar alcohols, stevia and ammonium glycyrrhizate glycerin and mixtures thereof.

Therefore, in some embodiments, the present application includes an intranasal pharmaceutical composition comprising about 3% to about 6% of 5-methoxy-N,N-dimethyltryptamine (5-MeO-DMT) or a pharmaceutically acceptable salt, thereof, and 2% to about 4% linoleic acid, about 55% to about 65% water, about 15% to about 25% of the polyoxyethylene (20) sorbitan monolaurate (polysorbate 20), about 4% to about 6% of (2-(2-ethoxyethoxy) ethanol and about 1% to about 3% sodium citrate and citric acid and about 4% to about 6% of xylitol by weight of the composition. In some embodiments, the intranasal pharmaceutical composition further optionally comprises one or more sweetening agents.

A person skilled in the art would appreciate that one excipient can serve more than one function. Further, it would be appreciated that multiple excipients serving the same function may be used.

In some embodiments, the one or more fatty acids such is linoleic acid, or a pharmaceutically acceptable salt, prodrug and/or solvate thereof increases the rate of absorption of the one or more hallucinogens such as 5-MeO-DMT or a pharmaceutically acceptable salt, prodrug and/or solvate thereof in plasma and cerebrospinal fluid (CSF) of a subject compared to an otherwise identical intranasal pharmaceutical composition except in the absence of the one or more fatty acids such as linoleic acid, or a pharmaceutically acceptable salt, prodrug and/or solvate thereof. In some embodiments, the one or more fatty acids such as linoleic acid, or a pharmaceutically acceptable salt, prodrug and/or solvate thereof increases the rate of absorption of the one or more hallucinogens such as 5-MeO-DMT or a pharmaceutically acceptable salt, prodrug and/or solvate thereof in cerebrospinal fluid (CSF) of a subject compared to an otherwise identical intranasal pharmaceutical composition except in the absence of the one or more fatty acids such as linoleic acid, or a pharmaceutically acceptable salt, prodrug and/or solvate thereof.

Therefore, in some embodiments, the intranasal pharmaceutical composition has increased mucosal delivery of the one or more hallucinogens such as 5-MeO-DMT or a pharmaceutically acceptable salt, prodrug and/or solvate thereof compared to an otherwise identical intranasal pharmaceutical composition except in the absence of the one or more fatty acids such as linoleic acid, or a pharmaceutically acceptable salt, prodrug and/or solvate thereof.

In some embodiments, the intranasal pharmaceutical composition provides a Cmax of 5-MeO-DMT or a pharmaceutically acceptable salt, prodrug and/or solvate thereof in plasma that is about 3 to about 6, or about 5-fold greater compared to the Cmax of an otherwise identical intranasal pharmaceutical composition except in the absence of the linoleic acid, or a pharmaceutically acceptable salt, prodrug and/or solvate thereof.

In some embodiments, the intranasal pharmaceutical composition provides a Cmax of 5-MeO-DMT or a pharmaceutically acceptable salt, prodrug and/or solvate thereof in cerebral spinal fluid (CSF) that is about 5 to about 20, about 10 to about 17, or about 10 fold greater compared to the Cmax of an otherwise identical intranasal pharmaceutical composition except in the absence of the linoleic acid, or a pharmaceutically acceptable salt, prodrug and/or solvate thereof

In some embodiments, the intranasal pharmaceutical composition attains Cmax in the CSF in about 3 to about 25 minutes, about 5 to about 20 minutes or about 5 to 15 minutes following administration of the intranasal pharmaceutical composition.

In some embodiments, the intranasal pharmaceutical composition provides an increase in bioavailability of 5-MeO-DMT or a pharmaceutically acceptable salt, prodrug and/or solvate thereof in plasma and in CSF when administered intranasally compared to an otherwise identical intranasal pharmaceutical composition except in the absence of the linoleic acid, or a pharmaceutically acceptable salt, prodrug and/or solvate thereof. In some embodiments, the increase in bioavailability of 5-MeO-DMT or a pharmaceutically acceptable salt, prodrug and/or solvate thereof in cerebral spinal fluid (CSF) signifies an increased bioavailability of 5-MeO-DMT or a pharmaceutically acceptable salt, prodrug and/or solvate thereof in the brain.

In some embodiments, the intranasal pharmaceutical composition provides about 4 to about 8, about 5 to about 7, or about 7-fold increase in bioavailability in plasma compared to an otherwise identical intranasal pharmaceutical composition except in the absence of the linoleic acid, or a pharmaceutically acceptable salt, prodrug and/or solvate thereof.

The term “increases (d) rate of absorption” as used herein in reference to one or more hallucinogens such as 5-MeO-DMT or a pharmaceutically acceptable salt, prodrug and/or solvate thereof as used herein means any detectable increase in the rate of absorption of the one or more hallucinogens such as 5-MeO-DMT or a pharmaceutically acceptable salt, prodrug and/or solvate thereof using the intranasal pharmaceutical composition of the application compared to the rate of absorption and bioavailability of the one or more hallucinogens such as 5-MeO-DMT or a pharmaceutically acceptable salt, prodrug and/or solvate thereof using an otherwise identical intranasal pharmaceutical composition except in the absence of the one or more fatty acids such as linoleic acid, or a pharmaceutically acceptable salt, prodrug and/or solvate thereof. The “rate of absorption” is estimated by comparison of the time (tmax) to reach the maximum concentration (Cmax).

The term “increased mucosal delivery” as used herein in reference to one or more hallucinogens such as 5-MeO-DMT or a pharmaceutically acceptable salt, prodrug and/or solvate thereof as used herein means any detectable increase in the mucosal delivery the one or more hallucinogens such as 5-MeO-DMT or a pharmaceutically acceptable salt, prodrug and/or solvate thereof using the intranasal pharmaceutical composition of the application compared to the mucosal delivery of the one or more hallucinogens such as 5-MeO-DMT or a pharmaceutically acceptable salt, prodrug and/or solvate thereof using an otherwise identical intranasal pharmaceutical composition except in the absence of the one or more fatty acids such as linoleic acid, or a pharmaceutically acceptable salt, prodrug and/or solvate thereof.

The term “increase in bioavailability” as used herein in reference to one or more hallucinogens such as 5-MeO-DMT or a pharmaceutically acceptable salt, prodrug and/or solvate thereof as used herein means any detectable increase in the bioavailability of the one or more hallucinogens such as 5-MeO-DMT or a pharmaceutically acceptable salt, prodrug and/or solvate thereof using the intranasal pharmaceutical composition of the application compared to the bioavailability of the one or more hallucinogens such as 5-MeO-DMT or a pharmaceutically acceptable salt, prodrug and/or solvate thereof using an otherwise identical intranasal pharmaceutical composition except in the absence of the one or more fatty acids such as linoleic acid, or a pharmaceutically acceptable salt, prodrug and/or solvate thereof.

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66 (1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate.

Illustrative organic acids which form suitable salts include mono-, di—and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono-or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

In some embodiments, the pharmaceutically acceptable salt of 5-MeO-DMT is a succinate.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, tripropylamine, ethanolamine, triethylamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts.

In some embodiments, in the case of an acid (—COOH) or alcohol group being present, pharmaceutically acceptable esters can be employed to modify the solubility or hydrolysis characteristics of a compound. In some embodiments, the esters are alkyl esters. In some embodiments, the alkyl esters are selected from isopropyl esters, methyl esters, ethyl esters, propyl esters and mixtures thereof.

Formation of a pharmaceutically acceptable salt may be achieved using standard techniques. For example, a neutral compound is treated with an acid or base in a suitable solvent and the formed salt is isolated by filtration, extraction or any other suitable method.

Examples of suitable solvate solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

In some embodiments, one or more hallucinogens, or a pharmaceutically acceptable salt, prodrug and/or solvate thereof, and the one or more fatty acids, or a pharmaceutically acceptable salt, prodrug and/or solvate thereof, in the intranasal compositions and kits of the present application are formulated in a single intranasal pharmaceutical composition, for administration to, or use in, subjects.

In some embodiments, the one or more hallucinogens, or a salt, prodrug and/or solvate thereof, and/or the one or more fatty acids, or a salt, prodrug and/or solvate thereof, are present in the compositions in an effective amount, for example an effective amount to treat or prevent a disease, disorder or condition that is treated by activation of a serotonin receptor. In some embodiments the effective amounts are determined as described in the Methods and Uses of the intranasal compositions of the application section below.

In some embodiments, the one or more hallucinogens, or a salt, prodrug and/or solvate thereof, and/or the one or more fatty acids, or a salt, prodrug and/or solvate thereof, are used or administered in an intranasal composition comprising an additional therapeutic agent. Therefore the present application also includes an intranasal pharmaceutical composition comprising the one or more hallucinogens, or a salt, prodrug and/or solvate thereof, and/or the one or more fatty acids, or a salt, prodrug and/or solvate thereof, thereof and an additional therapeutic agent, and optionally one or more pharmaceutically acceptable excipients. In some embodiments, the additional therapeutic agent is another known agent useful for treatment of a disease, disorder or condition by activation of a serotonin receptor. In some embodiments, the additional therapeutic agent is a psychoactive drug.

### ii) Methods and Uses of the Intranasal Compositions of the Application

The present application includes a method of treating or preventing a disease, disorder or condition that is treated by activation of a serotonin receptor comprising administering an effective amount of one or more intranasal formulations of the application to a subject in need thereof.

Also included in the present application is a use of one or more intranasal formulations of the application to treat or prevent a disease, disorder or condition that is treated by activation of a serotonin receptor as well as a use of one or more intranasal formulations of the application, in the preparation of a medicament to treat or prevent a disease, disorder or condition that is treated by activation of a serotonin receptor. Also included in the present application is one or more intranasal formulations of the application, for use to treat or prevent a disease, disorder or condition that is treated by activation of a serotonin receptor.

In some embodiments, the present application also includes a method of improving the efficacy of one or more hallucinogens, or a pharmaceutically acceptable salt, prodrug and/or solvate thereof, comprising administering an effective amount of the one or more intranasal formulations of the application, to a subject in need thereof.

Also included in the present application is a use of one or more intranasal formulations of the application to improve the efficacy of one or more hallucinogens, or a pharmaceutically acceptable salt, prodrug and/or solvate thereof, as well as a use of one or more intranasal formulations of the application in the preparation of a medicament to improve the efficacy of one or more hallucinogens, or a pharmaceutically acceptable salt, prodrug and/or solvate thereof. Also included in the present application one or more intranasal formulations of the application for use to improve the efficacy of one or more hallucinogens, or a pharmaceutically acceptable salt, prodrug and/or solvate thereof.

In some embodiments, the efficacy of the one or more hallucinogens, or a pharmaceutically acceptable salt, prodrug and/or solvate thereof, is improved in the treatment or prevention of a disease, disorder or condition that is treated by activation of a serotonin receptor.

In some embodiments, the serotonin receptor that is activated is 5-HT2A. In some embodiments, the disease, disorder or condition that is treated by activation of a serotonin receptor is as described above under the Methods and Uses of the application section above.

In the context of treating a disease, disorder or condition that is treated by activation of a serotonin receptor, an effective amount of the one or more intranasal formulations of the application is an amount that, for example, treats the disease, disorder or condition compared to the disease, disorder or conditions without administration of the intranasal formulations of the application. Further, in the context of improving the efficacy of one or more hallucinogens, or a salt, prodrug and/or solvate thereof, for the treatment of a disease, disorder or condition that is treated by activation of a serotonin receptor an effective amount of the one or more intranasal formulations of the application is, for example, an amount that, improves the efficacy of the one or more hallucinogens, or a salt, prodrug and/or solvate thereof compared to without administration of the one or more intranasal formulations of the application.

Effective amounts may vary according to factors such as the disease state, age, sex and/or weight of the subject. The amount of a given compound or composition that will correspond to such an amount will vary depending upon various factors, such as the given compound or composition, the pharmaceutical formulation, the route of administration, the type of condition, disease or disorder, the identity of the subject being treated, and the like, but can nevertheless be routinely determined by one skilled in the art.

The term “treated”, “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early depression can be treated to prevent progression, or alternatively a subject in remission can be treated with a composition of the application to prevent recurrence.

Treatment methods comprise administering to a subject the one or more the one or more, and optionally consists of a single administration, or alternatively comprises a series of administrations. The length of the treatment period depends on a variety of factors, such as the severity of the disease, disorder or condition, the age of the subject, the dosage of the one or more intranasal compositions of the application, the activity of one or more intranasal compositions of the application, or a combination thereof.

In some embodiments the one or more hallucinogens, or a salt, prodrug and/or solvate thereof, is administered or used according to treatment protocol that is known for the one or more hallucinogens in the treatment of the disease, disorder or condition that is treated by activation of a serotonin receptor.

In some embodiments, the dosage of the one or more fatty acids, or a salt, prodrug and/or solvate thereof, and/or the one or more hallucinogens, or a salt, prodrug and/or solvate thereof, varies depending on many factors such as the pharmacodynamic properties thereof, the mode of administration, the age, health and weight of the subject, the nature and extent of the symptoms, the frequency of the treatment and the type of concurrent treatment, if any, and the clearance rate in the subject to be treated. One of skill in the art can determine the appropriate dosage based on the above factors.

In some embodiments, the dosage the one or more hallucinogens, or a salt, prodrug and/or solvate thereof, is equal to or less than the dosage of such agents when used alone or without the one or more fatty acids, or a salt, prodrug and/or solvate thereof. Such dosages are known to or readily determined by those skilled in the art.

In some embodiments, the one or more intranasal compositions of the application are administered or used one, two, three or four times a year. In some embodiments, the one or more intranasal compositions of the application are administered or used at least once a week. In some embodiments, the one or more intranasal compositions of the application are administered or used from about one time per two weeks, three weeks or one month. In some embodiments, the one or more intranasal compositions of the application are administered about one time per week to about once daily. In another embodiment, the one or more intranasal compositions of the application, are administered or used 1, 2, 3, 4, 5 or 6 times daily. The length of the treatment period depends on a variety of factors, such as the severity of the disease, disorder or condition, the age of the subject, the concentration and/or the activity of the one or more intranasal compositions of the application, or a combination thereof. It will also be appreciated that the effective dosage of the one or more intranasal compositions of the application, used for the treatment or prevention may increase or decrease over the course of a particular treatment regime. Changes in dosage may result and become apparent by standard diagnostic assays known in the art. In some instances, chronic administration or use is required. For example, the one or more intranasal compositions of the application, are administered to the subject or used in an amount and for duration sufficient to treat the subject.

In some embodiments, the one or more hallucinogens, or a salt, prodrug and/or solvate thereof, in the intranasal compositions of the application are administered at doses that are hallucinogenic or psychotomimetic and taken in conjunction with psychotherapy or therapy. In some embodiments such psychotherapy or therapy occurs once, twice, three, or four times a year. In some embodiments, the intranasal compositions of the application are administered to the subject once daily, once every two days, once every 3 days, once a week, once every two weeks, once a month, once every two months, or once every three months and the one or more hallucinogens, or a salt, prodrug and/or solvate thereof, are administered or used at doses in the intranasal compositions of the application that are not hallucinogenic or psychotomimetic.

In some embodiments, the weight ratio of the amount or dosage of the one or more fatty acids, or a salt, prodrug and/or solvate thereof, to the one or more hallucinogens, or a salt, prodrug and/or solvate thereof, is about 0.1:1 to about 5:1. In some embodiments, the weight ratio of the amount or dosage of the one or more fatty acids, or a salt, prodrug and/or solvate thereof, to the one or more hallucinogens, or a salt, prodrug and/or solvate thereof, is about 0.1:1 to about 5:1, about 0.5:1 to about 1:1.5, about 0.75:1.25 to about 1:1.25 or about 1:1.2. In some embodiments, the weight ratio of the amount or dosage of the one or more fatty acids, or a salt, prodrug and/or solvate thereof, to the one or more hallucinogens, or a salt, prodrug and/or solvate thereof, about 0.5:1 to about 1:1.5, about 0.75:1.25 to about 1:1.25 or about 1:1.2.

In some embodiments, the one or more hallucinogens, or a salt, prodrug and/or solvate thereof, and/or the one or more fatty acids, or a salt, prodrug and/or solvate thereof, in the intranasal compositions of the application are used or administered in an effective amount which comprises administration of doses or dosage regimens that are devoid of clinically meaningful hallucinogenic/psychotomimetic actions. In some embodiments, the one or more hallucinogens, or a salt, prodrug and/or solvate thereof, and/or the one or more fatty acids, or a salt, prodrug and/or solvate thereof in the intranasal compositions of the application, are used or administered in an effective amount which comprises administration of doses or dosage regimens that provide clinical effects similar to those exhibited by a human plasma psilocin Cmax of about 1 ng/ml to about 5 ng/ml or less and/or human 5-HT2A human CNS receptor occupancy of 40% or less or those exhibited by a human plasma psilocin Cmax of 1 ng/ml or less and/or human 5-HT2A human CNS receptor occupancy of 30% or less. In some embodiments, the one or more hallucinogens, or a salt, prodrug and/or solvate thereof, and/or the one or more fatty acids, or a salt, prodrug and/or solvate thereof, in the intranasal compositions of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

In some embodiments the one or more hallucinogens, or a salt, prodrug and/or solvate thereof, and/or the one or more fatty acids, or a salt, prodrug and/or solvate thereof, in the intranasal compositions of the application are used or administered in an effective amount which comprises administration of doses or dosage regimens that provide clinical effects similar to those exhibited by a human plasma psilocin Cmax of 1 ng/ml or more and/or human 5-HT2A human CNS receptor occupancy of 40% or more or those exhibited by a human plasma psilocin Cmax of about 1 ng/ml to about 50 ng/ml, or about 20 ng/ml to about 50 ng/ml, or about 40 ng/ml to about 50 ng/mL.

The following non-limiting examples are illustrative of the present application:

### EXAMPLES

### Example 1: Mouse Head Twitch Experiments

### Methods

Male, C57BL/6J mice (body weight range 20-30 g) were dosed with the appropriate dose(s) of test compound, or combination of test compounds, and following pre-treatment, the animals were placed in individual observation chambers. Animals were visually assessed for the incidence head twitches continuously over a 1 hr period. Head twitches were defined as a rapid jerk of the head which was not elicited by an external tactile stimulus (Corne S J, Pickering R W (1967) Psychopharmacologia 11 (1): 65-78). Each head twitch was individually counted by a trained observer, and the data expressed as the mean±SEM of 4-10 mice per group. In addition to the total number of head twitches recorded over the 1 hr observation period, head twitches were also scored according to each specific 10 min time bin within the 1 hr observation period (i.e. 0-10 min, 10-20 min, 20-30 min, etc).

Two types of experiments were conducted. In one, the effect of linoleic acid in combination with a test compound (psilocybin or 1-4) was studied. In these experiments, both test compounds, or saline control was injected subcutaneously (SC) 1 min prior to testing. In the second set of experiments, the effect of the selective 5-HT2A receptor antagonist M100907 (Kehne J H, et al. (1996) J Pharmacol Exp Ther. 277 (2): 968-981) on test compound-induced head twitches was investigated. In these experiments, M100907 (0.5 mg/kg IP) was injected 30 min prior to a single dose of test compound (i.e. psilocybin or I-4). The dose of test compound was selected as producing a reliable head twitch response based on a prior dose response study. 1 min following SC administration of the test compound, the mice were placed in the observation chambers for measurement of head twitch response.

### Results

Effect of Linoleic Acid in Combination with Test Compound

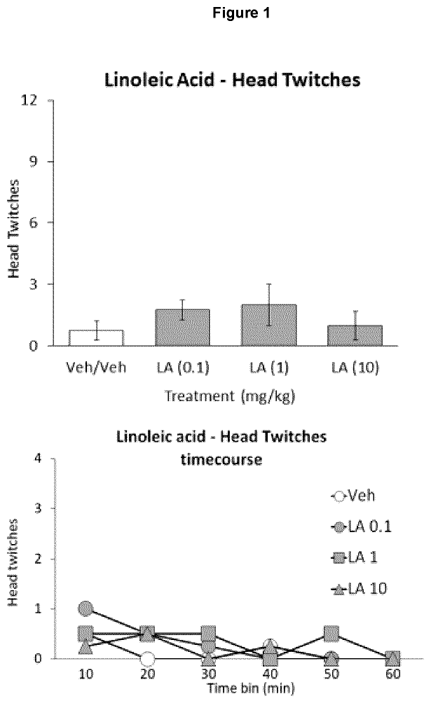

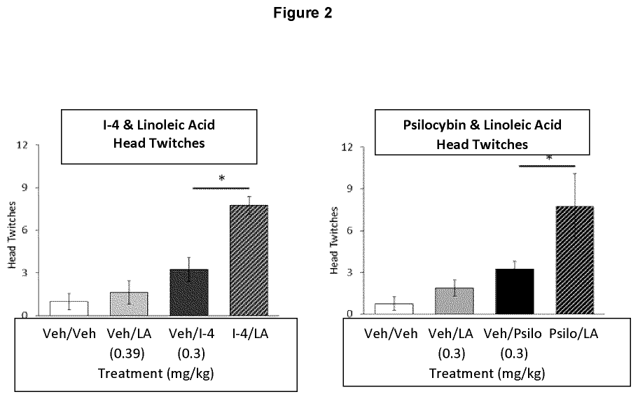

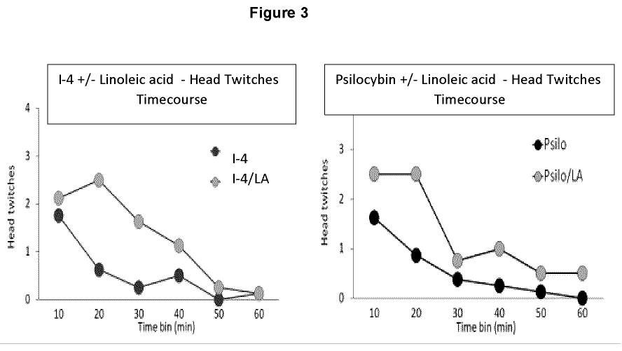

The administration of linoleic acid alone (0.1-10 mg/kg SC) did not induce head twitches to a significant degree relative to vehicle pretreated mice (e.g Vehicle: 0.8±0.5; linoleic acid 10 mg/kg: 1.0±0.7, FIG. 1). However, the combination of linoleic acid with an equimolar dose of test compound (1-4 or psilocybin) resulted in a greater head twitch response compared to the equivalent dose of test article alone (FIG. 2). For example, I-4 administered at 0.3 mg/kg SC+vehicle=3.3±0.8; I-4 administered at 0.3 mg/kg SC+linoleic acid at 0.39 mg/kg SC=7.8±0.6. This increase appeared to be the result of an increase in the duration of the head twitch response (FIG. 3).

Effect of M100907 in Combination with Test Compound

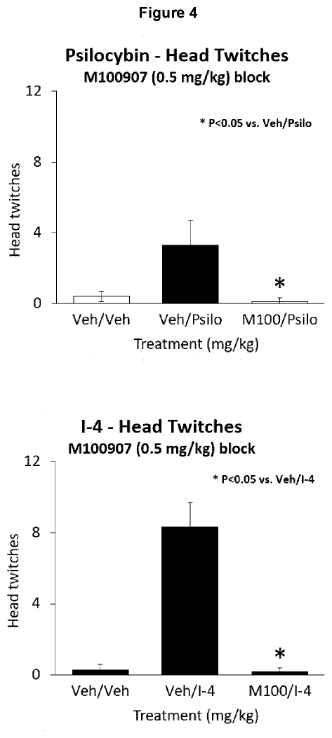

Pretreatment with M100907 (0.5 mg/kg IP) completely blocked the incidence of head twitches induced by either 1-4 (6.2 mg/kg SC) or psilocybin (3 mg/kg SC)(FIG. 4).

### Example 2: Intranasal and Subcutaneous 5-MeO-DMT (16) Formulations

### General Method and Materials

### Preparation of 5-MeO-DMT intranasal formulation

The following ingredients have been used in the process:

| | | | Quantity | |
|---|---|---|---|---|
| Ingredient # | Trade Name | INCI Names | (%) | Quantity (kg) |
| 1 | MSP-4015-SUCCINATE | 5-Methoxy-N,N- | 3.58% | 0.036 |
| | | Dimethyltryptamine | | |
| 2 | Linoleic Acid | Linoleic Acid | 3.00% | 0.03 |
| 3 | Tween 20 | Polysorbate 20 | 20.00% | 0.2 |
| 4 | Transcutol CG | Ethoxydiglycol | 5.00% | 0.05 |
| 5 | Xylitol | Xylitol | 5.00% | 0.05 |
| 6 | Citric Acid | Citric Acid | 0.99% | 0.01 |
| 7 | Sodium Citrate | Sodium Citrate | 1.85% | 0.018 |
| 8 | MagnaSweet MM110 F | Glycerin, Ammonium Glycyrrhizate | 0.05% | 0.001 |
| 9 | Optimizer Stevia 2.10 | Stevia Rebaudiana Leaf Extract | 0.05% | 0.001 |
| 10 | Water | Aqua/Water/Eau | 60.49% | 0.605 |

In a stainless container, ingredients #2, #3, and #4 were added and stirred until the mix is homogeneous (Phase B). In the main tank, ingredients #10, #5, #6, #7, #8 and #9 were added while stirring and continued to stir until the mix was homogeneous (Phase C). Then, in the main tank, Phase B was added to Phase C. The mixture stirred until it was homogeneous. In the next step, ingredient #1 (Phase A) was added and stirred until the mix was homogeneous (Phase A+B+C). The resulting mix was pale to dark yellow liquid with a pH of 5.00±0.50 (at 25° C.).

### Stability of the 5-MeO-DMT Intranasal Formulation

The stability of the product prepared according to the protocol described above was evaluated in a glass jar for 90 days at 45° C., for appearance, color, odor, pH and viscosity, when applicable. The results are shown in Table 1 below.

TABLE 1
Stability results

| | Temperature | | Viscosity | | | |
|---|---|---|---|---|---|---|
| Month | for analysis | pH | (T-rpm, cps) | Appearance | Color | Odor |
| 0 | 25° C. | 4.89 | N/A | Liquid | Pale | Characteristic |
| | | | | | yellow - | |
| | | | | | yellow | |
| 1 | 25° C. | 4.96 | N/A | Liquid | Yellow | Characteristic |
| 2 | 25° C. | 4.89 | N/A | Liquid | Yellow - | Characteristic |
| | | | | | darker | |
| 3 | 25° C. | 4.97 | N/A | Liquid | Yellow - | Characteristic |
| | | | | | darker | |

As can be seen from the data shown in Table 1, the product shows stability in terms of appearance, odor, color, and pH after 90 days at 45° C.

### Preparation of 5-MeO-DMT subcutaneous formulation

The following ingredients have been used in the process:

| | | | Quantity | |
|---|---|---|---|---|
| Ingredient # | Trade Name | INCI Names | (%) | Quantity (kg) |
| 1 | MSP-4015-SUCCINATE | 5-Methoxy-N,N- | 0.24% | 0.002 |
| | | Dimethyltryptamine | | |
| 2 | Linoleic Acid | Linoleic Acid | 3.00% | 0.03 |
| 3 | Tween 20 | Polysorbate 20 | 20.00% | 0.2 |
| 4 | Transcutol CG | Ethoxydiglycol | 5.00% | 0.05 |
| 5 | Xylitol | Xylitol | 5.00% | 0.05 |
| 6 | Citric Acid | Citric Acid | 0.99% | 0.01 |
| 7 | Sodium Citrate | Sodium Citrate | 1.85% | 0.018 |
| 8 | MagnaSweet MM110 F | Glycerin, Ammonium Glycyrrhizate | 0.05% | 0.001 |
| 9 | Optimizer Stevia 2.10 | Stevia Rebaudiana Leaf Extract | 0.05% | 0.001 |
| 10 | Water | Aqua/Water/Eau | 63.82% | 0.638 |

The product has been prepared using the protocol described above for intranasal formulation. The resulting mix was in color to yellow liquid with a pH of 5.00±0.50 (at 25° C.).

### Stability of the 5-MeO-DMT Subcutaneous Formulation

The stability of the product prepared according to the protocol described above was evaluated in a glass jar for 90 days at 45° C., for appearance, color, odor, pH and viscosity, when applicable. The results are shown in Table 2 below.

TABLE 2
Stability results

| | Temperature | | Viscosity | | | |
|---|---|---|---|---|---|---|
| Month | for analysis | pH | (T-rpm, cps) | Appearance | Color | Odor |
| 0 | 25° C. | 4.77 | N/A | Liquid | Incolor | Characteristic |
| 1 | 25° C. | 4.81 | N/A | Liquid | Yellow | Characteristic |
| 2 | 25° C. | 4.76 | N/A | Liquid | Yellow | Characteristic |
| 3 | 25° C. | 4.79 | N/A | Liquid | Yellow | Characteristic |

As can be seen from the data shown in Table 2, the product shows stability in terms of appearance, odor, color, and pH after 90 days at 45° C.

### Methods

### Animal Use Justification and Welfare

Procedures were designed to avoid or minimize discomfort, distress and pain to the animals in accordance with the principles of the Animal for Research Act of Ontario and the guidelines of Canadian Council on Animal Care (CCAC). The CCAC Guide for the Care and Use of Experimental Animals and related policies were followed.

The methods were reviewed and approved by the Study Facility's Institutional Animal Care and Use Committee (IACUC) before the start as per IACUC standard operating procedures.

### Animals

Twelve male Sprague-Dawley rats (250-325 g) from Charles River Laboratories were used. The animals were acclimatized to their new environment for a minimum of 5 days prior to surgery. Food and water were available ad libitum throughout the study.

### Health Observations

Animals were closely monitored until recovery from anaesthesia and at least twice daily during recovery from surgery. Only animals in good health were used for dosing. Animals were closely observed for 1 hour following dosing and at each sample collection time point for the remainder of the study. Any adverse reactions to the administration of the test material were noted and tabulated.

### Catheter Placement

Catheters were implanted in the carotid artery (CAC) for serial blood collection and blood sample volume replacement and in the cisterna magna (CMC) for serial cerebrospinal fluid (CSF) collection in all animals. All catheters were implanted at least one day prior to dosing according to standard operating procedures.

### Test Article

The test article, 5-MeO-DMT (16)(MW_{free base (fb)}=218.30 g/mol, MF_{fb}=C₁₃H₁₈N₂O; MWₛₐₗₜ=336.39 g/mol, MFₛₐₗₜ=C₁₇H₂₄N₂O₅, Batch No. VPI-62-021, Correction Factor=1.54) was stored at room temperature with desiccant and protected from light until use.

### Test Article Formulation

The test article was freshly prepared at the appropriate concentration in a standard formulation of 10% dimethyl sulfoxide (DMSO) and 90% (v/v) saline (Groups 1 & 2) and the sponsor formulation included 3% linoleic acid (Groups 3 & 4).

### Treatment Groups

There were four treatment groups with three animals in each group. The experimental parameters are summarized in the Table 3 below.

TABLE 3
Dosing parameters

| | Test | | | Dose | Dose | | Blood |
|---|---|---|---|---|---|---|---|
| Group | article | No. of | | (mg/kg | concentration | Dose | sampling |
| ID | ID | males | Route | free base) | (mg/mL free base) | volume | times (h) |
| 1 | Standard | 3 | i.n. | 3 | 22.5 | 20 μL/ | 0.0833, 0.25, |
| | Formulation | | | | | nostril | 0.5, 1, 2, |
| 2 | (10% DMSO | 3 | s.c. | 3 | 1.5 | 2 mL/kg | 4, 6 & 8 |
| | in saline) | | | | | | |
| 3 | Sponsor | 3 | i.n. | 3 | 22.5 | 20 μL/ | |
| | formulation | | | | | nostril | |
| 4 | (3% linoleic | 3 | s.c. | 3 | 1.5 | 2 mL/kg | |
| | acid) | | | | | | |

### Body Weights

Animals were weighed prior to surgery and on the day of test article administration for dose volume calculations.

### Test Article Administration

Animals were dosed with the appropriate test article formulation at least one day following recovery from surgery. Formulations were administered intranasally (i.n.) under light isoflurane anesthesia via pipette to each nape or subcutaneously (s.c.) at the nape of the neck or the flank according to Table 4 (A) and (B) below.

Table 4. Summary of mean (±SD, n=3) plasma PK parameters for 5-MeO-DMT and bufotenine following 3 mg/kg s.c. administration of 5-MeO-DMT in Standard (A) and Sponsor (B) formulations to groups of 3 male Sprague-Dawley rats.

### (A) Standard Formulation

| | Parameter estimate | | | |
|---|---|---|---|---|
| | Plasma | | CSF | |
| Parameter | 5-MeO-DMT | Bufotenine | 5-MeO-DMT | Bufotenine |
| Dose (mg/kg) | 3 | n/a^{a} | n/a | n/a |
| tₘₐₓ (h) | 0.333 ± 0.144 | 0.333 ± 0.144 | 0.333 ± 0.144 | n/a |
| Cₘₐₓ (ng/mL) | 207 ± 38.1 | 0.378 ± 0.196 | 48.2 ± 6.81 | n/a |
| Apparent t_{1/2} (h) | 0.366 (n = 2) | nc^{b} | nc | n/a |
| AUC₀₋ₜₗₐₛₜ(h*ng/mL) | 147 ± 8.34 | n/a | 58.6 ± 16.5 | n/a |
| F^{sc} (%)^{c} | 139 ± 7.95 | n/a | nc | n/a |
| CSF/plasma AUC_{0-inf} ratio | n/a | n/a | 0.403 ± 0.134 | n/a |

^{a}n/a denotes not applicable.
^{b}nc denotes not calculable.
^{c}Calculated from the AUC_{0-inf} for 5-MeO-DMT (35.3 h*ng/mL) normalized for dose following 1 mg/kg intravenous (i.v.) administration.

### (B) Sponsor Formulation

| | Parameter estimate | | | |
|---|---|---|---|---|
| | Plasma | | CSF | |
| Parameter | 5-MeO-DMT | Bufotenine | 5-MeO-DMT | Bufotenine |
| Dose (mg/kg) | 3 | n/a^{a} | n/a | n/a |
| tₘₐₓ (h) | 0.583 ± 0.382 | 0.833 ± 0.289 | 0.778 ± 1.06 | n/a |
| Cₘₐₓ (ng/mL) | 50.2 ± 8.14^{b} | 0.197 ± 0.0258 | 39.1 ± 42.5 | n/a |
| Apparent t_{1/2} (h) | 1.49 ± 0.238 | 2.34 (n = 1) | 2.35 (n = 1) | n/a |
| AUC₀₋ₜₗₐₛₜ (h*ng/mL) | 144 ± 29.3 | 0.670 ± 0.149 | 42.4 ± 9.71 | n/a |
| F^{sc} (%)^{c} | 141 ± 31.0 | n/a | n/a | n/a |
| CSF/plasma AUC_{0-inf} ratio | n/a | n/a | 0.318 ± 0.151 | n/a |

^{a}n/a denotes not applicable.
^{b}Significantly different from the Standard formulation.
^{c}Calculated from the AUC_{0-inf} for 5-MeO-DMT (35.3 h*ng/mL) normalized for dose following 1 mg/kg i.v. administration.

### Blood Collection

Approximately 0.25 mL blood was collected at each time point from the carotid artery (CAC) via 1 mL syringes and was immediately transferred into 0.8 mL K₂EDTA tubes and placed on wet ice. Following each sample collection, an equivalent aliquot of heparinized physiological saline was slowly injected into the CAC to replace the sample volume. Blood samples were centrifuged within 5 min of collection at 3200×g for 5 min at 4° C. to obtain plasma. Plasma was transferred into a 1.5 mL flip-top cryovial and one cryovial of plasma was sampled for each collection time point and stored at approximately−80° C. until analysis.

### Cerebrospinal Fluid (CSF) Collection

CSF (approximately 15 μL) was collected from the cisterna magna catheter, concurrently to the plasma sample collection, into labeled microcentrifuge tubes as per standard operating procedures. Samples were stored frozen at approximately−80° C. until shipped on dry ice to the Bioanalytical facility for analysis.

### Bioanalytical Methods and Sample Analysis

### Bioanalytical Method Qualification for the Test Compound in Rat Plasma and CSF

A LC-MS/MS method for the quantification of 5-MeO-DMT and it's active metabolite bufotenine (MW=204.27 g/mol, MF=C₁₂H₁₆N₂O) in rat plasma was used as follows. A method for the quantification of 5-MeO-DMT and bufotenine in rat CSF was developed and qualified. Method qualification and sample analysis were conducted using an AB Sciex A PI 4000 or 6500 Q-TRAP mass spectrometry (MS/MS) system equipped with a liquid chromatography (LC) system with a binary pump, a solvent degasser, a thermostatted column compartment and a multiplate autosampler.

### Method Development Included:

1. the development of sample cleanup method using artificial CSF spiked with the test article. The qualification of the method(s) included:
2. the determination of the calibration dynamic range (e.g. 0.25-2000 ng/mL) using at least 6 non-zero calibration standards in singlet, and including a blank sample (without internal standard (IS)) and a zero standard (with IS),
3. triplicate injections of a system suitability sample (neat solution containing the analytes and IS) bracketing the batch. Requirements for acceptance of the method(s) were:
1. at least 75% of the non-zero calibration standards must be included in the calibration curve with all back-calculated concentrations within +20% deviation from nominal concentrations (except for the lower level of quantification (LLOQ), where±25% deviation is acceptable),
2. the correlation coefficient (r) of the calibration curve must be greater than or equal to 0.99 using quadratic regression analysis (1/x² weighting),
3. the area ratio variation between the pre—and post-run injections of the system suitability samples is within +25%.

### Sample Analysis

Samples in each matrix were analyzed as one or more separate batches using the qualified LC-MS/MS methods. A sample batch consisted of the following: 3 replicates of a system suitability standard (containing the analyte and IS), calibration standards in ascending order including a blank sample (without IS), a zero sample (with IS) and at least 6 non-zero standards, the assay samples and dosing solutions diluted into blank matrix (plasma), followed by the 3 replicates of the system suitability sample.

Calibration standards bracketed an analysis batch of greater than 40 samples. The analysis batch was considered acceptable if the acceptance criteria stated in the method re-qualification above were met. Samples determined to be above the highest level of quantitation (AQL) were acceptable up to 25% above the highest calibration standard. Samples with concentrations greater than 25% above the highest calibration standard were diluted and re-analyzed along with a corresponding dilution quality control (QC) sample. Dilution standards were acceptable if they were within 30% accuracy of the target concentration.

### Data Analysis

The plasma and CSF concentrations of 5-MeO-DMT and bufotenine were analyzed by noncompartmental methods using Phoenix® WinNonlin 8.3 (Certara, Mountainview, CA). Areas under the plasma concentration versus time curves (AUCs) for each animal were calculated by the linear up/log down trapezoidal rule. AUCs represent the areas under the curve from the time of dosing: (1) to the time (tlast) of the last measurable concentration, Cₗₐₛₜ (AUC₀₋ₜₗₐₛₜ) and (2) extrapolated to infinity (AUC_{0-Inf}). AUC_{0-Inf} was estimated, where possible, by the addition of _{AUCO-tlast} and C_{last/λz}, where λ_{z} represents the terminal (or elimination) rate constant. λz was estimated by regression analysis of a minimum of 3 time-points from the terminal (log-linear) portion of the concentration versus time curve. Terminal half-lives (t_{1/2}) were calculated as In (2)/λz. Mean residence times (MRT) were calculated as: AUMC/AUC, where AUMC denotes the area under the first moment curve. The time, tₘₐₓ, to reach the maximum concentration, Cₘₐₓ, was determined from the nominal values.

### Results

This study evaluated the pharmacokinetics (PK) of 5 methoxy N,N dimethyltryptamine (5-MeO-DMT (16)) in plasma and cerebrospinal fluid (CSF) following intranasal (i.n.) and subcutaneous (s.c.) administration of 3 mg/kg 5-MeO-DMT in a Standard (10% DMSO in saline) or Sponsor-provided (3% linoleic acid formulation) formulation to groups of three male Sprague-Dawley rats. Blood plasma and CSF were serially sampled at 8 time-points over 8 hours from surgically placed carotid artery and cisterna magna catheters, respectively.

A liquid chromatography-tandem mass spectrometry (LC-MS/MS) method was re-qualified for the quantification of 5-MeO-DMT and the active metabolite, bufotenine, in rat plasma and cross-qualified for quantification in CSF as described above. The calibration dynamic ranges were 0.1 to 2500 ng/ml for 5-MeO-DMT and 0.05 to 1000 ng/mL for bufotenine in plasma and CSF. Both the method qualification and sample analysis batches passed the acceptance criteria. The PK parameters for each analyte were estimated from the plasma and CSF concentration versus time curves for each animal using Phoenix® WinNonlin 8.3 (Certara, Mountainview, CA). Since the i.n. dose was constant (0.9 mg/animal=0.04 mL*22.5 mg/mL), the mg/kg dose for each i.n. administered animal was calculated by dividing 0.9 mg by the body weight of each rat. Bioavailability (F) was calculated using the dose-normalized AUC_{0-inf} for 5-MeO-DMT following 1 mg/kg i.v. administration. To determine if statistically significant differences in 5-MeO-DMT plasma and CSF exposure (Cₘₐₓ and AUC) existed between the 2 formulations, a two-tailed T-test (equal variance assumed) was performed using GraphPad Prism® 9.3 software. A p-value of <0.05 was considered significant.

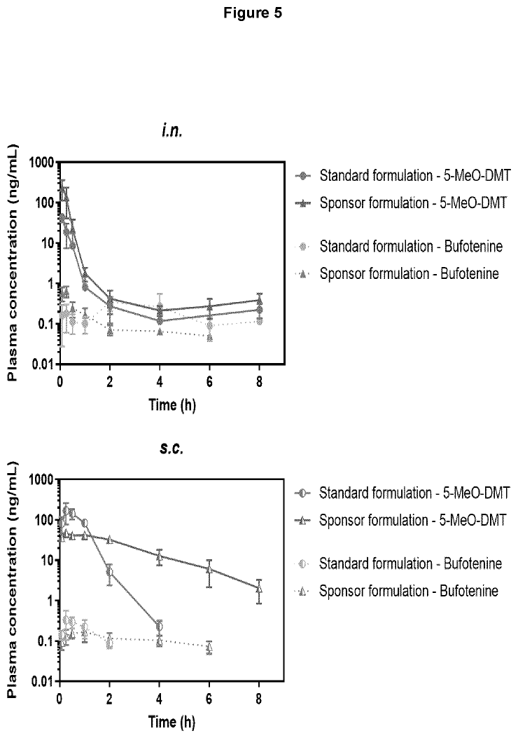

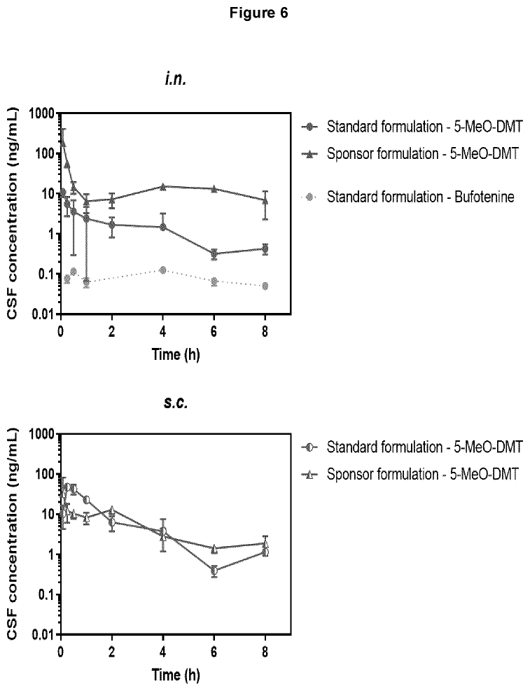

The mean plasma concentration versus time profiles are depicted in FIG. 5. Comparisons of the plasma and CSF concentration versus time profiles between the Standard and Sponsor formulations are depicted in FIGS. 6A-B and 7 A-B, respectively. Comparisons of the plasma and CSF concentration profiles between the routes of administration for each formulation are depicted in FIG. 8. In some instances, a direct comparison of the overall plasma exposure (AUC_{0-∞}) was not possible, as the AUC could not be extrapolated to infinity and thus a comparison of the AUC from 0 to the last measurable concentration (tₗₐₛₜ) was performed. Plasma and CSF PK parameters comparisons (fold change) between the Standard and Sponsor formulations are summarized in Table 5 below.

TABLE 5
Sponsor/Standard formulation fold change comparison
of 5-MeO-DMT plasma and CSF PK parameters.

| | |
|---|---|
| Intranasal | Subcutaneous |
| administration | administration |

| Parameter estimate | Plasma | CSF | Plasma | CSF |
|---|---|---|---|---|
| tₘₐₓ (h) | 1.0 | 1.7 | 1.8 | 2.3 |
| Cₘₐₓ (ng/mL) | 5.4 | 17 | 0.24 | 0.81 |
| Apparent t_{1/2} (h) | n/a^{a} | n/a | 4.1 | n/a |
| AUC₀₋ₜₗₐₛₜ (h*ng/mL) | 5.4 | 10 | 1.0 | 0.72 |
| AUC_{0-inf} (h*ng/mL) | n/a | n/a | 1.0 | n/a |
| MRT_{0-inf} (h) | n/a | n/a | 3.9 | n/a |
| F (%) | 6.6 | n/a | 1.0 | n/a |

^{a}n/a denotes not applicable as the parameter was not calculable for one or both groups.

Following i.n. administration of the Sponsor formulation of 5-MeO-DMT, mean maximum plasma concentrations (Cₘₐₓ=234 ng/ml) were achieved at the first sampling time point (0.0833 h) and were 5.4-fold higher (P=0.0538) than following administration of the Standard formulation (Cₘₐₓ=43.2 ng/ml). Overall plasma exposure as measured by bioavailability (Fⁱⁿ) was also 6.6-fold higher (P=0.0617) following administration of the Sponsor formulation (Fⁱⁿ=59.0%) compared to the Standard formulation (Fⁱⁿ=8.93%). The apparent elimination half-life (t_{1/2}) of 5-MeO-DMT in plasma was 0.566 h following administration of the Standard formulation, however, since plasma concentrations decreased below the lower level of quantitation at 6 h post-dose and then increased to measurable levels at 8 h post-dose (FIG. 5), this estimate may not adequately reflect the true t_{1/2}. A similar plasma profile was observed for the Sponsor formulation (FIGS. 5 and 6), for which estimation of t_{1/2} was not possible.

5-MeO-DMT appeared rapidly in CSF with the Cₘₐₓ attained between 0.0833—and 0.25-hours post-dose following i.n. administration (FIG. 6). Cₘₐₓ and AUC₀₋ₜₗₐₛₜ for CSF were 17—and 10-fold higher, respectively, after administration of the Sponsor formulation (Cₘₐₓ=186 ng/mL, P=0.2361; AUC=113 h*ng/ml, P=0.0008) compared to the Standard formulation (Cₘₐₓ=10.9 ng/ml; AUC=11.2 h*ng/ml)(FIG. 6). Although t_{1/2} in CSF could not be reliably estimated for 5-MeO-DMT, CSF concentrations were higher than plasma concentrations by 1 h post-dose and appeared to be cleared from CSF at a slower rate relative to plasma (FIG. 6). The CSF/plasma AUC₀₋ₜₗₐₛₜ ratio for 5-MeO-DMT was 2.89 and 0.953 following Sponsor and Standard formulation administration, respectively. Variability in this ratio for the Sponsor formulation was high as one animal (R09, CSF/plasma AUC₀₋ₜₗₐₛₜ ratio=6.18) had a similar CSF exposure to that of the other two, but its plasma exposure was low compared to that of the others. The CSF/plasma AUC₀₋ₜₗₐₛₜ ratio for the other 2 animals administered the Sponsor formulation i.n. was 1.24.

Very low levels of the 5-MeO-DMT metabolite, bufotenine, were observed in plasma following i.n. administration of both formulations (FIG. 5). Plasma bufotenine Cₘₐₓ levels were slightly higher following dosing of the Sponsor formulation (Cₘₐₓ=0.649 ng/ml) compared to the Standard formulation (Cₘₐₓ=0.390 ng/ml)(Table 6, FIG. 5A). CSF bufotenine levels were below the lower limit of quantitation (BLQ) following administration of the Sponsor formulation.

Table 6: Summary of mean (±SD, n=3) PK parameters for plasma and CSF for 5-MeO-DMT and bufotenine following 3.68 and 3.01 mg/kg i.n. administration of 5-MeO-DMT, respectively, in the Standard (A) and Sponsor (B) formulations to groups of 3 male Sprague-Dawley rats.

### (A) Standard Formulation

| | Parameter estimate | | | |
|---|---|---|---|---|
| | Plasma | | CSF | |
| Parameter | 5-MeO-DMT | Bufotenine | 5-MeO-DMT | Bufotenine |
| Dose (mg/kg) | 3.68 ± 0.226 | n/a^{a} | n/a | n/a |
| tₘₐₓ (h) | 0.0833 ± 0.00 | 2.08 ± 1.88 | 0.0833 ± 0.00 | 4.00 ± 0.00 |
| Cₘₐₓ (ng/mL) | 43.2 ± 10.9 | 0.390 ± 0.110 | 10.9 ± 2.62 | 0.126 ± 0.0210 |
| Apparent t_{1/2} (h) | 0.566 ± 0.485^{b} | nc^{c} | nc | nc |
| AUC₀₋ₜₗₐₛₜ (h*ng/mL) | 11.5 ± 0.920 | n/a | 11.2 ± 4.41 | n/a |
| Fⁱⁿ (%)^{d} | 8.93 ± 0.493 | n/a | n/a | n/a |
| CSF/plasma AUC_{0-inf} ratio | n/a | n/a | 0.953 ± 0.324 | n/a |

^{a}n/a denotes not applicable.
^{b}Estimate may not reflect true t_{1/2}, because plasma concentrations dipped below the lower level of quantitation at 6 h and then increased to measurable values at 8 h post-dose.
^{c}nc denotes not calculable.
^{d}Calculated from the AUC_{0-inf} for 5-MeO-DMT (35.3 h*ng/mL) normalized for dose following 1 mg/kg i.v. administration (IVS Study #: IVS220-21099-RK).

### (B) Sponsor Formulation

| | Parameter estimate | | | |
|---|---|---|---|---|
| | Plasma | | CSF | |
| Parameter | 5-MeO-DMT | Bufotenine | 5-MeO-DMT | Bufotenine |
| Dose (mg/kg) | 3.01 ± 0.0995 | n/a^{a} | n/a | n/a |
| tₘₐₓ (h) | 0.0833 ± 0.00 | 0.194 ± 0.0962 | 0.139 ± 0.0962 | n/a |
| Cₘₐₓ (ng/mL) | 234 ± 122 | 0.649 ± 0.196 | 186 ± 218 | n/a |
| Apparent t_{1/2} (h) | nc^{b} | 1.72 (n = 2) | nc | n/a |
| AUC₀₋ₜₗₐₛₜ (h*ng/mL) | 62.1 ± 34.8 | 0.516 ± 0.235 | 113 ± 19.2 | n/a |
| Fⁱⁿ (%)^{c} | 59.0 ± 33.7 | n/a | n/a | n/a |
| CSF/plasma AUC_{0-inf} ratio | n/a | n/a | 2.89 ± 2.85 | n/a |

^{a}n/a denotes not applicable.
^{b}nc denotes not calculable.
^{c}Calculated from the AUC_{0-inf} for 5-MeO-DMT (35.3 h*ng/mL) normalized for dose following 1 mg/kg i.v. administration (IVS Study #: IVS220-21099-RK).

In contrast to i.n. administration, the mean plasma Cₘₐₓ following s.c. administration of 5-MeO-DMT was 4-fold lower for the Sponsor formulation (50.2 ng/ml) compared to the Standard formulation (207 ng/ml, P=0.0022)(Tables 4 & 5). Cₘₐₓ levels were achieved at 0.33—and 0.58-hour post-dose for the Standard and Sponsor formulations, respectively (Table 4). The apparent plasma t_{1/2} of 5-MeO-DMT following administration of the Sponsor formulation (1.49 h) was ˜4-fold longer when compared to the Standard formulation (0.366 h, n=2), likely due to the presence of linoleic acid in the Sponsor formulation causing a slow-release “depot” effect. However, overall plasma exposure (AUCO-Inf) and bioavailability (F^{SC}) were comparable between the two formulations, 151 h*ng/ml and 139% for the Standard formulation and 149 h*ng/ML (P=0.8804) and 141% for the Sponsor formulation, respectively (Table 4).

Maximum CSF concentrations (Cₘₐₓ) following s.c. 5-MeO-DMT administration were attained between 0.25—and 0.5-hour post-dose for the Standard formulation and between 0.0833—and 2-hour post-dose for the Sponsor formulation. CSF Cₘₐₓ and AUCO-tₗₐₛₜ were comparable after administration of the Standard formulation (Cₘₐₓ=48.2 ng/ml; AUC=58.6 h*ng/ml) and the Sponsor formulation (Cₘₐₓ=39.1 ng/ml, P=0.7329; AUC=42.4 h*ng/ml, P=0.2173), and thus the overall CSF exposure of 5-MeO-DMT relative to total plasma exposure was also similar following administration of the Standard formulation (CSF/Plasma AUC ratio=0.403) and Sponsor formulation (0.318). Similar to that observed following i.n. administration, 5-MeO-DMT CSF concentrations declined at a slower rate relative to plasma (FIG. 5), although t_{1/2} in CSF could not be reliably estimated.

Following s.c. 5-MeO-DMT administration, the Cₘₐₓ of bufotenine attained in plasma was comparable between the Standard formulation (Cₘₐₓ=0.378 ng/ml) and the Sponsor formulation (Cₘₐₓ=0.197 ng/ml). Bufotenine concentrations in the CSF were BLQ following administration of both formulations.

A comparison between i.n. and s.c. routes of administration for each formulation shows that for the Standard formulation, plasma and CSF exposure of 5-MeO-DMT were lower following i.n. (AUCO-tₗₐₛₜ=11.5 and 11.2 h*ng/ml, respectively) compared to s.c. administration (AUCO-tₗₐₛₜ=147 and 58.6 h*ng/ml, respectively) (FIGS. 5 and 6). By contrast, for the Sponsor formulation, plasma exposure was lower (AUC₀₋ₜₗₐₛₜ=62.1 and 144 h*ng/ml, respectively), but CSF exposure was higher (AUC₀₋ₜₗₐₛₜ=113 and 42.4 h*ng/ml, respectively)(FIGS. 5 and 6) following i.n. compared to s.c. administration.

In summary, i.n. administration of the Sponsor formulation of 5-MeO-DMT resulted in higher exposure of 5-MeO-DMT in plasma (6.6-fold higher bioavailability (P=0.0617)) and CSF (10-fold higher AUC₀₋ₜₗₐₛₜ (P=0.0008)) compared to the Standard formulation, although only the higher CSF exposure reached the level of statistical significance. In contrast, s.c. administration of the Sponsor formulation of 5-MeO-DMT resulted in a 4-fold lower plasma Cₘₐₓ (P=0.0022) but longer half-life, likely due to the presence of linoleic acid in the Sponsor formulation causing a slow-release “depot” effect. Bioavailability of the 2 formulations, however, was similar, and as a consequence, CSF exposure was also comparable between the two formulations. Following both i.n. and s.c. administration, 5-MeO-DMT appeared to be cleared from CSF at a slower rate relative to plasma.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1-11. (canceled)

12. A kit for improving the efficacy of one or more hallucinogens, or a pharmaceutically acceptable salt, prodrug and/or solvate thereof, for treating or preventing a disease, disorder or condition that is treated by activation of a serotonin receptor, the kit comprising one or more hallucinogens, or a pharmaceutically acceptable salt, prodrug and/or solvate thereof, and one or more fatty acids, or a pharmaceutically acceptable salt, prodrug and/or solvate thereof, wherein the one or more hallucinogens, or a pharmaceutically acceptable salt, prodrug and/or solvate thereof, and the one or more fatty acids, or a pharmaceutically acceptable salt, prodrug and/or solvate thereof, are in a single pharmaceutical composition or are in separate pharmaceutical compositions, and the one or more fatty acids are present in amounts that are effective for improving the efficacy of the one or more hallucinogens, or a pharmaceutically acceptable salt, prodrug and/or solvate thereof, to treat or prevent the disease, disorder or condition that is treated by activation of a serotonin receptor, and
wherein the one or more hallucinogens are selected from one or more compounds of Formula II listed below II-1: (R)-3-((1-methylpyrrolidin-2-yl)methyl)-1H-indol-4-yl dihydrogen phosphate;
11-2: (R)-3-((1-(methyl-d3)pyrrolidin-2-yl)methyl)-1H-indol-4-ol;
II-3: (R)-3-(pyrrolidin-2-ylmethyl)-1H-indol-4-ol;
II-4: (S)-3-((1-methylpyrrolidin-2-yl)methyl)-1H-indol-4-yl dihydrogen phosphate;
II-5: (R)-3-(pyrrolidin-2-ylmethyl)-1H-indol-4-yl dihydrogen phosphate;
II-6: (S)-3-(pyrrolidin-2-ylmethyl)-1H-indol-4-yl dihydrogen phosphate;
11-7: (R)-3-((1-(methyl-d3)pyrrolidin-2-yl)methyl)-1H-indol-4-yl dihydrogen phosphate;
II-8: (S)-3-((1-(methyl-d3)pyrrolidin-2-yl)methyl)-1H-indol-4-yl dihydrogen phosphate;
11-9: (S)-3-((1-(methyl-d3)pyrrolidin-2-yl)methyl-d2)-1H-indol-4-yl dihydrogen phosphate;
II-10: (R)-(((3-((1-methylpyrrolidin-2-yl)methyl)-1H-indol-4-yl)oxy)methyl)phosphonic acid;
11-11: (R)-(((3-((1-(methyl-d3)pyrrolidin-2-yl)methyl)-1H-indol-4-yl)oxy)methyl)phosphonic acid;
II-12: (R)-((4-hydroxy-3-((1-methylpyrrolidin-2-yl)methyl)-1H-indol-1-yl)methyl)phosphonic acid;
11-13: (R)-((4-hydroxy-3-((1-(methyl-d3)pyrrolidin-2-yl)methyl)-1H-indol-1-yl)methyl)phosphonic acid;
II-14: (R)-((4-hydroxy-3-(pyrrolidin-2-ylmethyl)-1H-indol-1-yl)methyl)-phosphonic acid;
II-15: (R)-((3-((1-methylpyrrolidin-2-yl)methyl)-4-(phosphonooxy)-1H-indol-1-yl)methyl)phosphonic acid;
11-16: (1-((3-(((R)-1-methylpyrrolidin-2-yl)methyl)-1H-indol-4-yl)oxy)ethyl)phosphonic acid;
11-17: (1-((3-(((R)-pyrrolidin-2-yl)methyl)-1H-indol-4-yl)oxy)ethyl)phosphonic acid;
11-18: (R)-3-((1-(methyl-d3)pyrrolidin-2-yl)methyl)-1H-indol-4-yl glycinate;
II-19:3-(((R)-1-(methyl-d3)pyrrolidin-2-yl)methyl)-1H-indol-4-yl D-alaninate;
II-20: (R,Z)-4-((3-((1-(methyl-d3)pyrrolidin-2-yl)methyl)-1H-indol-4-yl)oxy)-4-oxobut-2-enoic acid;
II-21: (R,E)-4-((3-((1-(methyl-d3)pyrrolidin-2-yl)methyl)-1H-indol-4-yl)oxy)-4-oxobut-2-enoic acid;
II-22: (R)-4-((3-((1-(methyl-d3)pyrrolidin-2-yl)methyl)-1H-indol-4-yl)oxy)-4-oxobutanoic acid;
II-23: (R)-3-((1-(methyl-d3)pyrrolidin-2-yl)methyl)-1H-indol-4-yl acetate;
11-24: (R)-3-((1-(methyl-d3)pyrrolidin-2-yl)methyl-d2)-1H-indol-4-yl acetate;
II-25: (R)-3-((1-methylpyrrolidin-2-yl)methyl)-1H-indol-4-yl acetate;
II-26: (R)-((4-acetoxy-3-((1-methylpyrrolidin-2-yl)methyl)-1H-indol-1-yl)methyl)phosphonic acid;
11-27: 3-(((R)-1-methylpyrrolidin-2-yl)methyl)-1H-indol-4-yl (9Z,12Z)-octadeca-9,12-dienoate;
11-28:3-(((R)-1-(methyl-d3)pyrrolidin-2-yl)methyl)-1H-indol-4-yl (9Z, 12Z)-octadeca-9,12-dienoate-11,11-d2;
11-29:3-(((R)-1-(methyl-d3)pyrrolidin-2-yl)methyl-d2)-1H-indol-4-yl (9Z, 12Z)-octadeca-9,12-dienoate-11,11-d2;
II-30:3-(((R)-1-methylpyrrolidin-2-yl)methyl)-1H-indol-4-yl (9Z, 12Z)-octadeca-9,12-dienoate-11,11-d2;
II-31:3-(((R)-1-methylpyrrolidin-2-yl)methyl)-1H-indol-4-yl(S)-3-(aminomethyl)-5-methylhexanoate;
11-32:3-(((R)-1-(methyl-d3)pyrrolidin-2-yl)methyl)-1H-indol-4-yl(S)-3-(aminomethyl)-5-methylhexanoate;
11-33: (R)-3-((1-methylpyrrolidin-2-yl)methyl)-1H-indol-4-yl 2-(1-(aminomethyl)-cyclohexyl) acetate;
II-34: (R)-3-((1-(methyl-d3)pyrrolidin-2-yl)methyl)-1H-indol-4-yl 2-(1-(aminomethyl)cyclohexyl)-acetate;
II-35: (R)-3-((1-methylpyrrolidin-2-yl)methyl)-1H-indol-4-yl [1,4′-bipiperidine]-1′-carboxylate;
11-36: (R)-3-((1-(methyl-d3)pyrrolidin-2-yl)methyl)-1H-indol-4-yl dimethylcarbamate;
II-37: (R)-4-fluoro-3-((1-(methyl-d3)pyrrolidin-2-yl)methyl-d2)-1H-indole;
II-38: (R)-4-chloro-3-((1-(methyl-d3)pyrrolidin-2-yl)methyl-d2)-1H-indole;
II-39: (R)-4-methoxy-3-((1-(methyl-d3)pyrrolidin-2-yl)methyl-d2)-1H-indole;
II-40: (R)-4-fluoro-3-((1-(methyl-d3)pyrrolidin-2-yl)methyl)-1H-indole;
II-41: (R)-4-fluoro-3-(pyrrolidin-2-ylmethyl-d2)-1H-indole;
II-42: (S)-4-fluoro-3-((1-(methyl-d3)pyrrolidin-2-yl)methyl-d2)-1H-indole;
11-43: (R)-4-(benzyloxy)-3-((1-(methyl-d3)pyrrolidin-2-yl)methyl-d2)-1H-indole;
II-44: (R)-3-((1-(methyl-d3)pyrrolidin-2-yl)methyl-d2)-1H-indol-4-ol;
II-45: (R)-4-(benzyloxy)-3-((1-(methyl-d3)pyrrolidin-2-yl)methyl)-1H-indole;
II-46: (R)-4-(benzyloxy)-3-((1-methylpyrrolidin-2-yl)methyl)-1H-indole;
II-47: (R)-3-((1-methylpyrrolidin-2-yl)methyl)-1H-indol-4-ol;
II-48:3-(((R)-1-(methyl-d3)pyrrolidin-2-yl)methyl)-1H-indol-4-yl (9Z, 12Z)-octadeca-9,12-dienoate;
11-49:3-(((R)-1-(methyl-d3)pyrrolidin-2-yl)methyl-d2)-1H-indol-4-yl (9Z, 12Z)-octadeca-9,12-dienoate;
11-50: (R)-3-(pyrrolidin-2-ylmethyl-d2)-1H-indol-4-ol;
II-51: (S)-3-(pyrrolidin-2-ylmethyl-d2)-1H-indol-4-ol;
II-52: (S)-4-(benzyloxy)-3-((1-(methyl-d3)pyrrolidin-2-yl)methyl)-1H-indole;
11-53: (S)-3-((1-(methyl-d3)pyrrolidin-2-yl)methyl)-1H-indol-4-ol; and
II-54: (R)-3-((1-(methyl-d3)pyrrolidin-2-yl)methyl-d2)-1H-indol-4-yl dihydrogen phosphate;
or a pharmaceutically acceptable salt, prodrug and/or solvate thereof.

13-14. (canceled)

15. The kit of any one of claim 12, wherein the one or more fatty acids, or a pharmaceutically acceptable salt, prodrug and/or solvate thereof, are selected from any such acid derived from fats by hydrolysis and having from 4 to 30 carbon atoms, 6 to 28 carbon atoms or 6 to 24 carbon atoms.

16. A method of improving the efficacy of one or more hallucinogens selected from one or more compounds of Formula II as defined in claim 12, or a pharmaceutically acceptable salt, prodrug and/or solvate thereof, comprising administering an effective amount of the one or more hallucinogens, or a pharmaceutically acceptable salt, prodrug and/or solvate thereof, in combination with an effective amount of one or more fatty acids, or a pharmaceutically acceptable salt, prodrug and/or solvate thereof, to a subject in need thereof.

17-18. (canceled)

19. The method of any one of claim 16, wherein the one or more fatty acids, or a pharmaceutically acceptable salt, prodrug and/or solvate thereof, selected from any such acid derived from fats by hydrolysis and having from 4 to 30 carbon atoms, 6 to 28 carbon atoms or 6 to 24 carbon atoms.

20-26. (canceled)

27. An intranasal pharmaceutical composition comprising one or more hallucinogens, selected from one or more compounds of Formula II as defined in claim 12, or a pharmaceutically acceptable salt, prodrug and/or solvate thereof, and one or more fatty acids, or a pharmaceutically acceptable salt, prodrug and/or solvate thereof,
wherein the composition comprises a weight ratio of the amount of the one or more fatty acids, or a salt, prodrug and/or solvate thereof, to the one or more hallucinogens, or a salt, prodrug and/or solvate thereof of about 0.1:1 to about 5:1, about 0.5:1 to about 1:1.5, about 0.75:1.25 to about 1:1.25 or about 1:1.2.

28-30. (canceled)

31. The intranasal pharmaceutical composition of claim 27, wherein the one or more fatty acids, or a pharmaceutically acceptable salt, prodrug and/or solvate thereof, are selected from any such acid derived from fats by hydrolysis and having from 4 to 30 carbon atoms, 6 to 28 carbon atoms or 6 to 24 carbon atom.

32. (canceled)

33. The intranasal pharmaceutical composition of claim 31, wherein the one or more fatty acids or a pharmaceutically acceptable salt, prodrug and/or solvate thereof is linoleic acid or a pharmaceutically acceptable salt, prodrug and/or solvate thereof.

34. The intranasal pharmaceutical composition of claim 273, wherein the intranasal pharmaceutical composition is formulated as an aerosol, solution, spray, drop, gel or a powder formulation.

35-39. (canceled)

40. The intranasal pharmaceutical composition of claim 27, wherein the intranasal pharmaceutical composition further comprises water and is an aqueous intranasal pharmaceutical composition

41. (canceled)

42. (canceled)

43. The intranasal pharmaceutical composition of claim 40, wherein the water is present in an amount of about 50% to about 75%, about 50% to about 70%, about 50% to about 65%, about 33% to about 75%, about 55% to about 70% or about 55% to about 65% by weight of the composition.

44-49. (canceled)

50. The intranasal pharmaceutical composition of claim 43, wherein the intranasal pharmaceutical has a pH about 4 to about 7, about 4.5 to about 6, or about 4.5 to about 5.5.

51-53. (canceled)

54. The intranasal pharmaceutical composition of claim 50, wherein the intranasal pharmaceutical composition further comprises-one or more of:
about 10% to about 35%, or about 10% to about 30%, or about 10% to about 25%, or about 15% to about 25% of the one or more surfactants by weight of the composition;
about 1% to about 10%, about 2% to about 8%, about 2% to about 7%, about 3% to about 7%, about 3% to about 6%, or about 4% to about 6% of one or more co-solvents by weight of the composition;
about 1% to about 3% of one or more buffering agents by weight of the composition, and about 1% to about 10%, about 2% to about 8%, about 2% to about 7%, about 3% to about 7%, about 3% to about 6%, or about 4% to about 6% of one or more humectants by weight of the composition.

55. (canceled)

56. The intranasal pharmaceutical composition of claim 545,
wherein the one or more non-ionic surfactants are selected from tyloxapol, polyoxyethylene-sorbitan-fatty acid esters, polyoxyethylene products of hydrogenated vegetable oils, polyethoxylated castor oils, polyethoxylated hydrogenated castor oil, polyoxyethylene castor oil derivatives and poloxamers and mixtures thereof,
the one or more co-solvents are selected isopropyl alcohol; glycols such as propylene glycol, polyethylene glycol, polypropylene glycol, glycol ether, and glycerol;
polyoxyethylene alcohols; medium chain glycerides and diethylene glycol monoethyl ether (2-(2-ethoxyethoxy) ethanol) and mixtures thereof, and
the one or more humectants are selected from glycerin, sorbitol, mannitol and xylitol and mixtures thereof.

57. The intranasal pharmaceutical composition of claim 56,
wherein the polyoxyethylene sorbitan fatty esters are selected from polyethylene sorbitan monooleate (Polysorbate 80), polyoxyethylene (20) sorbitan monolaurate (polysorbate 20), polyoxyethylene (20) sorbitan tristearate (polysorbate 65), polyoxyethylene (20) sorbitan monooleate, polyoxyethylene (20) sorbitan monopalmitate, and polyoxyethylene (20) sorbitan monostearate and mixtures thereof.

58-60. (canceled)

61. The intranasal pharmaceutical composition of claim 56, wherein the co-solvent is (2-(2-ethoxyethoxy) ethanol.

62-67. (canceled)

68. The intranasal pharmaceutical composition of claim 54, wherein the intranasal pharmaceutical composition further comprises one or more sweetening agents.

69-80. (canceled)

81. The intranasal pharmaceutical composition claim 27, wherein the one or more fatty acids or a pharmaceutically acceptable salt, prodrug and/or solvate thereof increases the rate of absorption of the one or more hallucinogens or pharmaceutically acceptable salt, prodrug and/or solvate thereof in plasma and cerebrospinal fluid (CSF) of a subject compared to an otherwise identical intranasal pharmaceutical composition except in the absence of the one or more fatty acids or a pharmaceutically acceptable salt, prodrug and/or solvate thereof.

82. The intranasal pharmaceutical composition of claim 27, wherein the intranasal pharmaceutical composition provides a Cmax of the one or more hallucinogens or a pharmaceutically acceptable salt, prodrug and/or solvate thereof in plasma that is about 3 to about 6, or about 5-fold greater compared to the Cmax of an otherwise identical intranasal pharmaceutical composition except in the absence of the one or more fatty acids or a pharmaceutically acceptable salt, prodrug and/or solvate thereof.

83. The intranasal pharmaceutical composition of claim 27, wherein the intranasal pharmaceutical composition provides a Cmax of the one or more hallucinogens or a pharmaceutically acceptable salt, prodrug and/or solvate thereof in cerebral spinal fluid that is about 5 to about 20, about 10 to about 17, or about 10 fold greater compared to the Cmax of an otherwise identical intranasal pharmaceutical composition except in the absence of the one or more fatty acids, or a pharmaceutically acceptable salt, prodrug and/or solvate thereof.

84. The intranasal pharmaceutical composition of claim 27, wherein the intranasal pharmaceutical composition provides an increase in bioavailability of the one or more hallucinogens or a pharmaceutically acceptable salt, prodrug and/or solvate thereof in plasma and in CSF when administered intranasally compared to an otherwise identical intranasal pharmaceutical composition except in the absence of the one or more fatty acids, or a pharmaceutically acceptable salt, prodrug and/or solvate thereof.

85-89. (canceled)
